(19) United States
(12) Patent Application Publication
(10) Pub. No.: US 2017/0320848 A1
(43) Pub. Date: Nov. 9, 2017

(54) COMPOUNDS FOR USE IN ANTHELMINTHIC TREATMENT

(71) Applicant: Bayer Animal Health GMBH, Leverkusen (DE)

(72) Inventors: Claudia WELZ, Düsseldorf (DE); Adeline KÖHLER, Langenfeld (DE); Kirsten BÖRNGEN, Köln (DE); Daniel KULKE, Leverkusen (DE); Ulrich GÖRGENS, Ratingen (DE); Hans-Georg SCHWARZ, Dorsten (DE); Kerstin ILG, Köln (DE)

(73) Assignee: Bayer Animal Health GMBH, Leverkusen (DE)

(21) Appl. No.: 15/523,028

(22) PCT Filed: Oct. 26, 2015
(86) PCT No.: PCT/EP2015/074718

§ 371 Date: Apr. 28, 2017

(30) Foreign Application Priority Data
Oct. 28, 2014 (EP) 14190755.0

(51) Int. Cl. C07D 317/58; C07D 295/135; C07D 213/82; C07C 317/40; C07C 233/66; C07C 255/60; C07C 233/80; C07C 233/73; C07D 295/185; C07C 323/42

(52) U.S. Cl. CPC C07D 317/58; C07D 295/185; C07D 295/135; C07D 213/82; C07C 323/42; C07C 233/66; C07C 255/60; C07C 233/80; C07C 233/73; C07C 317/40

(57) ABSTRACT

Disclosed are compounds of formula (I) which possess anthelminthic properties wherein the structural elements have the meaning as indicated in the description. Further disclosed are such compounds for the control, treatment and/or prevention of infections with helminths in animals and humans.

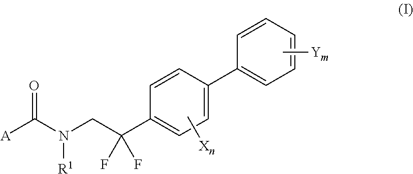

The present invention relates to certain bisphenyl-ethyl carboxamide derivatives. Further, the present invention relates to the use of certain bisphenyl-ethyl carboxamide derivatives for the control, treatment and/or prevention of infections with helminths in animals and humans, formulations containing such compounds and methods for the control, treatment and/or prevention of infections with helminths in animals and humans.

The occurrence of resistances against all commercial anthelmintics seems to be a growing problem in the area of veterinary medicine. Therefore, endoparasiticides with new molecular modes of action are urgently desired. The new active ingredients should perform with excellent efficacy against a broad spectrum of helminths, like nematodes, preferably without any adverse toxic effects to the treated organism. Endoparasiticides are pharmaceuticals for combat or suppression of endoparasites in animals or humans.

The use of certain N-2-(pyridyl)ethyl-carboxamide derivatives for controlling nematodes is described in WO 2007/108483 A1 and EP 2 132 987 A1.

The use of certain carboxamides as parasiticides is described in WO 2012/118139 A1, WO 2013/0676230 A1, WO 2014/034750 A1 and WO 2014/034751 A1.

Furthermore, certain carboxamides are described as pesticides in WO 2013/064518 A1, WO 2013/064519 A1, WO 2013/064520 A1, WO 2013/064521 A1 or as nematicides in WO 2013/064460 A1 and WO 2013/064461 A1.

It is an object of the present invention to provide compounds which can be used as endoparasiticides in the medical, especially veterinary, field with a satisfactory or improved anthelmintic activity against a broad spectrum of helminths, like nematodes, particularly at relatively low dosages, for the control, treatment and/or prevention of infections with helminths in animals and humans, preferably without any adverse toxic effects to the treated organism.

The present invention relates to compounds of formula (I)

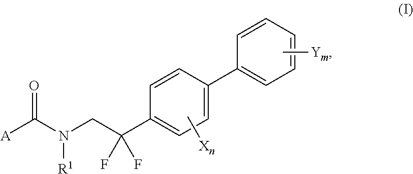

wherein
R¹ is selected from the group consisting of hydrogen, —CHO, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, C₃-C₆-halogenocycloalkyl having 1 to 5 halogen atoms, C₃-C₄-alkenyl, C₃-C₄-alkynyl, C₁-C₄-alkoxy-C₁-C₄-alkyl, C₃-C₆-cycloalkyl-C₁-C₃-alkyl, cyano-C₁-C₄-alkyl, amino-C₁-C₄-alkyl, C₁-C₄-alkylamino-C₁-C₄-alkyl, di-(C₁-C₄-alkyl)amino-C₁-C₄-alkyl, C₁-C₄-alkylcarbonyl, C₁-C₄-halogenoalkylcarbonyl having 1 to 5 halogen atoms, C₁-C₄-alkoxycarbonyl, benzyloxycarbonyl, C₁-C₄-alkoxy-C₁-C₄-alkylcarbonyl, —S(O)₂—C₁-C₄-alkyl, and —S(O)₂—C₁-C₄-halogenoalkyl having 1 to 5 halogen atoms,
n is 1, 2 or 3,
each X is independently selected from the group consisting of halogen, nitro, cyano, hydroxy, amino, —SH, —SF₅, —CHO, —OCHO, —NHCHO, —COOH, —CONH₂, —CONH(OH), —OCONH₂, (hydroxyimino)-C₁-C₆-alkyl, C₁-C₈-alkyl, C₁-C₈-halogenoalkyl having 1 to 5 halogen atoms, C₂-C₈-alkenyl, C₂-C₈-alkynyl, C₁-C₈-alkylamino, di-(C₁-C₈-alkyl)amino, C₁-C₈-alkoxy, C₁-C₈-halogenoalkoxy having 1 to 5 halogen atoms, C₂-C₈-alkenyloxy, C₂-C₈-halogenoalkenyloxy having 1 to 5 halogen atoms, C₃-C₈-alkynyloxy, C₃-C₈-halogenoalkynyloxy having 1 to 5 halogen atoms, C₃-C₈-cycloalkyl, C₃-C₈-halogenocycloalkyl having 1 to 5 halogen atoms, C₁-C₈-alkylcarbonyl, C₁-C₈-halogenoalkylcarbonyl having 1 to 5 halogen atoms, —CONH(C₁-C₈-alkyl), —CON(C₁-C₈-alkyl)₂, —CONH(OC₁-C₈-alkyl), —CON(OC₁-C₈-alkyl)(C₁-C₈-alkyl), C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, —OCONH(C₁-C₈-alkyl), —OCON(C₁-C₈-alkyl)₂, —OCONH(OC₁-C₈-alkyl), —OCO(OC₁-C₈-alkyl), —S—C₁-C₈-alkyl, —S—C₁-C₈-halogenoalkyl having 1 to 5 halogen atoms, —S(O)—C₁-C₈-alkyl, —S(O)—C₁-C₈-halogenoalkyl having 1 to 5 halogen atoms, —S(O)₂—C₁-C₈-alkyl, —S(O)₂—C₁-C₈-halogenoalkyl having 1 to 5 halogen atoms, (C₁-C₆-alkoxyimino)-C₁-C₆-alkyl, (C₂-C₆-alkenyloxyimino)-C₁-C₆-alkyl, (C₃-C₆-alkynyloxyimino)-C₁-C₆-alkyl, (benzyloxyimino)-C₁-C₆-alkyl, benzyloxy, —S-benzyl, benzylamino, phenoxy, —S-phenyl and phenylamino,
m is 0,1,2,3 or 4,
each Y is independently selected from the group consisting of hydrogen, halogen, nitro, cyano, hydroxy, amino, —SH, —SF₅, —CHO, —OCHO, —NHCHO, —COOH, —CONH₂, —CONH(OH), —OCONH₂, (hydroxyimino)-C₁-C₆-alkyl, C₁-C₈-alkyl, C₁-C₈-halogenoalkyl having 1 to 5 halogen atoms, C₂-C₈-alkenyl, C₂-C₈-alkynyl, C₁-C₈-alkylamino, di-(C₁-C₈-alkyl)amino, C₁-C₈-alkoxy, C₁-C₈-halogenoalkoxy having 1 to 5 halogen atoms, C₂-C₈-alkenyloxy, C₂-C₈-halogenoalkenyloxy having 1 to 5 halogen atoms, C₃-C₈-alkynyloxy, C₃-C₈-halogenoalkynyloxy having 1 to 5 halogen atoms, C₃-C₈-cycloalkyl, C₃-C₈-halogenocycloalkyl having 1 to 5 halogen atoms, C₁-C₈-alkylcarbonyl, C₁-C₈-halogenoalkylcarbonyl having 1 to 5 halogen atoms, —CONH(C₁-C₈-alkyl), —CON(C₁-C₈-alkyl)₂, —CONH(OC₁-C₈-alkyl), —CON(OC₁-C₈-alkyl)(C₁-C₈-alkyl), C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, —OCONH(C₁-C₈-alkyl), —OCON(C₁-C₈-alkyl)₂, —OCONH(OC₁-C₈-alkyl), —OCO(OC₁-C₈-alkyl), —S—C₁-C₈-alkyl, —S—C₁-C₈-halogenoalkyl having 1 to 5 halogen atoms, —S(O)—C₁-C₈-alkyl, —S(O)—C₁-C₈-halogenoalkyl having 1 to 5 halogen atoms, —S(O)₂—C₁-C₈-alkyl, —S(O)₂—C₁-C₈-halogenoalkyl having 1 to 5 halogen atoms, —CH₂—S—C₁-C₈-alkyl, —CH₂—S(O)—C₁-C₈-alkyl, —CH₂—S(O)₂-C₁-C₈-alkyl, (C₁-C₆-alkoxyimino)-C₁-C₆-alkyl, (C₂-C₆-alkenyloxyimino)-C₁-C₆-alkyl, (C₃-C₆-alkynyloxyimino)-C₁-C₆-alkyl, wherein two Y may additionally be selected from —O—C₁-C₃-alkoxy and —O—C₁-C₃-halogenalkoxy having 1 to 5 halogen atoms, with each oxy function being the connecting atom of the individual Y, thereby forming an annelated 5-, 6- or 7-membered ring with the phenyl moiety that they are connected to, or
each Y is independently selected from the group consisting of hydrogen, halogen, cyano, C₁-C₆-alkyl, C₁-C₆-halogenoalkyl having 1 to 5 halogen atoms, and a 4 to 7 membered heterocyclic ring system with 1 to 3 heteroatoms, but not O—O, O—S or 3 heteroatoms in a row, independently substituted by oxo, thiono or 1 to 12 substituents Z independently selected from the group consisting of hydrogen, halogen, cyano, hydroxy, amino, —SH, —CHO, —COOH, —CONH₂, —CONH(OH), —OCONH₂, (hydroxyimino)-C₁-C₆-alkyl, C₁-C₆-alkyl, C₁-C₆-halogenoalkyl having 1 to 5 halogen atoms, C₂-C₆-alkenyl, C₂-C₆-alkynyl, C₁-C₆-alkylamino, di-(C₁-C₆-alkyl)amino, C₁-C₆-alkoxy, C₁-C₆-halogenoalkoxy having 1 to 5 halogen atoms, C₂-C₆-alkenyloxy, C₂-C₆-halogenoalkenyloxy having 1 to 5 halogen atoms, C₃-C₆-alkynyloxy, C₃-C₆-halogenoalkynyloxy having 1 to 5 halogen atoms, C₃-C₆-cycloalkyl, C₃-C₆-halogenocycloalkyl having 1 to 5 halogen atoms, C₁-C₆-alkylcarbonyl, C₁-C₆-halogenoalkylcarbonyl having 1 to 5 halogen atoms, —CONH(C₁-C₆-alkyl), —CON(C₁-C₆-alkyl)₂, —CONH(OC₁-C₆-alkyl), —CON(OC₁-C₆-alkyl)(C₁-C₆-alkyl), C₁-C₆-alkoxycarbonyl, C₁-C₆-halogenoalkoxycarbonyl having 1 to 5 halogen atoms, C₁-C₆-alkylcarbonyloxy, C₁-C₆-halogenoalkylcarbonyloxy having 1 to 5 halogen atoms, C₁-C₆-alkylcarbonylamino, C₁-C₆-halogenoalkylcarbonylamino having 1 to 5 halogen atoms, —OCONH(C₁-C₆-alkyl), —OCON(C₁-C₆-alkyl)₂, —OCONH(OC₁-C₆-alkyl), —OCO(OC₁-C₆-alkyl), —S—C₁-C₆-alkyl, —S—C₁-C₆-halogenoalkyl having 1 to 5 halogen atoms, —S(O)—C₁-C₆-alkyl, —S(O)—C₁-C₆-halogenoalkyl having 1 to 5 halogen atoms, —S(O)₂—C₁-C₆-alkyl, —S(O)₂—C₁-C₆-halogenoalkyl having 1 to 5 halogen atoms, and
A represents a phenyl group of the formula (A1)

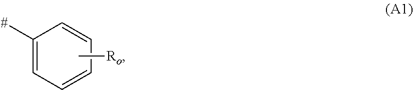

in which
# depicts the bond which connects A to the rest of the molecule,
o is 0, 1, 2, 3, 4 or 5, and
each R is independently selected from the group consisting of halogen, nitro, —OH, NH₂, SH, SF₅, CHO, OCHO, NHCHO, COOH, cyano, C₁-C₈-alkyl, C₁-C₈-halogenoalkyl having 1 to 9 halogen atoms, C₂-C₈-alkenyl, C₂-C₈-alkynyl, C₃-C₆-cycloalkyl, —S—C₁-C₈-alkyl, —S—C₁-C₈-halogenoalkyl having 1 to 5 halogen atoms, C₁-C₈-alkoxy, C₁-C₈-halogenoalkoxy having 1 to 5 halogen atoms, C₁-C₈-alkoxy-C₂-C₈-alkenyl, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, —S(O)—C₁-C₈-alkyl, —S(O)—C₁-C₈-halogenoalkyl having 1 to 5 halogen atoms, —S(O)₂—C₁-C₈-alkyl, —S(O)₂—C₁-C₈-halogenoalkyl having 1 to 5 halogen atoms, C₁-C₈-alkylsulfonamide, —NH(C₁-C₈-alkyl), N(C₁-C₈-alkyl)₂, phenyl (optionally substituted by C₁-C₆-alkoxy) and phenoxy, wherein two R bonded to adjacent carbon atoms may together represent —O(CH₂)ₚO—, wherein p represents 1 or 2, or
A represents a heterocycle of the formula (Het-1)

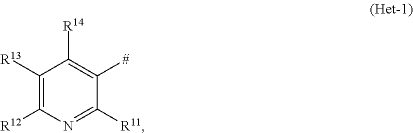

in which
# depicts the bond which connects A to the rest of the molecule,
R¹¹ is selected from the group consisting of hydrogen, halogen, hydroxy, cyano, C₁-C₄-alkyl, C₁-C₄-halogenoalkyl having 1 to 5 halogen atoms, C₁-C₄ alkoxy, —S—C₁-C₅-alkyl, S(O)—C₁-C₄-alkyl, —S(O)₂—C₁-C₄-alkyl, —S—C₂-C₅-alkenyl, —S—C₁-C₄-halogenoalkyl having 1 to 5 halogen atoms, C₁-C₄-halogenoalkoxy having 1 to 5 halogen atoms,
R¹², R¹³ and R¹⁴, which may be the same or be different, are selected from the group consisting of hydrogen, halogen, cyano, C₁-C₄-alkyl, C₁-C₄-halogenoalkyl having 1 to 5 halogen atoms, C₁-C₄-alkoxy, —S—C₁-C₄-alkyl, C₁-C₄-halogenoalkoxy having 1 to 5 halogen atoms, —S(O)—C₁-C₄-alkyl, —S(O)₂—C₁-C₄-alkyl,
A represents a heterocycle of the formula (Het-2)

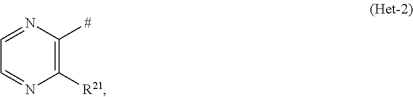

in which
# depicts the bond which connects A to the rest of the molecule, and
R²¹ is selected from the group consisting of hydrogen, halogen, C₁-C₄-alkyl and C₁-C₄-halogenoalkyl having 1 to 5 halogen atoms, or
A represents a heterocycle of the formula (Het-3)

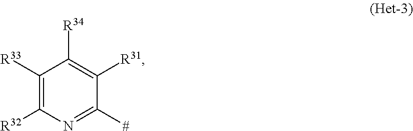

in which
# depicts the bond which connects A to the rest of the molecule,
R³¹ is selected from the group consisting of hydrogen, halogen, hydroxy, cyano, C₁-C₄-alkyl, C₁-C₄-halogenoalkyl having 1 to 5 halogen atoms, C₁-C₄ alkoxy, —S—C₁-C₅-alkyl, S(O)—C₁-C₄-alkyl, —S(O)₂—C₁-C₄-alkyl, —S—C₂-C₅-alkenyl, —S—C₁-C₄-halogenoalkyl having 1 to 5 halogen atoms, C₁-C₄-halogenoalkoxy having 1 to 5 halogen atoms,
R³², R³³ and R³⁴, which may be the same or be different, are selected from the group consisting of hydrogen, halogen, cyano, C₁-C₄-alkyl, C₁-C₄-halogenoalkyl having 1 to 5 halogen atoms, C₁-C₄-alkoxy, —S—C₁-C₄-alkyl, C₁-C₄-halogenoalkoxy having 1 to 5 halogen atoms, —S(O)—C₁-C₄-alkyl, —S(O)₂—C₁-C₄-alkyl,
   and salts, solvates, solvates of the salts, N-oxides, metal complexes or metalloid complexes thereof.

The present invention relates further to compounds of formula (I)

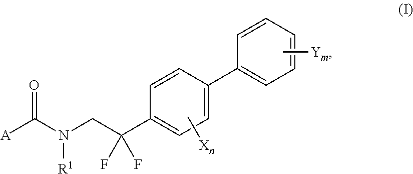

wherein
R¹ is selected from the group consisting of hydrogen, —CHO, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, C₃-C₆-halogenocycloalkyl having 1 to 5 halogen atoms, C₃-C₄-alkenyl, C₃-C₄-alkynyl, C₁-C₄-alkoxy-C₁-C₄-alkyl, C₃-C₆-cycloalkyl-C₁-C₃-alkyl, cyano-C₁-C₄-alkyl, amino-C₁-C₄-alkyl, C₁-C₄-alkylamino-C₁-C₄-alkyl, di-(C₁-C₄-alkyl)amino-C₁-C₄-alkyl, C₁-C₄-alkylcarbonyl, C₁-C₄-halogenoalkylcarbonyl having 1 to 5 halogen atoms, C₁-C₄-alkoxycarbonyl, benzyloxycarbonyl, C₁-C₄-alkoxy-C₁-C₄-alkylcarbonyl, —S(O)₂—C₁-C₄-alkyl, and —S(O)₂—C₁-C₄-halogenoalkyl having 1 to 5 halogen atoms,
n is 0, 1, 2 or 3,
each X is independently selected from the group consisting of hydrogen, halogen, nitro, cyano, hydroxy, amino, —SH, —SF₅, —CHO, —OCHO, —NHCHO, —COOH, —CONH₂, —CONH(OH), —OCONH₂, (hydroxyimino)-C₁-C₆-alkyl, C₁-C₈-alkyl, C₁-C₈-halogenoalkyl having 1 to 5 halogen atoms, C₂-C₈-alkenyl, C₂-C₈-alkynyl, C₁-C₈-alkylamino, di-(C₁-C₈-alkyl)amino, C₁-C₈-alkoxy, C₁-C₈-halogenoalkoxy having 1 to 5 halogen atoms, C₂-C₈-alkenyloxy, C₂-C₈-halogenoalkenyloxy having 1 to 5 halogen atoms, C₃-C₈-alkynyloxy, C₃-C₈-halogenoalkynyloxy having 1 to 5 halogen atoms, C₃-C₈-cycloalkyl, C₃-C₈-halogenocycloalkyl having 1 to 5 halogen atoms, C₁-C₈-alkylcarbonyl, C₁-C₈-halogenoalkylcarbonyl having 1 to 5 halogen atoms, —CONH(C₁-C₈-alkyl), —CON(C₁-C₈-alkyl)₂, —CONH(OC₁-C₈-alkyl), —CON(OC₁-C₈-alkyl)(C₁-C₈-alkyl), C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, —OCONH(C₁-C₈-alkyl), —OCON(C₁-C₈-alkyl)₂, —OCONH(OC₁-C₈-alkyl), —OCO(OC₁-C₈-alkyl), —S—C₁-C₈-alkyl, —S—C₁-C₈-halogenoalkyl having 1 to 5 halogen atoms, —S(O)—C₁-C₈-alkyl, —S(O)—C₁-C₈-halogenoalkyl having 1 to 5 halogen atoms, —S(O)₂—C₁-C₈-alkyl, —S(O)₂—C₁-C₈-halogenoalkyl having 1 to 5 halogen atoms, (C₁-C₆-alkoxyimino)-C₁-C₆-alkyl, (C₂-C₆-alkenyloxyimino)-C₁-C₆-alkyl, (C₃-C₆-alkynyloxyimino)-C₁-C₆-alkyl, (benzyloxyimino)-C₁-C₆-alkyl, benzyloxy, —S-benzyl, benzylamino, phenoxy, —S-phenyl and phenylamino,
m is 0, 1, 2, 3 or 4,
each Y is independently selected from the group consisting of hydrogen, halogen, nitro, cyano, hydroxy, amino, —SH, —SF₅, —CHO, —OCHO, —NHCHO, —COOH, —CONH₂, —CONH(OH), —OCONH₂, (hydroxyimino)-C₁-C₆-alkyl, C₁-C₈-alkyl, C₁-C₈-halogenoalkyl having 1 to 5 halogen atoms, C₂-C₈-alkenyl, C₂-C₈-alkynyl, C₁-C₈-alkylamino, di-(C₁-C₈-alkyl)amino, C₁-C₈-alkoxy, C₁-C₈-halogenoalkoxy having 1 to 5 halogen atoms, C₂-C₈-alkenyloxy, C₂-C₈-halogenoalkenyloxy having 1 to 5 halogen atoms, C₃-C₈-alkynyloxy, C₃-C₈-halogenoalkynyloxy having 1 to 5 halogen atoms, C₃-C₈-cycloalkyl, C₃-C₈-halogenocycloalkyl having 1 to 5 halogen atoms, C₁-C₈-alkylcarbonyl, C₁-C₈-halogenoalkylcarbonyl having 1 to 5 halogen atoms, —CONH(C₁-C₈-alkyl), —CON(C₁-C₈-alkyl)₂, —CONH(OC₁-C₈-alkyl), —CON(OC₁-C₈-alkyl)(C₁-C₈-alkyl), C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, —OCONH(C₁-C₈-alkyl), —OCON(C₁-C₈-alkyl)₂, —OCONH(OC₁-C₈-alkyl), —OCO(OC₁-C₈-alkyl), —S—C₁-C₈-alkyl, —S—C₁-C₈-halogenoalkyl having 1 to 5 halogen atoms, —S(O)—C₁-C₈-alkyl, —S(O)—C₁-C₈-halogenoalkyl having 1 to 5 halogen atoms, —S(O)₂—C₁-C₈-alkyl, —S(O)₂—C₁-C₈-halogenoalkyl having 1 to 5 halogen atoms, —CH₂—S—C₁-C₈-alkyl, —CH₂—S(O)—C₁-C₈-alkyl, —CH₂—S(O)₂—C₁-C₈-alkyl, (C₁-C₆-alkoxyimino)-C₁-C₆-alkyl, (C₂-C₆-alkenyloxyimino)-C₁-C₆-alkyl, (C₃-C₆-alkynyloxyimino)-C₁-C₆-alkyl, wherein two Y may additionally be selected from —O—C₁-C₃-alkoxy and —O—C₁-C₃-halogenalkoxy having 1 to 5 halogen atoms, with each oxy function being the connecting atom of the individual Y, thereby forming an annelated 5-, 6- or 7-membered ring with the phenyl moiety that they are connected to, or
each Y is independently selected from the group consisting of hydrogen, halogen, cyano, C₁-C₆-alkyl, C₁-C₆-halogenoalkyl having 1 to 5 halogen atoms, and a 4 to 7 membered heterocyclic ring system with 1 to 3 heteroatoms, but not O—O, O—S or 3 heteroatoms in a row, independently substituted by oxo, thiono or 1 to 12 substituents Z independently selected from the group consisting of hydrogen, halogen, cyano, hydroxy, amino, —SH, —CHO, —COOH, —CONH₂, —CONH(OH), —OCONH₂, (hydroxyimino)-C₁-C₆-alkyl, C₁-C₆-alkyl, C₁-C₆-halogenoalkyl having 1 to 5 halogen atoms, C₂-C₆-alkenyl, C₂-C₆-alkynyl, C₁-C₆-alkylamino, di-(C₁-C₆-alkyl)amino, C₁-C₆-alkoxy, C₁-C₆-halogenoalkoxy having 1 to 5 halogen atoms, C₂-C₆-alkenyloxy, C₂-C₆-halogenoalkenyloxy having 1 to 5 halogen atoms, C₃-C₆-alkynyloxy, C₃-C₆-halogenoalkynyloxy having 1 to 5 halogen atoms, C₃-C₆-cycloalkyl, C₃-C₆-halogenocycloalkyl having 1 to 5 halogen atoms, C₁-C₆-alkylcarbonyl, C₁-C₆-halogenoalkylcarbonyl having 1 to 5 halogen atoms, —CONH(C₁-C₆-alkyl), —CON(C₁-C₆-alkyl)₂, —CONH(OC₁-C₆-alkyl), —CON(OC₁-C₆-alkyl)(C₁-C₆-alkyl), C₁-C₆-alkoxycarbonyl, C₁-C₆-halogenoalkoxycarbonyl having 1 to 5 halogen atoms, C₁-C₆-alkylcarbonyloxy, C₁-C₆-halogenoalkylcarbonyloxy having 1 to 5 halogen atoms, C₁-C₆-alkylcarbonylamino, C₁-C₆-halogenoalkylcarbonylamino having 1 to 5 halogen atoms, —OCONH(C₁-C₆-alkyl), —OCON(C₁-C₆-alkyl)₂, —OCONH(OC₁-C₆-alkyl), —OCO(OC₁-C₆-alkyl), —S—C₁-C₆-alkyl, —S—C₁-C₆-halogenoalkyl having 1 to 5 halogen atoms, —S(O)—C₁-C₆-alkyl, —S(O)—C₁-C₆-halogenoalkyl having 1 to 5 halogen atoms, —S(O)₂—C₁-C₆-alkyl, —S(O)₂—C₁-C₆-halogenoalkyl having 1 to 5 halogen atoms, and
A represents a phenyl group of the formula (A1)

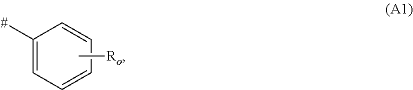

in which
# depicts the bond which connects A to the rest of the molecule,
o is 0, 1, 2, 3, 4 or 5, and
each R is independently selected from the group consisting of halogen, nitro, —OH, NH₂, SH, SF₅, CHO, OCHO, NHCHO, COOH, cyano, C₁-C₈-alkyl, C₁-C₈-halogenoalkyl having 1 to 9 halogen atoms, C₂-C₈-alkenyl, C₂-C₈-alkynyl, C₃-C₆-cycloalkyl, —S—C₁-C₈-alkyl, —S—C₁-C₈-halogenoalkyl having 1 to 5 halogen atoms, C₁-C₈-alkoxy, C₁-C₈-halogenoalkoxy having 1 to 5 halogen atoms, C₁-C₈-alkoxy-C₂-C₈-alkenyl, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, —S(O)—C₁-C₈-alkyl, —S(O)—C₁-C₈-halogenoalkyl having 1 to 5 halogen atoms, —S(O)₂—C₁-C₈-alkyl, —S(O)₂—C₁-C₈-halogenoalkyl having 1 to 5 halogen atoms, C₁-C₈-alkylsulfonamide, —NH(C₁-C₈-alkyl), N(C₁-C₈-alkyl)₂, phenyl (optionally substituted by C₁-C₆-alkoxy) and phenoxy, wherein two R bonded to adjacent carbon atoms may together represent —O(CH₂)ₚO—, wherein p represents 1 or 2, or
A represents a heterocycle of the formula (Het-1)

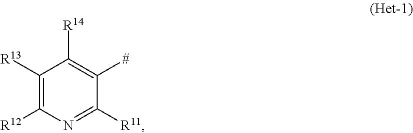

in which
# depicts the bond which connects A to the rest of the molecule,
R¹¹ is selected from the group consisting of hydrogen, halogen, hydroxy, cyano, C₁-C₄-alkyl, C₁-C₄-halogenoalkyl having 1 to 5 halogen atoms, C₁-C₄ alkoxy, —S—C₁-C₅-alkyl, S(O)—C₁-C₄-alkyl, —S(O)₂—C₁-C₄-alkyl, —S—C₂-C₅-alkenyl, —S—C₁-C₄-halogenoalkyl having 1 to 5 halogen atoms, C₁-C₄-halogenoalkoxy having 1 to 5 halogen atoms,
R¹², R¹³ and R¹⁴, which may be the same or be different, are selected from the group consisting of hydrogen, halogen, cyano, C₁-C₄-alkyl, C₁-C₄-halogenoalkyl having 1 to 5 halogen atoms, C₁-C₄-alkoxy, —S—C₁-C₄-alkyl, C₁-C₄-halogenoalkoxy having 1 to 5 halogen atoms, —S(O)—C₁-C₄-alkyl, —S(O)₂—C₁-C₄-alkyl,
A represents a heterocycle of the formula (Het-2)

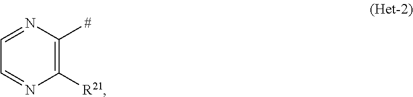

in which
# depicts the bond which connects A to the rest of the molecule, and
R²¹ is selected from the group consisting of hydrogen, halogen, C₁-C₄-alkyl and C₁-C₄-halogenoalkyl having 1 to 5 halogen atoms, or
A represents a heterocycle of the formula (Het-3)

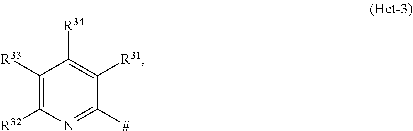

in which
# depicts the bond which connects A to the rest of the molecule,
R³¹ is selected from the group consisting of hydrogen, halogen, hydroxy, cyano, C₁-C₄-alkyl, C₁-C₄-halogenoalkyl having 1 to 5 halogen atoms, C₁-C₄ alkoxy, —S—C₁-C₅-alkyl, S(O)—C₁-C₄-alkyl, —S(O)₂—C₁-C₄-alkyl, —S—C₂-C₅-alkenyl, —S—C₁-C₄-halogenoalkyl having 1 to 5 halogen atoms, C₁-C₄-halogenoalkoxy having 1 to 5 halogen atoms,
R³², R³³ and R³⁴, which may be the same or be different, are selected from the group consisting of hydrogen, halogen, cyano, C₁-C₄-alkyl, C₁-C₄-halogenoalkyl having 1 to 5 halogen atoms, C₁-C₄-alkoxy, —S—C₁-C₄-alkyl, C₁-C₄-halogenoalkoxy having 1 to 5 halogen atoms, —S(O)—C₁-C₄-alkyl, —S(O)₂—C₁-C₄-alkyl,
   and salts, solvates, solvates of the salts, N-oxides, metal complexes or metalloid complexes thereof, for use in the control, treatment and/or prevention of infections with helminths in animals and humans.

In formulae (A1), (Het-1), (Het-2) and (Het-3) # depicts the bond which connects A to the C(O)NR¹-moiety in the compounds of formula (I), formula (I-1) or formula (I-2). In the context of the formulae describing the residue Y, # depicts the bond which connects Y to the phenyl-moiety of, for example, formula (I), formula (I-1), formula (I-2) or formula (VII). In general, in the present application # depicts the connecting bond of the structural element, unless otherwise indicated.

Compounds according to the invention are the compounds of the formula (I) and the salts, solvates and solvates of the salts thereof, the compounds of the formulae mentioned hereinafter and encompassed by formula (I) and the salts, solvates and solvates of the salts thereof, and the compounds which are mentioned hereinafter as exemplary embodiments and encompassed by formula (I) and the salts, solvates and solvates of the salts thereof, insofar as the compounds encompassed by formula (I) and mentioned hereinafter are not already salts, solvates and solvates of the salts.

Salts which are preferred for the purposes of the present invention are physiologically acceptable salts of the compounds according to the invention. Also encompassed are salts which are themselves unsuitable for pharmaceutical uses but can be used for example for isolating, purifying or storing the compounds according to the invention.

Physiologically acceptable salts of the compounds according to the invention include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, benzenesulphonic acid, toluenesulphonic acid, naphthalenedisulphonic acid, formic acid, acetic acid, trifluoroacetic acid, propionic acid, succinic acid, fumaric acid, maleic acid, lactic acid, tartaric acid, malic acid, citric acid, gluconic acid, benzoic acid and embonic acid.

Physiologically acceptable salts of the compounds according to the invention also include salts of conventional bases, such as, by way of example and preferably, alkali metal salts (e.g. sodium and potassium salts), alkaline earth metal salts (e.g. calcium and magnesium salts), zinc salts and ammonium salts derived from ammonia or organic amines having 1 to 16 C atoms, such as, by way of example and preferably, ethylamine, diethylamine, triethylamine, N,N-diisopropylethylamine, monoethanolamine, diethanolamine, triethanolamine, tromethamine, dimethylaminoethanol, diethylaminoethanol, choline, procaine, dicyclohexylamine, dibenzylamine, N-methylmorpholine, N-methylpiperidine, arginine, lysine and 1,2-ethylenediamine.

Solvates in the context of the invention are designated as those forms of the compounds according to the invention which form a complex in the solid or liquid state by coordination with solvent molecules. Hydrates are a specific form of solvates, in which the coordination takes place with water. Hydrates are preferred solvates in the context of the present invention.

The present invention also encompasses all suitable isotopic variants of the compounds according to the invention. An isotopic variant of a compound according to the invention is understood here to mean a compound in which at least one atom within the compound according to the invention has been exchanged for another atom of the same atomic number, but with a different atomic mass than the atomic mass which usually or predominantly occurs in nature. Examples of isotopes which can be incorporated into a compound according to the invention are those of hydrogen, carbon, nitrogen, oxygen, phosphorus, sulphur, fluorine, chlorine, bromine and iodine, such as ²H (deuterium), ³H (tritium), ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I. Particular isotopic variants of a compound according to the invention, especially those in which one or more radioactive isotopes have been incorporated, may be beneficial, for example, for the examination of the mechanism of action or of the active compound distribution in the body; due to comparatively easy preparability and detectability, especially compounds labelled with ³H or ¹⁴C isotopes are suitable for this purpose. In addition, the incorporation of isotopes, for example of deuterium, can lead to particular therapeutic benefits as a consequence of greater metabolic stability of the compound, for example an extension of the half-life in the body or a reduction in the active dose required; such modifications of the compounds according to the invention may therefore in some cases also constitute a preferred embodiment of the present invention. Isotopic variants of the compounds according to the invention can be prepared by generally customary processes known to those skilled in the art, for example by the methods described below and the methods described in the working examples, by using corresponding isotopic modifications of the respective reagents and/or starting materials therein.

The present invention moreover also includes prodrugs of the compounds according to the invention. The term “prodrugs” here designates compounds which themselves can be biologically active or inactive, but are converted (for example metabolically or hydrolytically) into compounds according to the invention during their dwell time in the body.

Any of the compounds according to the invention can exist in one or more optical or chiral isomer forms depending on the number of asymmetric centres in the compound. The invention thus relates equally to all the optical isomers and to their racemic or scalemic mixtures (the term “scalemic” denotes a mixture of enantiomers in different proportions), and to the mixtures of all the possible stereoisomers, in all proportions. The diastereoisomers and/or the optical isomers can be separated according to the methods which are known per se by the man ordinary skilled in the art.

Compounds of the present invention can also exist in one or more geometric isomer forms depending on the number of double bonds in the compound, especially all syn/anti (or cis/trans) isomers and to all possible syn/anti (or cis/trans) mixtures. The invention thus relates equally to all geometric isomers and to all possible mixtures, in all proportions. The geometric isomers can be separated according to general methods, which are known per se by the man ordinary skilled in the art.

Compounds of formula (I) may be found in its tautomeric form resulting from the shift of the proton of a hydroxy, sulfanyl or amino group. Such tautomeric forms of such compounds are also part of the present invention. More generally speaking, all tautomeric forms of compounds of formula (I), as well as the tautomeric forms of the compounds which can optionally be used as intermediates in the preparation processes and which will be defined in the description of these processes, are also part of the present invention.

Further, this invention is directed to pharmaceutical compositions comprising a compound of the invention. Furthermore, this invention is directed to pharmaceutical compositions comprising a compound of the invention for use in the control, treatment and/or prevention of infections with helminths in animals and humans. This invention also provides a composition comprising a compound of formula (I), or a salt, solvate, solvate of a salt, N-oxide, metal complex or metalloid complex thereof, and at least one pharmaceutically acceptable excepient. In one embodiment, this invention provides such a composition which further comprises at least one additional active ingredient, preferably a mixing partner as described below.

Further, this invention is directed to the use of compounds and/or compositions of the invention for the control, treatment and/or prevention of infections with helminths in animals and humans. This invention provides also a method for control, treatment and/or prevention of infections with helminths in animals and humans comprising administering a biologically effective amount of a compound of formula (I), or a salt, solvate, solvate of a salt, N-oxide, metal complex or metalloid complex thereof, or a composition described herein, to an animal or human in need. This invention also relates to such method wherein a composition comprising a biologically effective amount of a compound of formula (I), a salt, solvate, solvate of a salt, N-oxide, metal complex or metalloid complex thereof, and at least one pharmaceutically acceptable excipient, is administered to an animal or human in need, said composition optionally further comprising a biologically effective amount of at least one additional active ingredient, preferably a mixing partner as described below. According to the present invention, the described uses and methods are applicable in the context of the control, treatment and/or prevention of infections with helminths in animal and humans. If at any point any such use or method is only mentioned with regard to animals, this shall in general, and unless specifically indicated otherwise, refer to the use/method with regard to animals and humans and shall not be understood as a limitation. However, the uses and methods according to the present invention are in one preferred embodiment directed to the control, treatment and/or prevention of infections with helminths in non-human animals (only). In one embodiment, the methods according to the invention do not comprise methods for treatment of the human body by surgery or therapy and diagnostic methods practiced on the human body.

As used herein, the terms “comprises”, “comprising”, “includes”, “including”, “has”, “having”, “contains”, “containing”, “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components or elements, in addition to those literally disclosed, provided that these additional materials, steps, features, components or elements do not materially affect the basic and novel characteristic(s) of the claimed invention. The term “consisting essentially of” occupies a middle ground between “comprising” and “consisting of”.

Where applicants have defined an invention or a portion thereof with an open-ended term such as “comprising”, it should be readily understood that (unless otherwise stated) the description should be interpreted to also describe such an invention using the terms “consisting essentially of” or “consisting of”.

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

In the above recitations, the term “alkyl”, used either alone or in compound words such as “haloalkyl” includes straight-chain or branched alkyl, such as, methyl, ethyl, n-propyl, i-propyl, or the different butyl, pentyl or hexyl isomers. “Alkenyl” includes straight-chain or branched alkenes such as ethenyl, 1-propenyl, 2-propenyl, and the different butenyl, pentenyl and hexenyl isomers. “Alkenyl” also includes polyenes such as 1,2-propadienyl and 2,4-hexadienyl. “Alkynyl” includes straight-chain or branched alkynes such as ethynyl, 1-propynyl, 2-propynyl and the different butynyl, pentynyl and hexynyl isomers. “Alkynyl” can also include moieties comprised of multiple triple bonds such as 2,5-hexadiynyl.

“Alkoxy” includes, for example, methoxy, ethoxy, n-propyloxy, isopropyloxy and the different butoxy, pentoxy and hexyloxy isomers. “Alkoxyalkyl” denotes alkoxy substitution on alkyl. Examples of “alkoxyalkyl” include CH₃OCH₂, CH₃OCH₂CH₂, CH₃CH₂OCH₂, CH₃CH₂CH₂CH₂OCH₂ and CH₃CH₂OCH₂CH₂.

“Cycloalkyl” includes, for example, cyclopropyl, cyclobutyl, cyclopentyl and cyclohexyl. The term “cycloalkylalkyl” denotes cycloalkyl substitution on an alkyl moiety. Examples of “cycloalkylalkyl” include cyclopropylmethyl, cyclopentylethyl, and other cycloalkyl moieties bonded to straight-chain or branched alkyl groups. “Cycloalkenyl” includes groups such as cyclopentenyl and cyclohexenyl as well as groups with more than 10 one double bond such as 1,3- and 1,4-cyclohexadienyl. The term “cycloalkylcycloalkyl” denotes cycloalkyl substitution on another cycloalkyl ring, wherein each cycloalkyl ring independently has from 3 to 7 carbon atom ring members. Examples of cycloalkylcycloalkyl include cyclopropylcyclopropyl (such as 1,1′-bicyclopropyl-1-yl, 1,1′-bicyclopropyl-2-yl), cyclohexylcyclopentyl (such as 4-cyclopentylcyclohexyl) and cyclohexylcyclohexyl (such as 1,1′-bicyclohexyl-1-yl), and the different cis- and trans-cycloalkylcycloalkyl isomers, (such as (1R,2S)-1,1′-bicyclopropyl-2-yl and (1R,2R)-1,1′-bicyclopropyl-2-yl).

The term “halogen”, either alone or in compound words such as “haloalkyl”, or when used in descriptions such as “alkyl substituted with halogen” includes fluorine, chlorine, bromine or iodine. Further, when used in compound words such as “haloalkyl”, or when used in descriptions such as “alkyl substituted with halogen” said alkyl may be partially or fully substituted with halogen atoms which may be the same or different. Examples of “haloalkyl” or “alkyl substituted with halogen” include F₃C, ClCH₂, CF₃CH₂ and CF₃CCl₂. The terms “haloalkoxy”, “haloalkenyl”, “haloalkynyl”, and the like, are defined analogously to the term “haloalkyl”. Examples of “haloalkoxy” include CF₃O, CCl₃CH₂O, HCF₂CH₂CH₂O and CF₃CH₂O. Examples of “haloalkenyl” include (Cl)₂C═CHCH₂ and CF₃CH₂CH═CHCH₂. Examples of “haloalkynyl” include HC≡CCHCl, CF₃C≡C, CCl₃C≡C and FCH₂C≡CCH₂.

The chemical abbreviation C(O) as used herein represents a carbonyl moiety. For example, C(O)CH₃ represents an acetyl group. The chemical abbreviations CO₂ and C(O)O as used herein represent an ester moiety. For example, CO₂Me and C(O)OMe represent a methyl ester. CHO represents an aldehyde moiety.

“OCN” means —O—C≡N, and “SCN” means —S—C≡N.

The total number of carbon atoms in a substituent group is indicated by the “Ci-Cj” prefix where i and j are numbers from 1 to 14. C₂ alkoxyalkyl designates CH₃OCH₂; C₃ alkoxyalkyl designates, for example, CH₃CH(OCH₃), CH₃OCH₂CH₂ or CH₃CH₂OCH₂; and C₄ alkoxyalkyl designates the various isomers of an alkyl group substituted with an alkoxy group containing a total of four carbon atoms, examples including CH₃CH₂CH₂OCH₂ and CH₃CH₂OCH₂CH₂.

When a compound is substituted with a substituent bearing a subscript that indicates the number of said substituents can exceed 1, said substituents (when they exceed 1) are independently selected from the group of defined substituents, e.g. n=0, 1, 2, 3 or 4. When a group contains a substituent which can be hydrogen, for example R² or R³, then when this substituent is taken as hydrogen, it is recognized that this is equivalent to said group being unsubstituted.

Unless otherwise indicated, a “ring” or “ring system” as a component of formula (I) is carbocyclic or heterocyclic. The term “ring system” denotes two or more fused rings. The term “heterocyclic ring” denotes a ring in which at least one atom forming the ring backbone is not carbon, e.g., nitrogen, oxygen or sulfur. Typically a heterocyclic ring contains no more than 4 nitrogens, no more than 2 oxygens and no more than 2 sulfurs. Unless otherwise indicated, a heterocyclic ring can be a saturated, partially unsaturated, or fully unsaturated ring. The term “heterocyclic ring system” denotes a ring system in which at least one ring of the ring system is a heterocyclic ring. Unless otherwise indicated, heterocyclic rings and ring systems can be attached through any available carbon or nitrogen by replacement of a hydrogen on said carbon or nitrogen.

As used herein, the following definitions shall apply unless otherwise indicated. The term “optionally substituted” is used interchangeably with the phrase “substituted or unsubstituted” or with the term “(un)substituted”. The expression “optionally substituted with 1 to 4 substituents” means that no substituent is present (i.e. unsubstituted) or that 1, 2, 3 or 4 substituents are present (limited by the number of available bonding positions). Unless otherwise indicated, an optionally substituted group may have a substituent at each substitutable position of the group, and each substitution is independent of the other.

The following embodiments that relate to formula (I) shall be understood as referring to the compounds according to the present invention per se or to the compounds for use in the control, treatment and/or prevention of infections with helminths in animals and humans according to the present invention, or to both, unless indicated otherwise.

In one embodiment, the present invention provides compounds of formula (I), wherein
A represents a phenyl group of formula (A1)

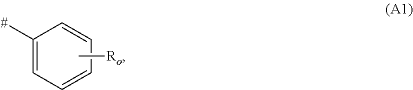

in which
# depicts the bond which connects A to the rest of the molecule,
o is 0, 1 or 2, and
each R is independently selected from the group consisting of halogen, nitro, C₁-C₄-halogenoalkyl having 1 to 5 halogen atoms, C₁-C₄-halogenoalkoxy having 1 to 5 halogen atoms, or
A represents a heterocycle of the formula (Het-1)

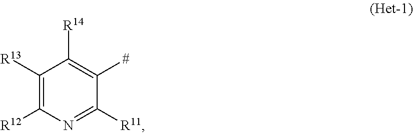

in which
# depicts the bond which connects A to the rest of the molecule,
R¹¹ is halogen or C₁-C₄-halogenoalkyl having 1 to 5 halogen atoms, and
R¹², R¹³ and R¹⁴ are hydrogen, or
A represents a heterocycle of the formula (Het-2)

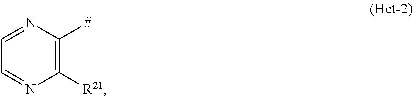

in which
# depicts the bond which connects A to the rest of the molecule, and
R²¹ is C₁-C₄-halogenoalkyl having 1 to 5 halogen atoms, or
A represents a heterocycle of the formula (Het-3)

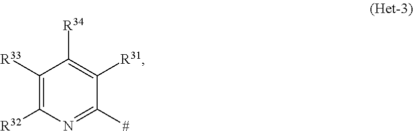

in which
# depicts the bond which connects A to the rest of the molecule,
R³¹ is C₁-C₄-halogenoalkyl having 1 to 5 halogen atoms, and
R³², R³³ and R³⁴ are hydrogen, and
R¹, n, X, m and Y have a meaning as described herein.

According to a further embodiment, the present invention provides compounds according to formula (I), wherein
A represents a phenyl group of formula (A1)

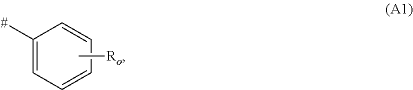

in which
# depicts the bond which connects A to the rest of the molecule,
o is 1 or 2, and
each R is independently selected from the group consisting of halogen, nitro, —CF₃, —OCF₃, —CHF₂, or
A represents a heterocycle of the formula (Het-1)

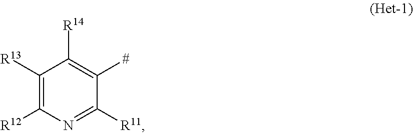

in which
# depicts the bond which connects A to the rest of the molecule,
R¹¹ is halogen or CF₃, and
R¹², R¹³ and R¹⁴ are hydrogen, or
A represents a heterocycle of the formula (Het-2)

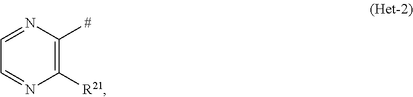

in which
# depicts the bond which connects A to the rest of the molecule, and
R²¹ is CF₃, or
A represents a heterocycle of the formula (Het-3)

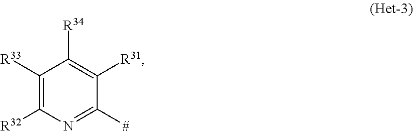

in which
# depicts the bond which connects A to the rest of the molecule,
R³¹ is CF₃, and
R³², R³³ and R³⁴ are hydrogen, and
R¹, n, X, m and Y have a meaning as described herein.

According to a still further embodiment, the present invention provides compounds according to formula (I), wherein
A is selected from the group consisting of

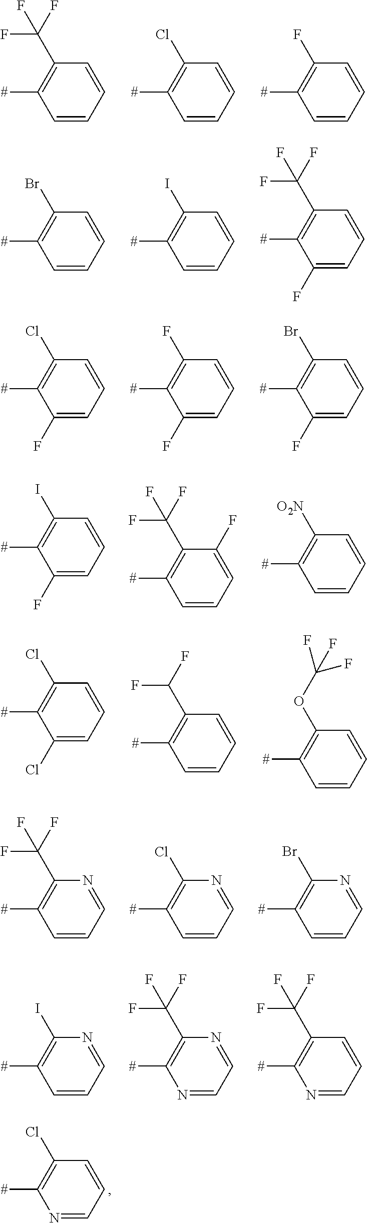

in which
# depicts the bond which connects A to the rest of the molecule, and
R¹, n, X, m and Y have a meaning as described herein.

According to a still further embodiment, the present invention provides compounds according to formula (I), wherein
A is selected from the group consisting of

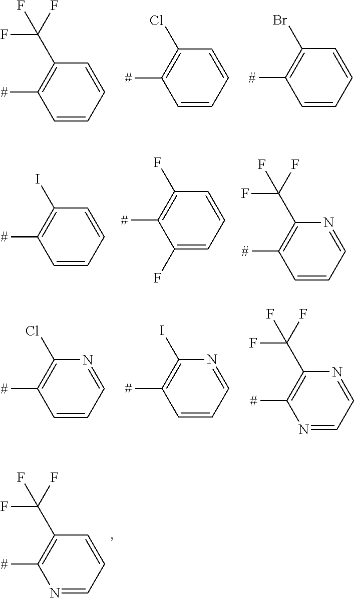

in which
# depicts the bond which connects A to the rest of the molecule, and
R¹, n, X, m and Y have a meaning as described herein.

According to a still further embodiment, the present invention provides compounds according to formula (I), wherein
A is selected from the group consisting of

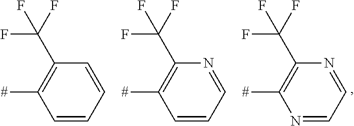

in which
# depicts the bond which connects A to the rest of the molecule, and
R¹, n, X, m and Y have a meaning as described herein.

According to a still further embodiment, the present invention provides compounds according to formula (I), wherein
A is selected from the group consisting of

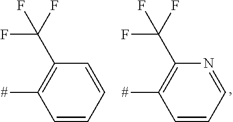

in which
# depicts the bond which connects A to the rest of the molecule, and
R¹, n, X, m and Y have a meaning as described herein.

In another embodiment, the present invention provides compounds according to formula (I), wherein
R¹ is selected from the group consisting of hydrogen, C₁-C₄-alkyl, C₃-C₄-alkynyl, C₃-C₆-cycloalkyl, C₁-C₄-alkylcarbonyl, C₁-C₄-alkoxy-C₁-C₄-alkyl, C₁-C₄-alkoxycarbonyl, and
A, n, X, m and Y have a meaning as described herein.

According to a further embodiment, the present invention provides compounds according to formula (I), wherein
R¹ is hydrogen, and
A, n, X, m and Y have a meaning as described herein.

In another embodiment, the present invention provides compounds according to formula (I), wherein
n is 1 or 2,
each X is independently selected from the group consisting of halogen, nitro, cyano, C₁-C₄-alkyl, C₁-C₄-halogenoalkyl having 1 to 5 halogen atoms, C₂-C₄-alkenyl, C₂-C₄-alkynyl, C₁-C₄-alkylamino, di-(C₁-C₄-alkyl)amino, C₁-C₄-alkoxy, C₁-C₄-halogenoalkoxy having 1 to 5 halogen atoms, C₂-C₄-alkenyloxy, C₂-C₄-halogenoalkenyloxy having 1 to 5 halogen atoms, C₃-C₄-alkynyloxy, C₃-C₄-halogenoalkynyloxy having 1 to 5 halogen atoms, C₃-C₆-cycloalkyl, C₃-C₆-halogenocycloalkyl having 1 to 5 halogen atoms, C₁-C₄-alkylcarbonyl, C₁-C₄-halogenoalkylcarbonyl having 1 to 5 halogen atoms, —CONH(C₁-C₄-alkyl), —CON(C₁-C₄-alkyl)₂, —CONH(OC₁-C₄-alkyl), —CON(OC₁-C₄-alkyl)(C₁-C₄-alkyl), C₁-C₄-alkoxycarbonyl, C₁-C₄-halogenoalkoxycarbonyl having 1 to 5 halogen atoms, C₁-C₄-alkylcarbonyloxy, C₁-C₄-halogenoalkylcarbonyloxy having 1 to 5 halogen atoms, C₁-C₄-alkylcarbonylamino, C₁-C₄-halogenoalkylcarbonylamino having 1 to 5 halogen atoms, —OCONH(C₁-C₄-alkyl), —OCON(C₁-C₄-alkyl)₂, —OCONH(OC₁-C₄-alkyl), —OCO(OC₁-C₄-alkyl), —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, (C₁-C₄-alkoxyimino)-C₁-C₄-alkyl, (C₂-C₆-alkenyloxyimino)-C₁-C₄-alkyl, (C₃-C₆-alkynyloxyimino)-C₁-C₄-alkyl, (benzyloxyimino)-C₁-C₆-alkyl, benzyloxy, —S-benzyl, benzylamino, phenoxy, —S-phenyl and phenylamino, and
A, R¹, m and Y have a meaning as described herein.

According to a further embodiment, the present invention provides compounds according to formula (I), wherein
n is 1 or 2,
each X is independently selected from the group consisting of halogen, cyano, C₁-C₄-alkyl, C₁-C₄-alkoxy and C₁-C₄-halogenoalkyl having 1 to 5 halogen atoms, and
A, R¹, m and Y have a meaning as described herein.

According to a still further embodiment, the present invention provides compounds according to formula (I), wherein
n is 1 or 2,
each X is independently selected from the group consisting of halogen, cyano, CH₃, OCH₃ and CF₃, and
A, R¹, m and Y have a meaning as described herein.

According to a still further embodiment, the present invention provides compounds according to formula (I), wherein
n is 1 or 2,
each X is independently selected from the group consisting of chloro, fluoro, cyano, CH₃, OCH₃ and CF₃, and
A, R¹, m and Y have a meaning as described herein.

According to a still further embodiment, the present invention provides compounds according to formula (I), wherein
n is 1 or 2,
each X is independently selected from the group consisting of chloro, fluoro, CH₃, OCH₃ and CF₃, and
A, R¹, m and Y have a meaning as described herein.

According to a still further embodiment, the present invention provides compounds according to formula (I), wherein
n is 1 or 2,
each X is independently selected from the group consisting of chloro, fluoro, CH₃ and CF₃, and
A, R¹, m and Y have a meaning as described herein.

In another embodiment, the present invention provides compounds for use in the control, treatment and/or prevention of infections with helminths in animals and humans according to formula (I), wherein
n is 1 or 2,
each X is independently selected from the group consisting of hydrogen, halogen, nitro, cyano, C₁-C₄-alkyl, C₁-C₄-halogenoalkyl having 1 to 5 halogen atoms, C₂-C₄-alkenyl, C₂-C₄-alkynyl, C₁-C₄-alkylamino, di-(C₁-C₄-alkyl)amino, C₁-C₄-alkoxy, C₁-C₄-halogenoalkoxy having 1 to 5 halogen atoms, C₂-C₄-alkenyloxy, C₂-C₄-halogenoalkenyloxy having 1 to 5 halogen atoms, C₃-C₄-alkynyloxy, C₃-C₄-halogenoalkynyloxy having 1 to 5 halogen atoms, C₃-C₆-cycloalkyl, C₃-C₆-halogenocycloalkyl having 1 to 5 halogen atoms, C₁-C₄-alkylcarbonyl, C₁-C₄-halogenoalkylcarbonyl having 1 to 5 halogen atoms, —CONH(C₁-C₄-alkyl), —CON(C₁-C₄-alkyl)₂, —CONH(OC₁-C₄-alkyl), —CON(OC₁-C₄-alkyl)(C₁-C₄-alkyl), C₁-C₄-alkoxycarbonyl, C₁-C₄-halogenoalkoxycarbonyl having 1 to 5 halogen atoms, C₁-C₄-alkylcarbonyloxy, C₁-C₄-halogenoalkylcarbonyloxy having 1 to 5 halogen atoms, C₁-C₄-alkylcarbonylamino, C₁-C₄-halogenoalkylcarbonylamino having 1 to 5 halogen atoms, —OCONH(C₁-C₄-alkyl), —OCON(C₁-C₄-alkyl)₂, —OCONH(OC₁-C₄-alkyl), —OCO(OC₁-C₄-alkyl), —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, (C₁-C₄-alkoxyimino)-C₁-C₄-alkyl, (C₂-C₆-alkenyloxyimino)-C₁-C₄-alkyl, (C₃-C₆-alkynyloxyimino)-C₁-C₄-alkyl, (benzyloxyimino)-C₁-C₆-alkyl, benzyloxy, —S-benzyl, benzylamino, phenoxy, —S-phenyl and phenylamino, and
A, R¹, m and Y have a meaning as described herein.

According to a further embodiment, the present invention provides compounds for use in the control, treatment and/or prevention of infections with helminths in animals and humans according to formula (I), wherein
n is 1 or 2,
each X is independently selected from the group consisting of halogen, cyano, C₁-C₄-alkyl, C₁-C₄-alkoxy and C₁-C₄-halogenoalkyl having 1 to 5 halogen atoms, and
A, R¹, m and Y have a meaning as described herein.

According to a still further embodiment, the present invention provides compounds for use in the control, treatment and/or prevention of infections with helminths in animals and humans according to formula (I), wherein
n is 1 or 2,
each X is independently selected from the group consisting of halogen, cyano, CH₃, OCH₃ and CF₃, and
A, R¹, m and Y have a meaning as described herein.

According to a still further embodiment, the present invention provides compounds for use in the control, treatment and/or prevention of infections with helminths in animals and humans according to formula (I), wherein
n is 1 or 2,
each X is independently selected from the group consisting of chloro, fluoro, cyano, CH₃, OCH₃ and CF₃, and
A, R¹, m and Y have a meaning as described herein.

According to a still further embodiment, the present invention provides compounds for use in the control, treatment and/or prevention of infections with helminths in animals and humans according to formula (I), wherein
n is 1 or 2,
each X is independently selected from the group consisting of chloro, fluoro, CH₃, OCH₃ and CF₃, and
A, R¹, m and Y have a meaning as described herein.

According to a still further embodiment, the present invention provides compounds for use in the control, treatment and/or prevention of infections with helminths in animals and humans according to formula (I), wherein
n is 1 or 2,
each X is independently selected from the group consisting of chloro, fluoro, CH₃ and CF₃, and
A, R¹, m and Y have a meaning as described herein.

In another embodiment, the present invention provides compounds according to formula (I), wherein
m is 1, 2 or 3, and
each Y is independently selected from the group consisting of hydrogen, halogen, nitro, cyano, hydroxy, amino, —SH, —SF₅, —CHO, —OCHO, —NHCHO, —COOH, —CONH₂, —CONH(OH), —OCONH₂, (hydroxyimino)-C₁-C₆-alkyl, C₁-C₆-alkyl, C₁-C₆-halogenoalkyl having 1 to 5 halogen atoms, C₂-C₆-alkenyl, C₂-C₆-alkynyl, C₁-C₆-alkylamino, di-(C₁-C₆-alkyl)amino, C₁-C₆-alkoxy, C₁-C₆-halogenoalkoxy having 1 to 5 halogen atoms, C₂-C₆-alkenyloxy, C₂-C₈-halogenoalkenyloxy having 1 to 5 halogen atoms, C₃-C₆-alkynyloxy, C₃-C₆-halogenoalkynyloxy having 1 to 5 halogen atoms, C₃-C₆-cycloalkyl, C₃-C₆-halogenocycloalkyl having 1 to 5 halogen atoms, C₁-C₆-alkylcarbonyl, C₁-C₆-halogenoalkylcarbonyl having 1 to 5 halogen atoms, —CONH(C₁-C₆-alkyl), —CON(C₁-C₆-alkyl)₂, —CONH(OC₁-C₆-alkyl), —CON(OC₁-C₆-alkyl)(C₁-C₆-alkyl), C₁-C₆-alkoxycarbonyl, C₁-C₆-halogenoalkoxycarbonyl having 1 to 5 halogen atoms, C₁-C₆-alkylcarbonyloxy, C₁-C₆-halogenoalkylcarbonyloxy having 1 to 5 halogen atoms, C₁-C₆-alkylcarbonylamino, C₁-C₆-halogenoalkylcarbonylamino having 1 to 5 halogen atoms, —OCONH(C₁-C₆-alkyl), —OCON(C₁-C₆-alkyl)₂, —OCONH(OC₁-C₆-alkyl), —OCO(OC₁-C₆-alkyl), —S—C₁-C₆-alkyl, —S—C₁-C₆-halogenoalkyl having 1 to 5 halogen atoms, —S(O)—C₁-C₆-alkyl, —S(O)—C₁-C₆-halogenoalkyl having 1 to 5 halogen atoms, —S(O)₂—C₁-C₆-alkyl, —S(O)₂—C₁-C₆-halogenoalkyl having 1 to 5 halogen atoms, —CH₂—S—C₁-C₆-alkyl, —CH₂—S(O)—C₁-C₆-alkyl, —CH₂—S(O)₂—C₁-C₆-alkyl, (C₁-C₄-alkoxyimino)-C₁-C₄-alkyl, (C₂-C₄-alkenyloxyimino)-C₁-C₄-alkyl, (C₃-C₆-alkynyloxyimino)-C₁-C₄-alkyl, wherein two Y may additionally be selected from —O—C₁-C₃-alkoxy and —O—C₁-C₃-halogenalkoxy having 1 to 5 halogen atoms, with each oxy function being the connecting atom of the individual Y, thereby forming an annelated 5-, 6- or 7-membered ring with the phenyl moiety that they are connected to, or
each Y is independently selected from the group consisting of hydrogen, halogen, cyano, C₁-C₆-alkyl, C₁-C₆-halogenoalkyl having 1 to 5 halogen atoms, and

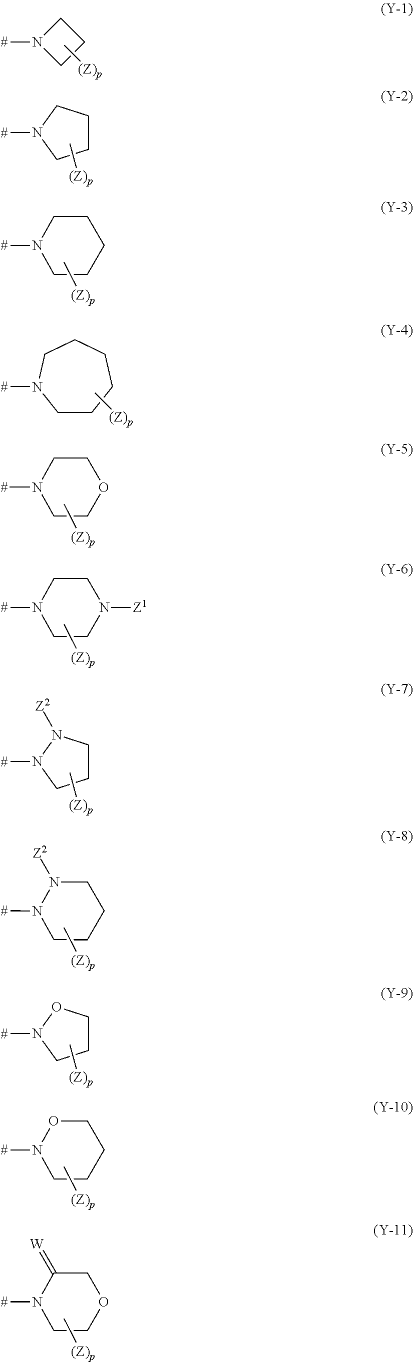

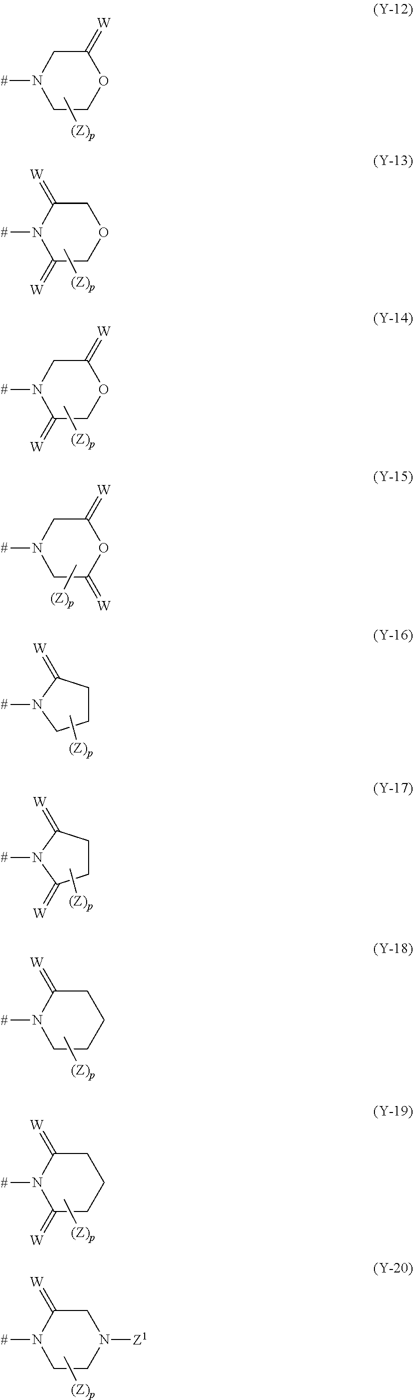

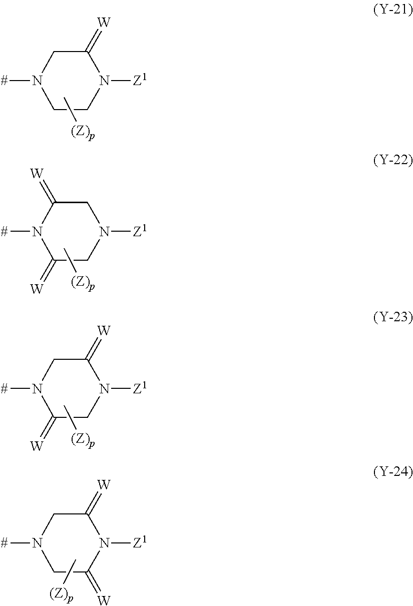

in which
# depicts the bond which connects Y to the rest of the molecule,
W is oxygen or sulfur,
p is 0, 1, 2, 3, 4, 5 or 6, and
Z is independently selected from the group consisting of hydrogen, halogen, cyano, hydroxy, amino, —SH, —CHO, —COOH, —CONH₂, —CONH(OH), —OCONH₂, (hydroxyimino)-C₁-C₆-alkyl, C₁-C₆-alkyl, C₁-C₆-halogenoalkyl having 1 to 5 halogen atoms, C₁-C₆-alkylamino, di-(C₁-C₆-alkyl)amino, C₁-C₆-alkoxy, C₁-C₆-halogenoalkoxy having 1 to 5 halogen atoms, C₃-C₆-cycloalkyl, C₃-C₆-halogenocycloalkyl having 1 to 5 halogen atoms, —CONH(C₁-C₆-alkyl), —CON(C₁-C₆-alkyl)₂, —S—C₁-C₆-alkyl, —S—C₁-C₆-halogenoalkyl having 1 to 5 halogen atoms, —S(O)—C₁-C₆-alkyl, —S(O)—C₁-C₆-halogenoalkyl having 1 to 5 halogen atoms, —S(O)₂—C₁-C₆-alkyl, —S(O)₂—C₁-C₆-halogenoalkyl having 1 to 5 halogen atoms,
Z¹ is independently selected from the group consisting of hydrogen, C₁-C₆-alkyl, C₁-C₆-halogenoalkyl having 1 to 5 halogen atoms, C₁-C₆-alkylamino, di-(C₁-C₆-alkyl)amino, C₁-C₆-alkoxy, C₁-C₆-alkylcarbonyl, C₃-C₆-cycloalkyl, C₃-C₆-halogenocycloalkyl having 1 to 5 halogen atoms, —CONH(C₁-C₆-alkyl), —CON(C₁-C₆-alkyl)₂,
Z² is independently selected from the group consisting of hydrogen, C₁-C₆-alkyl, C₁-C₆-halogenoalkyl having 1 to 5 halogen atoms, C₃-C₆-cycloalkyl, C₃-C₆-halogenocycloalkyl having 1 to 5 halogen atoms, —CONH(C₁-C₆-alkyl), —CON(C₁-C₆-alkyl)₂, and
A, R¹, n and X have a meaning as described herein.

According to a further embodiment, the present invention provides compounds according to formula (I), wherein
m is 1, 2 or 3, and
each Y is independently selected from the group consisting of hydrogen, halogen, nitro, cyano, hydroxy, amino, —SH, —SF₅, —CHO, —OCHO, —NHCHO, —COOH, —CONH₂, —CONH(OH), —OCONH₂, (hydroxyimino)-C₁-C₄-alkyl, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, C₃-C₆-cycloalkyl, C₃-C₆-halogenocycloalkyl having 1 to 5 halogen atoms, C₁-C₄-alkylcarbonyl, C₁-C₄-halogenoalkylcarbonyl having 1 to 5 halogen atoms, —CONH(C₁-C₄-alkyl), —CON(C₁-C₄-alkyl)₂, —CONH(OC₁-C₄-alkyl), —CON(OC₁-C₄-alkyl)(C₁-C₆-alkyl), C₁-C₄-alkoxycarbonyl, C₁-C₄-halogenoalkoxycarbonyl having 1 to 5 halogen atoms, C₁-C₄-alkylcarbonyloxy, C₁-C₄-halogenoalkylcarbonyloxy having 1 to 5 halogen atoms, C₁-C₄-alkylcarbonylamino, C₁-C₄-halogenoalkylcarbonylamino having 1 to 5 halogen atoms, —OCONH(C₁-C₄-alkyl), —OCON(C₁-C₄-alkyl)₂, —OCONH(OC₁-C₄-alkyl), —OCO(OC₁-C₄-alkyl), —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, —CH₂—S—C₁-C₄-alkyl, —CH₂—S(O)—C₁-C₄-alkyl, —CH₂—S(O)₂—C₁-C₄-alkyl, (C₁-C₄-alkoxyimino)-C₁-C₄-alkyl, (C₂-C₄-alkenyloxyimino)-C₁-C₄-alkyl, (C₃-C₆-alkynyloxyimino)-C₁-C₄-alkyl, wherein two Y may additionally be selected from —O—C₁-C₃-alkoxy and —O—C₁-C₃-halogenalkoxy having 1 to 5 halogen atoms, with each oxy function being the connecting atom of the individual Y, thereby forming an annelated 5-, 6- or 7-membered ring with the phenyl moiety that they are connected to, or
each Y is independently selected from the group consisting of hydrogen, halogen, cyano, C₁-C₄-alkyl, C₁-C₄-halogenoalkyl having 1 to 5 halogen atoms, and

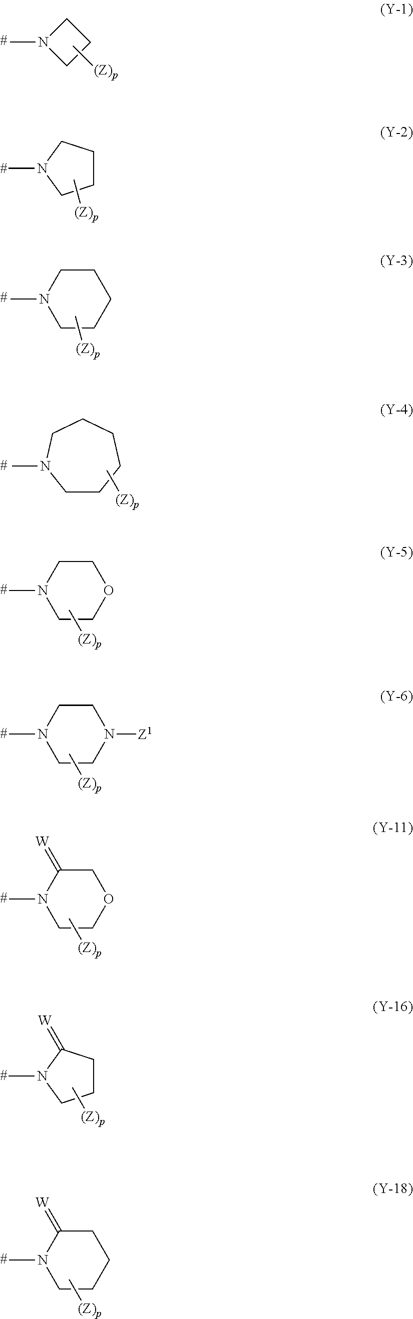

in which
# depicts the bond which connects Y to the rest of the molecule,
W is oxygen,
p is 0, 1, 2, 3, 4, 5 or 6,
Z is independently selected from the group consisting of hydrogen, halogen, cyano, hydroxy, amino, —SH, —CHO, —COOH, —CONH₂, —CONH(OH), —OCONH₂, (hydroxyimino)-C₁-C₄-alkyl, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, C₃-C₆-cycloalkyl, C₃-C₆-halogenocycloalkyl having 1 to 5 halogen atoms, —CONH(C₁-C₄-alkyl), —CON(C₁-C₄-alkyl)₂, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, and
Z¹ is independently selected from the group consisting of hydrogen, C₁-C₆-alkyl, C₁-C₆-halogenoalkyl having 1 to 5 halogen atoms, C₁-C₆-alkylamino, di-(C₁-C₆-alkyl)amino, C₁-C₆-alkoxy, C₁-C₆-alkylcarbonyl, C₃-C₆-cycloalkyl, C₃-C₆-halogenocycloalkyl having 1 to 5 halogen atoms, —CONH(C₁-C₆-alkyl), —CON(C₁-C₆-alkyl)₂, and
A, R¹, n and X have a meaning as described herein.

According to a still further embodiment, the present invention provides compounds according to formula (I), wherein
m is 1, 2 or 3, and
each Y is independently selected from the group consisting of hydrogen, halogen, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —CONH(C₁-C₄-alkyl), —CON(C₁-C₄-alkyl)₂, —CONH(OC₁-C₄-alkyl), —CON(C₁-C₄-alkyl)(C₁-C₆-alkyl), —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂-C₁-C₄-halogenoalkyl having 1 to 5 halogen atoms, C₁-C₄-alkylcarbonylamino, wherein two Y may additionally be selected from —O—C₁-C₂-alkoxy and —O—C₁-C₂-halogenalkoxy having 1 to 5 halogen atoms, with each oxy function being the connecting atom of the individual Y, thereby forming an annelated 5- or 6-membered ring with the phenyl moiety that they are connected to, or
each Y is independently selected from the group consisting of hydrogen, halogen, cyano, CF₃, and

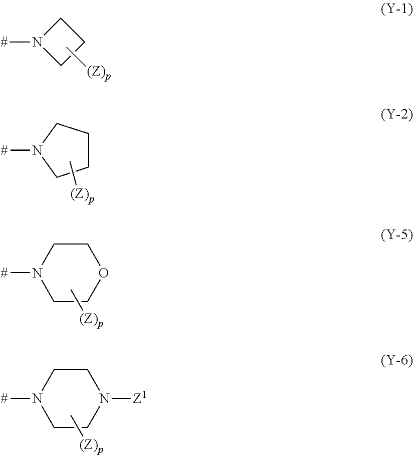

in which
# depicts the bond which connects Y to the rest of the molecule,
W is oxygen,
p is 0, 1, 2, 3, 4, 5 or 6,
Z is independently selected from the group consisting of hydrogen, halogen, cyano, hydroxy, amino, —SH, —CHO, —COOH, —CONH₂, —CONH(OH), —OCONH₂, (hydroxyimino)-C₁-C₄-alkyl, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, C₃-C₆-cycloalkyl, C₃-C₆-halogenocycloalkyl having 1 to 5 halogen atoms, —CONH(C₁-C₄-alkyl), —CON(C₁-C₄-alkyl)₂, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, and
Z¹ is independently selected from the group consisting of hydrogen, C₁-C₆-alkyl, C₁-C₆-halogenoalkyl having 1 to 5 halogen atoms, C₁-C₆-alkylamino, di-(C₁-C₆-alkyl)amino, C₁-C₆-alkoxy, C₁-C₆-alkylcarbonyl, C₃-C₆-cycloalkyl, C₃-C₆-halogenocycloalkyl having 1 to 5 halogen atoms, —CONH(C₁-C₆-alkyl), —CON(C₁-C₆-alkyl)₂, and
A, R¹, n and X have a meaning as described herein.

According to a still further embodiment, the present invention provides compounds according to formula (I), wherein
m is 1, 2 or 3, and
each Y is independently selected from the group consisting of hydrogen, halogen, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —CONH(C₁-C₄-alkyl), —CON(C₁-C₄-alkyl)₂, —CONH(OC₁-C₄-alkyl), —CON(OC₁-C₄-alkyl)(C₁-C₆-alkyl), —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino, wherein two Y may additionally be selected from —O—CH₂—O— and —O—C₁-halogenalkoxy having 1 or 2 halogen atoms, with each oxy function being the connecting atom of the individual Y, thereby forming an annelated 5-membered ring with the phenyl moiety that they are connected to, or
each Y is independently selected from the group consisting of hydrogen, halogen, cyano, CF₃, and

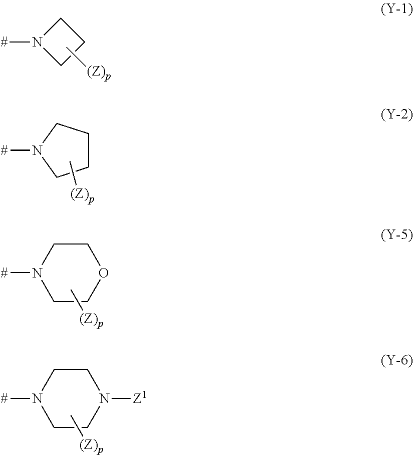

in which
# depicts the bond which connects Y to the rest of the molecule,
W is oxygen,
p is 0, 1, 2, 3, 4, 5 or 6,
Z is independently selected from the group consisting of hydrogen, halogen, cyano, hydroxy, amino, —SH, —CHO, —COOH, —CONH₂, —CONH(OH), —OCONH₂, (hydroxyimino)-C₁-C₄-alkyl, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, C₃-C₆-cycloalkyl, C₃-C₆-halogenocycloalkyl having 1 to 5 halogen atoms, —CONH(C₁-C₄-alkyl), —CON(C₁-C₄-alkyl)₂, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, and
Z¹ is independently selected from the group consisting of hydrogen, C₁-C₆-alkyl, C₁-C₆-halogenoalkyl having 1 to 5 halogen atoms, C₁-C₆-alkylcarbonyl, and
A, R¹, n and X have a meaning as described herein.

According to a still further embodiment, the present invention provides compounds according to formula (I), wherein
m is 1, 2 or 3,
each Y is independently selected from the group consisting of hydrogen, halogen, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino, wherein two Y may additionally be selected from —O—CH₂—O— and —O—CF₂—O—, with each oxy function being the connecting atom of the individual Y, thereby forming an annelated 5-membered ring with the phenyl moiety that they are connected to, or
each Y is independently selected from the group consisting of hydrogen, fluoro, chloro, cyano, CF₃, and

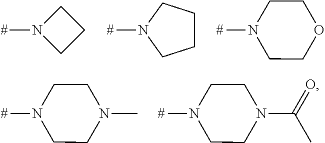

and
A, R¹, n and X have a meaning as described herein.

According to a still further embodiment, the present invention provides compounds according to formula (I), wherein
m is 1, 2 or 3,
each Y is independently selected from the group consisting of hydrogen, halogen, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino, and
A, R¹, n and X have a meaning as described herein.

According to a still further embodiment, the present invention provides compounds according to formula (I), wherein
m is 1, 2 or 3,
each Y is independently selected from the group consisting of hydrogen, fluoro, chloro, bromo, cyano, trifluoromethyl, difluoromethyl, methoxy, trifluoromethoxy, difluoromethoxy, methylamino, dimethylamino, methylsulfanyl, trifluoromethylsulfanyl, methylsulfinyl, trifluoromethylsulfinyl, methylsulfonyl, trifluoromethylsulfonyl, acetylamino, or
each Y is independently selected from the group consisting of hydrogen, fluoro, chloro, cyano, CF₃, and

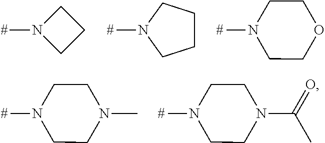

and
A, R¹, n and X have a meaning as described herein.

According to a still further embodiment, the present invention provides compounds according to formula (I), wherein
m is 1, 2 or 3,
each Y is independently selected from the group consisting of hydrogen, fluoro, chloro, bromo, cyano, trifluoromethyl, difluoromethyl, methoxy, trifluoromethoxy, difluoromethoxy, methylamino, dimethylamino, methylsulfanyl, trifluoromethylsulfanyl, methylsulfinyl, trifluoromethylsulfinyl, methylsulfonyl, trifluoromethylsulfonyl, acetylamino, and
A, R¹, n and X have a meaning as described herein.

According to a still further embodiment, the present invention provides compounds according to formula (I), wherein
m is 1, 2 or 3,
each Y is independently selected from the group consisting of hydrogen, fluoro, chloro, cyano, trifluoromethyl, methoxy, trifluoromethoxy, methylamino, dimethylamino, methylsulfanyl, trifluoromethylsulfanyl, methylsulfonyl, acetylamino, or
each Y is independently selected from the group consisting of fluoro and

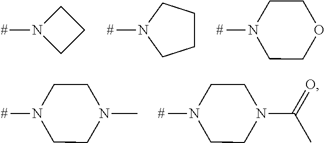

and
A, R¹, n and X have a meaning as described herein.

According to a still further embodiment, the present invention provides compounds according to formula (I), wherein
m is 1, 2 or 3,
each Y is independently selected from the group consisting of hydrogen, fluoro, chloro, cyano, trifluoromethyl, methoxy, trifluoromethoxy, methylamino, dimethylamino, methylsulfanyl, trifluoromethylsulfanyl, methylsulfonyl, acetylamino, and
A, R¹, n and X have a meaning as described herein.

Aside from the individual embodiments, any possible combination of the afore-mentioned individual embodiments provides compounds according to formula (I) within the scope of the present invention.

These combinations lead to additional particular embodiments within the scope of the present invention, some of which are illustrated by the following specific embodiments by way of example.

The present invention relates to compounds of formula (I)

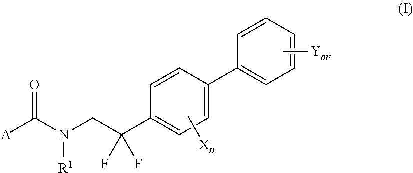

wherein
R¹ is selected from the group consisting of hydrogen, —CHO, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, C₃-C₆-halogenocycloalkyl having 1 to 5 halogen atoms, C₃-C₄-alkenyl, C₃-C₄-alkynyl, C₁-C₄-alkoxy-C₁-C₄-alkyl, C₃-C₆-cycloalkyl-C₁-C₃-alkyl, cyano-C₁-C₄-alkyl, amino-C₁-C₄-alkyl, C₁-C₄-alkylamino-C₁-C₄-alkyl, di-(C₁-C₄-alkyl)amino-C₁-C₄-alkyl, C₁-C₄-alkylcarbonyl, C₁-C₄-halogenoalkylcarbonyl having 1 to 5 halogen atoms, C₁-C₄-alkoxycarbonyl, benzyloxycarbonyl, C₁-C₄-alkoxy-C₁-C₄-alkylcarbonyl, —S(O)₂—C₁-C₄-alkyl, and —S(O)₂—C₁-C₄-halogenoalkyl having 1 to 5 halogen atoms,
n is 1, 2 or 3,
each X is independently selected from the group consisting of halogen, nitro, cyano, hydroxy, amino, —SH, —SF₅, —CHO, —OCHO, —NHCHO, —COOH, —CONH₂, —CONH(OH), —OCONH₂, (hydroxyimino)-C₁-C₆-alkyl, C₁-C₈-alkyl, C₁-C₈-halogenoalkyl having 1 to 5 halogen atoms, C₂-C₈-alkenyl, C₂-C₈-alkynyl, C₁-C₈-alkylamino, di-(C₁-C₈-alkyl)amino, C₁-C₈-alkoxy, C₁-C₈-halogenoalkoxy having 1 to 5 halogen atoms, C₂-C₈-alkenyloxy, C₂-C₈-halogenoalkenyloxy having 1 to 5 halogen atoms, C₃-C₈-alkynyloxy, C₃-C₈-halogenoalkynyloxy having 1 to 5 halogen atoms, C₃-C₈-cycloalkyl, C₃-C₈-halogenocycloalkyl having 1 to 5 halogen atoms, C₁-C₈-alkylcarbonyl, C₁-C₈-halogenoalkylcarbonyl having 1 to 5 halogen atoms, —CONH(C₁-C₈-alkyl), —CON(C₁-C₈-alkyl)₂, —CONH(OC₁-C₈-alkyl), —CON(OC₁-C₈-alkyl)(C₁-C₈-alkyl), C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, —OCONH(C₁-C₈-alkyl), —OCON(C₁-C₈-alkyl)₂, —OCONH(OC₁-C₈-alkyl), —OCO(OC₁-C₈-alkyl), —S—C₁-C₈-alkyl, —S—C₁-C₈-halogenoalkyl having 1 to 5 halogen atoms, —S(O)—C₁-C₈-alkyl, —S(O)—C₁-C₈-halogenoalkyl having 1 to 5 halogen atoms, —S(O)₂—C₁-C₈-alkyl, —S(O)₂—C₁-C₈-halogenoalkyl having 1 to 5 halogen atoms, (C₁-C₆-alkoxyimino)-C₁-C₆-alkyl, (C₂-C₆-alkenyloxyimino)-C₁-C₆-alkyl, (C₃-C₆-alkynyloxyimino)-C₁-C₆-alkyl, (benzyloxyimino)-C₁-C₆-alkyl, benzyloxy, —S-benzyl, benzylamino, phenoxy, —S-phenyl and phenylamino,
m is 0,1,2,3 or 4,
each Y is independently selected from the group consisting of hydrogen, halogen, nitro, cyano, hydroxy, amino, —SH, —SF₅, —CHO, —OCHO, —NHCHO, —COOH, —CONH₂, —CONH(OH), —OCONH₂, (hydroxyimino)-C₁-C₆-alkyl, C₁-C₈-alkyl, C₁-C₈-halogenoalkyl having 1 to 5 halogen atoms, C₂-C₈-alkenyl, C₂-C₈-alkynyl, C₁-C₈-alkylamino, di-(C₁-C₈-alkyl)amino, C₁-C₈-alkoxy, C₁-C₈-halogenoalkoxy having 1 to 5 halogen atoms, C₂-C₈-alkenyloxy, C₂-C₈-halogenoalkenyloxy having 1 to 5 halogen atoms, C₃-C₈-alkynyloxy, C₃-C₈-halogenoalkynyloxy having 1 to 5 halogen atoms, C₃-C₈-cycloalkyl, C₃-C₈-halogenocycloalkyl having 1 to 5 halogen atoms, C₁-C₈-alkylcarbonyl, C₁-C₈-halogenoalkylcarbonyl having 1 to 5 halogen atoms, —CONH(C₁-C₈-alkyl), —CON(C₁-C₈-alkyl)₂, —CONH(OC₁-C₈-alkyl), —CON(OC₁-C₈-alkyl)(C₁-C₈-alkyl), C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, —OCONH(C₁-C₈-alkyl), —OCON(C₁-C₈-alkyl)₂, —OCONH(OC₁-C₈-alkyl), —OCO(OC₁-C₈-alkyl), —S—C₁-C₈-alkyl, —S—C₁-C₈-halogenoalkyl having 1 to 5 halogen atoms, —S(O)—C₁-C₈-alkyl, —S(O)—C₁-C₈-halogenoalkyl having 1 to 5 halogen atoms, —S(O)₂—C₁-C₈-alkyl, —S(O)₂—C₁-C₈-halogenoalkyl having 1 to 5 halogen atoms, —CH₂—S—C₁-C₈-alkyl, —CH₂—S(O)—C₁-C₈-alkyl, —CH₂—S(O)₂—C₁-C₈-alkyl, (C₁-C₆-alkoxyimino)-C₁-C₆-alkyl, (C₂-C₆-alkenyloxyimino)-C₁-C₆-alkyl, (C₃-C₆-alkynyloxyimino)-C₁-C₆-alkyl, or
each Y is independently selected from a 4 to 7 membered heterocyclic ring system with 1 to 3 heteroatoms, but not O—O, O—S or 3 heteroatoms in a row, independently substituted by oxo, thiono or 1 to 12 substituents Z independently selected from the group consisting of hydrogen, halogen, cyano, hydroxy, amino, —SH, —CHO, —COOH, —CONH₂, —CONH(OH), —OCONH₂, (hydroxyimino)-C₁-C₆-alkyl, C₁-C₆-alkyl, C₁-C₆-halogenoalkyl having 1 to 5 halogen atoms, C₂-C₆-alkenyl, C₂-C₆-alkynyl, C₁-C₆-alkylamino, di-(C₁-C₆-alkyl)amino, C₁-C₆-alkoxy, C₁-C₆-halogenoalkoxy having 1 to 5 halogen atoms, C₂-C₆-alkenyloxy, C₂-C₆-halogenoalkenyloxy having 1 to 5 halogen atoms, C₃-C₆-alkynyloxy, C₃-C₆-halogenoalkynyloxy having 1 to 5 halogen atoms, C₃-C₆-cycloalkyl, C₃-C₆-halogenocycloalkyl having 1 to 5 halogen atoms, C₁-C₆-alkylcarbonyl, C₁-C₆-halogenoalkylcarbonyl having 1 to 5 halogen atoms, —CONH(C₁-C₆-alkyl), —CON(C₁-C₆-alkyl)₂, —CONH(OC₁-C₆-alkyl), —CON(OC₁-C₆-alkyl)(C₁-C₆-alkyl), C₁-C₆-alkoxycarbonyl, C₁-C₆-halogenoalkoxycarbonyl having 1 to 5 halogen atoms, C₁-C₆-alkylcarbonyloxy, C₁-C₆-halogenoalkylcarbonyloxy having 1 to 5 halogen atoms, C₁-C₆-alkylcarbonylamino, C₁-C₆-halogenoalkylcarbonylamino having 1 to 5 halogen atoms, —OCONH(C₁-C₆-alkyl), —OCON(C₁-C₆-alkyl)₂, —OCONH(OC₁-C₆-alkyl), —OCO(OC₁-C₆-alkyl), —S—C₁-C₆-alkyl, —S—C₁-C₆-halogenoalkyl having 1 to 5 halogen atoms, —S(O)—C₁-C₆-alkyl, —S(O)—C₁-C₆-halogenoalkyl having 1 to 5 halogen atoms, —S(O)₂—C₁-C₆-alkyl, —S(O)₂—C₁-C₆-halogenoalkyl having 1 to 5 halogen atoms, and
A represents a phenyl group of the formula (A1)

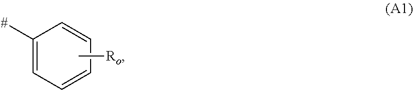

in which
# depicts the bond which connects A to the rest of the molecule,
o is 0, 1, 2, 3, 4 or 5, and
each R is independently selected from the group consisting of halogen, nitro, —OH, NH₂, SH, SF₅, CHO, OCHO, NHCHO, COOH, cyano, C₁-C₈-alkyl, C₁-C₈-halogenoalkyl having 1 to 9 halogen atoms, C₂-C₈-alkenyl, C₂-C₈-alkynyl, C₃-C₆-cycloalkyl, —S—C₁-C₈-alkyl, —S—C₁-C₈-halogenoalkyl having 1 to 5 halogen atoms, C₁-C₈-alkoxy, C₁-C₈-halogenoalkoxy having 1 to 5 halogen atoms, C₁-C₈-alkoxy-C₂-C₈-alkenyl, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, —S(O)—C₁-C₈-alkyl, —S(O)—C₁-C₈-halogenoalkyl having 1 to 5 halogen atoms, —S(O)₂—C₁-C₈-alkyl, —S(O)₂—C₁-C₈-halogenoalkyl having 1 to 5 halogen atoms, C₁-C₈-alkylsulfonamide, —NH(C₁-C₈-alkyl), N(C₁-C₈-alkyl)₂, phenyl (optionally substituted by C₁-C₆-alkoxy) and phenoxy, wherein two R bonded to adjacent carbon atoms may together represent —O(CH₂)ₚO—, wherein p represents 1 or 2, or
A represents a heterocycle of the formula (Het-1)

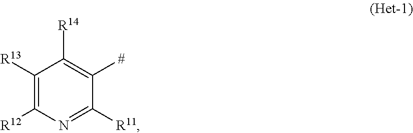

in which
# depicts the bond which connects A to the rest of the molecule,
R¹¹ is selected from the group consisting of hydrogen, halogen, hydroxy, cyano, C₁-C₄-alkyl, C₁-C₄-halogenoalkyl having 1 to 5 halogen atoms, C₁-C₄ alkoxy, —S—C₁-C₈-alkyl, S(O)—C₁-C₄-alkyl, —S(O)₂—C₁-C₄-alkyl, —S—C₂-C₅-alkenyl, —S—C₁-C₄-halogenoalkyl having 1 to 5 halogen atoms, C₁-C₄-halogenoalkoxy having 1 to 5 halogen atoms,
R¹², R¹³ and R¹⁴, which may be the same or be different, are selected from the group consisting of hydrogen, halogen, cyano, C₁-C₄-alkyl, C₁-C₄-halogenoalkyl having 1 to 5 halogen atoms, C₁-C₄-alkoxy, —S—C₁-C₄-alkyl, C₁-C₄-halogenoalkoxy having 1 to 5 halogen atoms, —S(O)—C₁-C₄-alkyl, —S(O)₂—C₁-C₄-alkyl,
A represents a heterocycle of the formula (Het-2)

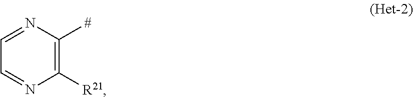

in which
# depicts the bond which connects A to the rest of the molecule, and
R²¹ is selected from the group consisting of hydrogen, halogen, C₁-C₄-alkyl and C₁-C₄-halogenoalkyl having 1 to 5 halogen atoms, or
A represents a heterocycle of the formula (Het-3)

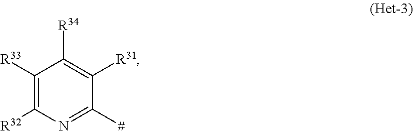

in which
# depicts the bond which connects A to the rest of the molecule,
R³¹ is selected from the group consisting of hydrogen, halogen, hydroxy, cyano, C₁-C₄-alkyl, C₁-C₄-halogenoalkyl having 1 to 5 halogen atoms, C₁-C₄ alkoxy, —S—C₁-C₅-alkyl, S(O)—C₁-C₄-alkyl, —S(O)₂—C₁-C₄-alkyl, —S—C₂-C₅-alkenyl, —S—C₁-C₄-halogenoalkyl having 1 to 5 halogen atoms, C₁-C₄-halogenoalkoxy having 1 to 5 halogen atoms,
R³², R³³ and R³⁴, which may be the same or be different, are selected from the group consisting of hydrogen, halogen, cyano, C₁-C₄-alkyl, C₁-C₄-halogenoalkyl having 1 to 5 halogen atoms, C₁-C₄-alkoxy, —S—C₁-C₄-alkyl, C₁-C₄-halogenoalkoxy having 1 to 5 halogen atoms, —S(O)—C₁-C₄-alkyl, —S(O)₂—C₁-C₄-alkyl,
   and salts, solvates, solvates of the salts, N-oxides, metal complexes or metalloid complexes thereof.

In still another embodiment, the present invention provides compounds according to formula (I), wherein
A represents a phenyl group of formula (A1)

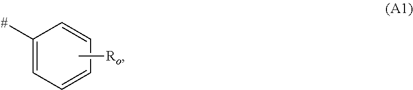

in which
# depicts the bond which connects A to the rest of the molecule,
o is 0, 1 or 2, and
each R is independently selected from the group consisting of halogen, nitro, C₁-C₄-halogenoalkyl having 1 to 5 halogen atoms, C₁-C₄-halogenoalkoxy having 1 to 5 halogen atoms, or
A represents a heterocycle of the formula (Het-1)

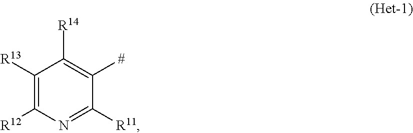

in which
# depicts the bond which connects A to the rest of the molecule,
R¹¹ is halogen or C₁-C₄-halogenoalkyl having 1 to 5 halogen atoms, and
R¹², R¹³ and R¹⁴ are hydrogen, or
A represents a heterocycle of the formula (Het-2)

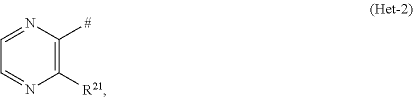

in which
# depicts the bond which connects A to the rest of the molecule, and
R²¹ is C₁-C₄-halogenoalkyl having 1 to 5 halogen atoms, or
A represents a heterocycle of the formula (Het-3)

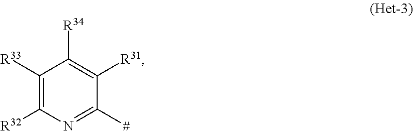

in which
# depicts the bond which connects A to the rest of the molecule,
R³¹ is C₁-C₄-halogenoalkyl having 1 to 5 halogen atoms, and
R³², R³³ and R³⁴ are hydrogen,
R¹ is selected from the group consisting of hydrogen, C₁-C₄-alkyl, C₃-C₄-alkynyl, C₃-C₆-cycloalkyl, C₁-C₄-alkylcarbonyl, C₁-C₄-alkoxy-C₁-C₄-alkyl, C₁-C₄-alkoxycarbonyl,
n is 1 or 2,
each X is independently selected from the group consisting of halogen, nitro, cyano, C₁-C₄-alkyl, C₁-C₄-halogenoalkyl having 1 to 5 halogen atoms, C₂-C₄-alkenyl, C₂-C₄-alkynyl, C₁-C₄-alkylamino, di-(C₁-C₄-alkyl)amino, C₁-C₄-alkoxy, C₁-C₄-halogenoalkoxy having 1 to 5 halogen atoms, C₂-C₄-alkenyloxy, C₂-C₄-halogenoalkenyloxy having 1 to 5 halogen atoms, C₃-C₄-alkynyloxy, C₃-C₄-halogenoalkynyloxy having 1 to 5 halogen atoms, C₃-C₆-cycloalkyl, C₃-C₆-halogenocycloalkyl having 1 to 5 halogen atoms, C₁-C₄-alkylcarbonyl, C₁-C₄-halogenoalkylcarbonyl having 1 to 5 halogen atoms, —CONH(C₁-C₄-alkyl), —CON(C₁-C₄-alkyl)₂, —CONH(OC₁-C₄-alkyl), —CON(OC₁-C₄-alkyl)(C₁-C₄-alkyl), C₁-C₄-alkoxycarbonyl, C₁-C₄-halogenoalkoxycarbonyl having 1 to 5 halogen atoms, C₁-C₄-alkylcarbonyloxy, C₁-C₄-halogenoalkylcarbonyloxy having 1 to 5 halogen atoms, C₁-C₄-alkylcarbonylamino, C₁-C₄-halogenoalkylcarbonylamino having 1 to 5 halogen atoms, —OCONH(C₁-C₄-alkyl), —OCON(C₁-C₄-alkyl)₂, —OCONH(OC₁-C₄-alkyl), —OCO(OC₁-C₄-alkyl), —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, (C₁-C₄-alkoxyimino)-C₁-C₄-alkyl, (C₂-C₆-alkenyloxyimino)-C₁-C₄-alkyl, (C₃-C₆-alkynyloxyimino)-C₁-C₄-alkyl, (benzyloxyimino)-C₁-C₆-alkyl, benzyloxy, —S-benzyl, benzylamino, phenoxy, —S-phenyl and phenylamino,
m is 1, 2 or 3, and
each Y is independently selected from the group consisting of hydrogen, halogen, nitro, cyano, hydroxy, amino, —SH, —SF₅, —CHO, —OCHO, —NHCHO, —COOH, —CONH₂, —CONH(OH), —OCONH₂, (hydroxyimino)-C₁-C₆-alkyl, C₁-C₆-alkyl, C₁-C₆-halogenoalkyl having 1 to 5 halogen atoms, C₂-C₆-alkenyl, C₂-C₆-alkynyl, C₁-C₆-alkylamino, di-(C₁-C₆-alkyl)amino, C₁-C₆-alkoxy, C₁-C₆-halogenoalkoxy having 1 to 5 halogen atoms, C₂-C₆-alkenyloxy, C₂-C₈-halogenoalkenyloxy having 1 to 5 halogen atoms, C₃-C₆-alkynyloxy, C₃-C₆-halogenoalkynyloxy having 1 to 5 halogen atoms, C₃-C₆-cycloalkyl, C₃-C₆-halogenocycloalkyl having 1 to 5 halogen atoms, C₁-C₆-alkylcarbonyl, C₁-C₆-halogenoalkylcarbonyl having 1 to 5 halogen atoms, —CONH(C₁-C₆-alkyl), —CON(C₁-C₆-alkyl)₂, —CONH(OC₁-C₆-alkyl), —CON(OC₁-C₆-alkyl)(C₁-C₆-alkyl), C₁-C₆-alkoxycarbonyl, C₁-C₆-halogenoalkoxycarbonyl having 1 to 5 halogen atoms, C₁-C₆-alkylcarbonyloxy, C₁-C₆-halogenoalkylcarbonyloxy having 1 to 5 halogen atoms, C₁-C₆-alkylcarbonylamino, C₁-C₆-halogenoalkylcarbonylamino having 1 to 5 halogen atoms, —OCONH(C₁-C₆-alkyl), —OCON(C₁-C₆-alkyl)₂, —OCONH(OC₁-C₆-alkyl), —OCO(OC₁-C₆-alkyl), —S—C₁-C₆-alkyl, —S—C₁-C₆-halogenoalkyl having 1 to 5 halogen atoms, —S(O)—C₁-C₆-alkyl, —S(O)—C₁-C₆-halogenoalkyl having 1 to 5 halogen atoms, —S(O)₂—C₁-C₆-alkyl, —S(O)₂—C₁-C₆-halogenoalkyl having 1 to 5 halogen atoms, —CH₂—S—C₁-C₆-alkyl, —CH₂—S(O)—C₁-C₆-alkyl, —CH₂—S(O)₂—C₁-C₆-alkyl, (C₁-C₄-alkoxyimino)-C₁-C₄-alkyl, (C₂-C₄-alkenyloxyimino)-C₁-C₄-alkyl, (C₃-C₆-alkynyloxyimino)-C₁-C₄-alkyl, wherein two Y may additionally be selected from —O—C₁-C₃-alkoxy and —O—C₁-C₃-halogenalkoxy having 1 to 5 halogen atoms, with each oxy function being the connecting atom of the individual Y, thereby forming an annelated 5-, 6- or 7-membered ring with the phenyl moiety that they are connected to, or
each Y is independently selected from the group consisting of hydrogen, halogen, cyano, C₁-C₆-alkyl, C₁-C₆-halogenoalkyl having 1 to 5 halogen atoms, and

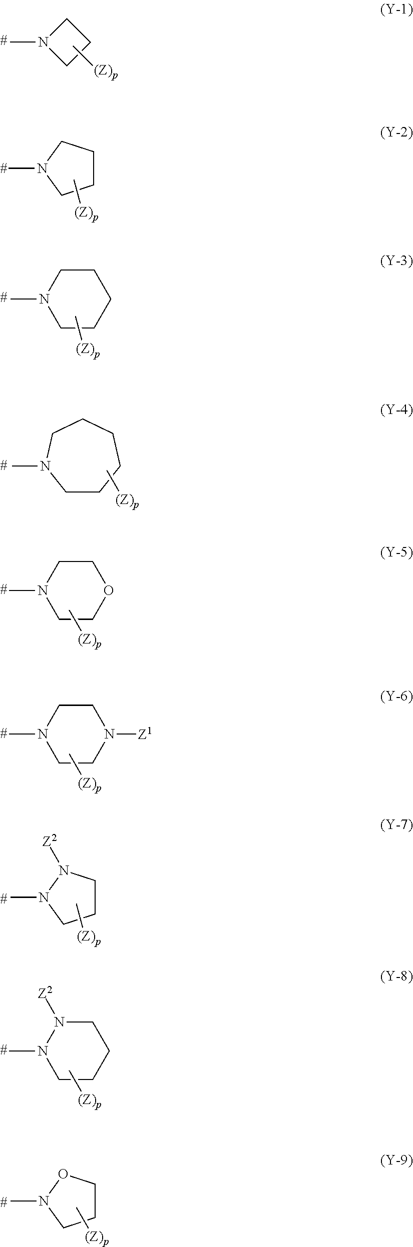

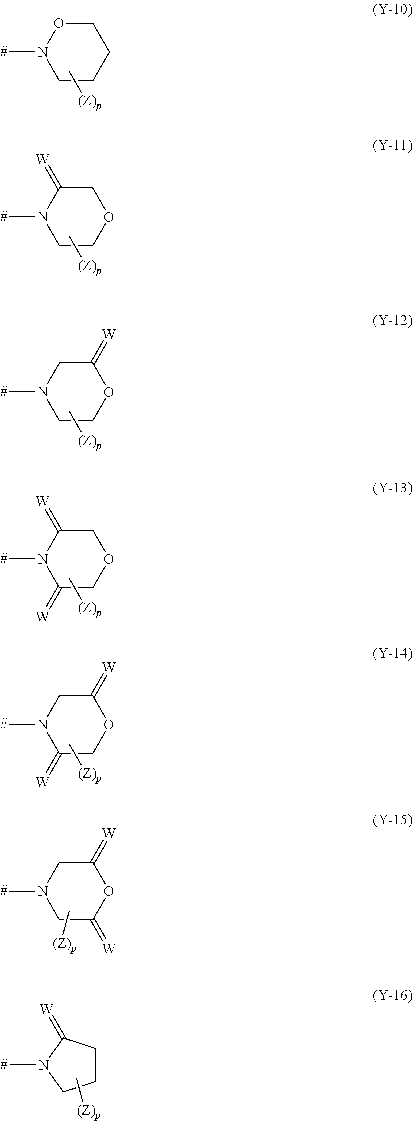

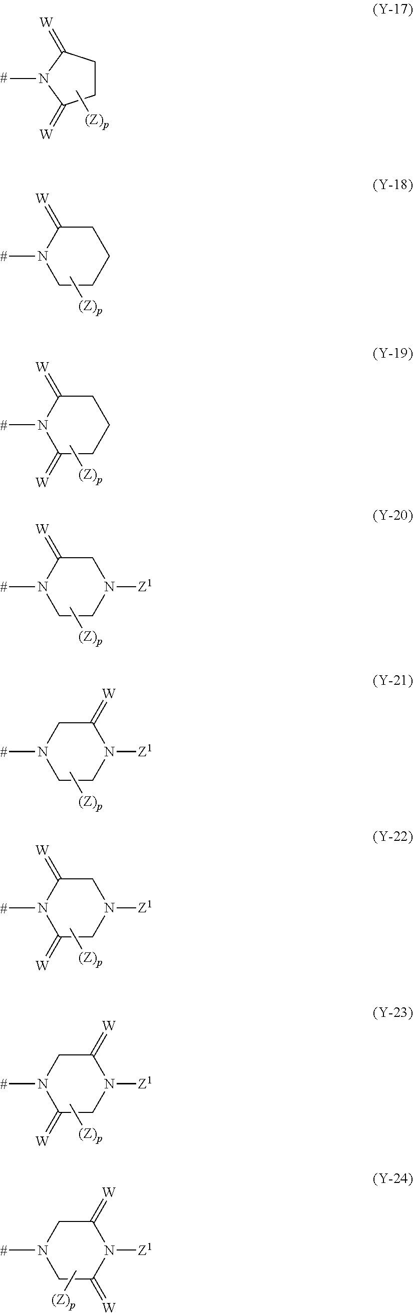

in which
# depicts the bond which connects Y to the rest of the molecule,
W is oxygen or sulfur,
p is 0, 1, 2, 3, 4, 5 or 6,
Z is independently selected from the group consisting of hydrogen, halogen, cyano, hydroxy, amino, —SH, —CHO, —COOH, —CONH₂, —CONH(OH), —OCONH₂, (hydroxyimino)-C₁-C₆-alkyl, C₁-C₆-alkyl, C₁-C₆-halogenoalkyl having 1 to 5 halogen atoms, C₁-C₆-alkylamino, di-(C₁-C₆-alkyl)amino, C₁-C₆-alkoxy, C₁-C₆-halogenoalkoxy having 1 to 5 halogen atoms, C₃-C₆-cycloalkyl, C₃-C₆-halogenocycloalkyl having 1 to 5 halogen atoms, —CONH(C₁-C₆-alkyl), —CON(C₁-C₆-alkyl)₂, —S—C₁-C₆-alkyl, —S—C₁-C₆-halogenoalkyl having 1 to 5 halogen atoms, —S(O)—C₁-C₆-alkyl, —S(O)—C₁-C₆-halogenoalkyl having 1 to 5 halogen atoms, —S(O)₂—C₁-C₆-alkyl, —S(O)₂—C₁-C₆-halogenoalkyl having 1 to 5 halogen atoms,
Z¹ is independently selected from the group consisting of hydrogen, C₁-C₆-alkyl, C₁-C₆-halogenoalkyl having 1 to 5 halogen atoms, C₁-C₆-alkylamino, di-(C₁-C₆-alkyl)amino, C₁-C₆-alkoxy, C₁-C₆-alkylcarbonyl, C₃-C₆-cycloalkyl, C₃-C₆-halogenocycloalkyl having 1 to 5 halogen atoms, —CONH(C₁-C₆-alkyl), —CON(C₁-C₆-alkyl)₂, and
Z² is independently selected from the group consisting of hydrogen, C₁-C₆-alkyl, C₁-C₆-halogenoalkyl having 1 to 5 halogen atoms, C₃-C₆-cycloalkyl, C₃-C₆-halogenocycloalkyl having 1 to 5 halogen atoms, —CONH(C₁-C₆-alkyl), —CON(C₁-C₆-alkyl)₂,
   and salts, solvates, solvates of the salts, N-oxides, metal complexes or metalloid complexes thereof.

In yet another embodiment, the present invention provides compounds according to formula (I), wherein
A represents a phenyl group of formula (A1)

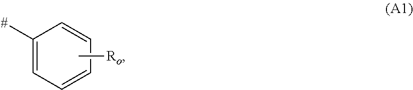

in which
# depicts the bond which connects A to the rest of the molecule,
o is 0, 1 or 2, and
each R is independently selected from the group consisting of halogen, nitro, C₁-C₄-halogenoalkyl having 1 to 5 halogen atoms, C₁-C₄-halogenoalkoxy having 1 to 5 halogen atoms, or
A represents a heterocycle of the formula (Het-1)

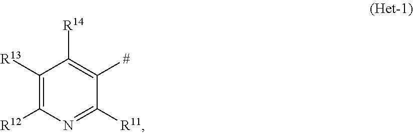

in which
# depicts the bond which connects A to the rest of the molecule,
R¹¹ is halogen or C₁-C₄-halogenoalkyl having 1 to 5 halogen atoms, and
R¹², R¹³ and R¹⁴ are hydrogen, or
A represents a heterocycle of the formula (Het-2)

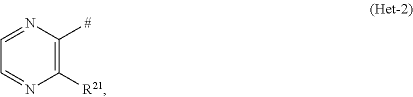

in which
# depicts the bond which connects A to the rest of the molecule, and
R²¹ is C₁-C₄-halogenoalkyl having 1 to 5 halogen atoms, or
A represents a heterocycle of the formula (Het-3)

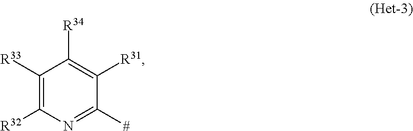

in which
# depicts the bond which connects A to the rest of the molecule,
R³¹ is C₁-C₄-halogenoalkyl having 1 to 5 halogen atoms, and
R³², R³³ and R³⁴ are hydrogen,
R¹ is selected from the group consisting of hydrogen, C₁-C₄-alkyl, C₃-C₄-alkynyl, C₃-C₆-cycloalkyl, C₁-C₄-alkylcarbonyl, C₁-C₄-alkoxy-C₁-C₄-alkyl, C₁-C₄-alkoxycarbonyl,
n is 1 or 2,
each X is independently selected from the group consisting of halogen, cyano, C₁-C₄-alkyl, C₁-C₄-alkoxy and C₁-C₄-halogenoalkyl having 1 to 5 halogen atoms,
m is 1, 2 or 3, and
each Y is independently selected from the group consisting of hydrogen, halogen, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —CONH(C₁-C₄-alkyl), —CON(C₁-C₄-alkyl)₂, —CONH(OC₁-C₄-alkyl), —CON(OC₁-C₄-alkyl)(C₁-C₆-alkyl), —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino, wherein two Y may additionally be selected from —O—C₁-C₂-alkoxy and —O—C₁-C₂-halogenalkoxy having 1 to 5 halogen atoms, with each oxy function being the connecting atom of the individual Y, thereby forming an annelated 5- or 6-membered ring with the phenyl moiety that they are connected to, or
each Y is independently selected from the group consisting of hydrogen, halogen, cyano, C₁-C₄-alkyl, C₁-C₄-halogenoalkyl having 1 to 5 halogen atoms, and

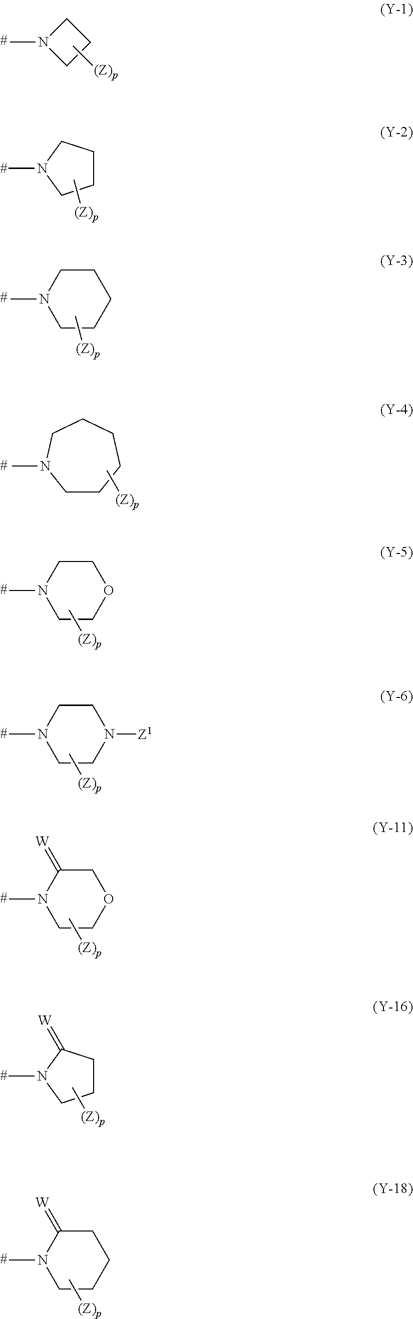

in which
# depicts the bond which connects Y to the rest of the molecule,
W is oxygen,
p is 0, 1, 2, 3, 4, 5 or 6,
Z is independently selected from the group consisting of hydrogen, halogen, cyano, hydroxy, amino, —SH, —CHO, —COOH, —CONH₂, —CONH(OH), —OCONH₂, (hydroxyimino)-C₁-C₄-alkyl, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, C₃-C₆-cycloalkyl, C₃-C₆-halogenocycloalkyl having 1 to 5 halogen atoms, —CONH(C₁-C₄-alkyl), —CON(C₁-C₄-alkyl)₂, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, and
Z¹ is independently selected from the group consisting of hydrogen, C₁-C₆-alkyl, C₁-C₆-halogenoalkyl having 1 to 5 halogen atoms, C₁-C₆-alkylamino, di (C₁-C₆-alkylamino, C₁-C₆-alkoxy, C₁-C₆-alkylcarbonyl, C₃-C₆-cycloalkyl, C₃-C₆-halogenocycloalkyl having 1 to 5 halogen atoms, —CONH(C₁-C₆-alkyl), —CON(C₁-C₆-alkyl)₂,
   and salts, solvates, solvates of the salts, N-oxides, metal complexes or metalloid complexes thereof.

In yet another embodiment, the present invention provides compounds according to formula (I), wherein
A represents a phenyl group of formula (A1)

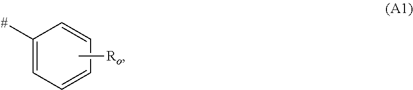

in which
depicts the bond which connects A to the rest of the molecule,
o is 1 or 2, and
each R is independently selected from the group consisting of halogen, nitro, —CF₃, —OCF₃, —CHF₂, or
A represents a heterocycle of the formula (Het-1)

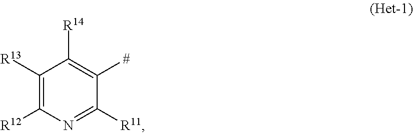

in which
# depicts the bond which connects A to the rest of the molecule,
R¹¹ is halogen or CF₃, and
R¹², R¹³ and R¹⁴ are hydrogen, or
A represents a heterocycle of the formula (Het-2)

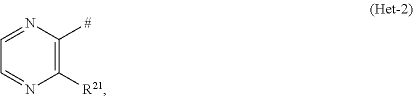

in which
# depicts the bond which connects A to the rest of the molecule, and
R²¹ is CF₃, or
A represents a heterocycle of the formula (Het-3)

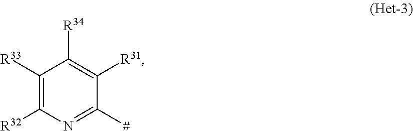

in which
# depicts the bond which connects A to the rest of the molecule,
R³¹ is CF₃, and
R³², R³³ and R³⁴ are hydrogen,
R¹ is hydrogen,
n is 1 or 2,
each X is independently selected from the group consisting of halogen, cyano, CH₃, OCH₃ and CF₃,
m is 1, 2 or 3, and
each Y is independently selected from the group consisting of hydrogen, halogen, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino, wherein two Y may additionally be selected from —O—CH₂—O— and —O—CF₂—O—, with each oxy function being the connecting atom of the individual Y, thereby forming an annelated 5-membered ring with the phenyl moiety that they are connected to, or
each Y is independently selected from the group consisting of hydrogen, fluoro, chloro, cyano, CF₃, and

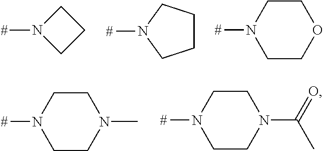

and salts, solvates, solvates of the salts, N-oxides, metal complexes or metalloid complexes thereof.

In yet another embodiment, the present invention provides compounds according to formula (I), wherein
A represents a phenyl group of formula (A1)

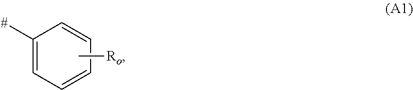

in which
# depicts the bond which connects A to the rest of the molecule,
o is 1 or 2, and
each R is independently selected from the group consisting of halogen, nitro, —CF₃, —OCF₃, —CHF₂, or
A represents a heterocycle of the formula (Het-1)

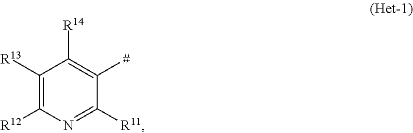

in which
# depicts the bond which connects A to the rest of the molecule,
R¹¹ is halogen or CF₃, and
R¹², R¹³ and R¹⁴ are hydrogen, or
A represents a heterocycle of the formula (Het-2)

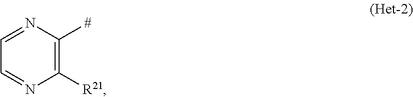

in which
# depicts the bond which connects A to the rest of the molecule, and
R²¹ is CF₃, or
A represents a heterocycle of the formula (Het-3)

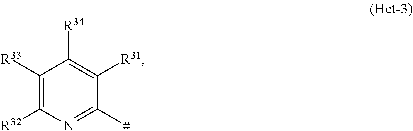

in which
# depicts the bond which connects A to the rest of the molecule,
R³¹ is CF₃, and
R³², R³³ and R³⁴ are hydrogen,
R¹ is hydrogen,
n is 1 or 2,
each X is independently selected from the group consisting of halogen, cyano, CH₃, OCH₃ and CF₃,
m is 1, 2 or 3, and
each Y is independently selected from the group consisting of hydrogen, halogen, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino,
   and salts, solvates, solvates of the salts, N-oxides, metal complexes or metalloid complexes thereof.

In yet another embodiment, the present invention provides compounds according to formula (I), wherein
A is selected from the group consisting of

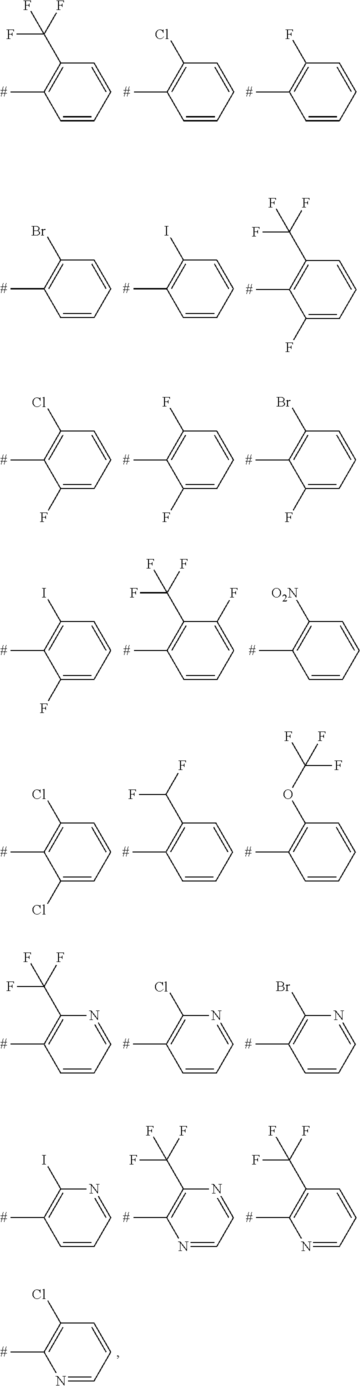

in which
# depicts the bond which connects A to the rest of the molecule,
R¹ is hydrogen,
n is 1 or 2,
each X is independently selected from the group consisting of chloro, fluoro, cyano, CH₃, OCH₃ and CF₃,
m is 1, 2 or 3, and
each Y is independently selected from the group consisting of hydrogen, halogen, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino, wherein two Y may additionally be selected from —O—CH₂—O— and —O—CF₂—O—, with each oxy function being the connecting atom of the individual Y, thereby forming an annelated 5-membered ring with the phenyl moiety that they are connected to, or
each Y is independently selected from the group consisting of hydrogen, fluoro, chloro, cyano, CF₃, and

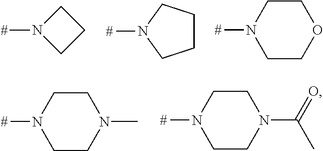

and salts, solvates, solvates of the salts, N-oxides, metal complexes or metalloid complexes thereof.

In yet another embodiment, the present invention provides compounds according to formula (I), wherein
A is selected from the group consisting of

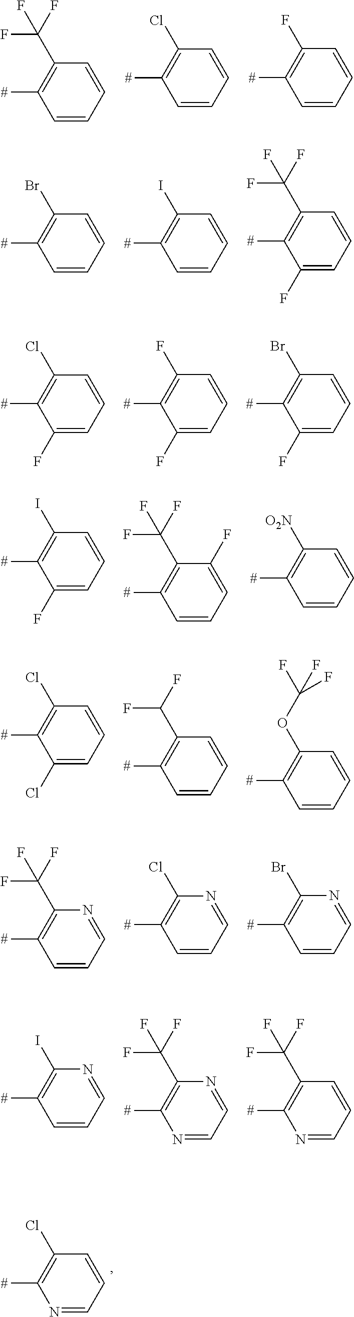

in which
# depicts the bond which connects A to the rest of the molecule,
R¹ is hydrogen,
n is 1 or 2,
each X is independently selected from the group consisting of chloro, fluoro, cyano, CH₃, OCH₃ and CF₃,
m is 1, 2 or 3, and
each Y is independently selected from the group consisting of hydrogen, halogen, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino,
   and salts, solvates, solvates of the salts, N-oxides, metal complexes or metalloid complexes thereof.

In yet another embodiment, the present invention provides compounds according to formula (I), wherein
A is selected from the group consisting of

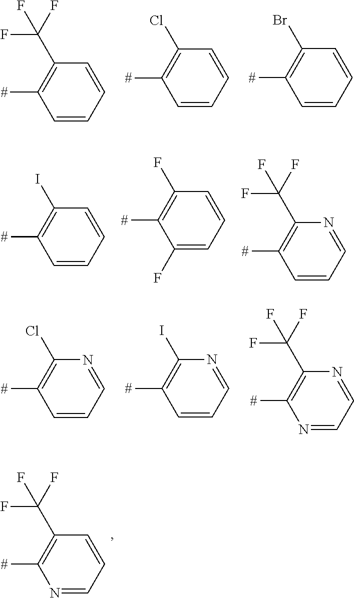

in which
# depicts the bond which connects A to the rest of the molecule,
R¹ is hydrogen,
n is 1 or 2,
each X is independently selected from the group consisting of chloro, fluoro, cyano, CH₃, OCH₃ and CF₃,
m is 1, 2 or 3, and
each Y is independently selected from the group consisting of hydrogen, fluoro, chloro, bromo, cyano, trifluoromethyl, difluoromethyl, methoxy, trifluoromethoxy, difluoromethoxy, methylamino, dimethylamino, methylsulfanyl, trifluoromethylsulfanyl, methylsulfinyl, trifluoromethylsulfinyl, methylsulfonyl, trifluoromethylsulfonyl, and acetylamino, or
each Y is independently selected from the group consisting of hydrogen, fluoro, chloro, cyano, CF₃, and

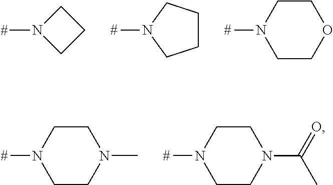

and salts, solvates, solvates of the salts, N-oxides, metal complexes or metalloid complexes thereof.

In yet another embodiment, the present invention provides compounds according to formula (I), wherein
A is selected from the group consisting of

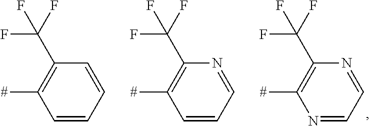

in which
# depicts the bond which connects A to the rest of the molecule,
R¹ is hydrogen,
n is 1 or 2,
each X is independently selected from the group consisting of chloro, fluoro, cyano, CH₃, OCH₃ and CF₃,
m is 1, 2 or 3, and
each Y is independently selected from the group consisting of hydrogen, fluoro, chloro, bromo, cyano, trifluoromethyl, difluoromethyl, methoxy, trifluoromethoxy, difluoromethoxy, methylamino, dimethylamino, methylsulfanyl, trifluoromethylsulfanyl, methylsulfinyl, trifluoromethylsulfinyl, methylsulfonyl, trifluoromethylsulfonyl, and acetylamino,
   and salts, solvates, solvates of the salts, N-oxides, metal complexes or metalloid complexes thereof.

In yet another embodiment, the present invention provides compounds according to formula (I), wherein
A is selected from the group consisting of

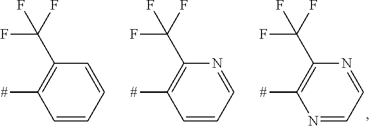

in which
# depicts the bond which connects A to the rest of the molecule,
R¹ is hydrogen,
n is 1 or 2,
each X is independently selected from the group consisting of chloro, fluoro, CH₃ and CF₃,
m is 1, 2 or 3, and
each Y is independently selected from the group consisting of hydrogen, fluoro and chloro,
   and salts, solvates, solvates of the salts, N-oxides, metal complexes or metalloid complexes thereof.

In yet another embodiment, the present invention provides compounds according to formula (I), wherein
A is selected from the group consisting of

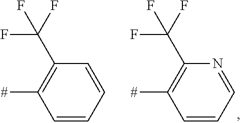

in which
   # depicts the bond which connects A to the rest of the molecule,
R¹ is hydrogen,
n is 1 or 2,
each X is independently selected from the group consisting of chloro, fluoro, CH₃, OCH₃ and CF₃,
m is 1, 2 or 3, and
each Y is independently selected from the group consisting of hydrogen, fluoro, chloro, cyano, trifluoromethyl, methoxy, trifluoromethoxy, methylamino, dimethylamino, methylsulfanyl, trifluoromethylsulfanyl, methylsulfonyl, and acetylamino,
   and salts, solvates, solvates of the salts, N-oxides, metal complexes or metalloid complexes thereof.

The present invention relates further to compounds of formula (I)

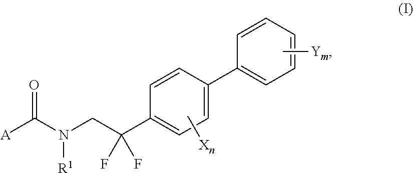

wherein
R¹ is selected from the group consisting of hydrogen, —CHO, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, C₃-C₆-halogenocycloalkyl having 1 to 5 halogen atoms, C₃-C₄-alkenyl, C₃-C₄-alkynyl, C₁-C₄-alkoxy-C₁-C₄-alkyl, C₃-C₆-cycloalkyl-C₁-C₃-alkyl, cyano-C₁-C₄-alkyl, amino-C₁-C₄-alkyl, C₁-C₄-alkylamino-C₁-C₄-alkyl, di-(C₁-C₄-alkyl)amino-C₁-C₄-alkyl, C₁-C₄-alkylcarbonyl, C₁-C₄-halogenoalkylcarbonyl having 1 to 5 halogen atoms, C₁-C₄-alkoxycarbonyl, benzyloxycarbonyl, C₁-C₄-alkoxy-C₁-C₄-alkylcarbonyl, —S(O)₂—C₁-C₄-alkyl, and —S(O)₂—C₁-C₄-halogenoalkyl having 1 to 5 halogen atoms,
n is 0, 1, 2 or 3,
each X is independently selected from the group consisting of hydrogen, halogen, nitro, cyano, hydroxy, amino, —SH, —SF₅, —CHO, —OCHO, —NHCHO, —COOH, —CONH₂, —CONH(OH), —OCONH₂, (hydroxyimino)-C₁-C₆-alkyl, C₁-C₈-alkyl, C₁-C₈-halogenoalkyl having 1 to 5 halogen atoms, C₂-C₈-alkenyl, C₂-C₈-alkynyl, C₁-C₈-alkylamino, di-(C₁-C₈-alkyl)amino, C₁-C₈-alkoxy, C₁-C₈-halogenoalkoxy having 1 to 5 halogen atoms, C₂-C₈-alkenyloxy, C₂-C₈-halogenoalkenyloxy having 1 to 5 halogen atoms, C₃-C₈-alkynyloxy, C₃-C₈-halogenoalkynyloxy having 1 to 5 halogen atoms, C₃-C₈-cycloalkyl, C₃-C₈-halogenocycloalkyl having 1 to 5 halogen atoms, C₁-C₈-alkylcarbonyl, C₁-C₈-halogenoalkylcarbonyl having 1 to 5 halogen atoms, —CONH(C₁-C₈-alkyl), —CON(C₁-C₈-alkyl)₂, —CONH(OC₁-C₈-alkyl), —CON(OC₁-C₈-alkyl)(C₁-C₈-alkyl), C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, —OCONH(C₁-C₈-alkyl), —OCON(C₁-C₈-alkyl)₂, —OCONH(OC₁-C₈-alkyl), —OCO(OC₁-C₈-alkyl), —S—C₁-C₈-alkyl, —S—C₁-C₈-halogenoalkyl having 1 to 5 halogen atoms, —S(O)—C₁-C₈-alkyl, —S(O)—C₁-C₈-halogenoalkyl having 1 to 5 halogen atoms, —S(O)₂—C₁-C₈-alkyl, —S(O)₂—C₁-C₈-halogenoalkyl having 1 to 5 halogen atoms, (C₁-C₆-alkoxyimino)-C₁-C₆-alkyl, (C₂-C₆-alkenyloxyimino)-C₁-C₆-alkyl, (C₃-C₆-alkynyloxyimino)-C₁-C₆-alkyl, (benzyloxyimino)-C₁-C₆-alkyl, benzyloxy, —S-benzyl, benzylamino, phenoxy, —S-phenyl and phenylamino,
m is 0, 1, 2, 3 or 4,
each Y is independently selected from the group consisting of hydrogen, halogen, nitro, cyano, hydroxy, amino, —SH, —SF₅, —CHO, —OCHO, —NHCHO, —COOH, —CONH₂, —CONH(OH), —OCONH₂, (hydroxyimino)-C₁-C₆-alkyl, C₁-C₈-alkyl, C₁-C₈-halogenoalkyl having 1 to 5 halogen atoms, C₂-C₈-alkenyl, C₂-C₈-alkynyl, C₁-C₈-alkylamino, di-(C₁-C₈-alkyl)amino, C₁-C₈-alkoxy, C₁-C₈-halogenoalkoxy having 1 to 5 halogen atoms, C₂-C₈-alkenyloxy, C₂-C₈-halogenoalkenyloxy having 1 to 5 halogen atoms, C₃-C₈-alkynyloxy, C₃-C₈-halogenoalkynyloxy having 1 to 5 halogen atoms, C₃-C₈-cycloalkyl, C₃-C₈-halogenocycloalkyl having 1 to 5 halogen atoms, C₁-C₈-alkylcarbonyl, C₁-C₈-halogenoalkylcarbonyl having 1 to 5 halogen atoms, —CONH(C₁-C₈-alkyl), —CON(C₁-C₈-alkyl)₂, —CONH(OC₁-C₈-alkyl), —CON(OC₁-C₈-alkyl)(C₁-C₈-alkyl), C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, —OCONH(C₁-C₈-alkyl), —OCON(C₁-C₈-alkyl)₂, —OCONH(OC₁-C₈-alkyl), —OCO(OC₁-C₈-alkyl), —S—C₁-C₈-alkyl, —S—C₁-C₈-halogenoalkyl having 1 to 5 halogen atoms, —S(O)—C₁-C₈-alkyl, —S(O)—C₁-C₈-halogenoalkyl having 1 to 5 halogen atoms, —S(O)₂—C₁-C₈-alkyl, —S(O)₂—C₁-C₈-halogenoalkyl having 1 to 5 halogen atoms, —CH₂—S—C₁-C₈-alkyl, —CH₂—S(O)—C₁-C₈-alkyl, —CH₂—S(O)₂—C₁-C₈-alkyl, (C₁-C₆-alkoxyimino)-C₁-C₆-alkyl, (C₂-C₆-alkenyloxyimino)-C₁-C₆-alkyl, (C₃-C₆-alkynyloxyimino)-C₁-C₆-alkyl, or
each Y is independently selected from a 4 to 7 membered heterocyclic ring system with 1 to 3 heteroatoms, but not O—O, O—S or 3 heteroatoms in a row, independently substituted by oxo, thiono or 1 to 12 substituents Z independently selected from the group consisting of hydrogen, halogen, cyano, hydroxy, amino, —SH, —CHO, —COOH, —CONH₂, —CONH(OH), —OCONH₂, (hydroxyimino)-C₁-C₆-alkyl, C₁-C₆-alkyl, C₁-C₆-halogenoalkyl having 1 to 5 halogen atoms, C₂-C₆-alkenyl, C₂-C₆-alkynyl, C₁-C₆-alkylamino, di-(C₁-C₆-alkyl)amino, C₁-C₆-alkoxy, C₁-C₆-halogenoalkoxy having 1 to 5 halogen atoms, C₂-C₆-alkenyloxy, C₂-C₆-halogenoalkenyloxy having 1 to 5 halogen atoms, C₃-C₆-alkynyloxy, C₃-C₆-halogenoalkynyloxy having 1 to 5 halogen atoms, C₃-C₆-cycloalkyl, C₃-C₆-halogenocycloalkyl having 1 to 5 halogen atoms, C₁-C₆-alkylcarbonyl, C₁-C₆-halogenoalkylcarbonyl having 1 to 5 halogen atoms, —CONH(C₁-C₆-alkyl), —CON(C₁-C₆-alkyl)₂, —CONH(OC₁-C₆-alkyl), —CON(OC₁-C₆-alkyl)(C₁-C₆-alkyl), C₁-C₆-alkoxycarbonyl, C₁-C₆-halogenoalkoxycarbonyl having 1 to 5 halogen atoms, C₁-C₆-alkylcarbonyloxy, C₁-C₆-halogenoalkylcarbonyloxy having 1 to 5 halogen atoms, C₁-C₆-alkylcarbonylamino, C₁-C₆-halogenoalkylcarbonylamino having 1 to 5 halogen atoms, —OCONH(C₁-C₆-alkyl), —OCON(C₁-C₆-alkyl)₂, —OCONH(OC₁-C₆-alkyl), —OCO(OC₁-C₆-alkyl), —S—C₁-C₆-alkyl, —S—C₁-C₆-halogenoalkyl having 1 to 5 halogen atoms, —S(O)—C₁-C₆-alkyl, —S(O)—C₁-C₆-halogenoalkyl having 1 to 5 halogen atoms, —S(O)₂—C₁-C₆-alkyl, —S(O)₂—C₁-C₆-halogenoalkyl having 1 to 5 halogen atoms, and
A represents a phenyl group of the formula (A1)

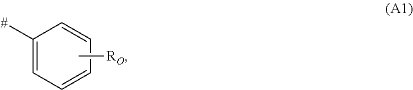

in which
# depicts the bond which connects A to the rest of the molecule,
o is 0, 1, 2, 3, 4 or 5, and
each R is independently selected from the group consisting of halogen, nitro, —OH, NH₂, SH, SF₅, CHO, OCHO, NHCHO, COOH, cyano, C₁-C₈-alkyl, C₁-C₈-halogenoalkyl having 1 to 9 halogen atoms, C₂-C₈-alkenyl, C₂-C₈-alkynyl, C₃-C₆-cycloalkyl, —S—C₁-C₈-alkyl, —S—C₁-C₈-halogenoalkyl having 1 to 5 halogen atoms, C₁-C₈-alkoxy, C₁-C₈-halogenoalkoxy having 1 to 5 halogen atoms, C₁-C₈-alkoxy-C₂-C₈-alkenyl, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, —S(O)—C₁-C₈-alkyl, —S(O)—C₁-C₈-halogenoalkyl having 1 to 5 halogen atoms, —S(O)₂—C₁-C₈-alkyl, —S(O)₂—C₁-C₈-halogenoalkyl having 1 to 5 halogen atoms, C₁-C₈-alkylsulfonamide, —NH(C₁-C₈-alkyl), N(C₁-C₈-alkyl)₂, phenyl (optionally substituted by C₁-C₆-alkoxy) and phenoxy, wherein two R bonded to adjacent carbon atoms may together represent —O(CH₂)ₚO—, wherein p represents 1 or 2, or
A represents a heterocycle of the formula (Het-1)

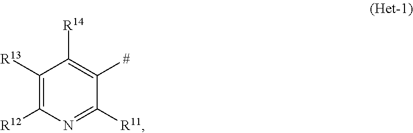

in which
# depicts the bond which connects A to the rest of the molecule,
R¹¹ is selected from the group consisting of hydrogen, halogen, hydroxy, cyano, C₁-C₄-alkyl, C₁-C₄-halogenoalkyl having 1 to 5 halogen atoms, C₁-C₄ alkoxy, —S—C₁-C₈-alkyl, S(O)—C₁-C₄-alkyl, —S(O)₂—C₁-C₄-alkyl, —S—C₂-C₅-alkenyl, —S—C₁-C₄-halogenoalkyl having 1 to 5 halogen atoms, C₁-C₄-halogenoalkoxy having 1 to 5 halogen atoms,
R¹², R¹³ and R¹⁴, which may be the same or be different, are selected from the group consisting of hydrogen, halogen, cyano, C₁-C₄-alkyl, C₁-C₄-halogenoalkyl having 1 to 5 halogen atoms, C₁-C₄-alkoxy, —S—C₁-C₄-alkyl, C₁-C₄-halogenoalkoxy having 1 to 5 halogen atoms, —S(O)—C₁-C₄-alkyl, —S(O)₂—C₁-C₄-alkyl,
A represents a heterocycle of the formula (Het-2)

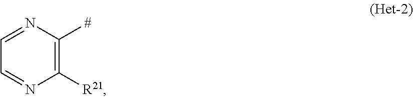

in which
# depicts the bond which connects A to the rest of the molecule, and
R²¹ is selected from the group consisting of hydrogen, halogen, C₁-C₄-alkyl and C₁-C₄-halogenoalkyl having 1 to 5 halogen atoms, or
A represents a heterocycle of the formula (Het-3)

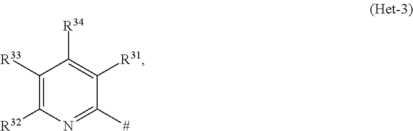

in which
# depicts the bond which connects A to the rest of the molecule,
R³¹ is selected from the group consisting of hydrogen, halogen, hydroxy, cyano, C₁-C₄-alkyl, C₁-C₄-halogenoalkyl having 1 to 5 halogen atoms, C₁-C₄ alkoxy, —S—C₁-C₅-alkyl, S(O)—C₁-C₄-alkyl, —S(O)₂—C₁-C₄-alkyl, —S—C₂-C₅-alkenyl, —S—C₁-C₄-halogenoalkyl having 1 to 5 halogen atoms, C₁-C₄-halogenoalkoxy having 1 to 5 halogen atoms,
R³², R³³ and R³⁴, which may be the same or be different, are selected from the group consisting of hydrogen, halogen, cyano, C₁-C₄-alkyl, C₁-C₄-halogenoalkyl having 1 to 5 halogen atoms, C₁-C₄-alkoxy, —S—C₁-C₄-alkyl, C₁-C₄-halogenoalkoxy having 1 to 5 halogen atoms, —S(O)—C₁-C₄-alkyl, —S(O)₂—C₁-C₄-alkyl,
   and salts, solvates, solvates of the salts, N-oxides, metal complexes or metalloid complexes thereof, for use in the control, treatment and/or prevention of infections with helminths in animals and humans.

In still another embodiment, the present invention provides compounds for use in the control, treatment and/or prevention of infections with helminths in animals and humans according to formula (I), wherein
A represents a phenyl group of formula (A1)

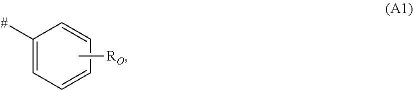

in which
# depicts the bond which connects A to the rest of the molecule,
o is 0, 1 or 2, and
each R is independently selected from the group consisting of halogen, nitro, C₁-C₄-halogenoalkyl having 1 to 5 halogen atoms, C₁-C₄-halogenoalkoxy having 1 to 5 halogen atoms, or
A represents a heterocycle of the formula (Het-1)

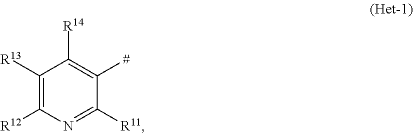

in which
# depicts the bond which connects A to the rest of the molecule,
R¹¹ is halogen or C₁-C₄-halogenoalkyl having 1 to 5 halogen atoms, and
R¹², R¹³ and R¹⁴ are hydrogen, or
A represents a heterocycle of the formula (Het-2)

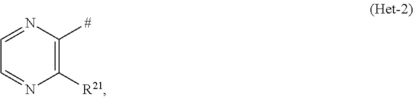

in which
# depicts the bond which connects A to the rest of the molecule, and
R²¹ is C₁-C₄-halogenoalkyl having 1 to 5 halogen atoms, or
A represents a heterocycle of the formula (Het-3)

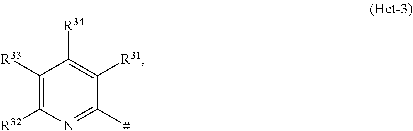

in which
# depicts the bond which connects A to the rest of the molecule,
R³¹ is C₁-C₄-halogenoalkyl having 1 to 5 halogen atoms, and
R³², R³³ and R³⁴ are hydrogen,
R¹ is selected from the group consisting of hydrogen, C₁-C₄-alkyl, C₃-C₄-alkynyl, C₃-C₆-cycloalkyl, C₁-C₄-alkylcarbonyl, C₁-C₄-alkoxy-C₁-C₄-alkyl, C₁-C₄-alkoxycarbonyl,
n is 1 or 2,
each X is independently selected from the group consisting of hydrogen, halogen, nitro, cyano, C₁-C₄-alkyl, C₁-C₄-halogenoalkyl having 1 to 5 halogen atoms, C₂-C₄-alkenyl, C₂-C₄-alkynyl, C₁-C₄-alkylamino, di-(C₁-C₄-alkyl)amino, C₁-C₄-alkoxy, C₁-C₄-halogenoalkoxy having 1 to 5 halogen atoms, C₂-C₄-alkenyloxy, C₂-C₄-halogenoalkenyloxy having 1 to 5 halogen atoms, C₃-C₄-alkynyloxy, C₃-C₄-halogenoalkynyloxy having 1 to 5 halogen atoms, C₃-C₆-cycloalkyl, C₃-C₆-halogenocycloalkyl having 1 to 5 halogen atoms, C₁-C₄-alkylcarbonyl, C₁-C₄-halogenoalkylcarbonyl having 1 to 5 halogen atoms, —CONH(C₁-C₄-alkyl), —CON(C₁-C₄-alkyl)₂, —CONH(OC₁-C₄-alkyl), —CON(OC₁-C₄-alkyl)(C₁-C₄-alkyl), C₁-C₄-alkoxycarbonyl, C₁-C₄-halogenoalkoxycarbonyl having 1 to 5 halogen atoms, C₁-C₄-alkylcarbonyloxy, C₁-C₄-halogenoalkylcarbonyloxy having 1 to 5 halogen atoms, C₁-C₄-alkylcarbonylamino, C₁-C₄-halogenoalkylcarbonylamino having 1 to 5 halogen atoms, —OCONH(C₁-C₄-alkyl), —OCON(C₁-C₄-alkyl)₂, —OCONH(OC₁-C₄-alkyl), —OCO(OC₁-C₄-alkyl), —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, (C₁-C₄-alkoxyimino)-C₁-C₄-alkyl, (C₂-C₆-alkenyloxyimino)-C₁-C₄-alkyl, (C₃-C₆-alkynyloxyimino)-C₁-C₄-alkyl, (benzyloxyimino)-C₁-C₆-alkyl, benzyloxy, —S-benzyl, benzylamino, phenoxy, —S-phenyl and phenylamino,
m is 1, 2 or 3, and
each Y is independently selected from the group consisting of hydrogen, halogen, nitro, cyano, hydroxy, amino, —SH, —SF₅, —CHO, —OCHO, —NHCHO, —COOH, —CONH₂, —CONH(OH), —OCONH₂, (hydroxyimino)-C₁-C₆-alkyl, C₁-C₆-alkyl, C₁-C₆-halogenoalkyl having 1 to 5 halogen atoms, C₂-C₆-alkenyl, C₂-C₆-alkynyl, C₁-C₆-alkylamino, di-(C₁-C₆-alkyl)amino, C₁-C₆-alkoxy, C₁-C₆-halogenoalkoxy having 1 to 5 halogen atoms, C₂-C₆-alkenyloxy, C₂-C₈-halogenoalkenyloxy having 1 to 5 halogen atoms, C₃-C₆-alkynyloxy, C₃-C₆-halogenoalkynyloxy having 1 to 5 halogen atoms, C₃-C₆-cycloalkyl, C₃-C₆-halogenocycloalkyl having 1 to 5 halogen atoms, C₁-C₆-alkylcarbonyl, C₁-C₆-halogenoalkylcarbonyl having 1 to 5 halogen atoms, —CONH(C₁-C₆-alkyl), —CON(C₁-C₆-alkyl)₂, —CONH(OC₁-C₆-alkyl), —CON(OC₁-C₆-alkyl)(C₁-C₆-alkyl), C₁-C₆-alkoxycarbonyl, C₁-C₆-halogenoalkoxycarbonyl having 1 to 5 halogen atoms, C₁-C₆-alkylcarbonyloxy, C₁-C₆-halogenoalkylcarbonyloxy having 1 to 5 halogen atoms, C₁-C₆-alkylcarbonylamino, C₁-C₆-halogenoalkylcarbonylamino having 1 to 5 halogen atoms, —OCONH(C₁-C₆-alkyl), —OCON(C₁-C₆-alkyl)₂, —OCONH(OC₁-C₆-alkyl), —OCO(OC₁-C₆-alkyl), —S—C₁-C₆-alkyl, —S—C₁-C₆-halogenoalkyl having 1 to 5 halogen atoms, —S(O)—C₁-C₆-alkyl, —S(O)—C₁-C₆-halogenoalkyl having 1 to 5 halogen atoms, —S(O)₂—C₁-C₆-alkyl, —S(O)₂—C₁-C₆-halogenoalkyl having 1 to 5 halogen atoms, —CH₂—S—C₁-C₆-alkyl, —CH₂—S(O)—C₁-C₆-alkyl, —CH₂—S(O)₂—C₁-C₆-alkyl, (C₁-C₄-alkoxyimino)-C₁-C₄-alkyl, (C₂-C₄-alkenyloxyimino)-C₁-C₄-alkyl, (C₃-C₆-alkynyloxyimino)-C₁-C₄-alkyl, wherein two Y may additionally be selected from —O—C₁-C₃-alkoxy and —O—C₁-C₃-halogenalkoxy having 1 to 5 halogen atoms, with each oxy function being the connecting atom of the individual Y, thereby forming an annelated 5-, 6- or 7-membered ring with the phenyl moiety that they are connected to, or
each Y is independently selected from the group consisting of hydrogen, halogen, cyano, C₁-C₆-alkyl, C₁-C₆-halogenoalkyl having 1 to 5 halogen atoms, and

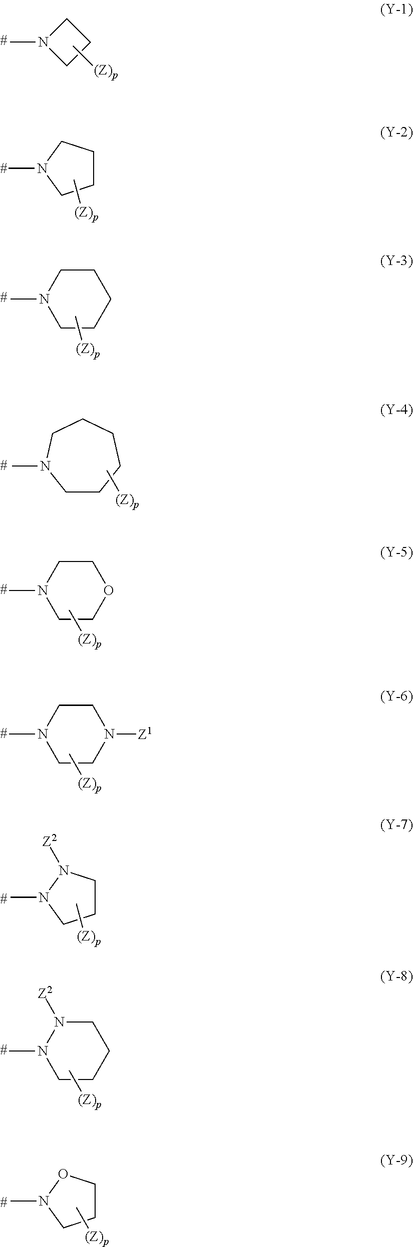

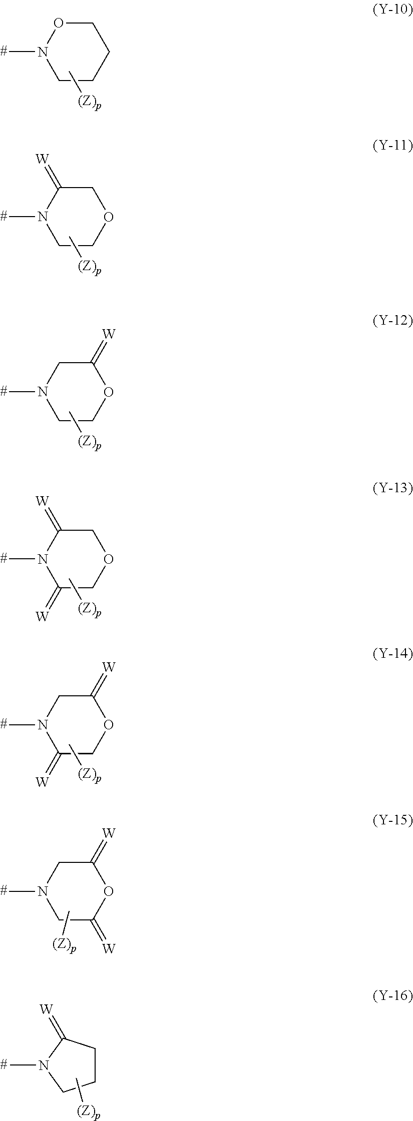

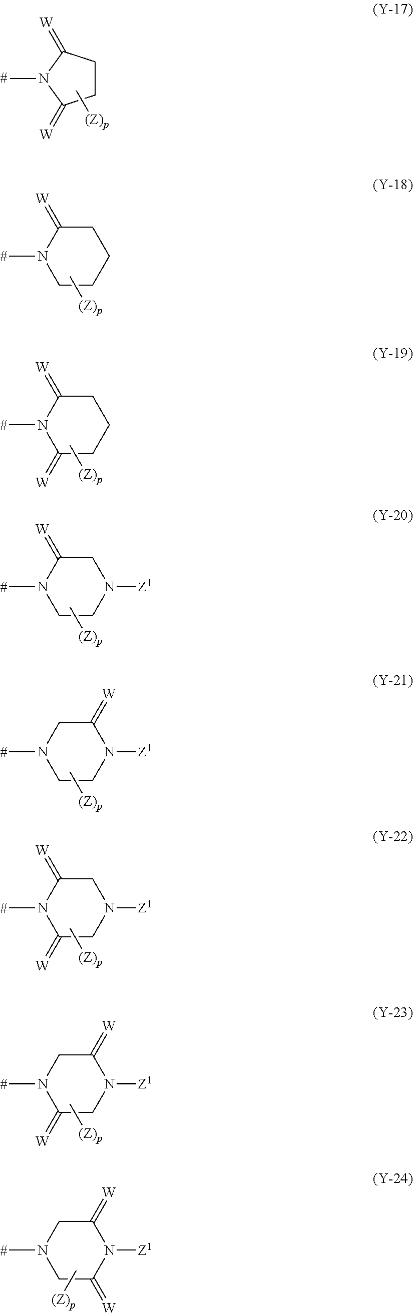

in which
# depicts the bond which connects Y to the rest of the molecule,
W is oxygen or sulfur,
p is 0, 1, 2, 3, 4, 5 or 6,
Z is independently selected from the group consisting of hydrogen, halogen, cyano, hydroxy, amino, —SH, —CHO, —COOH, —CONH₂, —CONH(OH), —OCONH₂, (hydroxyimino)-C₁-C₆-alkyl, C₁-C₆-alkyl, C₁-C₆-halogenoalkyl having 1 to 5 halogen atoms, C₁-C₆-alkylamino, di-(C₁-C₆-alkyl)amino, C₁-C₆-alkoxy, C₁-C₆-halogenoalkoxy having 1 to 5 halogen atoms, C₃-C₆-cycloalkyl, C₃-C₆-halogenocycloalkyl having 1 to 5 halogen atoms, —CONH(C₁-C₆-alkyl), —CON(C₁-C₆-alkyl)₂, —S—C₁-C₆-alkyl, —S—C₁-C₆-halogenoalkyl having 1 to 5 halogen atoms, —S(O)—C₁-C₆-alkyl, —S(O)—C₁-C₆-halogenoalkyl having 1 to 5 halogen atoms, —S(O)₂—C₁-C₆-alkyl, —S(O)₂—C₁-C₆-halogenoalkyl having 1 to 5 halogen atoms,
Z¹ is independently selected from the group consisting of hydrogen, C₁-C₆-alkyl, C₁-C₆-halogenoalkyl having 1 to 5 halogen atoms, C₁-C₆-alkylamino, di-(C₁-C₆-alkyl)amino, C₁-C₆-alkoxy, C₁-C₆-alkylcarbonyl, C₃-C₆-cycloalkyl, C₃-C₆-halogenocycloalkyl having 1 to 5 halogen atoms, —CONH(C₁-C₆-alkyl), —CON(C₁-C₆-alkyl)₂, and
Z² is independently selected from the group consisting of hydrogen, C₁-C₆-alkyl, C₁-C₆-halogenoalkyl having 1 to 5 halogen atoms, C₃-C₆-cycloalkyl, C₃-C₆-halogenocycloalkyl having 1 to 5 halogen atoms, —CONH(C₁-C₆-alkyl), —CON(C₁-C₆-alkyl)₂,
   and salts, solvates, solvates of the salts, N-oxides, metal complexes or metalloid complexes thereof.

In yet another embodiment, the present invention provides compounds for use in the control, treatment and/or prevention of infections with helminths in animals and humans according to formula (I), wherein
A represents a phenyl group of formula (A1)

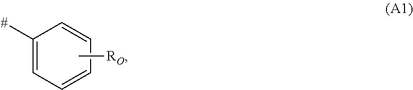

in which
# depicts the bond which connects A to the rest of the molecule,
o is 0, 1 or 2, and
each R is independently selected from the group consisting of halogen, nitro, C₁-C₄-halogenoalkyl having 1 to 5 halogen atoms, C₁-C₄-halogenoalkoxy having 1 to 5 halogen atoms, or
A represents a heterocycle of the formula (Het-1)

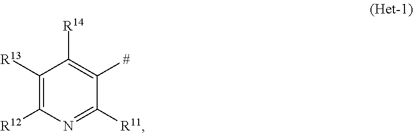

in which
# depicts the bond which connects A to the rest of the molecule,
R¹¹ is halogen or C₁-C₄-halogenoalkyl having 1 to 5 halogen atoms, and
R¹², R¹³ and R¹⁴ are hydrogen, or
A represents a heterocycle of the formula (Het-2)

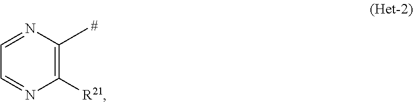

in which
# depicts the bond which connects A to the rest of the molecule, and
R²¹ is C₁-C₄-halogenoalkyl having 1 to 5 halogen atoms, or
A represents a heterocycle of the formula (Het-3)

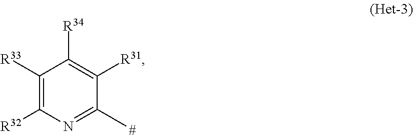

in which
# depicts the bond which connects A to the rest of the molecule,
R³¹ is C₁-C₄-halogenoalkyl having 1 to 5 halogen atoms, and
R³², R³³ and R³⁴ are hydrogen,
R¹ is selected from the group consisting of hydrogen, C₁-C₄-alkyl, C₃-C₄-alkynyl, C₃-C₆-cycloalkyl, C₁-C₄-alkylcarbonyl, C₁-C₄-alkoxy-C₁-C₄-alkyl, C₁-C₄-alkoxycarbonyl,
n is 1 or 2,
each X is independently selected from the group consisting of halogen, cyano, C₁-C₄-alkyl, C₁-C₄-alkoxy and C₁-C₄-halogenoalkyl having 1 to 5 halogen atoms,
m is 1, 2 or 3, and
each Y is independently selected from the group consisting of hydrogen, halogen, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —CONH(C₁-C₄-alkyl), —CON(C₁-C₄-alkyl)₂, —CONH(OC₁-C₄-alkyl), —CON(OC₁-C₄-alkyl)(C₁-C₆-alkyl), —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino, wherein two Y may additionally be selected from —O—C₁-C₂-alkoxy and —O—C₁-C₂-halogenalkoxy having 1 to 5 halogen atoms, with each oxy function being the connecting atom of the individual Y, thereby forming an annelated 5- or 6-membered ring with the phenyl moiety that they are connected to, or
each Y is independently selected from the group consisting of hydrogen, halogen, cyano, C₁-C₄-alkyl, C₁-C₄-halogenoalkyl having 1 to 5 halogen atoms, and

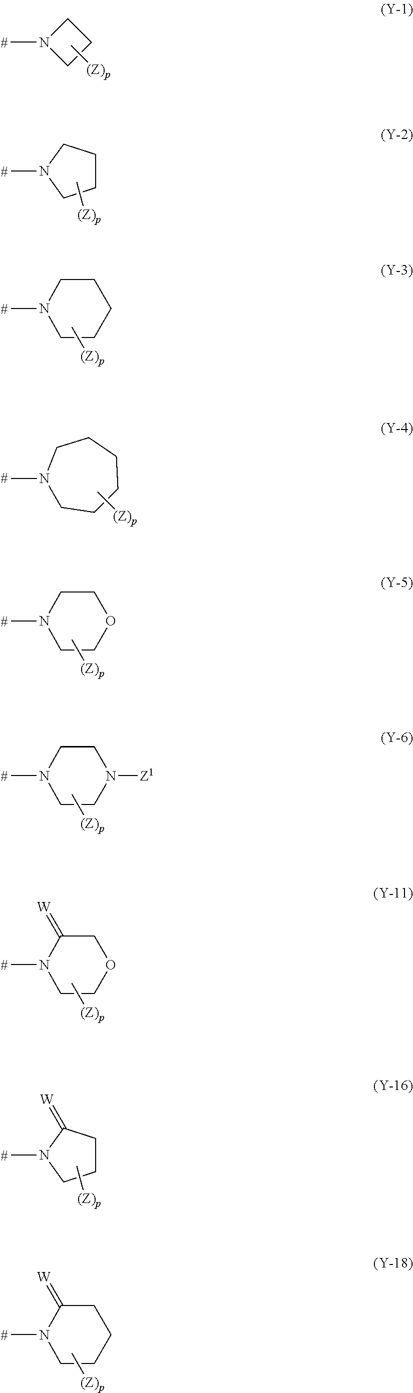

in which
# depicts the bond which connects Y to the rest of the molecule,
W is oxygen,
p is 0, 1, 2, 3, 4, 5 or 6,
Z is independently selected from the group consisting of hydrogen, halogen, cyano, hydroxy, amino, —SH, —CHO, —COOH, —CONH₂, —CONH(OH), —OCONH₂, (hydroxyimino)-C₁-C₄-alkyl, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, C₃-C₆-cycloalkyl, C₃-C₆-halogenocycloalkyl having 1 to 5 halogen atoms, —CONH(C₁-C₄-alkyl), —CON(C₁-C₄-alkyl)₂, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, and
Z¹ is independently selected from the group consisting of hydrogen, C₁-C₆-alkyl, C₁-C₆-halogenoalkyl having 1 to 5 halogen atoms, C₁-C₆-alkylamino, di-(C₁-C₆-alkyl)amino, C₁-C₆-alkoxy, C₁-C₆-alkylcarbonyl, C₃-C₆-cycloalkyl, C₃-C₆-halogenocycloalkyl having 1 to 5 halogen atoms, —CONH(C₁-C₆-alkyl), —CON(C₁-C₆-alkyl)₂,
   and salts, solvates, solvates of the salts, N-oxides, metal complexes or metalloid complexes thereof.

In yet another embodiment, the present invention provides compounds for use in the control, treatment and/or prevention of infections with helminths in animals and humans according to formula (I), wherein
A represents a phenyl group of formula (A1)

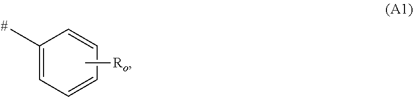

in which
# depicts the bond which connects A to the rest of the molecule,
o is 1 or 2, and
each R is independently selected from the group consisting of halogen, nitro, —CF₃, —OCF₃, —CHF₂, or
A represents a heterocycle of the formula (Het-1)

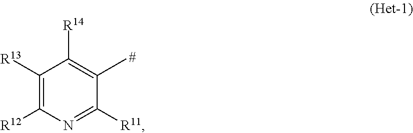

in which
# depicts the bond which connects A to the rest of the molecule,
R¹¹ is halogen or CF₃, and
R¹², R¹³ and R¹⁴ are hydrogen, or
A represents a heterocycle of the formula (Het-2)

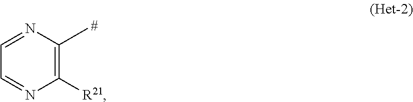

in which
# depicts the bond which connects A to the rest of the molecule, and
R²¹ is CF₃, or
A represents a heterocycle of the formula (Het-3)

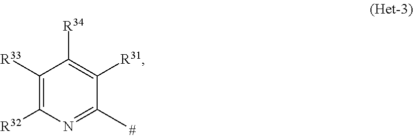

in which
# depicts the bond which connects A to the rest of the molecule,
R³¹ is CF₃, and
R³², R³³ and R³⁴ are hydrogen,
R¹ is hydrogen,
n is 1 or 2,
each X is independently selected from the group consisting of halogen, cyano, CH₃, OCH₃ and CF₃,
m is 1, 2 or 3, and
each Y is independently selected from the group consisting of hydrogen, halogen, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino, wherein two Y may additionally be selected from —O—CH₂—O— and —O—CF₂—O—, with each oxy function being the connecting atom of the individual Y, thereby forming an annelated 5-membered ring with the phenyl moiety that they are connected to, or
each Y is independently selected from the group consisting of hydrogen, fluoro, chloro, cyano, CF₃, and

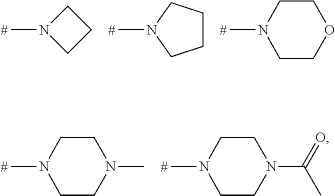

and salts, solvates, solvates of the salts, N-oxides, metal complexes or metalloid complexes thereof.

In yet another embodiment, the present invention provides compounds for use in the control, treatment and/or prevention of infections with helminths in animals and humans according to formula (I), wherein
A represents a phenyl group of formula (A1)

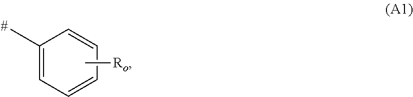

in which
# depicts the bond which connects A to the rest of the molecule,
o is 1 or 2, and
each R is independently selected from the group consisting of halogen, nitro, —CF₃, —OCF₃, —CHF₂, or
A represents a heterocycle of the formula (Het-1)

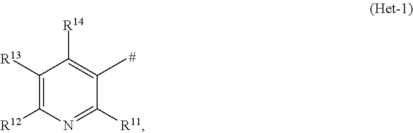

in which
# depicts the bond which connects A to the rest of the molecule,
R¹¹ is halogen or CF₃, and
R¹², R¹³ and R¹⁴ are hydrogen, or
A represents a heterocycle of the formula (Het-2)

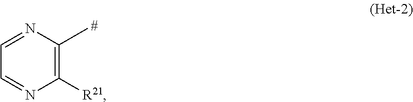

in which
# depicts the bond which connects A to the rest of the molecule, and
R²¹ is CF₃, or
A represents a heterocycle of the formula (Het-3)

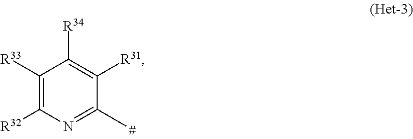

in which
# depicts the bond which connects A to the rest of the molecule,
R³¹ is CF₃, and
R³², R³³ and R³⁴ are hydrogen,
R¹ is hydrogen,
n is 1 or 2,
each X is independently selected from the group consisting of halogen, cyano, CH₃, OCH₃ and CF₃,
m is 1, 2 or 3, and
each Y is independently selected from the group consisting of hydrogen, halogen, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino,
   and salts, solvates, solvates of the salts, N-oxides, metal complexes or metalloid complexes thereof.

In yet another embodiment, the present invention provides compounds for use in the control, treatment and/or prevention of infections with helminths in animals and humans according to formula (I), wherein
A is selected from the group consisting of

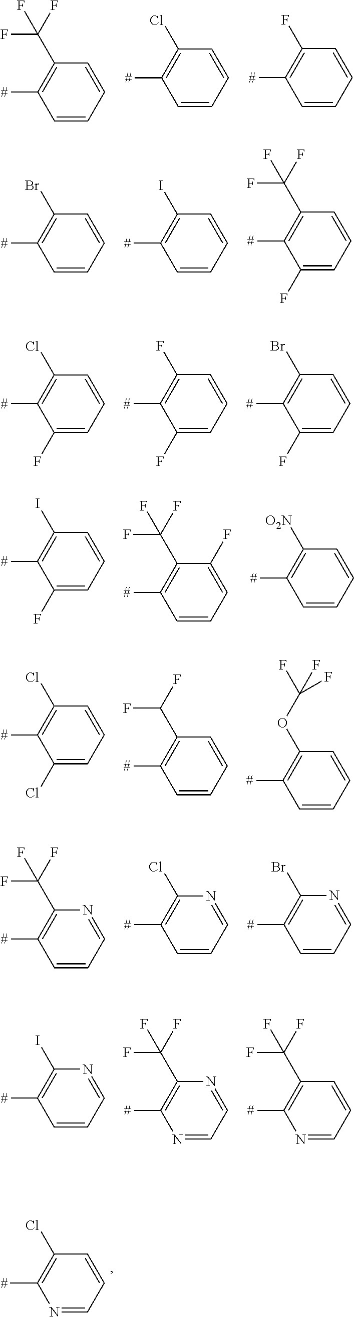

in which
# depicts the bond which connects A to the rest of the molecule,
R¹ is hydrogen,
n is 1 or 2,
each X is independently selected from the group consisting of chloro, fluoro, cyano, CH₃, OCH₃ and CF₃,
m is 1, 2 or 3, and
each Y is independently selected from the group consisting of hydrogen, halogen, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino, wherein two Y may additionally be selected from —O—CH₂—O— and —O—CF₂—O—, with each oxy function being the connecting atom of the individual Y, thereby forming an annelated 5-membered ring with the phenyl moiety that they are connected to, or
each Y is independently selected from the group consisting of hydrogen, fluoro, chloro, cyano, CF₃, and

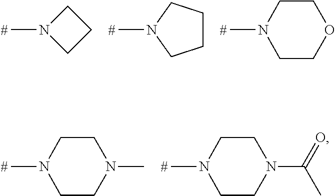

and salts, solvates, solvates of the salts, N-oxides, metal complexes or metalloid complexes thereof.

In yet another embodiment, the present invention provides compounds for use in the control, treatment and/or prevention of infections with helminths in animals and humans according to formula (I), wherein
A is selected from the group consisting of

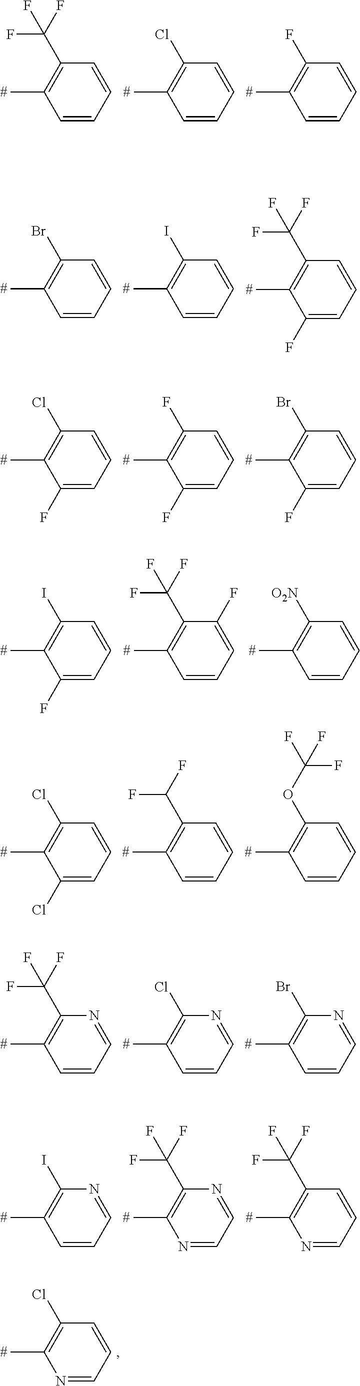

in which
# depicts the bond which connects A to the rest of the molecule,
R¹ is hydrogen,
n is 1 or 2,
each X is independently selected from the group consisting of chloro, fluoro, cyano, CH₃, OCH₃ and CF₃,
m is 1, 2 or 3, and
each Y is independently selected from the group consisting of hydrogen, halogen, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino,
   and salts, solvates, solvates of the salts, N-oxides, metal complexes or metalloid complexes thereof.

In yet another embodiment, the present invention provides compounds for use in the control, treatment and/or prevention of infections with helminths in animals and humans according to formula (I), wherein
A is selected from the group consisting of

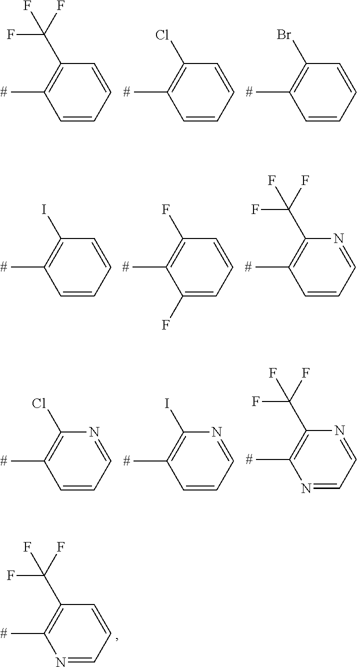

in which
# depicts the bond which connects A to the rest of the molecule,
R¹ is hydrogen,
n is 1 or 2,
each X is independently selected from the group consisting of chloro, fluoro, cyano, CH₃, OCH₃ and CF₃,
m is 1, 2 or 3, and
each Y is independently selected from the group consisting of hydrogen, fluoro, chloro, bromo, cyano, trifluoromethyl, difluoromethyl, methoxy, trifluoromethoxy, difluoromethoxy, methylamino, dimethylamino, methylsulfanyl, trifluoromethylsulfanyl, methylsulfinyl, trifluoromethylsulfinyl, methylsulfonyl, trifluoromethylsulfonyl, and acetylamino, or
each Y is independently selected from the group consisting of hydrogen, fluoro, chloro, cyano, CF₃, and

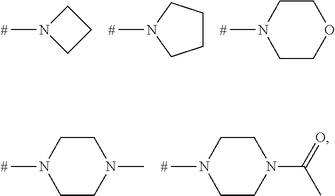

and salts, solvates, solvates of the salts, N-oxides, metal complexes or metalloid complexes thereof.

In yet another embodiment, the present invention provides compounds for use in the control, treatment and/or prevention of infections with helminths in animals and humans according to formula (I), wherein
A is selected from the group consisting of

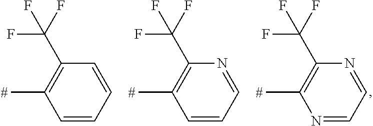

in which
# depicts the bond which connects A to the rest of the molecule,
R¹ is hydrogen,
n is 1 or 2,
each X is independently selected from the group consisting of chloro, fluoro, cyano, CH₃, OCH₃ and CF₃,
m is 1, 2 or 3, and
each Y is independently selected from the group consisting of hydrogen, fluoro, chloro, bromo, cyano, trifluoromethyl, difluoromethyl, methoxy, trifluoromethoxy, difluoromethoxy, methylamino, dimethylamino, methylsulfanyl, trifluoromethylsulfanyl, methylsulfinyl, trifluoromethylsulfinyl, methylsulfonyl, trifluoromethylsulfonyl, and acetylamino,
   and salts, solvates, solvates of the salts, N-oxides, metal complexes or metalloid complexes thereof.

In yet another embodiment, the present invention provides compounds for use in the control, treatment and/or prevention of infections with helminths in animals and humans according to formula (I), wherein
A is selected from the group consisting of

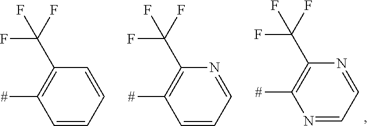

in which
# depicts the bond which connects A to the rest of the molecule,
R¹ is hydrogen,
n is 1 or 2,
each X is independently selected from the group consisting of chloro, fluoro, CH₃ and CF₃,
m is 1, 2 or 3, and
each Y is independently selected from the group consisting of hydrogen, fluoro and chloro,
   and salts, solvates, solvates of the salts, N-oxides, metal complexes or metalloid complexes thereof.

In yet another embodiment, the present invention provides compounds for use in the control, treatment and/or prevention of infections with helminths in animals and humans according to formula (I), wherein
A is selected from the group consisting of

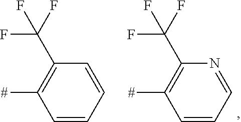

in which
# depicts the bond which connects A to the rest of the molecule,
R¹ is hydrogen,
n is 1 or 2,
each X is independently selected from the group consisting of chloro, fluoro, CH₃, OCH₃ and CF₃,
m is 1, 2 or 3, and
each Y is independently selected from the group consisting of hydrogen, fluoro, chloro, cyano, trifluoromethyl, methoxy, trifluoromethoxy, methylamino, dimethylamino, methylsulfanyl, trifluoromethylsulfanyl, methylsulfonyl, and acetylamino,
   and salts, solvates, solvates of the salts, N-oxides, metal complexes or metalloid complexes thereof.

In a further embodiment, the present invention provides compounds of the following formula (I-1)

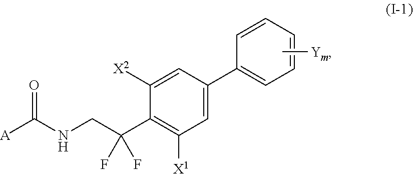

wherein
X¹ is selected from the group consisting of halogen, cyano, C₁-C₄-alkyl, C₁-C₄-alkoxy and C₁-C₄-halogenoalkyl having 1 to 5 halogen atoms,
X² is selected from the group consisting of hydrogen, halogen, cyano, C₁-C₄-alkyl, C₁-C₄-alkoxy and C₁-C₄-halogenoalkyl having 1 to 5 halogen atoms
m is 1, 2 or 3,
each Y is independently selected from the group consisting of hydrogen, halogen, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino, and
A is selected from the group consisting of

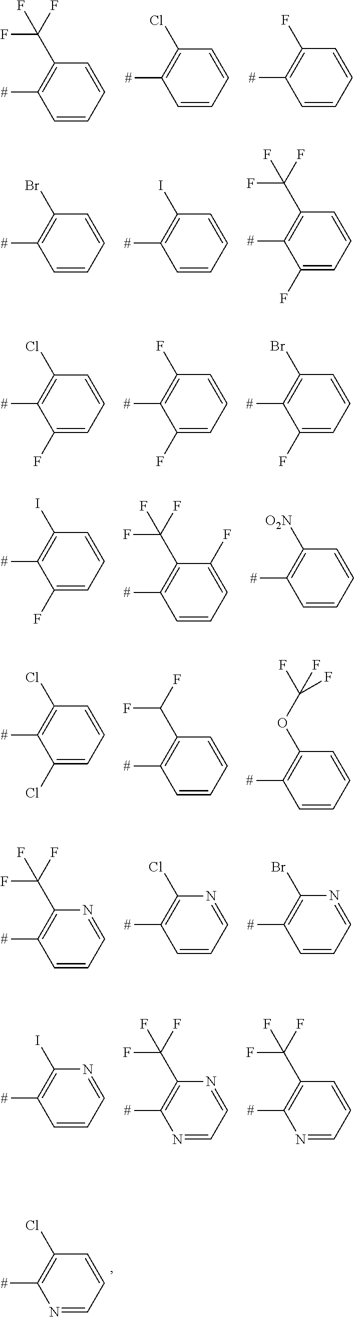

in which
# depicts the bond which connects A to the rest of the molecule,
   and salts, solvates, solvates of the salts, N-oxides, metal complexes or metalloid complexes thereof.

In another further embodiment, the present invention provides compounds of the following formula (I-1)

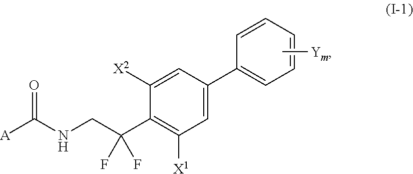

wherein
X¹ is selected from the group consisting of hydrogen, halogen, cyano, C₁-C₄-alkyl, C₁-C₄-alkoxy and C₁-C₄-halogenoalkyl having 1 to 5 halogen atoms,
X² is selected from the group consisting of hydrogen, halogen, cyano, C₁-C₄-alkyl, C₁-C₄-alkoxy and C₁-C₄-halogenoalkyl having 1 to 5 halogen atoms
m is 1, 2 or 3,
each Y is independently selected from the group consisting of hydrogen, halogen, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino, and
A is selected from the group consisting of

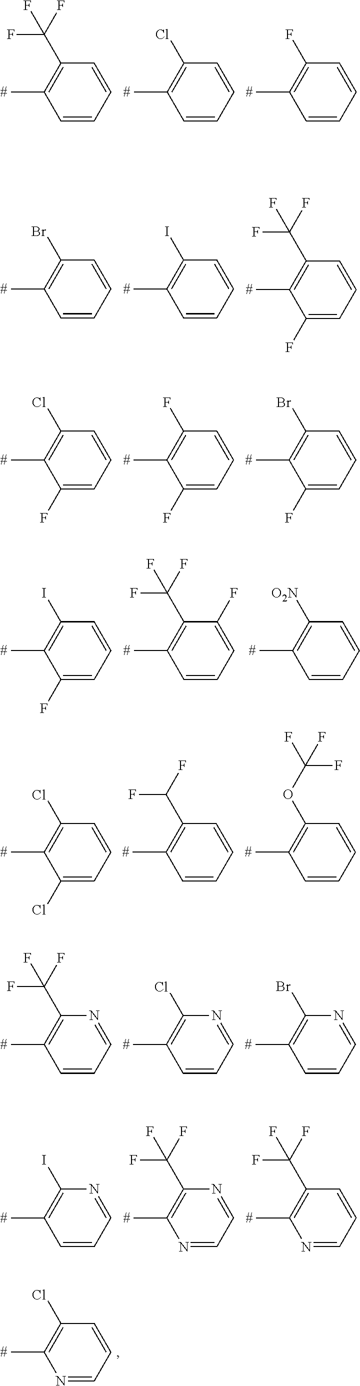

in which
# depicts the bond which connects A to the rest of the molecule,
   and salts, solvates, solvates of the salts, N-oxides, metal complexes or metalloid complexes thereof, for use in the control, treatment and/or prevention of infections with helminths in animals and humans.

The following embodiments that relate to formula (I-1) shall be understood as referring to the compounds according to the present invention per se or to the compounds for use in the control, treatment and/or prevention of infections with helminths in animals and humans according to the present invention, or to both, unless indicated otherwise.

In still a further embodiment, the present invention provides compounds of formula (I-1), wherein
A is selected from the group consisting of

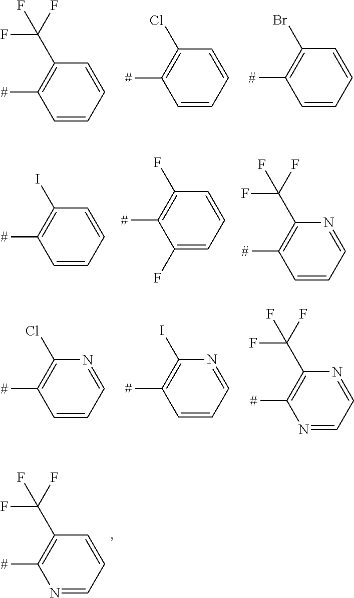

in which
# depicts the bond which connects A to the rest of the molecule, and
X¹, X², m, and Y have the meaning as described before for formula (I-1),
   and salts, solvates, solvates of the salts, N-oxides, metal complexes or metalloid complexes thereof.

In still a further embodiment, the present invention provides compounds of formula (I-1), wherein
A is selected from the group consisting of

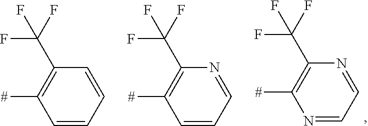

in which
# depicts the bond which connects A to the rest of the molecule, and
X¹, X², m, and Y have the meaning as described before for formula (I-1),
   and salts, solvates, solvates of the salts, N-oxides, metal complexes or metalloid complexes thereof.

In still a further embodiment, the present invention provides compounds of formula (I-1), wherein
X¹ is selected from the group consisting of chloro, fluoro, cyano, CH₃, OCH₃ and CF₃,
X² is hydrogen or fluoro, and
A, m and Y have the meaning as described before for formula (I-1),
   and salts, solvates, solvates of the salts, N-oxides, metal complexes or metalloid complexes thereof.

In still a further embodiment, the present invention provides compounds of formula (I-1), wherein
m is 1, 2 or 3,
each Y is independently selected from the group consisting of hydrogen, fluoro, chloro, bromo, cyano, trifluoromethyl, difluoromethyl, methoxy, trifluoromethoxy, difluoromethoxy, methylamino, dimethylamino, methylsulfanyl, trifluoromethylsulfanyl, methylsulfinyl, trifluoromethylsulfinyl, methylsulfonyl, trifluoromethylsulfonyl, acetylamino, and
A, X¹, and X² have the meaning as described before for formula (I-1),
   and salts, solvates, solvates of the salts, N-oxides, metal complexes or metalloid complexes thereof.

In a further embodiment, the present invention provides compounds of the following formula (I-2)

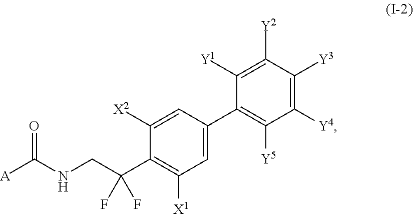

wherein
A is selected from the group consisting of

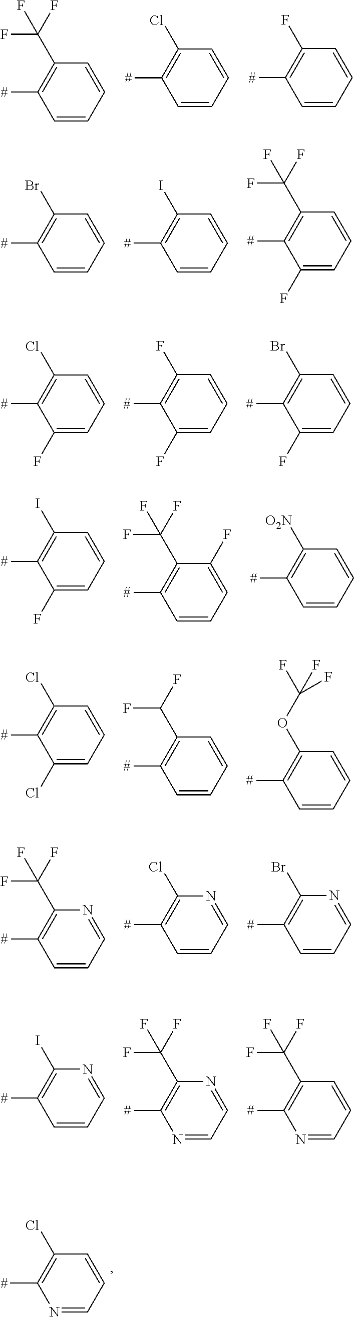

in which
# depicts the bond which connects A to the rest of the molecule,
X¹ is fluoro,
X² is selected from the group consisting of hydrogen, chloro, fluoro, cyano, CH₃, OCH₃ and CF₃,
Y¹ is selected from the group consisting of hydrogen, fluoro, chloro, bromo, iodo, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino,
Y² is selected from the group consisting of hydrogen, fluoro, chloro, bromo, iodo, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino,
Y³ is selected from the group consisting of hydrogen, fluoro, chloro, bromo, iodo, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino,
Y⁴ is selected from the group consisting of hydrogen, fluoro, chloro, bromo, iodo, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino, and
Y⁵ is selected from the group consisting of hydrogen, fluoro, chloro, bromo, iodo, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino,
with the proviso that
at least two of Y¹, Y², Y³, Y⁴ and Y⁵ are hydrogen,
   and salts, solvates, solvates of the salts, N-oxides, metal complexes or metalloid complexes thereof.

In a further embodiment, the present invention provides compounds of the following formula (I-2)

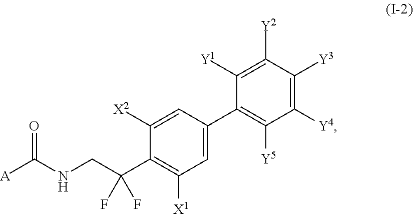

wherein
A is selected from the group consisting of

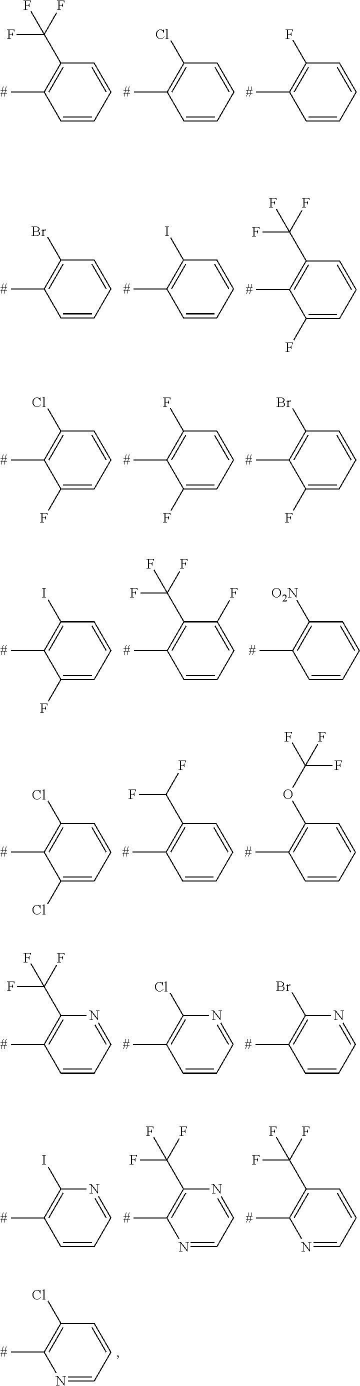

in which
# depicts the bond which connects A to the rest of the molecule,
X¹ is CF₃,
X² is selected from the group consisting of hydrogen, chloro, fluoro, cyano, CH₃, OCH₃ and CF₃,
Y¹ is selected from the group consisting of hydrogen, fluoro, chloro, bromo, iodo, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino,
Y² is selected from the group consisting of hydrogen, fluoro, chloro, bromo, iodo, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino,
Y³ is selected from the group consisting of hydrogen, fluoro, chloro, bromo, iodo, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino,
Y⁴ is selected from the group consisting of hydrogen, fluoro, chloro, bromo, iodo, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino, and
Y⁵ is selected from the group consisting of hydrogen, fluoro, chloro, bromo, iodo, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino,
with the proviso that
at least two of Y¹, Y², Y³, Y⁴ and Y⁵ are hydrogen,
   and salts, solvates, solvates of the salts, N-oxides, metal complexes or metalloid complexes thereof.

In a further embodiment, the present invention provides compounds of the following formula (I-2)

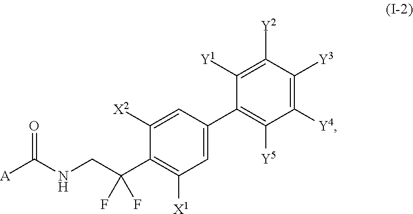

wherein
A is selected from the group consisting of

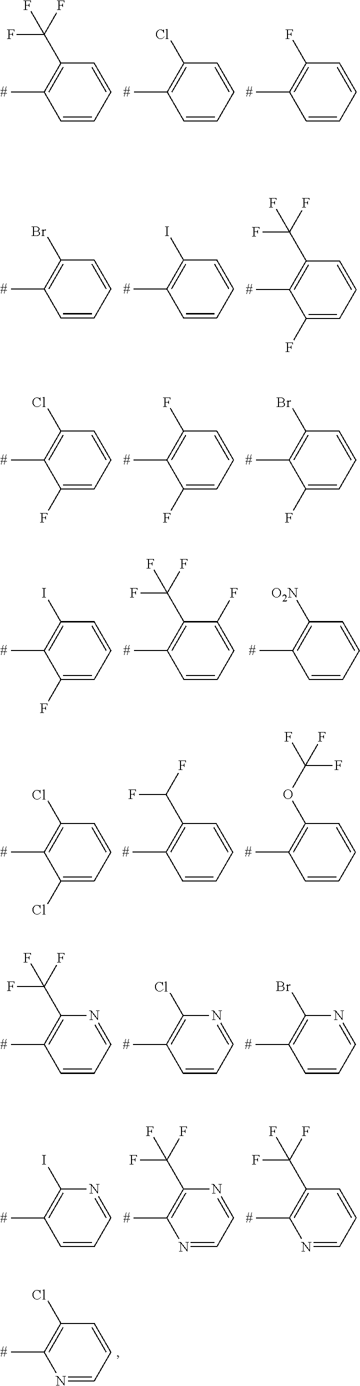

in which
# depicts the bond which connects A to the rest of the molecule,
X¹ is chloro,
X² is selected from the group consisting of hydrogen, chloro, fluoro, cyano, CH₃, OCH₃ and CF₃,
Y¹ is selected from the group consisting of hydrogen, fluoro, chloro, bromo, iodo, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino,
Y² is selected from the group consisting of hydrogen, fluoro, chloro, bromo, iodo, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino,
Y³ is selected from the group consisting of bromo, iodo, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino,
Y⁴ is selected from the group consisting of hydrogen, fluoro, chloro, bromo, iodo, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino, and
Y⁵ is selected from the group consisting of hydrogen, fluoro, chloro, bromo, iodo, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino,
with the proviso that
at least two of Y¹, Y², Y³, Y⁴ and Y⁵ are hydrogen,
   and salts, solvates, solvates of the salts, N-oxides, metal complexes or metalloid complexes thereof.

In a further embodiment, the present invention provides compounds of the following formula (I-2)

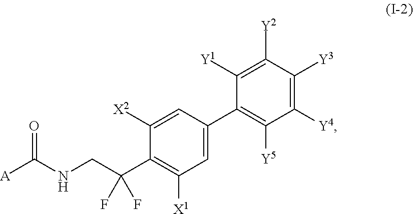

wherein
A is selected from the group consisting of

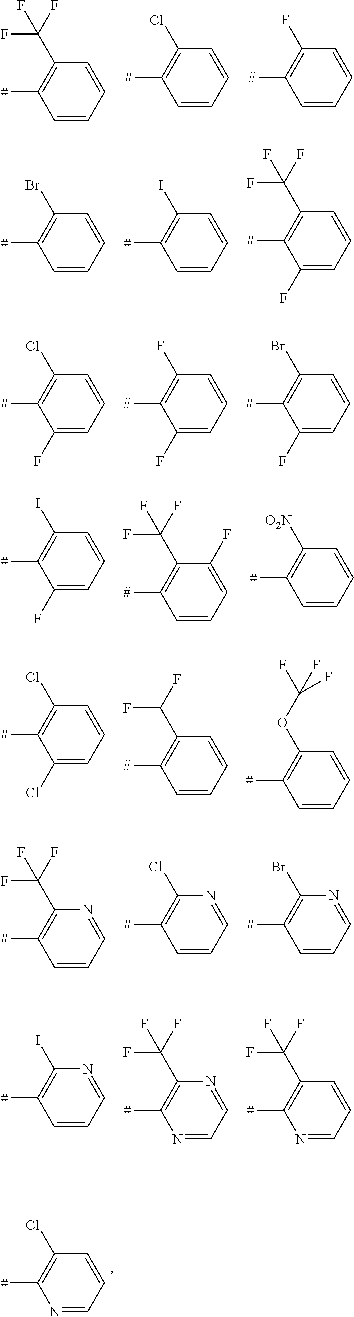

in which
# depicts the bond which connects A to the rest of the molecule,
X¹ is OCH₃,
X² is selected from the group consisting of hydrogen, chloro, fluoro, cyano, CH₃, OCH₃ and CF₃,
Y¹ is selected from the group consisting of hydrogen, fluoro, chloro, bromo, iodo, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino,
Y² is selected from the group consisting of hydrogen, fluoro, chloro, bromo, iodo, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino,
Y³ is selected from the group consisting of hydrogen, fluoro, chloro, bromo, iodo, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino,
Y⁴ is selected from the group consisting of hydrogen, fluoro, chloro, bromo, iodo, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino, and
Y⁵ is selected from the group consisting of hydrogen, fluoro, chloro, bromo, iodo, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino,
with the proviso that
at least two of Y¹, Y², Y³, Y⁴ and Y⁵ are hydrogen,
   and salts, solvates, solvates of the salts, N-oxides, metal complexes or metalloid complexes thereof.

In a further embodiment, the present invention provides compounds of the following formula (I-2)

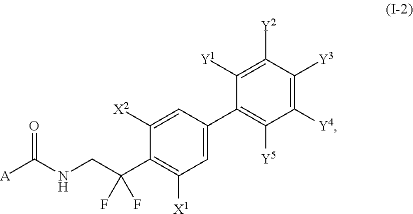

wherein
A is selected from the group consisting of

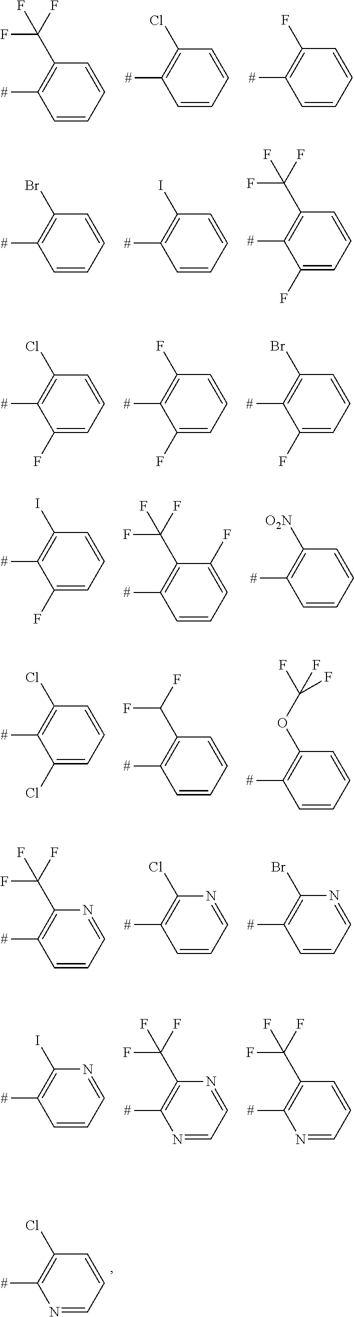

in which
# depicts the bond which connects A to the rest of the molecule,
X¹ is CH₃,
X² is selected from the group consisting of hydrogen, chloro, fluoro, cyano, CH₃, OCH₃ and CF₃,
Y¹ is selected from the group consisting of hydrogen, fluoro, chloro, bromo, iodo, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino,
Y² is selected from the group consisting of hydrogen, fluoro, chloro, bromo, iodo, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino,
Y³ is selected from the group consisting of hydrogen, fluoro, chloro, bromo, iodo, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino,
Y⁴ is selected from the group consisting of hydrogen, fluoro, chloro, bromo, iodo, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino, and
Y⁵ is selected from the group consisting of hydrogen, fluoro, chloro, bromo, iodo, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino,
with the proviso that
at least two of Y¹, Y², Y³, Y⁴ and Y⁵ are hydrogen,
   and salts, solvates, solvates of the salts, N-oxides, metal complexes or metalloid complexes thereof.

In a further embodiment, the present invention provides compounds of the following formula (I-2)

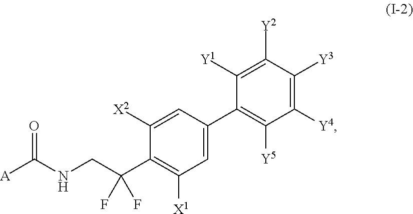

wherein
A is selected from the group consisting of

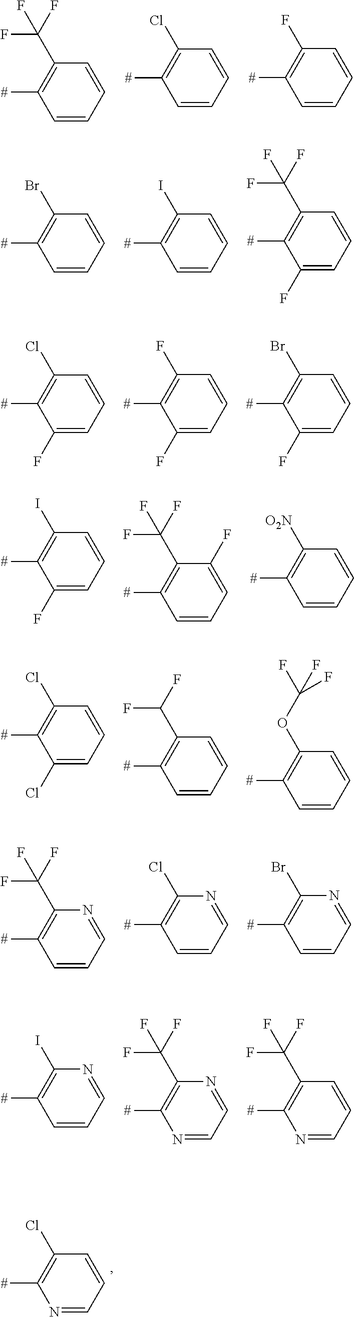

in which
# depicts the bond which connects A to the rest of the molecule,
X¹ is fluoro,
X² is fluoro,
Y¹ is selected from the group consisting of hydrogen, fluoro, chloro, bromo, iodo, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino,
Y² is selected from the group consisting of hydrogen, fluoro, chloro, bromo, iodo, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino,
Y³ is selected from the group consisting of hydrogen, fluoro, chloro, bromo, iodo, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino,
Y⁴ is selected from the group consisting of hydrogen, fluoro, chloro, bromo, iodo, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino, and
Y⁵ is selected from the group consisting of hydrogen, fluoro, chloro, bromo, iodo, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino,
with the proviso that
at least two of Y¹, Y², Y³, Y⁴ and Y⁵ are hydrogen,
   and salts, solvates, solvates of the salts, N-oxides, metal complexes or metalloid complexes thereof.

The present invention relates further to compounds of formula (VII)

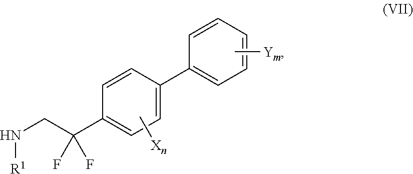

wherein
R¹ is selected from the group consisting of hydrogen, —CHO, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, C₃-C₆-halogenocycloalkyl having 1 to 5 halogen atoms, C₃-C₄-alkenyl, C₃-C₄-alkynyl, C₁-C₄-alkoxy-C₁-C₄-alkyl, C₃-C₆-cycloalkyl-C₁-C₃-alkyl, cyano-C₁-C₄-alkyl, amino-C₁-C₄-alkyl, C₁-C₄-alkylamino-C₁-C₄-alkyl, di-(C₁-C₄-alkyl)amino-C₁-C₄-alkyl, C₁-C₄-alkylcarbonyl, C₁-C₄-halogenoalkylcarbonyl having 1 to 5 halogen atoms, C₁-C₄-alkoxycarbonyl, benzyloxycarbonyl, C₁-C₄-alkoxy-C₁-C₄-alkylcarbonyl, —S(O)₂—C₁-C₄-alkyl, and —S(O)₂—C₁-C₄-halogenoalkyl having 1 to 5 halogen atoms,
n is 0, 1, 2 or 3,
each X is independently selected from the group consisting of hydrogen, halogen, nitro, cyano, hydroxy, amino, —SH, —SF₅, —CHO, —OCHO, —NHCHO, —COOH, —CONH₂, —CONH(OH), —OCONH₂, (hydroxyimino)-C₁-C₆-alkyl, C₁-C₈-alkyl, C₁-C₈-halogenoalkyl having 1 to 5 halogen atoms, C₂-C₈-alkenyl, C₂-C₈-alkynyl, C₁-C₈-alkylamino, di-(C₁-C₈-alkyl)amino, C₁-C₈-alkoxy, C₁-C₈-halogenoalkoxy having 1 to 5 halogen atoms, C₂-C₈-alkenyloxy, C₂-C₈-halogenoalkenyloxy having 1 to 5 halogen atoms, C₃-C₈-alkynyloxy, C₃-C₈-halogenoalkynyloxy having 1 to 5 halogen atoms, C₃-C₈-cycloalkyl, C₃-C₈-halogenocycloalkyl having 1 to 5 halogen atoms, C₁-C₈-alkylcarbonyl, C₁-C₈-halogenoalkylcarbonyl having 1 to 5 halogen atoms, —CONH(C₁-C₈-alkyl), —CON(C₁-C₈-alkyl)₂, —CONH(OC₁-C₈-alkyl), —CON(OC₁-C₈-alkyl)(C₁-C₈-alkyl), C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, —OCONH(C₁-C₈-alkyl), —OCON(C₁-C₈-alkyl)₂, —OCONH(OC₁-C₈-alkyl), —OCO(OC₁-C₈-alkyl), —S—C₁-C₈-alkyl, —S—C₁-C₈-halogenoalkyl having 1 to 5 halogen atoms, —S(O)—C₁-C₈-alkyl, —S(O)—C₁-C₈-halogenoalkyl having 1 to 5 halogen atoms, —S(O)₂—C₁-C₈-alkyl, —S(O)₂—C₁-C₈-halogenoalkyl having 1 to 5 halogen atoms, (C₁-C₆-alkoxyimino)-C₁-C₆-alkyl, (C₂-C₆-alkenyloxyimino)-C₁-C₆-alkyl, (C₃-C₆-alkynyloxyimino)-C₁-C₆-alkyl, (benzyloxyimino)-C₁-C₆-alkyl, benzyloxy, —S-benzyl, benzylamino, phenoxy, —S-phenyl and phenylamino,
m is 0, 1, 2, 3 or 4,
each Y is independently selected from the group consisting of hydrogen, halogen, nitro, cyano, hydroxy, amino, —SH, —SF₅, —CHO, —OCHO, —NHCHO, —COOH, —CONH₂, —CONH(OH), —OCONH₂, (hydroxyimino)-C₁-C₆-alkyl, C₁-C₈-alkyl, C₁-C₈-halogenoalkyl having 1 to 5 halogen atoms, C₂-C₈-alkenyl, C₂-C₈-alkynyl, C₁-C₈-alkylamino, di-(C₁-C₈-alkyl)amino, C₁-C₈-alkoxy, C₁-C₈-halogenoalkoxy having 1 to 5 halogen atoms, C₂-C₈-alkenyloxy, C₂-C₈-halogenoalkenyloxy having 1 to 5 halogen atoms, C₃-C₈-alkynyloxy, C₃-C₈-halogenoalkynyloxy having 1 to 5 halogen atoms, C₃-C₈-cycloalkyl, C₃-C₈-halogenocycloalkyl having 1 to 5 halogen atoms, C₁-C₈-alkylcarbonyl, C₁-C₈-halogenoalkylcarbonyl having 1 to 5 halogen atoms, —CONH(C₁-C₈-alkyl), —CON(C₁-C₈-alkyl)₂, —CONH(OC₁-C₈-alkyl), —CON(OC₁-C₈-alkyl)(C₁-C₈-alkyl), C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, —OCONH(C₁-C₈-alkyl), —OCON(C₁-C₈-alkyl)₂, —OCONH(OC₁-C₈-alkyl), —OCO(OC₁-C₈-alkyl), —S—C₁-C₈-alkyl, —S—C₁-C₈-halogenoalkyl having 1 to 5 halogen atoms, —S(O)—C₁-C₈-alkyl, —S(O)—C₁-C₈-halogenoalkyl having 1 to 5 halogen atoms, —S(O)₂—C₁-C₈-alkyl, —S(O)₂—C₁-C₈-halogenoalkyl having 1 to 5 halogen atoms, —CH₂—S—C₁-C₈-alkyl, —CH₂—S(O)—C₁-C₈-alkyl, —CH₂—S(O)₂—C₁-C₈-alkyl, (C₁-C₆-alkoxyimino)-C₁-C₆-alkyl, (C₂-C₆-alkenyloxyimino)-C₁-C₆-alkyl, (C₃-C₆-alkynyloxyimino)-C₁-C₆-alkyl, or
each Y is independently selected from a 4 to 7 membered heterocyclic ring system with 1 to 3 heteroatoms, but not O—O, O—S or 3 heteroatoms in a row, independently substituted by oxo, thiono or 1 to 12 substituents Z independently selected from the group consisting of hydrogen, halogen, cyano, hydroxy, amino, —SH, —CHO, —COOH, —CONH₂, —CONH(OH), —OCONH₂, (hydroxyimino)-C₁-C₆-alkyl, C₁-C₆-alkyl, C₁-C₆-halogenoalkyl having 1 to 5 halogen atoms, C₂-C₆-alkenyl, C₂-C₆-alkynyl, C₁-C₆-alkylamino, di-(C₁-C₆-alkyl)amino, C₁-C₆-alkoxy, C₁-C₆-halogenoalkoxy having 1 to 5 halogen atoms, C₂-C₆-alkenyloxy, C₂-C₆-halogenoalkenyloxy having 1 to 5 halogen atoms, C₃-C₆-alkynyloxy, C₃-C₆-halogenoalkynyloxy having 1 to 5 halogen atoms, C₃-C₆-cycloalkyl, C₃-C₆-halogenocycloalkyl having 1 to 5 halogen atoms, C₁-C₆-alkylcarbonyl, C₁-C₆-halogenoalkylcarbonyl having 1 to 5 halogen atoms, —CONH(C₁-C₆-alkyl), —CON(C₁-C₆-alkyl)₂, —CONH(OC₁-C₆-alkyl), —CON(OC₁-C₆-alkyl)(C₁-C₆-alkyl), C₁-C₆-alkoxycarbonyl, C₁-C₆-halogenoalkoxycarbonyl having 1 to 5 halogen atoms, C₁-C₆-alkylcarbonyloxy, C₁-C₆-halogenoalkylcarbonyloxy having 1 to 5 halogen atoms, C₁-C₆-alkylcarbonylamino, C₁-C₆-halogenoalkylcarbonylamino having 1 to 5 halogen atoms, —OCONH(C₁-C₆-alkyl), —OCON(C₁-C₆-alkyl)₂, —OCONH(OC₁-C₆-alkyl), —OCO(OC₁-C₆-alkyl), —S—C₁-C₆-alkyl, —S—C₁-C₆-halogenoalkyl having 1 to 5 halogen atoms, —S(O)—C₁-C₆-alkyl, —S(O)—C₁-C₆-halogenoalkyl having 1 to 5 halogen atoms, —S(O)₂—C₁-C₆-alkyl, —S(O)₂—C₁-C₆-halogenoalkyl having 1 to 5 halogen atoms,
   and salts, solvates, solvates of the salts, N-oxides, metal complexes or metalloid complexes thereof.

In still another embodiment, the present invention provides compounds according to formula (VII), wherein
R¹ is selected from the group consisting of hydrogen, C₁-C₄-alkyl, C₃-C₄-alkynyl, C₃-C₆-cycloalkyl, C₁-C₄-alkylcarbonyl, C₁-C₄-alkoxy-C₁-C₄-alkyl, C₁-C₄-alkoxycarbonyl,
n is 1 or 2,
each X is independently selected from the group consisting of halogen, cyano, C₁-C₄-alkyl, C₁-C₄-alkoxy and C₁-C₄-halogenoalkyl having 1 to 5 halogen atoms,
m is 1, 2 or 3, and
each Y is independently selected from the group consisting of hydrogen, halogen, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —CONH(C₁-C₄-alkyl), —CON(C₁-C₄-alkyl)₂, —CONH(OC₁-C₄-alkyl), —CON(OC₁-C₄-alkyl)(C₁-C₆-alkyl), —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino, or
each Y is selected from the group consisting of

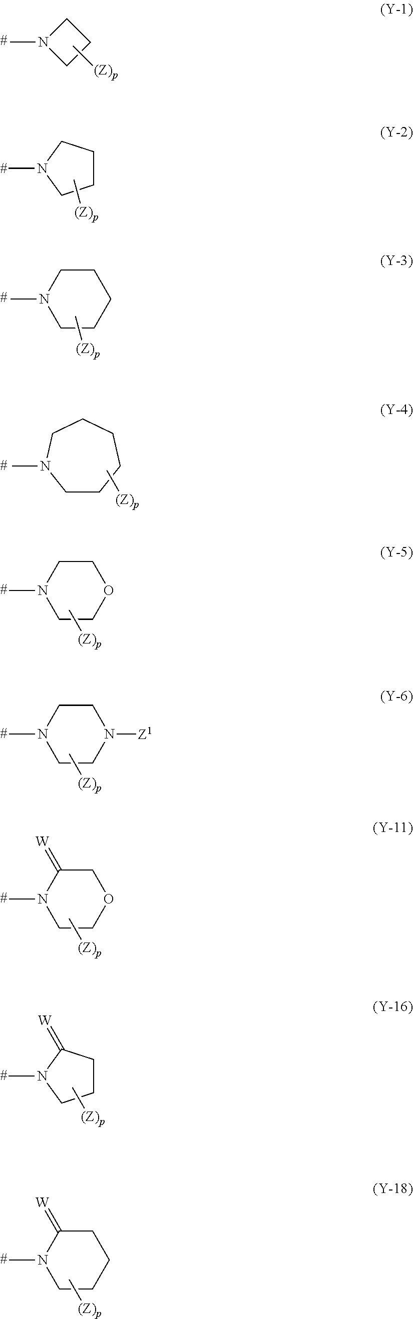

in which
# depicts the bond which connects Y to the rest of the molecule,
W is oxygen,
p is 0, 1, 2, 3, 4, 5 or 6,
Z is independently selected from the group consisting of hydrogen, halogen, cyano, hydroxy, amino, —SH, —CHO, —COOH, —CONH₂, —CONH(OH), —OCONH₂, (hydroxyimino)-C₁-C₄-alkyl, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, C₃-C₆-cycloalkyl, C₃-C₆-halogenocycloalkyl having 1 to 5 halogen atoms, —CONH(C₁-C₄-alkyl), —CON(C₁-C₄-alkyl)₂, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, and
Z¹ is independently selected from the group consisting of hydrogen, C₁-C₆-alkyl, C₁-C₆-halogenoalkyl having 1 to 5 halogen atoms, C₁-C₆-alkylamino, di-(C₁-C₆-alkyl)amino, C₁-C₆-alkoxy, C₃-C₆-cycloalkyl, C₃-C₆-halogenocycloalkyl having 1 to 5 halogen atoms, —CONH(C₁-C₆-alkyl), —CON(C₁-C₆-alkyl)₂,
   and salts, solvates, solvates of the salts, N-oxides, metal complexes or metalloid complexes thereof.

In yet another embodiment, the present invention provides compounds according to formula (VII), wherein
R¹ is hydrogen,
n is 1 or 2,
each X is independently selected from the group consisting of halogen, cyano, CH₃, OCH₃ and CF₃,
m is 1, 2 or 3, and
each Y is independently selected from the group consisting of hydrogen, halogen, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino,
   and salts, solvates, solvates of the salts, N-oxides, metal complexes or metalloid complexes thereof.

In yet another embodiment, the present invention provides compounds according to formula (VII), wherein
R¹ is hydrogen,
n is 1 or 2,
each X is independently selected from the group consisting of chloro, fluoro, cyano, CH₃, OCH₃ and CF₃,
m is 1, 2 or 3, and
each Y is independently selected from the group consisting of hydrogen, halogen, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino,
   and salts, solvates, solvates of the salts, N-oxides, metal complexes or metalloid complexes thereof.

In yet another embodiment, the present invention provides compounds according to formula (VII), wherein
R¹ is hydrogen,
n is 1 or 2,
each X is independently selected from the group consisting of chloro, fluoro, CH₃, OCH₃, and CF₃,
m is 1, 2 or 3, and
each Y is independently selected from the group consisting of hydrogen, fluoro, chloro, bromo, cyano, trifluoromethyl, difluoromethyl, methoxy, trifluoromethoxy, difluoromethoxy, methylamino, dimethylamino, methylsulfanyl, trifluoromethylsulfanyl, methylsulfinyl, trifluoromethylsulfinyl, methylsulfonyl, trifluoromethylsulfonyl, and acetylamino,
   and salts, solvates, solvates of the salts, N-oxides, metal complexes or metalloid complexes thereof.

In yet another embodiment, the present invention provides compounds according to formula (VII), wherein
R¹ is hydrogen,
n is 1 or 2,
each X is independently selected from the group consisting of chloro, fluoro, CH₃, OCH₃, and CF₃,
m is 1, 2 or 3, and
each Y is independently selected from the group consisting of hydrogen, fluoro, chloro, cyano, trifluoromethyl, methoxy, trifluoromethoxy, methylamino, dimethylamino, methylsulfanyl, trifluoromethylsulfanyl, methylsulfonyl, and acetylamino,
   and salts, solvates, solvates of the salts, N-oxides, metal complexes or metalloid complexes thereof.

The present invention relates further to a process of manufacturing a compound of formula (I)

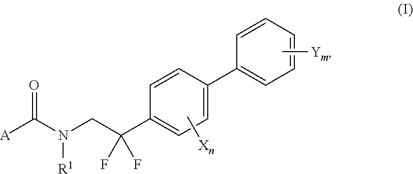

wherein R¹, n, X, m, Y and A have the meaning as described before for formula (I), comprising a step of reacting a compound of formula (VII)

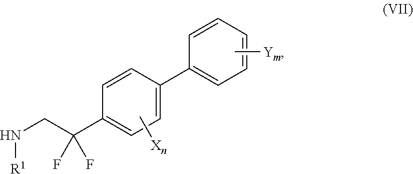

wherein R¹, n, X, m and Y have the meaning as described before for formula (VII),
[A] with a compound of formula (X)

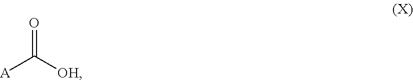

wherein A has the meaning as described before for formula (I),
in the presence of a dehydration reagent, or
[B] with a compound of formula (XI)

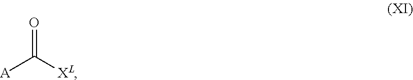

wherein
X^{L} is halogen, and
A has the meaning as described before for formula (I).

The reaction according to [A] is normally done in inert solvents, possibly in the presence of a base, in a specific embodiment in a temperature range between −30° C. and 50° C. under ambient pressure.

Inert solvents are, for example, halogen hydrocarbons, like dichloromethane, trichloromethane or chlorobenzene, hydrocarbons like benzene, toluene, n-pentane, n-hexane, cyclohexane, n-heptane, decaline or the like, furthermore, nitromethane, dioxane, dimethylformamide or acetonitril. Further, it is possible to use mixtures of the solvents mentioned before. In one specific embodiment, acetonitril is used.

Suitable dehydration reagents are, for example, carbodiimides like, for example, N,N′-diethyl-, N,N,′-dipropyl-, N,N′-diisopropyl-, N,N′-dicyclohexylcarbodiimide, N-(3-dimethylaminoisopropyl)-N′-ethyl-carbodiimide-hydrochloride (EDC), N-cyclohexylcarbodiimide-N′-propyloxymethyl-polystyrol (PS-carbodiimid) or carbonyl compounds, like carbonyldiimidazole, or 1,2-oxazolium compounds, like 2-ethyl-5-phenyl-1,2-oxazolium-3-sulfate or 2-tert-butyl-5-methyl-isoxazolium-perchlorate, or acylamino compounds, like 2-ethoxy-1-ethoxycarbonyl-1,2-dihydrochinoline, or propane phosphonic acid anhydride (T3P), or isobutyl chloroformate, or bis-(2-oxo-3-oxazolidinyl)-phosphorylchloride or benzotriazolyloxy-tri(dimethylamino)phosphoniumhexafluorophosphate, or O-(benzotriazol-1-yl)-N,N,N′,N′-tetra-methyluronium-hexafluorophosphate (HBTU), 2-(2-oxo-1-(2H)-pyridyl)-1,1,3,3-tetramethyl-uroniumtetrafluoro-borate (TPTU) or O-(7-azabenzotriazol-1-yl)-N,N,N′,N′-tetramethyl-uroniumhexafluorophosphate (HATU), or 1-hydroxybenzotriazole (HOBt), or benzotriazole-1-yloxytris(dimethylamino)-phosphoniumhexafluorophosphate (BOP), or N-hydroxysuccinimide, or mixtures of these, in combination with bases.

Bases are, for example, alkali carbonates, like, for example, sodium carbonate, potassium carbonate or caesium carbonate, or the respective hydrogencarbonate, or organic bases, like trialkylamines, e.g. triethylamine, N-methylmorpholine, N-methylpiperidine, 4-dimethylaminopyridine or diisopropylethylamine. In one embodiment, diisopropylethylamine is preferred.

The reaction according to [A] is in one specific embodiment realized by N-(3-dimethylaminoisopropyl)-N′-ethylcarbodiimide-hydrochloride (EDC). The reaction according to [A] is in another specific embodiment realized by 1-hydroxybenzotriazole (HOBt).

The reaction according to [A] is in a more specific embodiment realized by N-(3-dimethylaminoisopropyl)-N′-ethylcarbodiimide-hydrochloride (EDC) and 4-dimethylaminopyridine. The reaction according to [A] is in another more specific embodiment realized by 1-hydroxybenzotriazole (HOBt) and diisopropylethylamine.

The reaction according to [B] is normally done in inert solvents, in a specific embodiment in a temperature range between −30° C. and 50° C. under ambient pressure.

Inert solvents are, for example, halogen hydrocarbons, like dichloromethane, trichloromethane or chlorobenzene, hydrocarbons like benzene, toluene, n-pentane, n-hexane, cyclohexane, n-heptane, decaline or the like, furthermore, nitromethane, dioxane, dimethylformamide or acetonitril. Further, it is possible to use mixtures of the solvents mentioned before. In one specific embodiment, the inert solvent is selected from the group consisting of dichloromethane, dimethylformamide, dioxane, acetonitril, toluene, and mixtures thereof.

In one specific embodiment of the reaction according to [B], X^{L} is chloro or fluoro.

In a more specific embodiment of the reaction according to [B], X^{L} is chloro.

The definitions of radicals, and explanations, that are given above in general or in ranges of preference or further embodiments may be combined arbitrarily with one another, thus including combinations between the respective ranges and ranges of preference/embodiments. The definitions and explanations apply to the end products and also to the precursors and intermediates accordingly.

The invention further relates to a pharmaceutical composition comprising at least one compound of formula (I) according to anyone of the embodiments mentioned before.

The invention further relates to a pharmaceutical composition comprising at least one compound of formula (I) according to anyone of the embodiments mentioned before for the control, treatment and/or prevention of infections with helminths in animals and humans.

The invention further relates to the use of a compound of formula (I) of anyone of the embodiments mentioned before for the control, treatment and/or prevention of infections with helminths in animals and humans.

The invention further relates to the use of a pharmaceutical composition as mentioned before for the control, treatment and/or prevention of infections with helminths in animals and humans.

The invention further relates to the use of a compound of formula (I) of anyone of the embodiments mentioned before for the manufacturing of a medicament for the control, treatment and/or prevention of infections with helminths in animals and humans.

The invention further relates to a method for the control, treatment and/or prevention of infections with helminths in animals and humans, comprising the step of administering an effective amount of a compound of formula (I) of the embodiments mentioned before, or a pharmaceutical composition as mentioned before, to an animal or human in need thereof.

Saturated or unsaturated hydrocarbon radicals such as alkyl, alkanediyl or alkenyl may in each case, both alone and in conjunction with heteroatoms, as in alkoxy, for example, be—where possible—either straight-chain or branched.

Any substituted radicals may, unless indicated otherwise, be substituted one or more times, and the substituents in the case of multiple substitutions may be alike or different.

In the definitions of radicals that are stated as being preferred, halogen (halo) is fluoro, chloro, bromo and iodo, very preferably fluoro, chloro and bromo, and especially preferably fluoro and chloro.

Procedures and Methods

The synthesis of the compounds of the formula (I) can be performed according to or in analogy to Scheme 1, Scheme 2 or Scheme 3. The required starting materials are known or accessible via generally known procedures which are described in more detail in WO 2007/141009, WO 2013/064460 or WO 2014/004064.

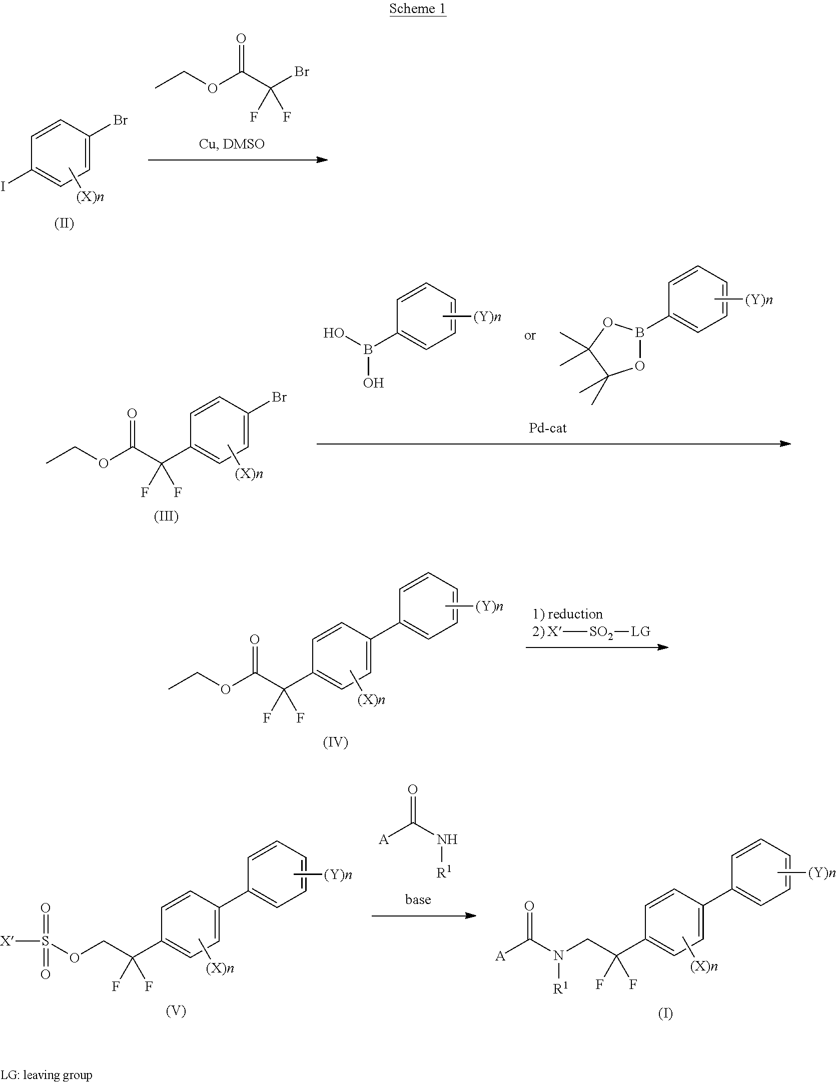

4-Bromoaryliodide (II) can react with ethyl bromodifluoroacetate in the presence of copper powder to give compound (III), such reactions are described for example in Journal of Fluorine Chemistry 2004, 125, 509-513. A Suzuki cross coupling reaction of arylbromide (III) with boronic acids or esters delivers biaryl derivative (IV) as described in Chem. Soc. Rev. 2014, 43, 412-443 and Indo Global Journal of Pharmaceutical Sciences 2012, 2, 315-367.

Ester (IV) can be reduced into the corresponding alcohol, which is then transformed into a good leaving group (X′═CF₃ or X′=Me, for example) to give compound (V) which can be transformed into (I) via nucleophilic substitution with an amide in presence of a base.

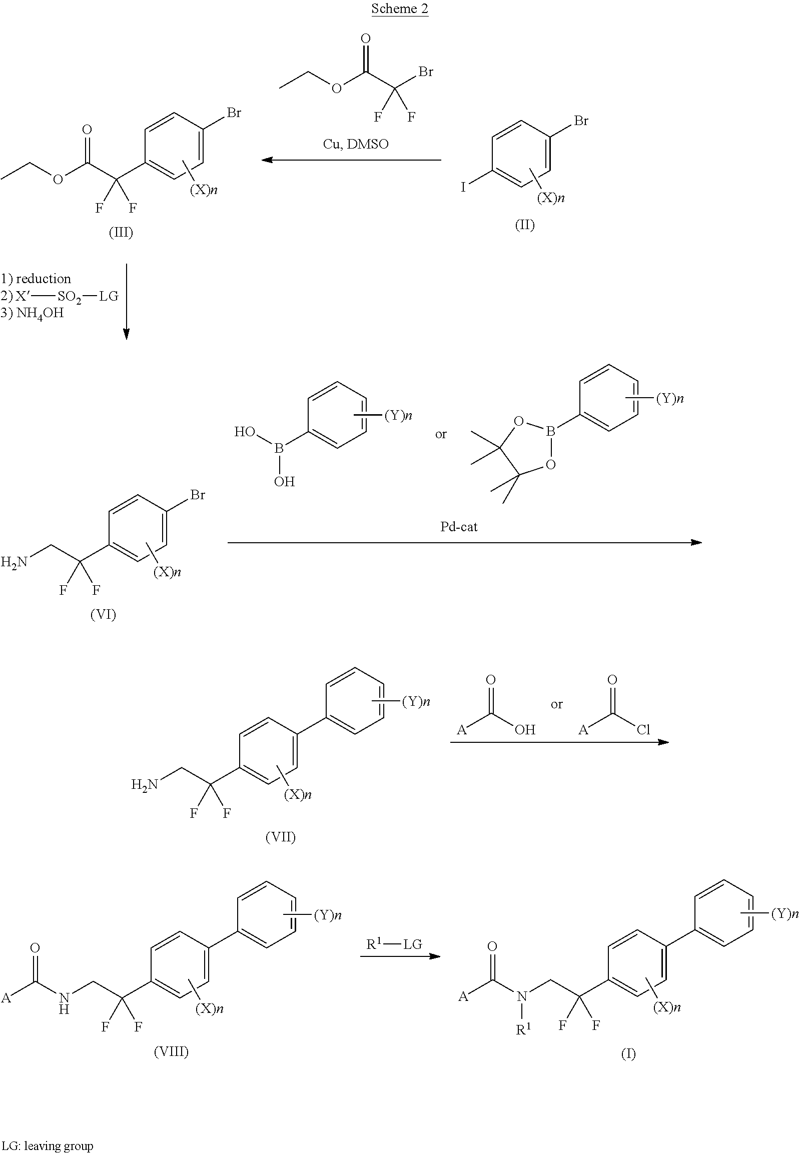

Ester (III) can be reduced into the corresponding alcohol, which is then transformed into a good leaving group (X′═CF₃ or X′=Me, for example), subsequent amination gives amine (VI).

Arylbromide (VI) can react in a Suzuki coupling reaction with boronic acids or esters to deliver biaryl derivatives (VII) as described in Chem. Soc. Rev. 2014, 43, 412-443 and Indo Global Journal of Pharmaceutical Sciences 2012, 2, 315-367. Amine (VII) can be then transformed into Amide (VIII), which gives then (I).

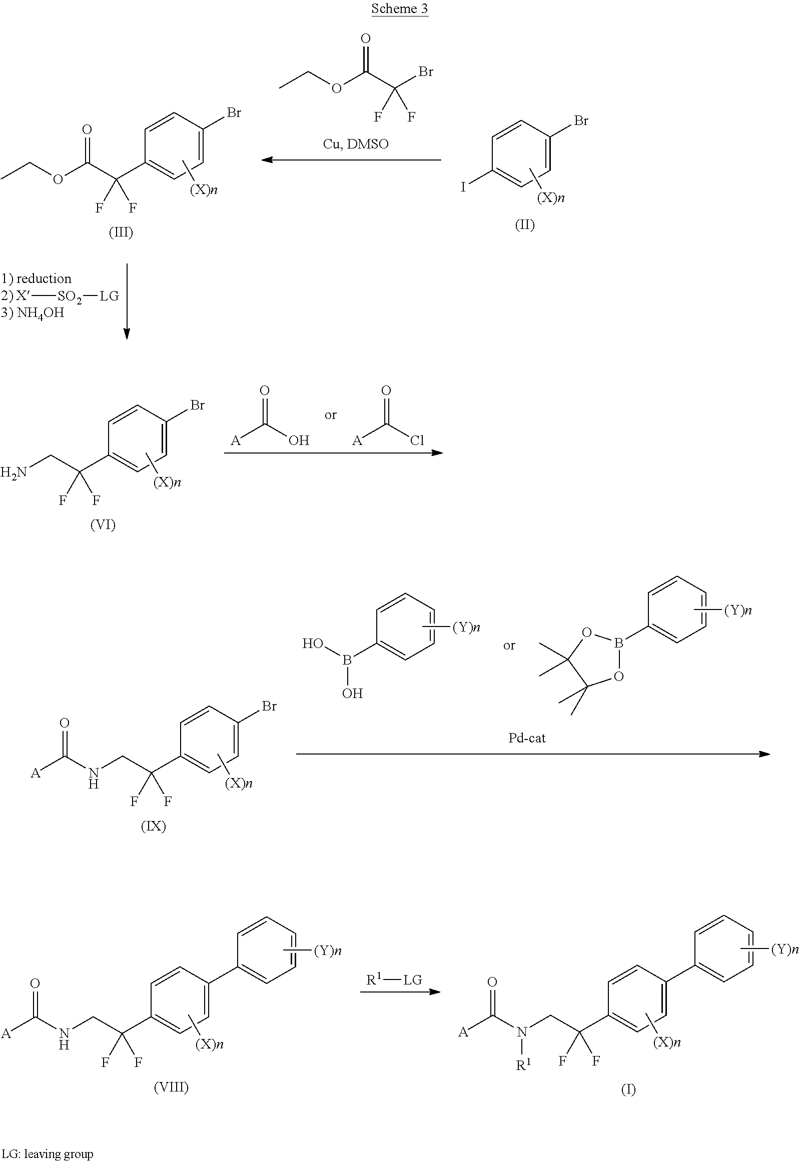

Amine (VI) can be acylated to give amide (IX). Arylbromide (IX) can react in a Suzuki coupling reaction with boronic acids or esters to deliver biaryl derivatives (VIII) as described in Chem. Soc. Rev. 2014, 43, 412-443 and Indo Global Journal of Pharmaceutical Sciences 2012, 2, 315-367.

The compounds according to the present invention can be prepared according to the processes described above. It will nevertheless be understood that, on the basis of his general knowledge and of available publications, the skilled worker will be able to adapt this method according to the specifics of each of the compounds, which it is desired to synthesize.

The compounds of the invention can be used as endoparasiticides. At least within the context of the present invention, the use as endoparasiticide shall comprise the use for the control, treatment and/or prevention of infections with helminths in animals and humans, preferably in non-human animals.

The compounds of the present invention act as anthelmintic agents against endoparasites in animals and humans.

The present invention further relates to medicaments that contain at least one compound according to the invention, usually together with one or more inert, non-toxic, pharmaceutically suitable excipients, and use thereof for the aforementioned purposes.

The compounds according to the invention can have systemic and/or local action. For this purpose they can be applied in a suitable way, e.g. by oral, parenteral, sublingual, lingual, buccal, rectal, dermal, or transdermal administration.

For these routes of application, the compounds according to the invention can be administered in suitable dosage forms.

Dosage forms functioning according to the prior art, which contain the compounds according to the invention in crystalline and/or amorphized and/or dissolved form, e.g. tablets (uncoated or coated tablets), tablets or films/wafers that disintegrate rapidly in the oral cavity, films/lyophilizates, capsules (for example hard or soft gelatin capsules), sugar-coated pills, granules, pellets, chewables, powders, emulsions, suspensions, aerosols or solutions, are suitable for oral administration.

Parenteral administration can take place with avoidance of an absorption step (e.g. intravenous, intraarterial, intracardiac, intraspinal or intralumbar) or with inclusion of an absorption (e.g intramuscular, subcutaneous, intracutaneous, percutaneous, or intraperitoneal). Administration forms suitable for parenteral administration are, inter alia, preparations for injection and infusion in the form of solutions, suspensions, emulsions, lyophilizates or sterile powders.

Suitable for the other routes of administration are, for example, tablets for lingual, sublingual or buccal administration, vaginal capsules, aqueous suspensions (lotions, shaking mixtures), lipophilic suspensions, ointments, creams, transdermal therapeutic systems (for example patches), milk, pastes, foams, or dusting powders.

The compounds according to the invention can be converted into the stated administration forms. This can take place in a manner known per se by mixing with inert, non-toxic, pharmaceutically suitable excipients. These excipients include inter alia carriers, solvents, emulsifiers and dispersants or wetting agents, binders, synthetic and natural polymers, stabilizers, and masking flavours and/or odours.

In the veterinary field and in animal keeping, the administration of the active compounds according to the invention is carried out in the known manner directly or enterally, parenterally, dermally or nasally in the form of suitable preparations. Administration can be carried out prophylactically or therapeutically.

In the animal health field, i.e. in the field of veterinary medicine, the compounds according to the present invention are active against animal parasites, in particular endoparasites. The term endoparasite includes in particular helminths and protozoae, such as coccidia. The compounds of formula (I) are preferably active against helminths.

In the field of veterinary medicine the compounds according to the invention are suitable, with favourable warm blood toxicity, for controlling parasites, preferably helminths, which occur in animal breeding and animal husbandry in livestock, breeding, zoo, laboratory, experimental and domestic animals. They are active against all or specific stages of development of the parasites.

Agricultural livestock include, for example mammals, such as, sheep, goats, horses, donkeys, camels, buffaloes, rabbits, reindeers, fallow deers, and in particular cattle and pigs; or poultry such as turkeys, ducks, geese, and in particular chickens; or fish or crustaceans e.g. in aquaculture; or as the case may be insects such as bees.

Domestic animals include, for example mammals, such as hamsters, guinea pigs, rats, mice, chinchillas, ferrets or in particular dogs, cats; cage birds; reptiles; amphibians or aquarium fish.

According to a particular embodiment, the compounds according to the invention are administered to mammals.

According to another particular embodiment, the compounds according to the invention are administered to birds, namely cage birds or in particular poultry.

By using the active compounds according to the invention to control animal parasites, preferably helminths, it is intended to reduce or prevent illness, cases of deaths and performance reductions (in the case of meat, milk, wool, hides, eggs, honey and the like), so that more economical and simpler animal keeping is made possible and better animal well-being is achievable.

The term “control” or “controlling” as used herein with regard to the animal health field means that the active compounds are effective in reducing the numbers of the respective parasites in an animal infected with such parasites to innocuous levels. More specifically, “controlling”, as used herein, means that the active compound is effective in killing the respective parasites, inhibiting their growth, and/or inhibiting their proliferation.

Exemplary pathogenic endoparasites of humans and animals, which are helminths, include platyhelmintha (e.g. monogenea, cestodes and trematodes), nematodes, acanthocephala, and pentastoma. Additional exemplary helminths include—, without any limitation:

Monogenea: e.g.: Gyrodactylus spp., Dactylogyrus spp., Polystoma spp.

Cestodes: From the order of the Pseudophyllidea for example: Diphyllobothrium spp., Spirometra spp., Schistocephalus spp., Ligula spp., Bothridium spp., Diplogonoporus spp.

From the order of the Cyclophyllida for example: Mesocestoides spp., Anoplocephala spp., Paranoplocephala spp., Moniezia spp., Thysanosoma spp., Thysaniezia spp., Avitellina spp., Stilesia spp., Cittotaenia spp., Andyra spp., Bertiella spp., Taenia spp., Echinococcus spp., Hydatigera spp., Davainea spp., Raillietina spp., Hymenolepis spp., Echinolepis spp., Echinocotyle spp., Diorchis spp., Dipylidium spp., Joyeuxiella spp., Diplopylidium spp.

Trematodes: From the class of the Digenea for example: Diplostomum spp., Posthodiplostomum spp., Schistosoma spp., Trichobilharzia spp., Ornithobilharzia spp., Austrobilharzia spp., Gigantobilharzia spp., Leucochloridium spp., Brachylaima spp., Echinostoma spp., Echinoparyphium spp., Echinochasmus spp., Hypoderaeum spp., Fasciola spp., Fasciolides spp., Fasciolopsis spp., Cyclocoelum spp., Typhlocoelum spp., Paramphistomum spp., Calicophoron spp., Cotylophoron spp., Gigantocotyle spp., Fischoederius spp., Gastrothylacus spp., Notocotylus spp., Catatropis spp., Plagiorchis spp., Prosthogonimus spp., Dicrocoelium spp., Eurytrema spp., Troglotrema spp., Paragonimus spp., Collyriclum spp., Nanophyetus spp., Opisthorchis spp., Clonorchis spp. Metorchis spp., Heterophyes spp., Metagonimus spp.

Nematodes: Trichinellida for example: Trichuris spp., Capillaria spp., Paracapillaria spp., Trichomosoides spp., Trichinella spp., Eucoleus spp.

From the order of the Tylenchida for example: Micronema spp., Strongyloides spp.

From the order of the Rhabditina for example: Strongylus spp., Triodontophorus spp., Oesophagodontus spp., Trichonema spp., Gyalocephalus spp., Cylindropharynx spp., Poteriostomum spp., Cyclococercus spp., Cylicostephanus spp., Oesophagostomum spp., Chabertia spp., Stephanurus spp., Necator spp., Ancylostoma spp., Uncinaria spp., Bunostomum spp., Globocephalus spp., Syngamus spp., Cyathostoma spp., Metastrongylus spp., Dictyocaulus spp., Muellerius spp., Protostrongylus spp., Neostrongylus spp., Cystocaulus spp., Pneumostrongylus spp., Spicocaulus spp., Elaphostrongylus spp. Parelaphostrongylus spp., Crenosoma spp., Paracrenosoma spp., Angiostrongylus spp., Aelurostrongylus spp., Filaroides spp., Parafilaroides spp., Oslerus spp., Trichostrongylus spp., Haemonchus spp., Ostertagia spp., Teladorsagia spp., Marshallagia spp., Cooperia spp., Nematodirus spp., Hyostrongylus spp., Obeliscoides spp., Amidostomum spp., Ollulanus spp.; Heligmosomoides spp., Nippostrongylus spp.

From the order of the Spirurida for example: Oxyuris spp., Enterobius spp., Passalurus spp., Syphacia spp., Aspiculuris spp., Heterakis spp.; Ascaris spp., Toxascaris spp., Toxocara spp., Baylisascaris spp., Parascaris spp., Anisakis spp., Ascaridia spp.; Gnathostoma spp., Physaloptera spp., Thelazia spp., Gongylonema spp., Habronema spp., Parabronema spp., Draschia spp., Dracunculus spp.; Stephanofilaria spp., Parafilaria spp., Setaria spp., Loa spp., Dirofilaria spp., Litomosoides spp., Brugia spp., Wuchereria spp., Onchocerca spp., Spirocerca spp.

Acantocephala: From the order of the Oligacanthorhynchid, for example: Macracanthorhynchus spp., Prosthenorchis spp.; from the order of the Polymorphida for example: Filicollis spp.; from the order of the Moniliformida for example: Moniliformis spp.

From the order of the Echinorhynchida, for example: Acanthocephalus spp., Echinorhynchus spp., Leptorhynchoides spp.

Pentastoma: From the order of the Porocephalida, for example: Linguatula spp.

Thus, one embodiment of the present invention refers to compounds according to the invention for use as a medicament.

Another aspect refers to compounds according to the invention for use as an antiendoparasitical agent, in particular an anthelminthic agent. For example, compounds according to the invention can be used as an antiendoparasitical agent, in particular an anthelminthic agent, e.g., in animal husbandry, in animal breeding, in animal housing, in the hygiene sector.

In a particular embodiment, for the animal health field, mixtures with other anthelmintics are also provided.

Exemplary mixing partners include, without any limitation:

Anthelmintic actives, including nematicidal, trematicidal and cestocidal actives:

From the class of macrocyclic lactones, for example:

abamectin, doramectin, emamectin, eprinomectin, ivermectin, milbemycin, moxidectin, nemadectin, selamectin;

from the class of benzimidazoles and probenzimidazoles, for example:

albendazole, albendazole sulfoxide, cambendazole, cyclobendazole, febantel, fenbendazole, flubendazole, mebendazole, netobimin, oxfendazole, oxibendazole, parbendazole, thiabendazole, thiophanate, triclabendazole;

from the class of cyclooctadepsipeptides, for example:

emodepside, PF1022;

from the class of aminoacetonitrile derivatives, for example:

monepantel;

from the class of tetrahydropyrimidines, for example:

morantel, pyrantel, oxantel;

from the class of imidazothiazoles, for example:

butamisole, levamisole, tetramisole;

from the class of salicylanilides, for example:

bromoxanide, brotianide, clioxanide, closantel, niclosamide, oxyclozanide, rafoxanide, tribromsalan;

from the class of paraherquamides, for example:

derquantel, paraherquamide;

from the class of aminophenylamidines, for example:

amidantel, deacylated amidantel (dAMD), tribendimidine;

from the class of organophosphates, for example:

coumaphos, crufomate, dichlorvos, haloxon, naphthalofos, trichlorfon;

from the class of substituted phenols, for example:

bithionole, disophenol, hexachlorophen, niclofolan, meniclopholan, nitroxynil;

from the class of piperazinones, for example:

praziquantel, epsiprantel;

from the class of carbanilides, for example:

imidocarb;

from the class of quinazolinone alkaloid, for example:

halofuginon;

from the class of sulfonamides, for example:

sulfaclozin;

from the class of triazines, for example:

diclazuril, toltrazuril;

from diverse other classes, for example:

amoscanate, bephenium, bunamidine, clonazepam, clorsulon, diamfenetide, dichlorophen, diethylcarbamazine, emetine, hetolin, hycanthone, lucanthone, miracil, mirasan, niclosamide, niridazole, nitroxynile, nitroscanate, oltipraz, omphalotin, oxamniquine, paromomycin, piperazine, resorantel.

All named mixing partners can, if their functional groups enable this, optionally form salts with suitable bases or acids.

In another particular embodiment, for the animal health field, mixtures with ectoparasiticides are also provided.

Exemplary mixing partners include, without any limitation:

from the class of amidine derivatives, for example:

amitraz, chlormebuform, cymiazole, demiditraz;

from the class of arylisoxazolines, not excluding related classes with pyrroline or pyrrolidine moiety replacing the isoxazoline ring, for example:

afoxolaner, fluralaner;

from the class of bacillus thuringiensis strains, for example:

bacillus thuringiensis strains;

from the class of benzoylureas, for example:

bistrifluron, chlofluazuron, chlorfluazuron, diflubenzuron, fluazuron, flucycloxuron, flufenoxuron, hexaflumuron, lufenuron, novaluron, noviflumuron, penfluron, teflubenzuron, triflumuron;

from the class of beta-ketonitrile derivatives, for example:

cyenopyrafen, cyflumetofen;

from the class of carbamates, for example:

alanycarb, aldicarb, aldoxycarb, allyxycarb, aminocarb, bendiocarb, benfuracarb, bufencarb, butacarb, butocarboxim, butoxycarboxim, carbaryl, carbofuran, carbosulfan, cloethocarb, dimetilan, ethiofencarb, fenobucarb, fenothiocarb, formetanate, formparanate, furathiocarb, isoprocarb, metam-sodium, methiocarb, methomyl, metolcarb, oxamyl, pirimicarb, promecarb, propoxur, thiodicarb, thiofanox, triazamate, trimethacarb, xmc, xylylcarb;

from the class of chloronicotinyls, for example:

acetamiprid, clothianidin, dinotefuran, flupyradifurone, imidacloprid, nicotine, nitenpyram, nithiazine, thiacloprid, thiamethoxam;

from the class of diacylhydrazines, for example:

chromafenozide, halofenozide, methoxyfenozide, tebufenozide;

from the class of diamides, for example:

chlorantraniliprole, cyantraniliprole;

from the class of dicarboxamides, for example:

flubendiamide;

from the class of dinitrophenols, for example:

binapacyrl, dinobuton, dinocap, dnoc;

from the class of feeding inhibitors, for example:

cryolite, flonicamid, pymetrozine;

from the class of fumigants, for example:

aluminium phosphide, methyl bromide, sulphuryl fluoride;

from the class of halogenated carbonhydrogen compounds (hch), for example:

ddt, methoxychlor;

from the class of macrocyclic lactones, for example:

moxidectin, emamectin benzoate, latidectin, lepimectin;

from the class of microorganisms, for example:

bacillus spec., beauveria spec., metarrhizium spec., paecilomyces spec., verticillium spec.;

from the class of mite growth inhibitors, for example:

amidoflumet, benclothiaz, benzoximate, bifenazate, bromopropylate, chlordimeform, chlorobenzilate, chloropicrin, clofentezine, clothiazoben, cycloprene, dicyclanil, etoxazole, fenoxacrim, fentrifanil, flubenzimine, flufenerim, flutenzin, gossyplure, hexythiazox, hydramethylnone, japonilure, metoxadiazone, petroleum, potassium oleate, pyridalyl, quinomethionate, tetrasul, triarathene;

from the class of natural products, for example:

codlemone, essential oils, thuringiensin;

from the class of neem components, for example:

azadirachtin a;

from the class of nereistoxin analogues, for example:

bensultap, cartap, sulfoxaflor, thiocyclam, thiocyclam hydrogen oxalate, thiosultap sodium, thiosultap-sodium;

from the class of organic acids, for example:

formic acid, oxalic acid;

from the class of organochlorines, for example:

camphechlor, chlordane, endosulfan, gamma-hch, hch, heptachlor, lindane;

from the class of organophosphates, for example:

acephate, aromfenvinfos (-methyl), aromophos-ethyl, autathiofos, azamethiphos, azinphos (-methyl, -ethyl), cadusafos, carbophenothion, chlorethoxyfos, chlorfenvinphos, chlormephos, chlorpyrifos (-methyl/-ethyl), cyanofenphos, cyanophos, demeton-s-methyl, demeton-s-methylsulphone, dialifos, diazinon, dichlofenthion, dichlorvos/ddvp, dicrotophos, dimethoate, dimethylvinphos, dioxabenzofos, disulfoton, epn, ethion, ethoprophos, etrimfos, famphur, fenamiphos, fenitrothion, fensulfothion, fenthion, flupyrazofos, fonofos, formothion, fosmethilan, fosthiazate, heptenophos, iodofenphos, iprobenfos, isazofos, isofenphos, isopropyl o-salicylate, isoxathion, malathion, mecarbam, methacrifos, methamidophos, methidathion, mevinphos, monocrotophos, naled, omethoate, oxydemeton-methyl, parathion (-methyl/-ethyl), phenthoate, phorate, phosalone, phosmet, phosphamidone, phosphocarb, phoxim, pirimiphos (-methyl/-ethyl), profenofos, propaphos, propetamphos, prothiofos, prothoate, pyraclofos, pyridaphenthion, pyridathion, quinalphos, sebufos, sulfotep, sulprofos, tebupirimfos, temephos, terbufos, tetrachlorvinphos, thiometon, triazophos, triclorfon, vamidothion;

from the class of organotin compounds, for example:

azocyclotin, cyhexatin, fenbutatin-oxide;

from the class of other decouplers, for example:

sulfluramid;

from the class of other inhibitors of cuticle development, for example:

buprofezin, cyromazine;

from the class of other inhibitors of cuticle development, for example:

buprofezin, cyromazine;

from the class of others, for example:

chinomethionat, pyrifluquinazon;

from the class of oxadiazines, for example:

indoxacarb;

from the class of phenylpyrazoles, for example:

acetoprole, ethiprole, fipronil, pyrafluprole, pyriprole, vaniliprole;

from the class of pyrethroids, for example:

acrinathrin, allethrin (d-cis-trans, d-trans-), beta-cyfluthrin, bifenthrin, bioallethrin, bioallethrin-s-cyclopentyl-isomer, bioethanomethrin, biopermethrin, bioresmethrin, chlovaporthrin, cis-cypermethrin, cis-permethrin, cis-resmethrin, clocythrin, cycloprothrin, cyfluthrin, cyhalothrin (lambda-), cypermethrin (alpha-, beta-, theta-, zeta-), cyphenothrin, deltamethrin, empenthrin (1r-isomer), esfenvalerate, etofenprox, fenfluthrin, fenpropathrin, fenpyrithrin, fenvalerate, flubrocythrinate, flucythrinate, flufenprox, flumethrin, fluvalinate, fubfenprox, gamma-cyhalothrin, imiprothrin, kadethrin, lambda-cyhalothrin, metofluthrin, permethrin (cis-, trans-), phenothrin (1r-trans isomer), prallethrin, profluthrin, protrifenbute, pyresmethrin, pyrethrins (pyrethrum), resmethrin, ru 15525, silafluofen, tau-fluvalinate, tefluthrin, terallethrin, tetramethrin (-1r- isomer), tralomethrin, transfluthrin, zxi 8901;

from the class of pyrroles, for example:

chlorfenapyr;

from the class of quinones, for example:

acequinocyl;

from the class of rotenone, for example:

rotenone;

from the class of semicarbazones, for example:

metaflumizone;

from the class of spinosynes, for example:

spinetoram, spinosad;

from the class of tetronic and tetramic acids, for example:

spirodiclofen, spiromesifen, spirotetramat;

from the class of nereistoxin analogues, for example:

bensultap, cartap, sulfoxaflor, thiocyclam, thiocyclam hydrogen oxalate, thiosultap sodium, thiosultap-sodium;

from diverse other classes, for example:

amoscanate, bephenium, bunamidine, clonazepam, clorsulon, diamfenetide, dichlorophen, diethylcarbamazine, emetine, hetolin, hycanthone, lucanthone, miracil, mirasan, niclosamide, niridazole, nitroxynile, nitroscanate, oltipraz, omphalotin, oxamniquine, paromomycin, piperazine, resorantel.

Salts like hydrochlorides, tartrates, citrates, embonates/pamoates or benzoates are included.

The invention further relates to the use of a compound of formulae (I), (I-1) or (I-2) of anyone of the embodiments mentioned before for the control of nematodes in agricultures.

Agriculture shall encompass the production of food and feed crops, forestry, the protection of stored products including food, feed but also other materials. Preferably agriculture shall encompass the production of food and feed crops, forestry, the protection of stored products being food, feed, and materials of plant origin.

The compounds of the formulae (I), (I-1) or (I-2) can be used for curative or protective control of phytopathogenic nematodes. The invention therefore also relates to curative and protective methods for controlling phytopathogenic nematodes by the use of these compounds or of compositions comprinsing the same, which are applied to the the phytopathogenic nematodes, their habitat, the plant, the seed thereof, plant parts, plant propagation material or the soil on which the plants are grown or intended to be grown.

The invention further relates to the use of a compound of formulae (I), (I-1) or (I-2) of anyone of the embodiments mentioned before for controlling phytopathogenic nematodes.

The invention further relates to a method for controlling phytopathogenic nematodes comprising the step of applying the compounds of formulae (I), (I-1) or (I-2) of anyone of the embodiments mentioned before to the plant or plant parts.

The invention also relates to a method for controlling phytopathogenic nematodes comprising the step of applying the compounds of formulae (I), (I-1) or (I-2) of anyone of the embodiments mentioned before to plant propagation material.

The invention also relates to a method for controlling phytopathogenic nematodes comprising the step of applying the compounds of formulae (I), (I-1) or (I-2) of anyone of the embodiments mentioned before to the seed.

The invention also relates to a method for controlling phytopathogenic nematodes comprising the step of applying the compounds of formulae (I), (I-1) or (I-2) of anyone of the embodiments mentioned before to the soil on which the plants are grown or intended to be grown.

The invention further relates to the use of a compound of formulae (I), (I-1) or (I-2) of anyone of the embodiments mentioned before for the control of nematodes in plants or seeds.

The invention further relates to a method for the control of nematodes in agricultures comprising the step of applying a compound of formulae (I), (I-1) or (I-2) of anyone of the embodiments mentioned before to a seed or a plant which is infected with nematodes, or which is at risk for being infected with nematodes.

The invention further relates to the use of a compound of formulae (I), (I-1) or (I-2) of anyone of the embodiments mentioned before for treating seed for the purpose of protecting the seed and the resultant plant against nematodes.

The invention further relates to a method for protecting seeds and germinating plants from attack by nematodes, comprising the step of treating the seed with a compound of formulae (I), (I-1) or (I-2) of anyone of the embodiments mentioned before.

All plants and plant parts can be treated in accordance with the invention. Plants are understood here to mean all plants and plant populations, such as desired and undesired wild plants or crop plants (including naturally occurring crop plants). Crop plants may be plants which can be obtained by conventional breeding and optimization methods or by biotechnological and genetic engineering methods or combinations of these methods, including the transgenic plants and including the plant cultivars which are protectable and non-protectable by plant breeders' rights. Plant parts are understood to mean all parts and organs of plants above and below the ground, such as shoot, leaf, flower and root, examples of which include leaves, needles, stalks, stems, flowers, fruit bodies, fruits and seeds, and also roots, tubers and rhizomes. The plant parts also include harvested material and vegetative and generative propagation material, for example cuttings, tubers, rhizomes, slips, seedlings and seeds.

Plants which can be treated in accordance with the invention include the following plants from the group of the useful plants, ornamentals, turfs, generally used trees which are employed as ornamentals in the public and domestic sectors, and forestry trees. Forestry trees comprise trees for the production of timber, cellulose, paper and products made from parts of the trees.

The term useful plants as used in the present context refers to crop plants which are employed as plants for obtaining foodstuffs, feedstuffs, fuels or for industrial purposes.

The useful plants include for example the following types of plants: turf, vines, cereals, for example wheat, barley, rye, oats, rice, maize and millet/sorghum, corn, maize; beet, for example sugar beet and fodder beet; fruits, for example pome fruit, stone fruit and soft fruit, for example apples, pears, plums, peaches, almonds, cherries and berries, for example strawberries, raspberries, blackberries; legumes, for example beans, lentils, peas and soybeans; oil crops, for example oilseed rape, winter oilseed rape, spring oilseed rap, canola, mustard, poppies, olives, sunflowers, coconuts, castor oil plants, cacao and peanuts; cucurbits, for example pumpkin/squash, cucumbers and melons; fibre plants, for example cotton, flax, hemp and jute; citrus fruit, for example oranges, lemons, grapefruit and tangerines; vegetables, for example spinach, lettuce, asparagus, cabbage species, carrots, onions, tomatoes, potatoes and bell peppers; Lauraceae, for example avocado, Cinnamomum, camphor, or else plants such as tobacco, nuts, coffee, aubergine, sugar cane, tea, pepper, grapevines, hops, bananas, latex plants and ornamentals, for example flowers, shrubs, deciduous trees and coniferous trees. This enumeration is no limitation.

The following plants are considered to be particularly suitable target crops: cotton, aubergine, turf, pome fruit, stone fruit, soft fruit, maize, wheat, barley, cucumber, tobacco, vines, rice, cereals, pear, beans, soybeans, oilseed rape, tomato, bell pepper, melons, cabbage, potato and apple.

Examples of trees are: Abies sp., Eucalyptus sp., Picea sp., Pinus sp., Aesculus sp., Platanus sp., Tilia sp., Acer sp., Tsuga sp., Fraxinus sp., Sorbus sp., Betula sp., Crataegus sp., Ulmus sp., Quercus sp., Fagus sp., Salix sp., Populus sp.

Examples of turf grasses are including cool-season turf grasses and warm-season turf grasses.

Cold-season turf grasses are bluegrasses (Poa spp.), such as Kentucky bluegrass (Poa pratensis L.), rough bluegrass (Poa trivialis L.), Canada bluegrass (Poa compressa L.), annual bluegrass (Poa annua L.), upland bluegrass (Poa glaucantha Gaudin), wood bluegrass (Poa nemoralis L.) and bulbous bluegrass (Poa bulbosa L.); bentgrasses (Agrostis spp.) such as creeping bentgrass (Agrostis palustris Huds.), colonial bentgrass (Agrostis tenuis Sibth.), velvet bentgrass (Agrostis canina L.), South German mixed bentgrass (Agrostis spp. including Agrostis tenuis Sibth., Agrostis canina L., and Agrostis palustris Huds.), and redtop (Agrostis alba L.);

fescues (Festuca spp.), such as red fescue (Festuca rubra L. spp. rubra), creeping fescue (Festuca rubra L.), chewings fescue (Festuca rubra commutata Gaud.), sheep fescue (Festuca ovina L.), hard fescue (Festuca longifolia Thuill.), hair fescue (Festucu capillata Lam.), tall fescue (Festuca arundinacea Schreb.) and meadow fescue (Festuca elanor L.);

ryegrasses (Lolium spp.), such as annual ryegrass (Lolium multiflorum Lam.), perennial ryegrass (Lolium perenne L.) and Italian ryegrass (Lolium multiflorum Lam.);

and wheatgrasses (Agropyron spp.), such as fairway wheatgrass (Agropyron cristatum (L.) Gaertn.), crested wheatgrass (Agropyron desertorum (Fisch.) Schult.) and western wheatgrass (Agropyron smithii Rydb.);

and further cool-season turf grasses like beachgrass (Ammophila breviligulata Fern.), smooth bromegrass (Bromus inermis Leyss.), cattails such as timothy (Phleum pratense L.), sand cattail (Phleum subulatum L.), orchardgrass (Dactylis glomerata L.), weeping alkaligrass (Puccinellia distans (L.) Parl.) and crested dog's-tail (Cynosurus cristatus L.).

Warm-season turf grasses are Bermuda grass (Cynodon spp. L. C. Rich), zoysia grass (Zoysia spp. Willd.), St. Augustine grass (Stenotaphrum secundatum Walt Kuntze), centipede grass (Eremochloa ophiuroides Munro Hack.), carpetgrass (Axonopus affinis Chase), Bahia grass (Paspalum notatum Flugge), Kikuyu grass (Pennisetum clandestinum Hochst. ex Chiov.), buffalo grass (Buchloe dactyloids (Nutt.) Engelm.), blue grama (Bouteloua gracilis (H.B.K.) Lag. ex Griffiths), seashore paspalum (Paspalum vaginatum Swartz) and sideoats grama (Bouteloua curtipendula (Michx. Torr.).

In the present context, the term “nematodes” may not only refer to nematodes damaging humans or animals, but also to nematodes causing damages in agriculture, e.g. nematodes damaging plants and seeds.

In the present context, the term “nematodes” comprises all species of the phylum Nematoda, including species acting as parasites on plants or fungi (for example species of the order Aphelenchida, Meloidogyne, Tylenchida and others) or else on humans and animals as described before and causing damage in or on these living organisms, and also other parasitic helminths.

A nematicide in crop protection, as described herein, is capable of controlling nematodes.

The term “controlling nematodes” means killing the nematodes or preventing or impeding their development or their growth or preventing or impeding their penetration into or their sucking on plant tissue.

Here, the efficacy of the compounds is determined by comparing mortalities, gall formation, cyst formation, nematode density per volume of soil, nematode density per root, number of nematode eggs per soil volume, mobility of the nematodes between a plant or plant part treated with one of the compounds of the formulae (I), (I-1) or (I-2) or the treated soil and an untreated plant or plant part or the untreated soil (100%). Preferably, the reduction achieved is 25-50% in comparison to an untreated plant, plant part or the untreated soil, particularly preferably 51-79% and very particularly preferably the complete kill or the complete prevention of development and growth of the nematodes by a reduction of 80 to 100%. The control of nematodes as described herein also comprises the control of proliferation of the nematodes (development of cysts and/or eggs). Compounds of the formulae (I), (I-1) or (I-2) can also be used to keep the plants or animals healthy, and they can be employed curatively, preventatively or systemically for the control of nematodes.

The person skilled in the art knows methods for determining mortalities, gall formation, cyst formation, nematode density per volume of soil, nematode density per root, number of nematode eggs per volume of soil, mobility of the nematodes.

The use of a compound of the formulae (I), (I-1) or (I-2) may keep the plant healthy and also comprises a reduction of the damage caused by nematodes and an increase of the harvest yield.

In the present context, the term “nematodes” refers to plant nematodes which comprise all nematodes which damage plants. Plant nematodes comprise phytoparasitic nematodes and soil-borne nematodes. The phytoparasitic nematodes include ectoparasites such as Xiphinema spp., Longidorus spp. and Trichodorus spp.; semiparasites such as Tylenchulus spp.; migratory endoparasites such as Pratylenchus spp., Radopholus spp. and Scutellonema spp.; non-migratory parasites such as Heterodera spp., Globodera spp. and Meloidogyne spp., and also stem and leaf endoparasites such as Ditylenchus spp., Aphelenchoides spp. and Hirschmaniella spp. Particularly damaging root-parasitic soil nematodes are, for example, cyst-forming nematodes of the genera Heterodera or Globodera, and/or root gall nematodes of the genus Meloidogyne. Damaging species of these genera are, for example, Meloidogyne incognita, Heterodera glycines (soya bean cyst nematode), Globodera pallida and Globodera rostochiensis (yellow potato cyst nematode), these species being controlled effectively by the compounds described in the present text. However, the use of the compounds described in the present text is by no means restricted to these genera or species, but also extends in the same manner to other nematodes.

The plant nematodes include, for example, Aglenchus agricola, Anguina tritici, Aphelenchoides arachidis, Aphelenchoides fragaria, and the stem and leaf endoparasites Aphelenchoides spp., Belonolaimus gracilis, Belonolaimus longicaudatus, Belonolaimus nortoni, Bursaphelenchus cocophilus, Bursaphelenchus eremus, Bursaphelenchus xylophilus and Bursaphelenchus spp., Cacopaurus pestis, Criconemella curvata, Criconemella onoensis, Criconemella ornata, Criconemella rusium, Criconemella xenoplax (=Mesocriconema xenoplax) and Criconemella spp., Criconemoides ferniae, Criconemoides onoense, Criconemoides ornatum and Criconemoides spp., Ditylenchus destructor, Ditylenchus dipsaci, Ditylenchus myceliophagus and also the stem and leaf endoparasites Ditylenchus spp., Dolichodorus heterocephalus, Globodera pallida (=Heterodera pallida), Globodera rostochiensis (yellow potato cyst nematode), Globodera solanacearum, Globodera tabacum, Globodera virginia and the non-migratory cyst-forming parasites Globodera spp., Helicotylenchus digonicus, Helicotylenchus dihystera, Helicotylenchus erythrine, Helicotylenchus multicinctus, Helicotylenchus nannus, Helicotylenchus pseudorobustus and Helicotylenchus spp., Hemicriconemoides, Hemicycliophora arenaria, Hemicycliophora nudata, Hemicycliophora parvana, Heterodera avenae, Heterodera cruciferae, Heterodera glycines (soya bean cyst nematode), Heterodera oryzae, Heterodera schachtii, Heterodera zeae and the non-migratory cyst-forming parasites Heterodera spp., Hirschmaniella gracilis, Hirschmaniella oryzae, Hirschmaniella spinicaudata and the stem and leaf endoparasites Hirschmaniella spp., Hoplolaimus aegyptii, Hoplolaimus californicus, Hoplolaimus columbus, Hoplolaimus galeatus, Hoplolaimus indicus, Hoplolaimus magnistylus, Hoplolaimus pararobustus, Longidorus africanus, Longidorus breviannulatus, Longidorus elongatus, Longidorus laevicapitatus, Longidorus vineacola and the ectoparasites Longidorus spp., Meloidogyne acronea, Meloidogyne africana, Meloidogyne arenaria, Meloidogyne arenaria thamesi, Meloidogyne artiella, Meloidogyne chitwoodi, Meloidogyne coffeicola, Meloidogyne ethiopica, Meloidogyne exigua, Meloidogyne fallax, Meloidogyne graminicola, Meloidogyne graminis, Meloidogyne hapla, Meloidogyne incognita, Meloidogyne incognita acrita, Meloidogyne javanica, Meloidogyne kikuyensis, Meloidogyne minor, Meloidogyne naasi, Meloidogyne paranaensis, Meloidogyne thamesi and the non-migratory parasites Meloidogyne spp., Meloinema spp., Nacobbus aberrans, Neotylenchus vigissi, Paraphelenchus pseudoparietinus, Paratrichodorus allius, Paratrichodorus lobatus, Paratrichodorus minor, Paratrichodorus nanus, Paratrichodorus porosus, Paratrichodorus teres and Paratrichodorus spp., Paratylenchus hamatus, Paratylenchus minutus, Paratylenchus projectus and Paratylenchus spp., Pratylenchus agilis, Pratylenchus alleni, Pratylenchus andinus, Pratylenchus brachyurus, Pratylenchus cerealis, Pratylenchus coffeae, Pratylenchus crenatus, Pratylenchus delattrei, Pratylenchus giibbicaudatus, Pratylenchus goodeyi, Pratylenchus hamatus, Pratylenchus hexincisus, Pratylenchus loosi, Pratylenchus neglectus, Pratylenchus penetrans, Pratylenchus pratensis, Pratylenchus scribneri, Pratylenchus teres, Pratylenchus thornei, Pratylenchus vulnus, Pratylenchus zeae and the migratory endoparasites Pratylenchus spp., Pseudohalenchus minutus, Psilenchus magnidens, Psilenchus tumidus, Punctodera chalcoensis, Quinisulcius acutus, Radopholus citrophilus, Radopholus similis, the migratory endoparasites Radopholus spp., Rotylenchulus borealis, Rotylenchulus parvus, Rotylenchulus reniformis and Rotylenchulus spp., Rotylenchus laurentinus, Rotylenchus macrodoratus, Rotylenchus robustus, Rotylenchus uniformis and Rotylenchus spp., Scutellonema brachyurum, Scutellonema bradys, Scutellonema clathricaudatum and the migratory endoparasites Scutellonema spp., Subanguina radiciola, Tetylenchus nicotianae, Trichodorus cylindricus, Trichodorus minor, Trichodorus primitivus, Trichodorus proximus, Trichodorus similis, Trichodorus sparsus and the ectoparasites Trichodorus spp., Tylenchorhynchus agri, Tylenchorhynchus brassicae, Tylenchorhynchus clarus, Tylenchorhynchus claytoni, Tylenchorhynchus digitatus, Tylenchorhynchus ebriensis, Tylenchorhynchus maximus, Tylenchorhynchus nudus, Tylenchorhynchus vulgaris and Tylenchorhynchus spp., Tylenchulus semipenetrans and the semiparasites Tylenchulus spp., Xiphinema americanum, Xiphinema brevicolle, Xiphinema dimorphicaudatum, Xiphinema index and the ectoparasites Xiphinema spp.

Nematodes for the control of which a compound of the formulae (I), (I-1) or (I-2) may be used include nematodes of the genus Meloidogyne such as the Southern root-knot nematode (Meloidogyne incognita), the Javanese root-knot nematode (Meloidogyne javanica), the Northern root-knot nematode (Meloidogyne hapla) and the peanut root-knot nematode (Meloidogyne arenaria); nematodes of the genus Ditylenchus such as the potato rot nematode (Ditylenchus destructor) and stem and bulb eelworm (Ditylenchus dipsaci); nematodes of the genus Pratylenchus such as the cob root-lesion nematode (Pratylenchus penetrans), the chrysanthemum root-lesion nematode (Pratylenchus fallax), the coffee root nematode (Pratylenchus coffeae), the tea root nematode (Pratylenchus loosi) and the walnut root-lesion nematode (Pratylenchus vulnus); nematodes of the genus Globodera such as the yellow potato cyst nematode (Globodera rostochiensis) and the white potato cyst nematode (Globodera pallida); nematodes of the genus Heterodera such as the soya bean cyst nematode (Heterodera glycines) and beet cyst eelworm (Heterodera schachtii); nematodes of the genus Aphelenchoides such as the rice white-tip nematode (Aphelenchoides besseyi), the chrysanthemum nematode (Aphelenchoides ritzemabosi) and the strawberry nematode (Aphelenchoides fragariae); nematodes of the genus Aphelenchus such as the fungivorous nematode (Aphelenchus avenae); nematodes of the genus Radopholus, such as the burrowing nematode (Radopholus similis); nematodes of the genus Tylenchulus such as the citrus root nematode (Tylenchulus semipenetrans); nematodes of the genus Rotylenchulus such as the reniform nematode (Rotylenchulus reniformis); tree-dwelling nematodes such as the pine wood nematode (Bursaphelenchus xylophilus) and the red ring nematode (Bursaphelenchus cocophilus) and the like.

Plants for the protection of which a compound of the formulae (I), (I-1) or (I-2) can be used include plants such as cereals (for example rice, barley, wheat, rye, oats, maize and the like), beans (soya bean, aduki bean, bean, broadbean, peas, peanuts and the like), fruit trees/fruits (apples, citrus species, pears, grapevines, peaches, Japanese apricots, cherries, walnuts, almonds, bananas, strawberries and the like), vegetable species (cabbage, tomato, spinach, broccoli, lettuce, onions, spring onion, pepper and the like), root crops (carrot, potato, sweet potato, radish, lotus root, turnip and the like), plant for industrial raw materials (cotton, hemp, paper mulberry, mitsumata, rape, beet, hops, sugar cane, sugar beet, olive, rubber, palm trees, coffee, tobacco, tea and the like), cucurbits (pumpkin, cucumber, water melon, melon and the like), meadow plants (cocksfoot, sorghum, timothy-grass, clover, alfalfa and the like), lawn grasses (mascarene grass, bentgrass and the like), spice plants etc. (lavender, rosemary, thyme, parsley, pepper, ginger and the like) and flowers (chrysanthemums, rose, orchid and the like).

In one specific embodiment the compounds of the formulae (I), (I-1) or (I-2) are suitable for controlling coffee nematodes, in particular Pratylenchus brachyurus, Pratylenchus coffeae, Meloidogyne exigua, Meloidogyne incognita, Meloidogyne coffeicola, Helicotylenchus spp. and also Meloidogyne paranaensis, Rotylenchus spp., Xiphinema spp., Tylenchorhynchus spp. and Scutellonema spp.

In another specific embodiment the compounds of the formulae (I), (I-1) or (I-2) are suitable for controlling potato nematodes, in particular Pratylenchus brachyurus, Pratylenchus pratensis, Pratylenchus scribneri, Pratylenchus penetrans, Pratylenchus coffeae, Ditylenchus dipsaci and of Pratylenchus alleni, Pratylenchus andinus, Pratylenchus cerealis, Pratylenchus crenatus, Pratylenchus hexincisus, Pratylenchus loosi, Pratylenchus neglectus, Pratylenchus teres, Pratylenchus thornei, Pratylenchus vulnus, Belonolaimus longicaudatus, Trichodorus cylindricus, Trichodorus primitivus, Trichodorus proximus, Trichodorus similis, Trichodorus sparsus, Paratrichodorus minor, Paratrichodorus allius, Paratrichodorus nanus, Paratrichodorus teres, Meloidogyne arenaria, Meloidogyne fallax, Meloidogyne hapla, Meloidogyne thamesi, Meloidogyne incognita, Meloidogyne chitwoodi, Meloidogyne javanica, Nacobbus aberrans, Globodera rostochiensis, Globodera pallida, Ditylenchus destructor, Radopholus similis, Rotylenchulus reniformis, Neotylenchus vigissi, Paraphelenchus pseudoparietinus, Aphelenchoides fragariae and Meloinema spp.

In another specific embodiment the compounds of the formulae (I), (I-1) or (I-2) are suitable for controlling tomato nematodes, in particular Meloidogyne arenaria, Meloidogyne hapla, Meloidogyne javanica, Meloidogyne incognita, Pratylenchus penetrans and also Pratylenchus brachyurus, Pratylenchus coffeae, Pratylenchus scribneri, Pratylenchus vulnus, Paratrichodorus minor, Meloidogyne exigua, Nacobbus aberrans, Globodera solanacearum, Dolichodorus heterocephalus and Rotylenchulus reniformis.

In another specific embodiment the compounds of the formulae (I), (I-1) or (I-2) are suitable for controlling cucumber plant nematodes, in particular Meloidogyne arenaria, Meloidogyne hapla, Meloidogyne javanica, Meloidogyne incognita, Rotylenchulus reniformis and Pratylenchus thornei.

In another specific embodiment the compounds of the formulae (I), (I-1) or (I-2) are suitable for controlling cotton nematodes, in particular Belonolaimus longicaudatus, Meloidogyne incognita, Hoplolaimus columbus, Hoplolaimus galeatus and Rotylenchulus reniformis.

In another specific embodiment the compounds of the formulae (I), (I-1) or (I-2) are suitable for controlling maize nematodes, in particular Belonolaimus longicaudatus, Paratrichodorus minor and also Pratylenchus brachyurus, Pratylenchus delattrei, Pratylenchus hexincisus, Pratylenchus penetrans, Pratylenchus zeae, (Belonolaimus gracilis), Belonolaimus nortoni, Longidorus breviannulatus, Meloidogyne arenaria, Meloidogyne arenaria thamesi, Meloidogyne graminis, Meloidogyne incognita, Meloidogyne incognita acrita, Meloidogyne javanica, Meloidogyne naasi, Heterodera avenae, Heterodera oryzae, Heterodera zeae, Punctodera chalcoensis, Ditylenchus dipsaci, Hoplolaimus aegyptii, Hoplolaimus magnistylus, Hoplolaimus galeatus, Hoplolaimus indicus, Helicotylenchus digonicus, Helicotylenchus dihystera, Helicotylenchus pseudorobustus, Xiphinema americanum, Dolichodorus heterocephalus, Criconemella ornata, Criconemella onoensis, Radopholus similis, Rotylenchulus borealis, Rotylenchulus parvus, Tylenchorhynchus agri, Tylenchorhynchus clarus, Tylenchorhynchus claytoni, Tylenchorhynchus maximus, Tylenchorhynchus nudus, Tylenchorhynchus vulgaris, Quinisulcius acutus, Paratylenchus minutus, Hemicycliophora parvana, Aglenchus agricola, Anguina tritici, Aphelenchoides arachidis, Scutellonema brachyurum and Subanguina radiciola.

In another specific embodiment the compounds of the formulae (I), (I-1) or (I-2) are suitable for controlling soya bean nematodes, in particular Pratylenchus brachyurus, Pratylenchus pratensis, Pratylenchus penetrans, Pratylenchus scribneri, Belonolaimus longicaudatus, Heterodera glycines, Hoplolaimus columbus and also Pratylenchus coffeae, Pratylenchus hexincisus, Pratylenchus neglectus, Pratylenchus crenatus, Pratylenchus alleni, Pratylenchus agilis, Pratylenchus zeae, Pratylenchus vulnus, (Belonolaimus gracilis), Meloidogyne arenaria, Meloidogyne incognita, Meloidogyne javanica, Meloidogyne hapla, Hoplolaimus columbus, Hoplolaimus galeatus and Rotylenchulus reniformis.

In another specific embodiment the compounds of the formulae (I), (I-1) or (I-2) are suitable for controlling tobacco nematodes, in particular Meloidogyne incognita, Meloidogyne javanica and also Pratylenchus brachyurus, Pratylenchus pratensis, Pratylenchus hexincisus, Pratylenchus penetrans, Pratylenchus neglectus, Pratylenchus crenatus, Pratylenchus thornei, Pratylenchus vulnus, Pratylenchus zeae, Longidorus elongatu, Paratrichodorus lobatus, Trichodorus spp., Meloidogyne arenaria, Meloidogyne hapla, Globodera tabacum, Globodera solanacearum, Globodera virginiae, Ditylenchus dipsaci, Rotylenchus spp., Helicotylenchus spp., Xiphinema americanum, Criconemella spp., Rotylenchulus reniformis, Tylenchorhynchus claytoni, Paratylenchus spp. and Tetylenchus nicotianae.

In another specific embodiment the compounds of the formulae (I), (I-1) or (I-2) are suitable for controlling citrus nematodes, in particular Pratylenchus coffeae and also Pratylenchus brachyurus, Pratylenchus vulnus, Belonolaimus longicaudatus, Paratrichodorus minor, Paratrichodorus porosus, Trichodorus, Meloidogyne incognita, Meloidogyne incognita acrita, Meloidogyne javanica, Rotylenchus macrodoratus, Xiphinema americanum, Xiphinema brevicolle, Xiphinema index, Criconemella spp., Hemicriconemoides, Radopholus similis and Radopholus citrophilus, Hemicycliophora arenaria, Hemicycliophora nudata and Tylenchulus semipenetrans.

In another specific embodiment the compounds of the formulae (I), (I-1) or (I-2) are suitable for controlling banana nematodes, in particular Pratylenchus coffeae, Radopholus similis and also Pratylenchus giibbicaudatus, Pratylenchus loosi, Meloidogyne spp., Helicotylenchus multicinctus, Helicotylenchus dihystera and Rotylenchulus spp.

In another specific embodiment the compounds of the formulae (I), (I-1) or (I-2) are suitable for controlling pineapple nematodes, in particular Pratylenchus zeae, Pratylenchus pratensis, Pratylenchus brachyurus, Pratylenchus goodeyi., Meloidogyne spp., Rotylenchulus reniformis and also Longidorus elongatus, Longidorus laevicapitatus, Trichodorus primitivus, Trichodorus minor, Heterodera spp., Ditylenchus myceliophagus, Hoplolaimus californicus, Hoplolaimus pararobustus, Hoplolaimus indicus, Helicotylenchus dihystera, Helicotylenchus nannus, Helicotylenchus multicinctus, Helicotylenchus erythrine, Xiphinema dimorphicaudatum, Radopholus similis, Tylenchorhynchus digitatus, Tylenchorhynchus ebriensis, Paratylenchus minutus, Scutellonema clathricaudatum, Scutellonema bradys, Psilenchus tumidus, Psilenchus magnidens, Pseudohalenchus minutus, Criconemoides ferniae, Criconemoides onoense and Criconemoides ornatum.

In another specific embodiment the compounds of the formulae (I), (I-1) or (I-2) are suitable for controlling grapevine nematodes, in particular Pratylenchus vulnus, Meloidogyne arenaria, Meloidogyne incognita, Meloidogyne javanica, Xiphinema americanum, Xiphinema index and also Pratylenchus pratensis, Pratylenchus scribneri, Pratylenchus neglectus, Pratylenchus brachyurus, Pratylenchus thornei and Tylenchulus semipenetrans.

In another specific embodiment the compounds of the formulae (I), (I-1) or (I-2) are suitable for controlling nematodes in tree crops—pome fruit, in particular Pratylenchus penetrans and also Pratylenchus vulnus, Longidorus elongatus, Meloidogyne incognita and Meloidogyne hapla.

In another specific embodiment the compounds of the formulae (I), (I-1) or (I-2) are suitable for controlling nematodes in tree crops—stone fruit, in particular Pratylenchus penetrans, Pratylenchus vulnus, Meloidogyne arenaria, Meloidogyne hapla, Meloidogyne javanica, Meloidogyne incognita, Criconemella xenoplax and of Pratylenchus brachyurus, Pratylenchus coffeae, Pratylenchus scribneri, Pratylenchus zeae, Belonolaimus longicaudatus, Helicotylenchus dihystera, Xiphinema americanum, Criconemella curvata, Tylenchorhynchus claytoni, Paratylenchus hamatus, Paratylenchus projectus, Scutellonema brachyurum and Hoplolaimus galeatus.

In another specific embodiment the compounds of the formulae (I), (I-1) or (I-2) are suitable for controlling nematodes in tree crops, sugar cane and rice, in particular Trichodorus spp., Criconemella spp. and also Pratylenchus spp., Paratrichodorus spp., Meloidogyne spp., Helicotylenchus spp., Tylenchorhynchus spp., Aphelenchoides spp., Heterodera spp, Xiphinema spp. and Cacopaurus pestis.

The present invention further relates to a composition for controlling phytopathogenic microorganisms, in particular phytopathogenic fungi comprising at least one of the compounds of the formulae (I), (I-1) or (I-2). These are preferably fungicidal compositions which comprise agriculturally suitable auxiliaries, solvents, carriers, surfactants or extenders.

According to the invention, a carrier is a natural or synthetic, organic or inorganic substance with which the active ingredients are mixed or combined for better applicability, in particular for application to plants or plant parts or seed. The carrier, which may be solid or liquid, is generally inert and should be suitable for use in agriculture.

Useful solid carriers include: for example ammonium salts and natural rock flours, such as kaolins, clays, talc, chalk, quartz, attapulgite, montmorillonite or diatomaceous earth, and synthetic rock flours, such as finely divided silica, alumina and silicates; useful solid carriers for granules include: for example, crushed and fractionated natural rocks such as calcite, marble, pumice, sepiolite and dolomite, and also synthetic granules of inorganic and organic flours, and granules of organic material such as paper, sawdust, coconut shells, maize cobs and tobacco stalks; useful emulsifiers and/or foam-formers include: for example nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates and also protein hydrolysates; suitable dispersants are nonionic and/or ionic substances, for example from the classes of the alcohol-POE and/or -POP ethers, acid and/or POP POE esters, alkylaryl and/or POP POE ethers, fat and/or POP POE adducts, POE- and/or POP-polyol derivatives, POE- and/or POP-sorbitan or -sugar adducts, alkyl or aryl sulphates, alkyl- or arylsulphonates and alkyl or aryl phosphates or the corresponding PO-ether adducts. Additionally suitable are oligo- or polymers, for example those derived from vinylic monomers, from acrylic acid, from EO and/or PO alone or in combination with, for example, (poly)alcohols or (poly)amines. It is also possible to use lignin and its sulphonic acid derivatives, unmodified and modified celluloses, aromatic and/or aliphatic sulphonic acids and also their adducts with formaldehyde.

The active ingredients can be converted to the customary formulations, such as solutions, emulsions, wettable powders, water- and oil-based suspensions, powders, dusts, pastes, soluble powders, soluble granules, granules for broadcasting, suspoemulsion concentrates, natural products impregnated with active ingredient, synthetic substances impregnated with active ingredient, fertilizers and also microencapsulations in polymeric substances.

The active ingredients can be applied as such, in the form of their formulations or the use forms prepared therefrom, such as ready-to-use solutions, emulsions, water- or oil-based suspensions, powders, wettable powders, pastes, soluble powders, dusts, soluble granules, granules for broadcasting, suspoemulsion concentrates, natural products impregnated with active ingredient, synthetic substances impregnated with active ingredient, fertilizers and also microencapsulations in polymeric substances. Application is accomplished in a customary manner, for example by watering, spraying, atomizing, broadcasting, dusting, foaming, spreading-on and the like. It is also possible to deploy the active ingredients by the ultra-low volume method or to inject the active ingredient preparation/the active ingredient itself into the soil. It is also possible to treat the seed of the plants.

The formulations mentioned can be prepared in a manner known per se, for example by mixing the active ingredients with at least one customary extender, solvent or diluent, emulsifier, dispersant and/or binder or fixing agent, wetting agent, a water repellent, if appropriate siccatives and UV stabilizers and if appropriate dyes and pigments, antifoams, preservatives, secondary thickeners, stickers, gibberellins and also other processing auxiliaries.

The present invention includes not only formulations which are already ready for use and can be deployed with a suitable apparatus to the plant or the seed, but also commercial concentrates which have to be diluted with water prior to use.

The compounds of the formulae (I), (I-1) or (I-2) may be present as such or in their (commercial) formulations and in the use forms prepared from these formulations as a mixture with other (known) active ingredients, such as insecticides, attractants, sterilants, bactericides, acaricides, nematicides, fungicides, growth regulators, herbicides, fertilizers, safeners and/or semiochemicals.

The auxiliaries used may be those substances which are suitable for imparting particular properties to the composition itself or and/or to preparations derived therefrom (for example spray liquors, seed dressings), such as certain technical properties and/or also particular biological properties. Typical auxiliaries include: extenders, solvents and carriers.

Suitable extenders are, for example, water, polar and nonpolar organic chemical liquids, for example from the classes of the aromatic and nonaromatic hydrocarbons (such as paraffins, alkylbenzenes, alkylnaphthalenes, chlorobenzenes), the alcohols and polyols (which may optionally also be substituted, etherified and/or esterified), the ketones (such as acetone, cyclohexanone), esters (including fats and oils) and (poly)ethers, the unsubstituted and substituted amines, amides, lactams (such as N-alkylpyrrolidones) and lactones, the sulphones and sulphoxides (such as dimethyl sulphoxide).

Liquefied gaseous extenders or carriers are understood to mean liquids which are gaseous at standard temperature and under standard pressure, for example aerosol propellants such as halohydrocarbons, or else butane, propane, nitrogen and carbon dioxide.

In the formulations it is possible to use tackifiers such as carboxymethylcellulose, natural and synthetic polymers in the form of powders, granules or latices, such as gum arabic, polyvinyl alcohol and polyvinyl acetate, or else natural phospholipids such as cephalins and lecithins and synthetic phospholipids. Further additives may be mineral and vegetable oils.

If the extender used is water, it is also possible to use, for example, organic solvents as auxiliary solvents. Useful liquid solvents are essentially: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example petroleum fractions, alcohols such as butanol or glycol and their ethers and esters, ketones such as acetone, methyl ethyl ketone, methyl isobutyl ketone or cyclohexanone, strongly polar solvents such as dimethylformamide and dimethyl sulphoxide, or else water.

Compositions comprising compounds of the formulae (I), (I-1) or (I-2) may additionally comprise further components, for example surfactants. Suitable surfactants are emulsifiers and/or foam formers, dispersants or wetting agents having ionic or nonionic properties, or mixtures of these surfactants. Examples thereof are salts of polyacrylic acid, salts of lignosulphonic acid, salts of phenolsulphonic acid or naphthalenesulphonic acid, polycondensates of ethylene oxide with fatty alcohols or with fatty acids or with fatty amines, substituted phenols (preferably alkylphenols or arylphenols), salts of sulphosuccinic esters, taurine derivatives (preferably alkyl taurates), phosphoric esters of polyethoxylated alcohols or phenols, fatty esters of polyols, and derivatives of the compounds containing sulphates, sulphonates and phosphates, for example alkylaryl polyglycol ethers, alkylsulphonates, alkyl sulphates, arylsulphonates, protein hydrolysates, lignosulphite waste liquors and methylcellulose. The presence of a surfactant is necessary if one of the active ingredients and/or one of the inert carriers is insoluble in water and when application is effected in water. The proportion of surfactants is between 5 and 40 percent by weight of the inventive composition.

It is possible to use dyes such as inorganic pigments, for example iron oxide, titanium oxide and Prussian Blue, and organic dyes such as alizarin dyes, azo dyes and metal phthalocyanine dyes, and trace nutrients such as salts of iron, manganese, boron, copper, cobalt, molybdenum and zinc.

Further additives may be perfumes, mineral or vegetable, optionally modified oils, waxes and nutrients (including trace nutrients), such as salts of iron, manganese, boron, copper, cobalt, molybdenum and zinc.

Additional components may be stabilizers, such as cold stabilizers, preservatives, antioxidants, light stabilizers, or other agents which improve chemical and/or physical stability.

If appropriate, other additional components may also be present, for example protective colloids, binders, adhesives, thickeners, thixotropic substances, penetrants, stabilizers, sequestering agents, complex formers. In general, the active ingredients can be combined with any solid or liquid additive commonly used for formulation purposes.

The formulations contain generally between 0.05 and 99% by weight, 0.01 and 98% by weight, preferably between 0.1 and 95% by weight, more preferably between 0.5 and 90% of active ingredient, most preferably between 10 and 70 percent by weight.

The formulations described above can be used for controlling phytopathogenic microorganisms, in which the compositions comprising compounds of the formulae (I), (I-1) or (I-2) are applied to the phytopathogenic microorganisms and/or in their habitat.

The invention furthermore includes a method for treating seed.

A further aspect of the present invention relates in particular to seeds (dormant, primed, pregerminated or even with emerged roots and leaves) treated with at least one of the compounds of the formulae (I), (I-1) or (I-2). The inventive seeds are used in methods for protection of seeds and emerged plants from the seeds from phytopathogenic microorganisms, in particular phytopathogenic fungi. In these methods, seed treated with at least one inventive active ingredient is used.

The compounds of the formulae (I), (I-1) or (I-2) are also suitable for the treatment of seeds and young seedlings. A large part of the damage to crop plants caused by harmful organisms is triggered by the infection of the seeds before sowing or after germination of the plant. This phase is particularly critical since the roots and shoots of the growing plant are particularly sensitive, and even small damage may result in the death of the plant. Accordingly, there is great interest in protecting the seed and the germinating plant by using appropriate compositions.

It is also desirable to optimize the amount of the active ingredient used so as to provide the best possible protection for the seeds, the germinating plants and emerged seedlings from attack by phytopathogenic fungi, but without damaging the plants themselves by the active ingredient used. In particular, methods for the treatment of seed should also take into consideration the intrinsic phenotypes of transgenic plants in order to achieve optimum protection of the seed and the germinating plant with a minimum of crop protection compositions being employed.

The present invention therefore also relates to a method for protecting seeds, germinating plants and emerged seedlings against attack by animal pests and/or phytopathogenic microorganisms, in particular phytopathogenic fungi by treating the seeds with an inventive composition. The invention also relates to the use of the compositions according to the invention for treating seeds for protecting the seeds, the germinating plants and emerged seedlings against animal pests and/or phytopathogenic microorganisms, in particular phytopathogenic fungimicro. The invention further relates to seeds which have been treated with an inventive composition for protection from animal pests and/or phytopathogenic microorganisms, in particular phytopathogenic fungi.

One of the advantages of the present invention is that the treatment of the seeds with these compositions not only protects the seed itself, but also the resulting plants after emergence, from animal pests and/or phytopathogenic harmful microorganisms. In this way, the immediate treatment of the crop at the time of sowing or shortly thereafter protect plants as well as seed treatment in prior to sowing. It is likewise considered to be advantageous that the inventive active ingredients or compositions can be used especially also for transgenic seed, in which case the plant which grows from this seed is capable of expressing a protein which acts against pests, herbicidal damage or abiotic stress. The treatment of such seeds with the inventive active ingredients or compositions, for example an insecticidal protein, can result in control of certain pests. Surprisingly, a further synergistic effect can be observed in this case, which additionally increases the effectiveness for protection against attack by pests, microorganisms, weeds or abiotic stress.

The compounds of the formulae (I), (I-1) or (I-2) are suitable for protection of seed of any plant variety which is used in agriculture, in the greenhouse, in forests or in horticulture. More particularly, the seed is that of cereals (such as wheat, barley, rye, millet and oats), oilseed rape, maize, cotton, soybeen, rice, potatoes, sunflower, beans, coffee, beet (e.g. sugar beet and fodder beet), peanut, vegetables (such as tomato, cucumber, onions and lettuce), lawns and ornamental plants. Of particular significance is the treatment of the seed of wheat, soybean, oilseed rape, maize and rice.

As also described below, the treatment of transgenic seed with the inventive active ingredients or compositions is of particular significance. This refers to the seed of plants containing at least one heterologous gene which allows the expression of a polypeptide or protein, e.g. having insecticidal properties. These heterologous genes in transgenic seeds may originate, for example, from microorganisms of the species Bacillus, Rhizobium, Pseudomonas, Serratia, Trichoderma, Clavibacter, Glomus or Gliocladium. These heterologous genes preferably originate from Bacillus sp., in which case the gene product is effective against the European corn borer and/or the Western corn rootworm. Particularly preferably, the heterologous genes originate from Bacillus thuringiensis.

In the context of the present invention, the inventive composition is applied to seeds either alone or in a suitable formulation. Preferably, the seed is treated in a state in which it is sufficiently stable for no damage to occur in the course of treatment. In general, seeds can be treated at any time between harvest and some time after sowing. It is customary to use seed which has been separated from the plant and freed from cobs, shells, stalks, coats, hairs or the flesh of the fruits. For example, it is possible to use seed which has been harvested, cleaned and dried down to a moisture content of less than 15% by weight. Alternatively, it is also possible to use seed which, after drying, for example, has been treated with water and then dried again, or seeds just after priming, or seeds stored in primed conditions or pregerminated seeds, or seeds sown on nursery trays, tapes or paper.

When treating the seeds, it generally has to be ensured that the amount of the inventive composition applied to the seed and/or the amount of further additives is selected such that the germination of the seed is not impaired, or that the resulting plant is not damaged. This must be ensured particularly in the case of active ingredients which can exhibit phytotoxic effects at certain application rates.

The compounds of the formulae (I), (I-1) or (I-2) can be applied directly, i.e. without containing any other components and without having been diluted. In general, it is preferable to apply the compositions to the seed in the form of a suitable formulation. Suitable formulations and methods for seed treatment are known to those skilled in the art. The compounds of the formulae (I), (I-1) or (I-2) can be converted to the customary formulations relevant to on-seed applications, such as solutions, emulsions, suspensions, powders, foams, slurries or combined with other coating compositions for seed, such as film forming materials, pelleting materials, fine iron or other metal powders, granules, coating material for inactivated seeds, and also ULV formulations.

These formulations are prepared in a known manner, by mixing the active ingredients or active ingredient combinations with customary additives, for example customary extenders and solvents or diluents, dyes, wetting agents, dispersants, emulsifiers, antifoams, preservatives, secondary thickeners, adhesives, gibberellins, and also water.

Useful dyes which may be present in the seed dressing formulations usable in accordance with the invention are all dyes which are customary for such purposes. It is possible to use either pigments, which are sparingly soluble in water, or dyes, which are soluble in water. Examples include the dyes known by the names Rhodamine B, C.I. Pigment Red 112 and C.I. Solvent Red 1.

Useful wetting agents which may be present in the seed dressing formulations usable in accordance with the invention are all substances which promote wetting and which are conventionally used for the formulation of active agrochemical ingredients. Usable with preference are alkylnaphthalenesulphonates, such as diisopropyl- or diisobutylnaphthalenesulphonates.

Useful dispersants and/or emulsifiers which may be present in the seed dressing formulations usable in accordance with the invention are all nonionic, anionic and cationic dispersants conventionally used for the formulation of active agrochemical ingredients. Usable with preference are nonionic or anionic dispersants or mixtures of nonionic or anionic dispersants. Useful nonionic dispersants include especially ethylene oxide/propylene oxide block polymers, alkylphenol polyglycol ethers and tristryrylphenol polyglycol ether, and the phosphated or sulphated derivatives thereof. Suitable anionic dispersants are especially lignosulphonates, polyacrylic acid salts and arylsulphonate/formaldehyde condensates.

Antifoams which may be present in the seed dressing formulations usable in accordance with the invention are all foam-inhibiting substances conventionally used for the formulation of active agrochemical ingredients. Silicone antifoams and magnesium stearate can be used with preference.

Preservatives which may be present in the seed dressing formulations usable in accordance with the invention are all substances usable for such purposes in agrochemical compositions. Examples include dichlorophene and benzyl alcohol hemiformal.

Secondary thickeners which may be present in the seed dressing formulations usable in accordance with the invention are all substances usable for such purposes in agrochemical compositions. Preferred examples include cellulose derivatives, acrylic acid derivatives, xanthan, modified clays and finely divided silica.

Adhesives which may be present in the seed dressing formulations usable in accordance with the invention are all customary binders usable in seed dressing products. Preferred examples include polyvinylpyrrolidone, polyvinyl acetate, polyvinyl alcohol and tylose.

The formulations for on-seed applications usable in accordance with the invention can be used to treat a wide variety of different kinds of seed either directly or after prior dilution with water. For instance, the concentrates or the preparations obtainable therefrom by dilution with water can be used to dress the seed of cereals, such as wheat, barley, rye, oats, and triticale, and also seeds of maize, soybean, rice, oilseed rape, peas, beans, cotton, sunflowers, and beets, or else a wide variety of different vegetable seeds. The formulations usable in accordance with the invention, or the dilute preparations thereof, can also be used for seeds of transgenic plants. In this case, additional synergistic effects may also occur in interaction with the substances formed by expression.

For treatment of seeds with the formulations usable in accordance with the invention, or the preparations prepared therefrom by adding water, all mixing units usable customarily for on-seed applications are useful. Specifically, the procedure in on-seed applications is to place the seeds into a mixer, to add the particular desired amount of the formulations, either as such or after prior dilution with water, and to mix everything until all applied formulations are distributed homogeneously on the seeds. If appropriate, this is followed by a drying operation.

The application rate of the formulations usable in accordance with the invention can be varied within a relatively wide range. It is guided by the particular content of the active ingredients in the formulations and by the seeds. The application rates of each single active ingredient are generally between 0.001 and 15 g per kilogram of seed, preferably between 0.01 and 5 g per kilogram of seed.

When using the compounds of the formulae (I), (I-1) or (I-2) as phyto-nematicides, the application rates can be varied within a relatively wide range, depending on the kind of application. The application rate of the inventive active ingredients is
in the case of treatment of plant parts, for example leaves: from 0.1 to 10 000 g/ha, preferably from 10 to 1000 g/ha, more preferably from 50 to 300 g/ha (in the case of application by watering or dripping, it is even possible to reduce the application rate, especially when inert substrates such as rockwool or perlite are used);
in the case of seed treatment: from 0.1 to 200 g per 100 kg of seed, preferably from 1 to 150 g per 100 kg of seed, more preferably from 2.5 to 25 g per 100 kg of seed, even more preferably from 2.5 to 12.5 g per 100 kg of seed;
in the case of soil treatment: from 0.1 to 10 000 g/ha, preferably from 1 to 5000 g/ha.

### PREPARATION EXAMPLES

LC-MS

Method L0:

Measurement of LogP values was performed according to EEC directive 79/831 Annex V.A8 by HPLC (High Performance Liquid Chromatography) on reversed phase columns with the following methods:
^{[a]} LogP value is determined by measurement of LC-UV, in an acidic range, with 0.1% formic acid in water and acetonitrile as eluent (linear gradient from 10% acetonitrile to 95% acetonitrile).
^{[b]} LogP value is determined by measurement of LC-UV, in a neutral range, with 0.001 molar ammonium acetate solution in water and acetonitrile as eluent (linear gradient from 10% acetonitrile to 95% acetonitrile).

Calibration was done with straight-chain alkan-2-ones (with 3 to 16 carbon atoms) with known LogP values (measurement of LogP values using retention times with linear interpolation between successive alkanones). Lambda-max-values were determined using UV-spectra from 200 nm to 400 nm and the peak values of the chromatographic signals.

In table 1, M+1 (or M+H) means the molecular ion peak, plus or minus 1 a.m.u. (atomic mass unit) respectively, as observed in mass spectroscopy by electrospray ionization (ESI + or −).

Method L1:

MS instrument type: Agilent Technologies 6130 Quadrupole LC-MS; HPLC instrument type: Agilent Technologies 1260 Infinity; column: Waters XSelect (C18, 30×2.1 mm, 3.5μ); flow: 1 mL/min; column temp: 35° C.; eluent A: 0.1% formic acid in acetonitrile; eluent B: 0.1% formic acid in water; lin. gradient: t=0 min 5% A, t=1.6 min 98% A, t=3 min 98% A; detection: DAD (220-320 nm); detection: MSD (ESI pos/neg) mass range: 100-800; detection: ELSD (PL-ELS 2100): gas flow 1.2 mL/min, gas temp: 70° C., neb: 50° C.

Method L2:

MS instrument type: Agilent Technologies 6130 Quadrupole LC-MS; HPLC instrument type: Agilent Technologies 1260 Infinity; column: Waters XSelect (C18, 50×2.1 mm, 3.5μ); flow: 0.8 mL/min; column temp: 35° C.; eluent A: 0.1% formic acid in acetonitrile; eluent B: 0.1% formic acid in water; lin. gradient: t=0 min 5% A, t=3.5 min 98% A, t=6 min 98% A; detection: DAD (220-320 nm); detection: MSD (ESI pos/neg) mass range: 100-800; detection: ELSD (PL-ELS 2100): gas flow 1.2 mL/min, gas temp: 70° C., neb: 50° C.

Method L3:

MS instrument type: Agilent Technologies LC/MSD SL; HPLC instrument type: Agilent Technologies 1100 Series; column: Waters XSelect (C18, 30×2.1 mm, 3.5μ); flow: 1 mL/min; column temp: 25° C., eluent A: 95% acetonitrile+5% ammoniumbicarbonate in water, eluent B: 10 mmM ammoniumbicarbonate in water pH=9.0; lin. gradient: t=0 min 5% A, t=1.6 min 98% A, t=3 min 98% A; detection: DAD (220-320 nm); detection: MSD (ESI pos/neg) mass range: 100-800.

Method L4:

MS instrument type: Agilent Technologies LC/MSD SL; HPLC instrument type: Agilent Technologies 1100 Series; column: Waters XSelect (C18, 50×2.1 mm, 3.5μ; flow: 0.8 mL/min; column temp: 25° C.; eluent A: 95% acetonitrile+5% ammoniumbicarbonate in water; eluent B: 10 mmM ammoniumbicarbonate in water pH=9.0; lin. gradient: t=0 min 5% A, t=3.5 min 98% A, t=6 min 98% A; detection: DAD (220-320 nm); detection: MSD (ESI pos/neg) mass range: 100-800.

Method L5:

instrument type: Reveleris™ Flash Chromatography System; columns: Reveleris™ C18 Flash Cartridge; 4 g, flow 18 mL/min; 12 g, flow 30 mL/min; 40 g, flow 40 mL/min; 80 g, flow 60 mL/min; 120 g, flow 80 mL/min; eluent A: 0.1% formic acid in acetonitrile; eluent B: 0.1% formic acid in water; gradient: t=0 min 5% A, t=1 min 5% A, t=13 min 100% A, t=16 min 100% A; detection: UV (200-360 nm), ELSD.

Method L6:

instrument type: Reveleris™ Flash Chromatography System; columns: GraceResolv™ Silica Cartridge; 4 g, flow 15 mL/min; 12 g, flow 28 mL/min; 40 g, flow 40 mL/min; 80 g, flow 55 mL/min; 120 g, flow 80 mL/min and Davisil™ Chromatographic Silica Media (LC60A 20-45 micron); 300 g, flow 70 mL/min; 500 g, flow 70 mL/min; eluents: see experiment; detection: UV (200-360 nm), ELSD.

Method L7:

instrument type: Büchi Pump Manager C-615, Büchi Pump Module C-601; columns: GraceResolv™ Silica Cartridge; 4 g, flow 15 mL/min; 12 g, flow 28 mL/min; 40 g, flow 40 mL/min; 80 g, flow 55 mL/min; 120 g, flow 80 mL/min and Davisil™ Chromatographic Silica Media (LC60A 20-45 micron); 300 g, flow 70 mL/min; 500 g, flow 70 mL/min; eluents: see experiment; detection: TLC plates; TLC Silica gel 60 F₂₅₄ (Merck).

Method L8:

MS instrument type: Agilent Technologies G1956B Quadrupole MS; HPLC instrument type: Agilent Technologies 1200 preparative LC; column: Waters XSelect (C18, 150×19 mm, 5μ); flow: 25 mL/min; column temp: room temperature; eluent A: 0.1% formic acid in acetonitrile; eluent B: 0.1% formic acid in water; a) lin. gradient: t=0 min 20% A, t=2.5 min 20% A t=11 min 60% A, t=13.5 min 100% A, t=17 min 100% A, b) lin. gradient: t=0 min 10% A, t=12 min 60% A, t=13.5 min 100% A, t=17 min 100% A; detection: DAD (220-320 nm); detection: MSD (ESI pos/neg) mass range: 100-800; fraction collection based on MS and DAD.

¹H-NMR Data

¹H-NMR-data were determined with a Bruker Avance 400 (Method M1) equipped with a flow cell (60 μl volume) or with a Bruker AVIII 400 equipped with 1.7 mm cryo CPTCI probe head or with a Bruker AVII 600 (600.13 MHz) equipped with a 5 mm cryo TCI probe head or with a Bruker AVIII 600 (601.6 MHz) equipped with a 5 mm cryo CPMNP probe head with tetramethylsilane as reference (0.0) and the solvents CD₃CN, CDCl₃ or D₆-DMSO.

Alternatively ¹H-NMR-data were determined with a Bruker DMX300 (¹H-NMR: 300 MHz) using tetramethylsilane as reference standard (Method M2).

### Preparation Example 1

### Step 1: 2-(4-bromo-2-chloro-phenyl)-2,2-difluoro-ethanamine

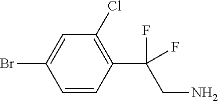 [SMILES: NCC(F)(F)c1ccc(Br)cc1Cl]

Synthesis of 2-(4-bromo-2-chloro-phenyl)-2,2-difluoro-ethanamine was performed in analogy to WO 2013/064460 A1 (referred as intermediates IIa-14 and IIa-15).

¹H-NMR (400 MHz, d₆-DMSO, Method M1); δ 7.91 (s, 1H), 7.74 (d, 1H), 7.57 (d, 1H), 4.68 (bs, 2H, NH₂), 3.46 (t, 2H).

### Step 2a: N-[2-(4-bromo-2-chloro-phenyl)-2,2-difluoro-ethyl]-2-(trifluoromethyl)benzamide

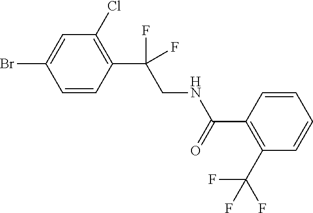 [SMILES: O=C(NCC(F)(F)c1ccc(Br)cc1Cl)c1ccccc1C(F)(F)F]

1.395 g (13.7 mmol) of triethylamine were added to a solution of 1.49 g (5.51 mmol) of 2-(4-bromo-2-chloro-phenyl)-2,2-difluoro-ethanamine (from step 1) in 30 mL dichloromethane at room temperature. 1.15 g (5.51 mmol) of 2-(trifluoromethyl)benzoyl chloride in 10 mL dichloromethane were slowly added to the reaction mixture at room temperature. After completion of reaction, the reaction mixture was diluted with dichloromethane and washed with water. The combined organic layers were evaporated under reduced pressure. The remaining residue was purified by flash silica gel chromatography resulting in 1.08 g as colorless crystalline solid (yield: 43.8%).

¹H-NMR (400 MHz, d₆-DMSO, Method M1); δ 8.98 (t, 1H, NH), 7.91 (s, 1H), 7.77-7.56 (m, 5H), 7.36 (d, 1H), 4.17 (dt, 2H).

### Step 2b: 2-[2-chloro-4-(4-fluorophenyl)phenyl]-2,2-difluoro-ethanamine (intermediate VII-1)

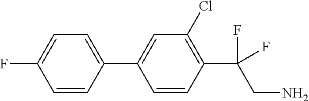 [SMILES: NCC(F)(F)c1ccc(-c2ccc(F)cc2)cc1Cl]

42 mg (0.3 mmol) (4-fluorophenyl)boronic acid and 100 mg (0.3 mmol) 2-(4-bromo-2-chloro-phenyl)-2,2-difluoro-ethanamine from step 1 were dissolved in 4.6 mL dioxane followed by addition of 195.5 mg (0.6 mmol) cesium carbonate in 0.58 mL water and 22.14 mg (0.03 mmol) dichloro-bis(tricyclohexylphosphine)-palladium-(II) catalyst. The reaction mixture was kept under stirring in a closed vial for 16 h at 100° C. The reaction mixture was filtered via a silica gel/sodium sulfate cartridge, solvents have been evaporated under reduced pressure and the remaining yellow-coloured solid was purified by MPLC on silicagel (solvent gradient cyclohexane/ethylacetate) to afford 73 mg the title compound as colorless crystalline solid (yield: 68%).

LC-MS (Method L0): IgP^{b}=3.10; M+1=286.0

¹H-NMR (400 MHz, d₆-DMSO, Method M1); δ 7.85-7.52 (m, 5H), 7.36-7.30 (t, 2H), 3.35-3.28 (broad m, NH₂, 2H).

### Step 3: N-[2-[2-chloro-4-(4-fluorophenyl)phenyl]-2,2-difluoro-ethyl]-2-(trifluoromethyl)benzamide (Example 3)

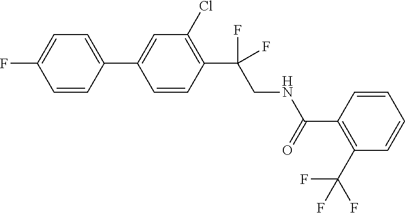 [SMILES: O=C(NCC(F)(F)c1ccc(-c2ccc(F)cc2)cc1Cl)c1ccccc1C(F)(F)F]

0.724 g (5.17 mmol) (4-fluorophenyl)boronic acid and 2.9 g (5.17 mmol) N-[2-(4-bromo-2-chloro-phenyl)-2,2-difluoro-ethyl]-2-(trifluoromethyl)benzamide from step 2a were dissolved in 80 mL dioxane followed by addition of 3.37 g (10.3 mmol) cesium carbonate in 10 mL water and 37.6 mg (0.51 mmol) (1,1′-bis(diphenylphosphino)-ferrocen)-palladium-dichloromethan complex. The reaction mixture is treated with microwaves (Biotage Initiator) at 100° C. for 30 min. The reaction mixture was filtered via a silica gel/sodium sulfate cartridge, solvents have been evaporated under reduced pressure and the remaining dark coloured oil was purified by MPLC on silicagel (solvent gradient cyclohexane/ethylacetate) to afford 1.95 g the title compound as colorless crystalline solid (yield: 80.4%).

LC-MS (Method L0): IgP^{a}=4.14; M+1=438.0 (target mass−HF)

¹H-NMR (400 MHz, d₆-DMSO, Method M1); δ 9.02 (t, NH), 7.90 (s, 1H), 7.84-7.62 (m, 6H), 7.39-7.30 (m, 3H), 4.25-4.17 (m, 2H).

### Preparation Example 2

### Step 1: Ethyl 2-(4-bromo-2-fluorophenyl)-2,2-difluoroacetate

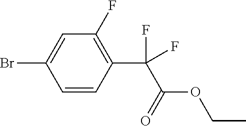 [SMILES: CCOC(=O)C(F)(F)c1ccc(Br)cc1F]

Ethyl bromodifluoroacetate (14.2 g, 70.0 mmol, 9.0 mL) and copper-tin alloy (25.3 g, 139.0 mmol) were added to a solution of 1-bromo-3-fluoro-4-iodobenzene (19.9 g, 66.0 mmol) in dimethyl sulfoxide (150 mL). The mixture was stirred at 50° C. for 5 h. A solution of monobasic potassium phosphate (11.7 g, 86.0 mmol) in water (150 mL) was added and the mixture was stirred for 18 h. The mixture was filtered over kieselguhr and the residue was washed with ethyl acetate. Water (100 mL) was added to the filtrate and the mixture was extracted with ethyl acetate (3×100 mL). The combined organic layers were washed with brine and dried with sodium sulfate. Solvents were removed in vacuo. Purification by flash column chromatography (Method L7; 80 g; heptane, 0%-10% ethyl acetate) afforded 14.4 g (41.2 mmol; 62% of theory) of the title compound with a purity of 85% according to LC-MS.

LC-MS (Method L1): Rₜ=2.21 min; no mass detected

¹H NMR (300 MHz, DMSO-d₆, Method M2) δ 7.88-7.82 (m, 1H), 7.71-7.61 (m, 2H), 4.35 (q, J=7.1 Hz, 2H), 1.23 (t, J=7.1 Hz, 3H).

### Step 2: 2-(4-Bromo-2-fluorophenyl)-2,2-difluoroethanol

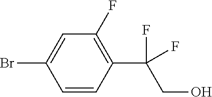 [SMILES: OCC(F)(F)c1ccc(Br)cc1F]

At 0° C. sodium borohydride (1.4 g, 36.4 mmol) was added portion wise to a solution of ethyl 2-(4-bromo-2-fluorophenyl)-2,2-difluoroacetate (14.4 g, 48.5 mmol) in ethanol (100 mL). The mixture was stirred at temperatures below 10° C. for 2 h. Aqueous hydrochloric acid (1M; 30 mL) and water (100 mL) were added and the mixture was extracted with ethyl acetate (3×100 mL). The combined organic layers were washed with brine and dried with sodium sulfate. Solvents were removed in vacuo. Purification by flash column chromatography (Method L7; 120 g; heptane, 0%-20% ethyl acetate) afforded 11.6 g (45.6 mmol; 94% of theory) of the title compound.

LC-MS (Method L1): Rₜ=1.93 min; no mass detected

¹H NMR (300 MHz, DMSO-d₆, Method M2) δ 7.79-7.68 (m, 1H), 7.61-7.44 (m, 2H), 5.71 (t, J=6.5 Hz, 1H), 3.98-3.78 (m, 2H).

### Step 3: 2-(4-Bromo-2-fluorophenyl)-2,2-difluoroethanamine

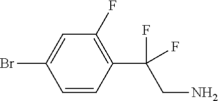 [SMILES: NCC(F)(F)c1ccc(Br)cc1F]

To a degassed (argon, 5 min) solution of 2-(4-bromo-2-fluorophenyl)-2,2-difluoroethanol (11.6 g, 45.5 mmol) in acetonitrile (600 mL) was added pyridine (5.8 g, 72.8 mmol, 5.9 mL). The mixture was cooled to 0° C. Trifluoromethanesulfonic acid anhydride (14.1 g, 50.0 mmol, 8.3 mL) was added drop wise while keeping the temperature below 8° C. The mixture was stirred at 0° C. for 30 min. To this solution aqueous ammonia (35%; 55.3 g, 1137 mmol, 63 mL) was added. The mixture was stirred at room temperature overnight. Brine (50 mL) was added and the reaction mixture was extracted with ethyl acetate (3×50 mL). The combined organic layers were washed with brine (50 mL) and dried with sodium sulfate. Solvents were removed in vacuo. The crude product was absorbed on isolute. Flash column chromatography (Method L6; 120 g; heptane, 0%-50% ethyl acetate) afforded 8.8 g (34.6 mmol; 76% of theory) of the title compound.

LC-MS (Method L3): Rₜ=2.00 min; m/z=254/256 (M+H)⁺

### Step 4: N-(2-(4-Bromo-2-fluorophenyl)-2,2-difluoroethyl)-2-(trifluoromethyl)benzamide

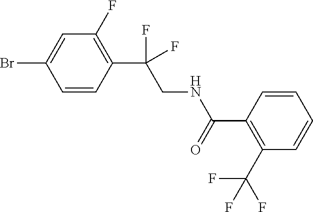 [SMILES: O=C(NCC(F)(F)c1ccc(Br)cc1F)c1ccccc1C(F)(F)F]

N-(3-dimethylaminopropyl)-3-ethylcarbodiimide hydrochloride (7.3 g, 38.1 mmol) and 1-hydroxy-7-azabenzotriazole (0.9 g, 6.9 mmol) were added to a solution of 2-(4-bromo-2-fluorophenyl)-2,2-difluoroethanamine (8.8 g, 34.6 mmol) and 2-(trifluoromethyl)benzoic acid (6.6 g, 34.6 mmol) in N,N-dimethylformamide (500 mL). The mixture was stirred at room temperature for 16 h. Solvents were removed in vacuo. The residue was partitioned between ethyl acetate and water. The organic layer was washed with brine and dried with sodium sulfate. Solvents were removed in vacuo. The crude product was absorbed on isolute. Flash column chromatography (Method L6; 120 g; heptane, 0%-50% ethyl acetate) and subsequent recrystallization from 2-propanol afforded 7.9 g (18.5 mmol; 53% of theory) of the title compound.

LC-MS (Method L1): Rₜ=2.17 min; m/z=426/428 (M+H)⁺

¹H NMR (300 MHz, DMSO-d₆, Method M2) δ 9.00 (t, J=6.2 Hz, 1H), 7.80-7.48 (m, 6H), 7.36 (d, J=7.2 Hz, 1H), 4.14-3.98 (m, 2H).

### Step 5: N-(2-(3,4′-Difluoro-[1,1′-biphenyl]-4-yl)-2,2-difluoroethyl)-2-(trifluoromethyl)benzamide (Example 7)

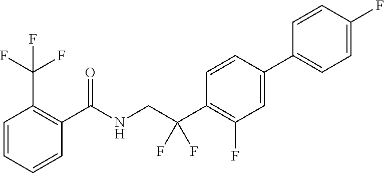 [SMILES: O=C(NCC(F)(F)c1ccc(-c2ccc(F)cc2)cc1F)c1ccccc1C(F)(F)F]

To a degassed (argon, 5 min) solution of N-(2-(4-bromo-2-fluorophenyl)-2,2-difluoroethyl)-2-(trifluoromethyl)benzamide (150 mg, 0.35 mmol), (4-fluorophenyl)boronic acid (54 mg, 0.39 mmol) and saturated aqueous sodium carbonate (1.06 mmol, 0.52 mL) in 1,2-dimethoxyethane (2 mL) was added bis(triphenylphosphine)palladium(II) chloride (12 mg, 0.02 mmol). The mixture stirred at 100° C. for 16 h and was allowed to cool to room temperature. Dichloromethane (5 mL) was added. The layers were separated in a phase separator and the organic layer was dried with sodium sulfate. Solvents were removed in vacuo. The crude product was purified by flash column chromatography (Method L6; heptane, 0%-100% ethyl acetate). 110 mg (0.25 mmol; 70% of theory) of the title compound were obtained.

LC-MS (Method L2): Rₜ=3.79 min; m/z=440 (M−H)⁻

¹H NMR (300 MHz, DMSO-d₆, Method M2) δ 9.12-8.90 (m, 1H), 7.92-7.11 (m, 11H), 4.22-3.95 (m, 2H).

### Preparation Example 3

### Step 1: Ethyl 2-(4-bromo-2-(trifluoromethyl)phenyl)-2,2-difluoroacetate

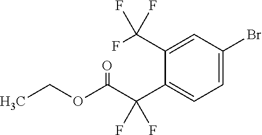 [SMILES: CCOC(=O)C(F)(F)c1ccc(Br)cc1C(F)(F)F]

Ethyl bromodifluoroacetate (15.2 g, 75.0 mmol, 9.6 mL) and copper-tin alloy (27.2 g, 149.0 mmol) were added to a solution of 4-bromo-1-iodo-2-(trifluoromethyl)benzene (25.0 g, 71.0 mmol) in dimethyl sulfoxide (500 mL). The mixture was stirred at 50° C. for 5 h. A solution of monobasic potassium phosphate (12.5 g, 92.0 mmol) in water (500 mL) was added and the mixture was stirred for 18 h. The mixture was filtered over kieselguhr and the residue was washed with ethyl acetate. Water (100 mL) was added to the filtrate and the mixture was extracted with ethyl acetate (3×100 mL). The combined organic layers were washed with brine and dried with sodium sulfate. Solvents were removed in vacuo. Purification by flash column chromatography (Method L6; 300 g; heptane, 0%-30% ethyl acetate) afforded 14.8 g (42.6 mmol; 51% of theory) of the title compound with a purity of 86% according to LC-MS.

LC-MS (Method L1): Rₜ=2.29 min; no mass detected

### Step 2: 2-(4-Bromo-2-(trifluoromethyl)phenyl)-2,2-difluoroethanol

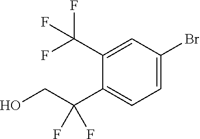 [SMILES: OCC(F)(F)c1ccc(Br)cc1C(F)(F)F]

At 0° C. sodium borohydride (1.6 g, 42.6 mmol) was added portion wise to a solution of ethyl 2-(4-bromo-2-fluorophenyl)-2,2-difluoroacetate (14.8 g, 42.6 mmol) in ethanol (250 mL). The mixture was stirred at temperatures below 10° C. for 2 h. Aqueous hydrochloric acid (1M; 30 mL) and water (200 mL) were added. Solids were filtered off and dried in vacuo at 40° C. 11.4 g (37 mmol, 88% of theory) of the title compound were obtained.

LC-MS (Method L1): Rₜ=2.05 min; no mass detected

¹H NMR (300 MHz, DMSO-d₆, Method M2) δ 8.12-7.99 (m, 2H), 7.67 (d, J=8.4 Hz, 1H), 5.77 (t, J=6.5 Hz, 1H), 3.88 (td, J=14.2, 6.4 Hz, 2H).

### Step 3: 2-(4-Bromo-2-(trifluoromethyl)phenyl)-2,2-difluoroethanamine

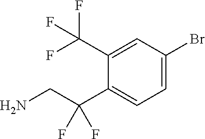 [SMILES: NCC(F)(F)c1ccc(Br)cc1C(F)(F)F]

To a degassed (argon, 5 min) solution of 2-(4-bromo-2-(trifluoromethyl)phenyl)-2,2-difluoroethanol (11.4 g, 37 mmol) in acetonitrile (600 mL) was added pyridine (4.8 g, 60 mmol, 4.8 mL). The mixture was cooled to 0° C. Trifluoromethanesulfonic acid anhydride (11.6 g, 41 mmol, 6.8 mL) was added drop wise while keeping the temperature below 8° C. The mixture was stirred at 0° C. for 30 min. To this solution aqueous ammonia (35%; 46.0 g, 935 mmol, 52 mL) was added. The mixture was stirred at room temperature overnight. Brine (50 mL) was added and the reaction mixture was extracted with ethyl acetate (3×50 mL). The combined organic layers were washed with brine (50 mL) and dried with sodium sulfate. Solvents were removed in vacuo. The crude product was absorbed on isolute. Flash column chromatography (Method L6; 120 g; heptane, 0%-50% ethyl acetate) afforded 6.8 g (22 mmol; 59% of theory) of the title compound.

LC-MS (Method L3): Rₜ=2.11 min; m/z=304/306 (M+H)⁺

¹H NMR (300 MHz, DMSO-d₆, Method M2) δ 8.04 (d, J=7.2 Hz, 2H), 7.68 (d, J=9.0 Hz, 1H), 3.16 (t, J=15.1 Hz, 2H), 1.77 (s, 2H).

### Step 4: N-(2-(4-Bromo-2-(trifluoromethyl)phenyl)-2,2-difluoroethyl)-2-(trifluoromethyl)benzamide

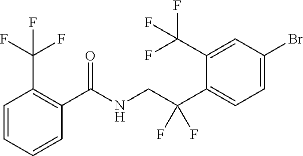 [SMILES: O=C(NCC(F)(F)c1ccc(Br)cc1C(F)(F)F)c1ccccc1C(F)(F)F]

N-(3-dimethylaminopropyl)-3-ethylcarbodiimide hydrochloride (2.1 g, 10.8 mmol) and 1-hydroxy-7-azabenzotriazole (0.3 g, 2.0 mmol) were added to a solution of 2-(4-bromo-2-(trifluoromethyl)phenyl)-2,2-difluoroethanamine (3.0 g, 9.9 mmol) and 2-(trifluoromethyl)-benzoic acid (1.9 g, 9.9 mmol) in N,N-dimethylformamide (100 mL). The mixture was stirred at room temperature for 16 h. Solvents were removed in vacuo. The residue was partitioned between ethyl acetate and water. The organic layer was washed with brine and dried with sodium sulfate. Solvents were removed in vacuo. The crude product was purified by reversed phase flash column chromatography (Method L5; 120 g). The product containing fractions were combined. Organic solvents were removed in vacuo. A precipitate formed, was filtered off and dried in vacuo at 40° C. 4.1 g (8.6 mmol; 87% of theory) of the title compound were obtained.

LC-MS (Method L1): Rₜ=2.23 min; m/z=476/478 (M+H)⁺

¹H NMR (300 MHz, DMSO-d₆, Method M2) δ 9.06 (t, J=6.3 Hz, 1H), 8.09 (d, J=7.6 Hz, 2H), 7.81-7.61 (m, 4H), 7.43 (d, J=7.3 Hz, 1H), 4.13-3.97 (m, 2H).

### Step 5: N-(2,2-Difluoro-2-(4′-fluoro-3-(trifluoromethyl)-[1,1′-biphenyl]-4-yl)ethyl)-2-(trifluoromethyl)benzamide (Example 12)

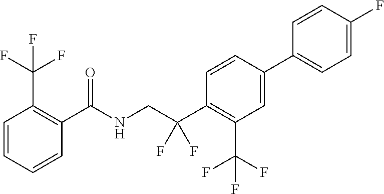 [SMILES: O=C(NCC(F)(F)c1ccc(-c2ccc(F)cc2)cc1C(F)(F)F)c1ccccc1C(F)(F)F]

To a degassed (argon, 5 min) solution of N-(2-(4-bromo-2-(trifluoromethyl)phenyl)-2,2-difluoroethyl)-2-(trifluoromethyl)benzamide (150 mg, 0.32 mmol), (4-fluorophenyl)boronic acid (48.5 mg, 0.35 mmol) and saturated aqueous sodium carbonate (0.95 mmol, 0.47 mL) in 1,2-dimethoxyethane (2 mL) was added bis(triphenylphosphine)palladium(II) chloride (11.1 mg, 0.02 mmol). The mixture was stirred at 100° C. for 16 h and was allowed to cool to room temperature. Dichloromethane (5 mL) was added. The layers were separated in a phase separator and the organic layer was dried with sodium sulfate. Solvents were removed in vacuo. The crude product was triturated in diethylether. The solid was filtered off and dried at 40° C. in vacuo. 155 mg (0.31 mmol; 100% of theory) of the title compound were obtained.

LC-MS (Method L2): Rₜ=3.71 min; m/z=492 (M+H)⁺

¹H NMR (300 MHz, DMSO-d₆, Method M2) δ 9.10 (t, J=6.2 Hz, 1H), 8.12 (d, J=11.1 Hz, 2H), 7.91-7.81 (m, 3H), 7.81-7.61 (m, 3H), 7.45 (d, J=7.4 Hz, 1H), 7.41-7.32 (m, 2H), 4.17-4.01 (m, 2H).

### Preparation Example 4

### Step 1: N-(2-(4-Bromo-2-(trifluoromethyl)phenyl)-2,2-difluoroethyl)-2-(trifluoromethyl)nicotinamide

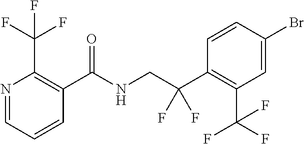 [SMILES: O=C(NCC(F)(F)c1ccc(Br)cc1C(F)(F)F)c1cccnc1C(F)(F)F]

N-(3-Dimethylaminopropyl)-3-ethylcarbodiimide hydrochloride (1.26 g, 6.6 mmol) and 1-hydroxy-7-azabenzotriazole (0.9 g, 6.6 mmol) were added to a solution of 2-(4-bromo-2-(trifluoromethyl)phenyl)-2,2-difluoroethanamine (2.0 g, 6.6 mmol) and 2-(trifluoromethyl)-nicotinic acid (1.4 g, 7.2 mmol) in N,N-dimethylformamide (50 mL). The mixture was stirred at room temperature for 16 h. Solvents were removed in vacuo. The residue was partitioned between ethyl acetate and water. The organic layer was washed with brine and dried with sodium sulfate. Solvents were removed in vacuo. The crude product was purified by reversed phase flash column chromatography (Method L5; 120 g). 1.75 g (3.7 mmol; 55% of theory) of the title compound were obtained.

LC-MS (Method L2): Rₜ=3.24 min; m/z=477/479 (M+H)⁺

¹H NMR (300 MHz, DMSO-d₆, Method M2) δ 9.23 (s, 1H), 8.80 (d, J=4.7 Hz, 1H), 8.09 (d, J=8.7 Hz, 2H), 7.91 (d, J=7.8 Hz, 1H), 7.82-7.69 (m, 2H), 4.15-3.99 (m, 2H).

### Step 2: N-(2,2-Difluoro-2-(4′-fluoro-3-(trifluoromethyl)-[1,1′-biphenyl]-4-yl)ethyl)-2-(trifluoromethyl)-nicotinamide (Example 17)

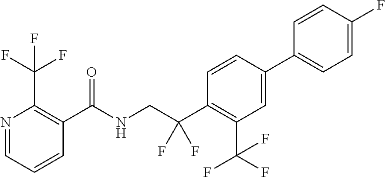 [SMILES: O=C(NCC(F)(F)c1ccc(-c2ccc(F)cc2)cc1C(F)(F)F)c1cccnc1C(F)(F)F]

To a degassed (argon, 5 min) solution of N-(2-(4-bromo-2-(trifluoromethyl)phenyl)-2,2-difluoroethyl)-2-(trifluoromethyl)nicotinamide (150 mg, 0.31 mmol), (4-fluorophenyl)boronic acid (48 mg, 0.35 mmol) and saturated aqueous sodium carbonate (0.94 mmol, 0.47 mL) in 1,2-dimethoxyethane (2 mL) was added bis(triphenylphosphine)palladium(II) chloride (11 mg, 0.02 mmol). The mixture was stirred at 100° C. for 16 h and was allowed to cool to room temperature. Dichloromethane (5 mL) was added. The layers were separated in a phase separator and the organic layer was dried with sodium sulfate. Solvents were removed in vacuo. The crude product was triturated in diethylether. The solid was filtered off and dried at 40° C. in vacuo. 127 mg (0.25 mmol; 82% of theory) of the title compound were obtained.

LC-MS (Method L2): Rₜ=3.44 min; m/z=493 (M+H)⁺

¹H NMR (300 MHz, DMSO-d₆, Method M2) δ 9.27 (t, J=5.9 Hz, 1H), 8.80 (s, 1H), 8.17-8.08 (m, 2H), 7.96-7.75 (m, 5H), 7.42-7.31 (m, 2H), 4.20-4.04 (m, 2H).

TABLE 1
Examples 1 to 38 show compounds according to formula (I)

| | |
|---|---|
| | (I) |
| 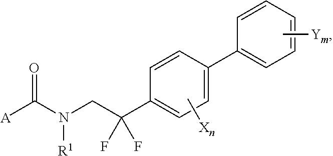 | |

with A, R¹, n, X, m and Y as defined by each individual structure:

| Example | | logP | | |
|---|---|---|---|---|
| Nr. | Structure | (HCOOH)^{[a]} | LC-MS | NMR Data |
| 1 | 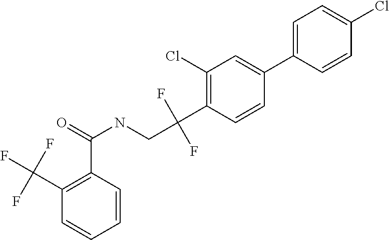 [SMILES: O=C(NCC(F)(F)c1ccc(-c2ccc(Cl)cc2)cc1Cl)c1ccccc1C(F)(F)F] | 4.58 | (Method L0) Rt = 1.64 min; m/z = 453.0; 454.0 (M + H)+ | ¹H-NMR (400 MHz, d₆- DMSO, Method M1); δ 9.02 (t, NH), 7.92 (s, 1H), 7.82- 7.79 (d, 2H), 7.77-7.62 (m, 5H), 7.57-7.55 (d, 2H), 7.38-7.37 (d, 1H), 4.26- 4.17 (dt, 2H). |
| 2 | 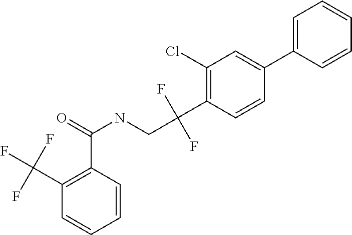 [SMILES: O=C(NCC(F)(F)c1ccc(-c2ccccc2)cc1Cl)c1ccccc1C(F)(F)F] | 4.05 | (Method L0) Rt = 1.48 min; m/z = 420.1 (M + H)+ | ¹H-NMR (400 MHz, d₆- DMSO, Method M1); δ 9.02 (t, NH), 7.89 (s, 1H), 7.81- 7.61 (m, 7H), 7.53-7.42 (m, 3H), 7.39-7.37 (d, 1H), 4.26-4.17 (dt, 2H). |
| 3 | 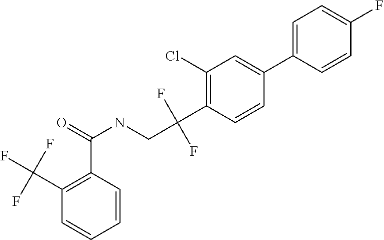 [SMILES: O=C(NCC(F)(F)c1ccc(-c2ccc(F)cc2)cc1Cl)c1ccccc1C(F)(F)F] | 4.10 | (Method L0) Rt = 1.51 min; m/z = 438.1 (−HF) (M + H)+ | ¹H-NMR (400 MHz, d₆- DMSO, Method M1); δ 9.02 (t, NH), 7.90 (s, 1H), 7.84- 7.62 (m, 6H), 7.39-7.30 (m, 3H), 4.25-4.17 (m, 2H). |
| 4 | 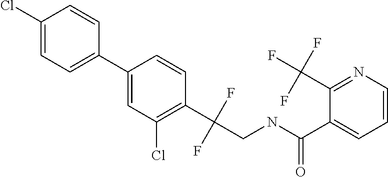 [SMILES: O=C(NCC(F)(F)c1ccc(-c2ccc(Cl)cc2)cc1Cl)c1cccnc1C(F)(F)F] | 4.14 | (Method L0) Rt = 1.55 min; m/z = 474.9; 476.9 (M + H)+ | see NMR peak list (Method M1) |
| 5 | 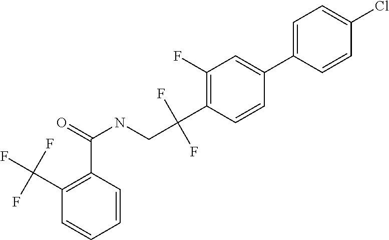 [SMILES: O=C(NCC(F)(F)c1ccc(-c2ccc(Cl)cc2)cc1F)c1ccccc1C(F)(F)F] | | (Method L4) Rₜ = 4.19 min; m/z = 456 (M − H)⁻ | ¹H NMR (300 MHz, DMSO-d₆, Method M2) δ 9.03 (t, J = 6.1 Hz, 1H), 7.86-7.6 (m, 8H), 7.61- 7.52 (m, 2H), 7.38 (d, J = 7.3 Hz, 1H), 4.20-4.03 (m, 2H). |
| 6 | 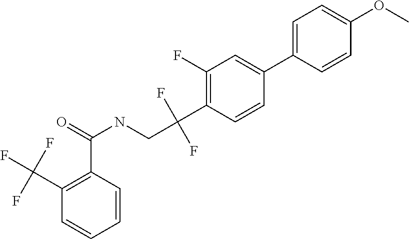 [SMILES: COc1ccc(-c2ccc(C(F)(F)CNC(=O)c3ccccc3C(F)(F)F)c(F)c2)cc1] | | (Method L2) Rₜ = 3.50 min; m/z = 454 (M + H)⁺ | ¹H NMR (300 MHz, DMSO-d₆, Method M2) δ 9.02 (t, J = 6.2 Hz, 1H), 7.78-7.57 (m, 8H), 7.38 (d, J = 7.3 Hz, 1H), 7.09-7.01 (m, 2H), 4.18-4.02 (m, 2H), 3.81 (s, 3H). |
| 7 | 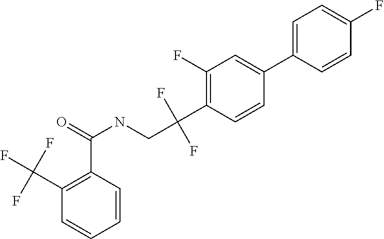 [SMILES: O=C(NCC(F)(F)c1ccc(-c2ccc(F)cc2)cc1F)c1ccccc1C(F)(F)F] | | (Method L2) Rₜ = 3.79 min; m/z = 440 (M − H)⁻ | ¹H NMR (300 MHz, DMSO-d₆, Method M2) δ 9.12-8.90 (m, 1H), 7.92- 7.11 (m, 11H), 4.22-3.95 (m, 2H). |
| 8 | 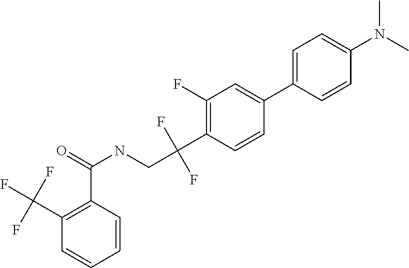 [SMILES: CN(C)c1ccc(-c2ccc(C(F)(F)CNC(=O)c3ccccc3C(F)(F)F)c(F)c2)cc1] | | (Method L2) Rₜ = 3.52 min; m/z = 467 (M + H)⁺ | ¹H NMR (300 MHz, DMSO-d₆, Method M2) δ 9.01 (t, J = 6.3 Hz, 1H), 7.82-7.51 (m, 8H), 7.38 (d, J = 7.5 Hz, 1H), 6.80 (d, J = 8.8 Hz, 2H), 4.02- 3.97 (m, 2H), 2.96 (s, 6H). |
| 9 | 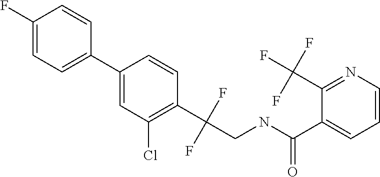 [SMILES: O=C(NCC(F)(F)c1ccc(-c2ccc(F)cc2)cc1Cl)c1cccnc1C(F)(F)F] | 3.68 | (Method L0) Rt = 1.45 min; m/z = 459.1 (M + H)+ | see NMR peak list (Method M1) |
| 10 | 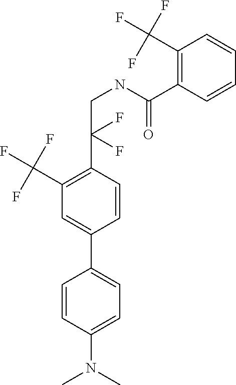 [SMILES: CN(C)c1ccc(-c2ccc(C(F)(F)CNC(=O)c3ccccc3C(F)(F)F)c(C(F)(F)F)c2)cc1] | | (Method L2): Rₜ = 3.75 min; m/z = 517 (M + H)⁺ | ¹H NMR (300 MHz, DMSO-d₆, Method M2) δ 9.07 (t, J = 6.3 Hz, 1H), 8.07-7.99 (m, 2H), 7.80- 7.61 (m, 6H), 7.44 (d, J = 7.5 Hz, 1H), 6.83 (d, J = 8.9 Hz, 2H), 4.16-3.98 (m, 2H), 2.97 (s, 6H). |
| 11 | 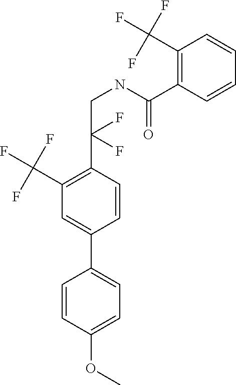 [SMILES: COc1ccc(-c2ccc(C(F)(F)CNC(=O)c3ccccc3C(F)(F)F)c(C(F)(F)F)c2)cc1] | | (Method L2): Rₜ = 3.68 min; m/z = 504 (M + H)⁺ | ¹H NMR (300 MHz, DMSO-d₆, Method M2) δ 9.09 (t, J = 6.1 Hz, 1H), 8.13-8.04 (m, 2H), 7.85- 7.61 (m, 6H), 7.45 (d, J = 7.5 Hz, 1H), 7.08 (d, J = 8.7 Hz, 2H), 4.16-4.00 (m, 2H), 3.82 (s, 3H). |
| 12 | 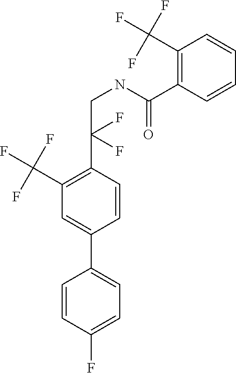 [SMILES: O=C(NCC(F)(F)c1ccc(-c2ccc(F)cc2)cc1C(F)(F)F)c1ccccc1C(F)(F)F] | | (Method L2): Rₜ = 3.71 min; m/z = 492 (M + H)⁺ | ¹H NMR (300 MHz, DMSO-d₆, Method M2) δ 9.10 (t, J = 6.2 Hz, 1H), 8.12 (d, J = 11.1 Hz, 2H), 7.91-7.81 (m, 3H), 7.81- 7.61 (m, 3H), 7.45 (d, J = 7.4 Hz, 1H), 7.41-7.32 (m, 2H), 4.17-4.01 (m, 2H). |
| 13 | 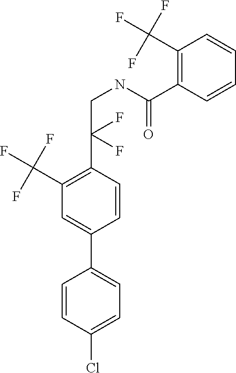 [SMILES: O=C(NCC(F)(F)c1ccc(-c2ccc(Cl)cc2)cc1C(F)(F)F)c1ccccc1C(F)(F)F] | | (Method L2): Rₜ = 3.86 min; m/z = 508 (M + H)⁺ | ¹H NMR (300 MHz, DMSO-d₆, Method M2) δ 9.10 (t, J = 6.2 Hz, 1H), 8.15 (d, J = 10.9 Hz, 2H), 7.91-7.55 (m, 8H), 7.45 (d, J = 7.4 Hz, 1H), 4.17- 4.01 (m, 2H). |
| 14 | 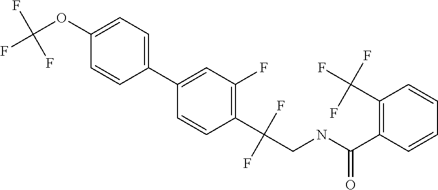 [SMILES: O=C(NCC(F)(F)c1ccc(-c2ccc(OC(F)(F)F)cc2)cc1F)c1ccccc1C(F)(F)F] | | (Method L2): Rₜ = 4.19 min; m/z = 506 (M − H)⁻ | ¹H NMR (300 MHz, DMSO-d₆, Method M2) δ 9.04 (t, J = 6.2 Hz, 1H), 7.92-7.85 (m, 2H), 7.80- 7.59 (m, 6H), 7.49 (d, J = 8.0 Hz, 2H), 7.38 (d, J = 7.2 Hz, 1H), 4.20-4.04 (m, 2H). |
| 15 | 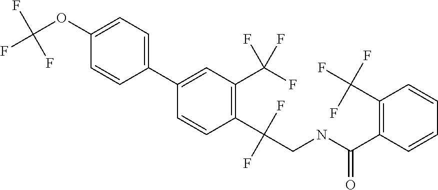 [SMILES: O=C(NCC(F)(F)c1ccc(-c2ccc(OC(F)(F)F)cc2)cc1C(F)(F)F)c1ccccc1C(F)(F)F] | | (Method L2): Rₜ = 4.30 min m/z = 556 (M − H)⁻ | ¹H NMR (300 MHz, DMSO-d₆, Method M2) δ 9.11 (t, J = 6.2 Hz, 1H), 8.16 (d, J = 9.1 Hz, 2H), 7.98-7.84 (m, 3H), 7.81-7.61 (m, 3H), 7.56- 7.41 (m, 3H), 4.22-3.98 (m, 2H). |
| 16 | 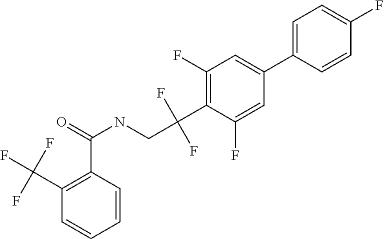 [SMILES: O=C(NCC(F)(F)c1c(F)cc(-c2ccc(F)cc2)cc1F)c1ccccc1C(F)(F)F] | 3.95 | (Method L0) Rt = 1.52 min; m/z = 440.0 (−HF) (M + H)+ | see NMR peak list (Method M1) |
| 17 | 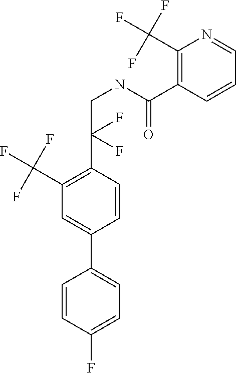 [SMILES: O=C(NCC(F)(F)c1ccc(-c2ccc(F)cc2)cc1C(F)(F)F)c1cccnc1C(F)(F)F] | | (Method L2): Rₜ = 3.44 min; m/z = 493 (M + H)⁺ | ¹H NMR (300 MHz, DMSO-d₆, Method M2) δ 9.27 (t, J = 5.9 Hz, 1H), 8.80 (s, 1H), 8.17-8.08 (m, 2H), 7.96-7.75 (m, 5H), 7.42-7.31 (m, 2H), 4.20-4.04 (m, 2H). |
| 18 | 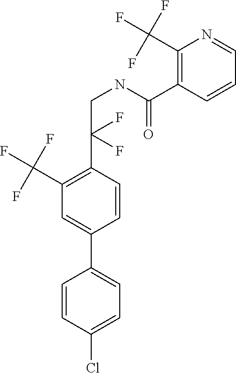 [SMILES: O=C(NCC(F)(F)c1ccc(-c2ccc(Cl)cc2)cc1C(F)(F)F)c1cccnc1C(F)(F)F] | | (Method L2): Rₜ = 3.60 min; m/z = 509 (M + H)⁺ | ¹H NMR (300 MHz, DMSO-d₆, Method M2) δ 9.27 (t, J = 6.0 Hz, 1H), 8.80 (d, J = 4.2 Hz, 1H), 8.15 (d, J = 9.1 Hz, 2H), 7.95-7.75 (m, 5H), 7.62-7.56 (m, 2H), 4.22- 4.00 (m, 2H). |
| 19 | 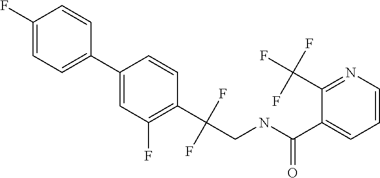 [SMILES: O=C(NCC(F)(F)c1ccc(-c2ccc(F)cc2)cc1F)c1cccnc1C(F)(F)F] | | (Method L2): Rt = 3.31 min; m/z = 443 (M + H)+ | ¹H NMR (300 MHz, DMSO-d₆) δ 9.20 (t, J = 6.1 Hz, 1H), 8.78 (d, J = 4.2 Hz, 1H), 7.87-7.64 (m, 5H), 7.59-7.56 (m, 2H), 7.33 (m, 2H), 4.14 (td, J = 14.6, 6.0 Hz, 2H). |
| 20 | 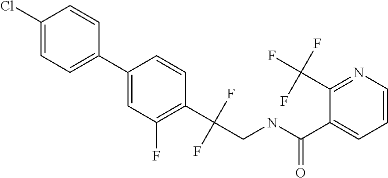 [SMILES: O=C(NCC(F)(F)c1ccc(-c2ccc(Cl)cc2)cc1F)c1cccnc1C(F)(F)F] | | (Method L2): Rt = 3.48 min; m/z = 459 (M + H)+ | ¹H NMR (300 MHz, DMSO-d₆) δ 9.20 (t, J = 6.1 Hz, 1H), 8.79 (d, J = 4.2 Hz, 1H), 7.89-7.71 (m, 5H), 7.67 (d, J = 4.2 Hz, 2H), 7.56 (d, J = 8.6 Hz, 2H), 4.14 (td, J = 14.6, 6.0 Hz, 2H). |
| 21 | 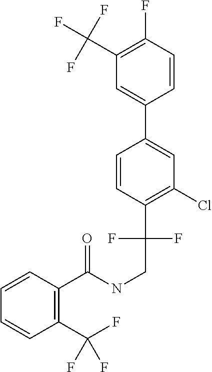 [SMILES: O=C(NCC(F)(F)c1ccc(-c2ccc(F)c(C(F)(F)F)c2)cc1Cl)c1ccccc1C(F)(F)F] | 4.55 | (Method L0) Rt = 1.61 min; m/z = 525.9 (M + H)+ | see NMR peak list |
| 22 | 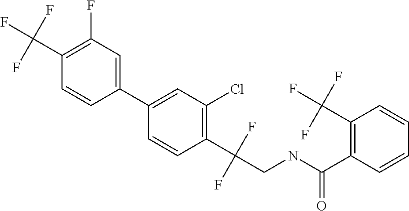 [SMILES: O=C(NCC(F)(F)c1ccc(-c2ccc(C(F)(F)F)c(F)c2)cc1Cl)c1ccccc1C(F)(F)F] | 4.62 | (Method L0) Rt = 1.68 min; m/z = 526.0 (M + H)+ | see NMR peak list |
| 23 | 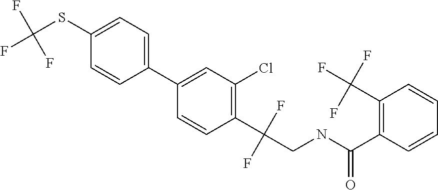 [SMILES: O=C(NCC(F)(F)c1ccc(-c2ccc(SC(F)(F)F)cc2)cc1Cl)c1ccccc1C(F)(F)F] | 4.98 | (Method L0) Rt = 1.68 min; m/z = 540.2 (M + H)+ | see NMR peak list |
| 24 | 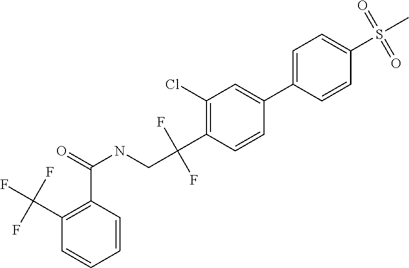 [SMILES: CS(=O)(=O)c1ccc(-c2ccc(C(F)(F)CNC(=O)c3ccccc3C(F)(F)F)c(Cl)c2)cc1] | 2.99 | (Method L0) Rt = 1.24 min; m/z = 518.2 (M + H)+ | see NMR peak list |
| 25 | 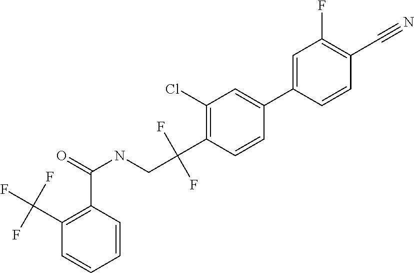 [SMILES: N#Cc1ccc(-c2ccc(C(F)(F)CNC(=O)c3ccccc3C(F)(F)F)c(Cl)c2)cc1F] | 3.77 | (Method L0) Rt = 1.51 min; m/z = 483.0 (M + H)+ | see NMR peak list |
| 26 | 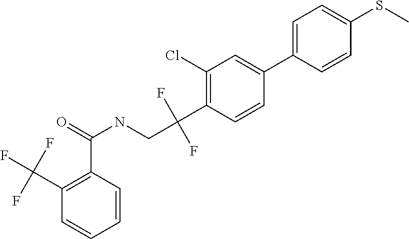 | 4.43 | (Method L0) Rt = 1.65 min; m/z = 466.0 (−MHF) (M + H)+ | see NMR peak list |
| 27 | 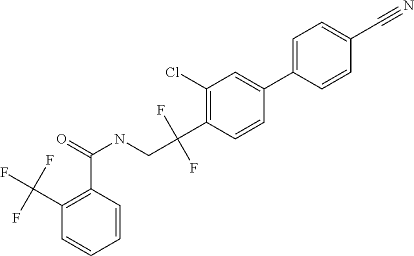 [SMILES: N#Cc1ccc(-c2ccc(C(F)(F)CNC(=O)c3ccccc3C(F)(F)F)c(Cl)c2)cc1] | 3.62 | (Method L0) Rt = 1.47 min; m/z = 464.9 (M + H)+ | see NMR peak list |
| 28 | 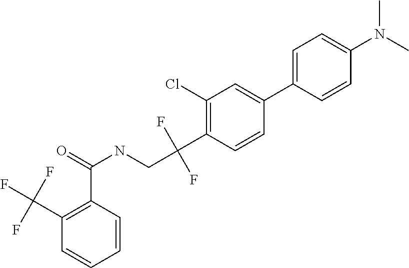 [SMILES: CN(C)c1ccc(-c2ccc(C(F)(F)CNC(=O)c3ccccc3C(F)(F)F)c(Cl)c2)cc1] | 4.28 | (Method L0) Rt = 1.62 min; m/z = 483.0 (M + H)+ | see NMR peak list |
| 29 | 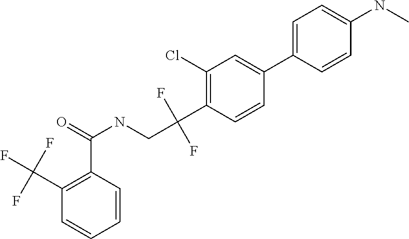 [SMILES: CNc1ccc(-c2ccc(C(F)(F)CNC(=O)c3ccccc3C(F)(F)F)c(Cl)c2)cc1] | 3.59 | (Method L0) Rt = 1.47 min; m/z = 469.0 (M + H)+ | see NMR peak list |
| 30 | 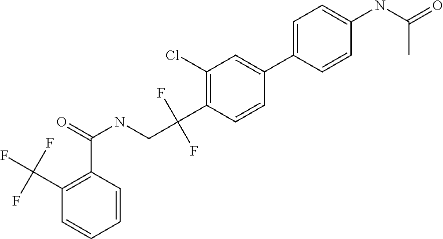 | 2.93 | (Method L0) Rt = 1.30 min; m/z = 476.9 (−HF) (M + H)+ | see NMR peak list |
| 31 | 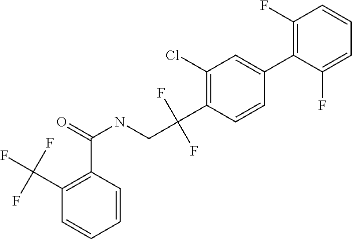 [SMILES: O=C(NCC(F)(F)c1ccc(-c2c(F)cccc2F)cc1Cl)c1ccccc1C(F)(F)F] | 3.99 | (Method L0) Rt = 1.48 min; m/z = 476.2 (M + H)+ | see NMR peak list |
| 32 | 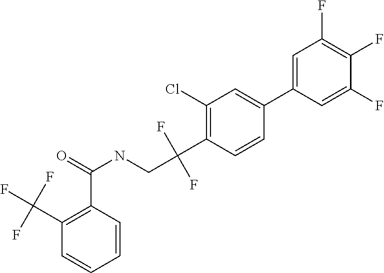 [SMILES: O=C(NCC(F)(F)c1ccc(-c2cc(F)c(F)c(F)c2)cc1Cl)c1ccccc1C(F)(F)F] | 4.44 | (Method L0) Rt = 1.61 min; m/z = 493.9 (M + H)+ | see NMR peak list |
| 33 | 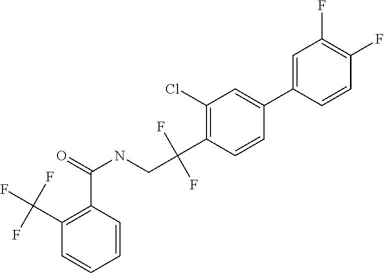 [SMILES: O=C(NCC(F)(F)c1ccc(-c2ccc(F)c(F)c2)cc1Cl)c1ccccc1C(F)(F)F] | 4.26 | (Method L0) Rt = 1.57 min; m/z = 475.9 (M + H)+ | see NMR peak list |
| 34 | 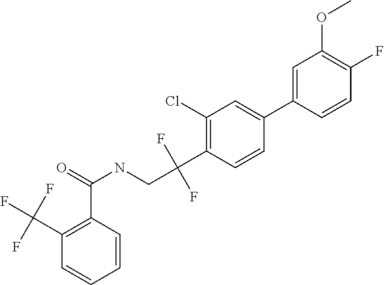 | 4.09 | (Method L0) Rt = 1.53 min; m/z = 467.9 (−HF) (M + H)+ | see NMR peak list |
| 35 | 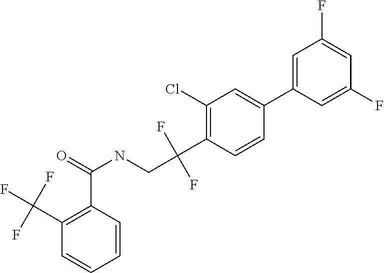 [SMILES: O=C(NCC(F)(F)c1ccc(-c2cc(F)cc(F)c2)cc1Cl)c1ccccc1C(F)(F)F] | 4.30 | (Method L0) Rt = 1.58 min; m/z = 475.9 (M + H)+ | see NMR peak list |
| 36 | 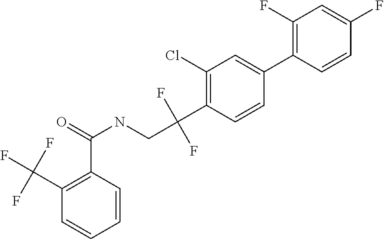 [SMILES: O=C(NCC(F)(F)c1ccc(-c2ccc(F)cc2F)cc1Cl)c1ccccc1C(F)(F)F] | 4.22 | (Method L0) Rt = 1.56 min; m/z = 475.9 (M + H)+ | see NMR peak list |
| 37 | 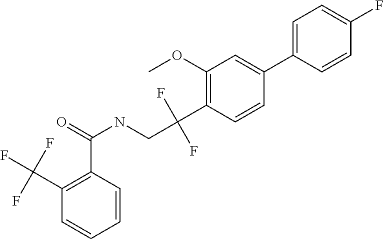 | 3.89 | (Method L0) Rt = 1.49 min; m/z = 434.1 (−HF) (M + H)+ | see NMR peak list |
| 38 | 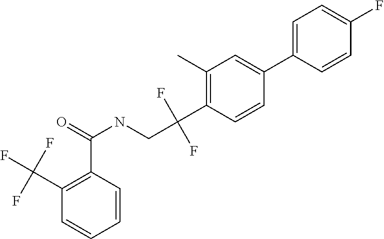 | 4.16 | (Method L0) Rt = 1.55 min; m/z = 418.0 (−HF) (M + H)+ | see NMR peak list |

Examples 1 to 128 show compounds according to formula (I)

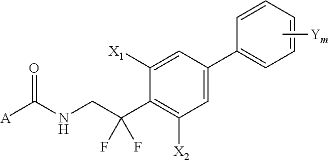

with A, X₁, X₂, Y and m as defined by each individual structure:

| Example | | | | | logP | |
|---|---|---|---|---|---|---|
| Nr. | A | X1 | X2 | Ph[Ym] | (HCOOH)^{[a]} | LC-MS |
| 1 | Ph[2-CF3] | Cl | H | Ph[4-Cl] | 4.58 | (Method L0) Rt = 1.64 min; |
| | | | | | | m/z = 453.0; 454.0 (M + H)+ |
| 2 | Ph[2-CF3] | Cl | H | Ph | 4.05 | (Method L0) Rt = 1.48 min; |
| | | | | | | m/z = 420.1 (M + H)+ |
| 3 | Ph[2-CF3] | Cl | H | Ph[4-F] | 4.10 | (Method L0) Rt = 1.51 min; |
| | | | | | | m/z = 438.1 (−HF) (M + H)+ |
| 4 | 3-Pyridyl[2-CF3] | Cl | H | Ph[4-Cl] | 4.14 | (Method L0) Rt = 1.55 min; |
| | | | | | | m/z = 474.9; 476.9 (M + H)+ |
| 5 | Ph[2-CF3] | F | H | Ph[4-Cl] | | (Method L4) Rₜ = 4.19 min; |
| | | | | | | m/z = 456 (M − H)⁻ |
| 6 | Ph[2-CF3] | F | H | Ph[4-OMe] | | (Method L2) Rₜ = 3.50 min; |
| | | | | | | m/z = 454 (M + H)⁺ |
| 7 | Ph[2-CF3] | F | H | Ph[4-F] | | (Method L2) Rₜ = 3.79 min; |
| | | | | | | m/z 440 (M − H)⁻ |
| 8 | Ph[2-CF3] | F | H | Ph[4-NMe2] | | (Method L2) Rₜ = 3.52 min; |
| | | | | | | m/z = 467 (M + H)⁺ |
| 9 | 3-Pyridyl[2-CF3] | Cl | H | Ph[4-F] | 3.68 | (Method L0) Rt = 1.45 min; |
| | | | | | | m/z = 459.1 (M + H)+ |
| 10 | Ph[2-CF3] | CF3 | H | Ph[4-NMe2] | | (Method L2): Rₜ = 3.75 min; |
| | | | | | | m/z = 517 (M + H)⁺ |
| 11 | Ph[2-CF3] | CF3 | H | Ph[4-OMe] | | (Method L2): Rₜ = 3.68 min; |
| | | | | | | m/z = 504 (M + H)⁺ |
| 12 | Ph[2-CF3] | CF3 | H | Ph[4-F] | | (Method L2): Rₜ = 3.71 min; |
| | | | | | | m/z = 492 (M + H)⁺ |
| 13 | Ph[2-CF3] | CF3 | H | Ph[4-Cl] | | (Method L2): Rₜ = 3.86 min; |
| | | | | | | m/z = 508 (M + H)⁺ |
| 14 | Ph[2-CF3] | F | H | Ph[4-OCF3] | | (Method L2): Rₜ = 4.19 min; |
| | | | | | | m/z = 506 (M − H)⁻ |
| 15 | Ph[2-CF3] | CF3 | H | Ph[4-OCF3] | | (Method L2): Rₜ = 4.30 min; |
| | | | | | | m/z = 556 (M − H)⁻ |
| 16 | Ph[2-CF3] | F | F | Ph[4-F] | 3.95 | (Method L0) Rt = 1.52 min; |
| | | | | | | m/z 440.0 (−HF) (M + H)+ |
| 17 | 3-Pyridyl[2-CF3] | CF3 | H | Ph[4-F] | | (Method L2): Rₜ = 3.44 min; |
| | | | | | | m/z = 493 (M + H)⁺ |
| 18 | 3-Pyridyl[2-CF3] | CF3 | H | Ph[4-Cl] | | (Method L2): Rₜ = 3.60 min; |
| | | | | | | m/z = 509 (M + H)⁺ |
| 19 | 3-Pyridyl[2-CF3] | F | H | Ph[4-F] | | (Method L2): Rₜ = 3.31 min; |
| | | | | | | m/z = 443 (M + H)⁺ |
| 20 | 3-Pyridyl[2-CF3] | F | H | Ph[4-Cl] | | (Method L2): Rₜ = 3.48 min; |
| | | | | | | m/z = 459 (M + H)⁺ |
| 21 | Ph[2-CF3] | Cl | H | Ph[3-CF3/4-F] | 4.55 | (Method L0) Rt = 1.61 min; |
| | | | | | | m/z = 525.9 (M + H)+ |
| 22 | Ph[2-CF3] | Cl | H | Ph[4-CF3/3-F] | 4.62 | (Method L0) Rt = 1.68 min; |
| | | | | | | m/z = 526.0 (M + H)+ |
| 23 | Ph[2-CF3] | Cl | H | Ph[4-SCF3] | 4.98 | (Method L0) Rt = 1.68 min; |
| | | | | | | m/z = 540.2 (M + H)+ |
| 24 | Ph[2-CF3] | Cl | H | Ph[4-SO2Me] | 2.99 | (Method L0) Rt = 1.24 min; |
| | | | | | | m/z = 518.2 (M + H)+ |
| 25 | Ph[2-CF3] | Cl | H | Ph[4-CN/3-F] | 3.77 | (Method L0) Rt = 1.51 min; |
| | | | | | | m/z = 483.0 (M + H)+ |
| 26 | Ph[2-CF3] | Cl | H | Ph[4-SMe] | 4.43 | (Method L0) Rt = 1.65 min; |
| | | | | | | m/z = 466.0 (−HF) (M + H)+ |
| 27 | Ph[2-CF3] | Cl | H | Ph[4-CN] | 3.62 | (Method L0) Rt = 1.47 min; |
| | | | | | | m/z = 464.9 (M + H)+ |
| 28 | Ph[2-CF3] | Cl | H | Ph[4-NMe2] | 4.28 | (Method L0) Rt = 1.62 min; |
| | | | | | | m/z = 483.0 (M + H)+ |
| 29 | Ph[2-CF3] | Cl | H | Ph[4-NHMe] | 3.59 | (Method L0) Rt = 1.47 min; |
| | | | | | | m/z = 469.0 (M + H)+ |
| 30 | Ph[2-CF3] | Cl | H | Ph[4-NHAc] | 2.93 | (Method L0) Rt = 1.30 min; |
| | | | | | | m/z = 476.9 (−HF) (M + H)+ |
| 31 | Ph[2-CF3] | Cl | H | Ph[2-F/6-F] | 3.99 | (Method L0) Rt = 1.48 min; |
| | | | | | | m/z = 476.2 (M + H)+ |
| 32 | Ph[2-CF3] | Cl | H | Ph[3-F/4-F/5-F] | 4.44 | (Method L0) Rt = 1.61 min; |
| | | | | | | m/z = 493.9 (M + H)+ |
| 33 | Ph[2-CF3] | Cl | H | Ph[3-F/4-F] | 4.26 | (Method L0) Rt = 1.57 min; |
| | | | | | | m/z = 475.9 (M + H)+ |
| 34 | Ph[2-CF3] | Cl | H | Ph[3-OMe/4-F] | 4.09 | (Method L0) Rt = 1.53 min; |
| | | | | | | m/z = 467.9 (−HF) (M + H)+ |
| 35 | Ph[2-CF3] | Cl | H | Ph[3-F/5-F] | 4.30 | (Method L0) Rt = 1.58 min; |
| | | | | | | m/z = 475.9 (M + H)+ |
| 36 | Ph[2-CF3] | Cl | H | Ph[2-F/4-F] | 4.22 | (Method L0) Rt = 1.56 min; |
| | | | | | | m/z = 475.9 (M + H)+ |
| 37 | Ph[2-CF3] | OMe | H | Ph[4-F] | 3.89 | (Method L0) Rt = 1.49 min; |
| | | | | | | m/z = 434.1 (−HF) (M + H)+ |
| 38 | Ph[2-CF3] | Me | H | Ph[4-F] | 4.16 | (Method L0) Rt = 1.55 min; |
| | | | | | | m/z = 418.0 (−HF) (M + H)+ |
| 39 | Ph[2-CF3] | Cl | H | Ph[4-OCF3] | 4.67 | Method L0 |
| 40 | Ph[2-CF3] | Cl | H | Ph[4-CHF2] | 3.99 | Method L0 |
| 41 | Ph[2-CF3] | Cl | H | Ph[4-CF3] | 4.56 | Method L0 |
| 42 | Ph[2-CF3] | Cl | H | Ph[3-OCF3] | 4.61 | Method L0 |
| 43 | Ph[2-CF3] | Cl | H | Ph[3-CHF2] | 4.03 | Method L0 |
| 44 | Ph[2-CF3] | OMe | H | Ph[4-SCF3] | 4.74 | Method L0 |
| 45 | Ph[2-CF3] | Cl | H | Ph[4-SO2CF3] | 4.32 | Method L0 |
| 46 | Ph[2-CF3] | F | F | Ph[4-CN | 3.53 | Method L0 |
| 47 | Ph[2-CF3] | F | F | Ph[4-SCF3] | 4.82 | Method L0 |
| 48 | Ph[2-CF3] | F | F | Ph[4-OCF3] | 4.56 | Method L0 |
| 49 | Ph[2-CF3] | Me | H | Ph[4-CN] | 3.7 | Method L0 |
| 50 | Ph[2-CF3] | Me | H | Ph[4-SCF3] | 5.08 | Method L0 |
| 51 | Ph[2-CF3] | OMe | H | Ph[4-CN] | 3.43 | Method L0 |
| 52 | 2-Pyrazinyl[3-CF3] | CF3 | H | Ph[4-F] | | LC-MS (Method L2): Rₜ = |
| | | | | | | 3.52 min; m/z = 494 (M + H)⁺ |
| 53 | 2-Pyrazinyl[3-CF3] | CF3 | H | Ph[4-Cl] | | LC-MS (Method L2): Rₜ = |
| | | | | | | 3.67 min; m/z = 510 (M + H)⁺ |
| 54 | 2-Pyrazinyl[3-CF3] | F | H | Ph[4-F] | | LC-MS (Method L2): Rₜ = |
| | | | | | | 3.38 min; m/z = 442 (M − H)⁻ |
| 55 | 2-Pyrazinyl[3-CF3] | F | H | Ph[4-Cl] | | LC-MS (Method L2): Rₜ = |
| | | | | | | 3.53 min; m/z = 458 (M − H)⁻ |
| 56 | Ph[2-CF3] | Cl | H | Ph[4-SOCF3] | 3.87 | Method L0 |
| 57 | Ph[2-I] | Cl | H | Ph[4-F] | 4.17 | Method L0 |
| 58 | Ph[2-Br] | Cl | H | Ph[4-F] | 4.05 | Method L0 |
| 59 | Ph[2-Cl] | Cl | H | Ph[4-F] | 4.0 | Method L0 |
| 60 | Ph[2-F/6-F] | Cl | H | Ph[4-F] | 3.85 | Method L0 |
| 61 | 2-Pyrazinyl[3- | Cl | H | Ph[4-F] | 3.85 | Method L0 |
| | CF3] | | | | | |
| 62 | 2-Pyridyl[3-CF3] | Cl | H | Ph[4-F] | 4.05 | Method L0 |
| 63 | 3-Pyridyl[2-Cl] | Cl | H | Ph[4-F] | 3.46 | Method L0 |
| 64 | 3-Pyridyl[2-I] | Cl | H | Ph[4-F] | 3.56 | Method L0 |
| 65 | Ph[2-CF3] | Cl | H | Ph[3-OCHF2] | 4.08 | Method L0 |
| 66 | Ph[2-CF3] | Cl | H | Ph[3-F/4- | 4.20 | Method L0 |
| | | | | OCHF2/5-F] | | |
| 67 | Ph[2-CF3] | Cl | H | Ph[3-CF3] | 4.55 | Method L0 |
| 68 | Ph[2-CF3] | Cl | H | Ph[4-OCHF2] | 4.03 | Method L0 |
| 69 | Ph[2-CF3] | Cl | H | Ph[3-Cl] | 4.6 | Method L0 |
| 70 | Ph[2-CF3] | Cl | H | Ph[3-Cl/4-Cl] | 4.93 | Method L0 |
| 71 | Ph[2-CF3] | Cl | H | 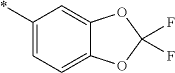 [SMILES: *c1ccc2c(c1)OC(F)(F)O2] | 4.57 | Method L0 |
| 72 | Ph[2-CF3] | CN | H | Ph[4-OCF3] | 4.23 | Method L0 |
| 73 | Ph[2-CF3] | CN | H | Ph[4-CN] | 1.33 | Method L0 |
| 74 | Ph[2-CF3] | CN | H | Ph[4-F] | 3.63 | Method L0 |
| 75 | Ph[2-CF3] | Cl | H | Ph[4- | 3.83 | Method L0 |
| | | | | Morpholinyl] | | |
| 76 | Ph[2-CF3] | Me | H | Ph[4-OCF3] | 4.68 | Method L0 |
| 77 | Ph[2-CF3] | OMe | H | Ph[4-OCF3] | 4.42 | Method L0 |
| 78 | Ph[2-CF3] | Cl | H | 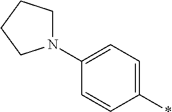 [SMILES: *c1ccc(N2CCCC2)cc1] | 5.08 | Method L0 |
| 79 | Ph[2-CF3] | CN | H | Ph[3-CHF2] | 3.59 | Method L0 |
| 80 | Ph[2-CF3] | Me | H | Ph[4-CHF2] | 4.06 | Method L0 |
| 81 | Ph[2-CF3] | F | F | Ph[4-CHF2] | 3.9 | Method L0 |
| 82 | Ph[2-CF3] | F | F | Ph[3-CHF2] | 3.83 | Method L0 |
| 83 | Ph[2-CF3] | CN | H | Ph[4-SCF3] | 4.5 | Method L0 |
| 84 | Ph[2-CF3] | Cl | H | 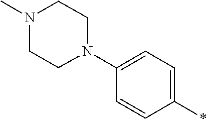 [SMILES: *c1ccc(N2CCN(C)CC2)cc1] | 1.84 | Method L0 |
| 85 | Ph[2-CF3] | CN | H | Ph[4-CF3] | 4.07 | Method L0 |
| 86 | Ph[2-CF3] | F | F | Ph[4-CF3] | 4.38 | Method L0 |
| 87 | Ph[2-CF3] | OMe | H | Ph[4-CF3] | 4.33 | Method L0 |
| 88 | Ph[2-CF3] | Me | H | Ph[4-CF3] | 4.62 | Method L0 |
| 89 | Ph[2-CF3] | CN | H | Ph[4-CHF2] | 3.59 | Method L0 |
| 90 | Ph[2-CF3] | Me | H | Ph[3-CHF2] | 4.07 | Method L0 |
| 91 | Ph[2-CF3] | OMe | H | Ph[4-CHF2] | 3.81 | Method L0 |
| 92 | Ph[2-CF3] | OMe | H | Ph[3-CHF2] | 3.81 | Method L0 |
| 93 | Ph[2-CF3] | Cl | H | Ph[2-F/4-CN] | 3.76 | Method L0 |
| 94 | Ph[2-CF3] | Cl | H | Ph[3-Cl/4-F] | 4.57 | Method L0 |
| 95 | Ph[2-CF3] | Cl | H | Ph[3-CN/4-Cl] | 4.07 | Method L0 |
| 96 | Ph[2-CF3] | Cl | H | Ph[3-CN/4-F] | 3.81 | Method L0 |
| 97 | Ph[2-CF3] | Cl | H | Ph[4-CN/3-CF3] | 4.12 | Method L0 |
| 98 | Ph[2-CF3] | CF3 | H | Ph[3-F/5-F] | | LC-MS (Method L2): Rₜ = |
| | | | | | | 3.99 min; m/z = 510 (M + H)⁺ |
| 99 | Ph[2-CF3] | CF3 | H | Ph[4-CN] | | LC-MS (Method L2): Rₜ = |
| | | | | | | 3.78 min; m/z = 499 (M + H)⁺ |
| 100 | Ph[2-CF3] | CF₃ | H | Ph[3-F/4-F/5-F] | | LC-MS (Method L2): Rₜ = |
| | | | | | | 4.03 min; m/z = 528 (M + H)⁺ |
| 101 | Ph[2-CF3] | F | H | Ph[3-F/5-F] | | LC-MS (Method L2): Rₜ = |
| | | | | | | 3.88 min; m/z = 460 (M + H)⁺ |
| 102 | Ph[2-CF3] | F | H | Ph[4-CN] | | LC-MS (Method L2): Rₜ = |
| | | | | | | 3.65 min; m/z = 449 (M + H)⁺ |
| 103 | Ph[2-CF3] | F | H | Ph[3-F/4-F/5-F] | | LC-MS (Method L2): Rₜ = |
| | | | | | | 3.89 min; m/z = 478 (M + H)⁺ |
| 104 | Ph[2-CF3] | Cl | H | 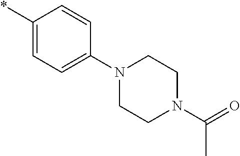 [SMILES: *c1ccc(N2CCN(C(C)=O)CC2)cc1] | 3.17 | Method L0 |
| 105 | Ph[2-CF3] | Cl | H | Ph[4-CN/3-Cl] | 4.02 | Method L0 |
| 106 | Ph[2-CF3] | Cl | H | Ph[4-OCF3/3-F] | 4.8 | Method L0 |
| 107 | Ph[2-CF3] | CN | H | Ph[3-F/4-F/5-F] | 3.89 | Method L0 |
| 108 | Ph[2-CF3] | F | F | Ph[3-F/4-F/5-F] | 4.2 | Method L0 |
| 109 | Ph[2-CF3] | Me | H | Ph[3-F/4-F/5-F] | 4.48 | Method L0 |
| 110 | Ph[2-CF3] | Cl | H | Ph[3-F/4-Cl/5-F] | 4.77 | Method L0 |
| 111 | 2-Pyrazinyl[3-CF3] | Cl | H | Ph[3-F/4-F/5-F] | 4.05 | Method L0 |
| 112 | 2Pyridyl[3-CF3] | Cl | H | Ph[3-F/4-F/5-F] | 4.23 | Method L0 |
| 113 | Ph[2-CF3] | Cl | H | 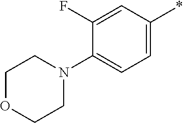 [SMILES: *c1ccc(N2CCOCC2)c(F)c1] | 4.05 | Method L0 |
| 114 | Ph[2-I] | Cl | H | Ph[3-F/4-F/5-F] | 4.34 | Method L0 |
| 115 | Ph[2-Cl] | Cl | H | Ph[3-F/4-F/5-F] | 4.23 | Method L0 |
| 116 | Ph[2-Br] | Cl | H | Ph[3-F/4-F/5-F] | 4.28 | Method L0 |
| 117 | Ph[2-F/6-F] | Cl | H | Ph[3-F/4-F/5-F] | 4.05 | Method L0 |
| 118 | Ph[2-CF3] | OMe | H | Ph[3-F/4-F/5-F] | 4.23 | Method L0 |
| 119 | Ph[2-CF3] | Me | H | Ph[2-F/4-F] | 4.15 | Method L0 |
| 120 | Ph[2-CF3] | Me | H | Ph[3-F/4-F] | 4.2 | Method L0 |
| 121 | Ph[2-CF3] | Me | H | Ph[3-F/4-F] | 4.3 | Method L0 |
| 122 | Ph[2-CF3] | Me | H | Ph[3-Cl/4-Cl] | 4.98 | Method L0 |
| 123 | Ph[2-CF3] | Me | H | Ph[3-Cl] | 4.56 | Method L0 |
| 124 | Ph[2-CF3] | Me | H | Ph[3-Cl/4-F] | 4.56 | Method L0 |
| 125 | Ph[2-CF3] | Me | H | Ph[3-F/4-Cl] | 4.61 | Method L0 |
| 126 | Ph[2-CF3] | Me | H | Ph[3-F/4-Cl/5-F] | 4.77 | Method L0 |
| 127 | Ph[2-CF3] | Me | H | Ph[2-F/3-F] | 4.1 | Method L0 |
| 128 | Ph[2-CF3] | Me | H | Ph[3-Cl/4-F/5-Cl] | 5.08 | Method L0 |

| Example | |
|---|---|
| Nr. | NMR Data |
| 5 | 1H NMR (300 MHz, DMSO-d6, Method M2) δ 9.03 (t, J = 6.1 Hz, 1H), 7.86-7.61 (m, 8H), 7.61-7.52 (m, 2H), 7.38 (d, J = 7.3 |
| | Hz, 1H), 4.20-4.03 (m, 2H). |
| 6 | 1H NMR (300 MHz, DMSO-d6, Method M2) δ 9.02 (t, J = 6.2 Hz, 1H), 7.78-7.57 (m, 8H), 7.38 (d, J = 7.3 Hz, 1H), 7.09-7.01 |
| | (m, 2H), 4.18-4.02 (m, 2H), 3.81 (s, 3H). |
| 7 | 1H NMR (300 MHz, DMSO-d6, Method M2) δ 9.12-8.90 (m, 1H), 7.92-7.11 (m, 11H), 4.22-3.95 (m, 2H). |
| 8 | 1H NMR (300 MHz, DMSO-d6, Method M2) δ 9.01 (t, J = 6.3 Hz, 1H), 7.82-7.51 (m, 8H), 7.38 (d, J = 7.5 Hz, 1H), 6.80 |
| | (d, J = 8.8 Hz, 2H), 4.20-3.97 (m, 2H), 2.96 (s, 6H). |
| 10 | 1H NMR (300 MHz, DMSO-d6, Method M2) δ 9.07 (t, J = 6.3 Hz, 1H), 8.07-7.99 (m, 2H), 7.80-7.61 (m, 6H), 7.44 (d, |
| | J = 7.5 Hz, 1H), 6.83 (d, J = 8.9 Hz, 2H), 4.16-3.98 (m, 2H), 2.97 (s, 6H). |
| 11 | 1H NMR (300 MHz, DMSO-d6, Method M2) δ 9.09 (t, J = 6.1 Hz, 1H), 8.13-8.04 (m, 2H), 7.85-7.61 (m, 6H), 7.45 (d, |
| | J = 7.5 Hz, 1H), 7.08 (d, J = 8.7 Hz, 2H), 4.16-4.00 (m, 2H), 3.82 (s, 3H). |
| 12 | 1H NMR (300 MHz, DMSO-d6, Method M2) δ 9.10 (t, J = 6.2 Hz, 1H), 8.12 (d, J = 11.1 Hz, 2H), 7.91-7.81 (m, 3H), 7.81- |
| | 7.61 (m, 3H), 7.45 (d, J = 7.4 Hz, 1H), 7.41-7.32 (m, 2H), 4.17-4.01 (m, 2H). |
| 13 | 1H NMR (300 MHz, DMSO-d6, Method M2) δ 9.10 (t, J = 6.2 Hz, 1H), 8.15 (d, J = 10.9 Hz, 2H), 7.91-7.55 (m, 8H), 7.45 |
| | (d, J = 7.4 Hz, 1H), 4.17-4.01 (m, 2H). |
| 14 | 1H NMR (300 MHz, DMSO-d6, Method M2) δ 9.04 (t, J = 6.2 Hz, 1H), 7.92-7.85 (m, 2H), 7.80-7.59 (m, 6H), 7.49 |
| | (d, J = 8.0 Hz, 2H), 7.38 (d, J = 7.2 Hz, 1H), 4.20-4.04 (m, 2H). |
| 15 | 1H NMR (300 MHz, DMSO-d6, Method M2) δ 9.11 (t, J = 6.2 Hz, 1H), 8.16 (d, J = 9.1 Hz, 2H), 7.98-7.84 (m, 3H), 7.81- |
| | 7.61 (m, 3H), 7.56-7.41 (m, 3H), 4.22-3.98 (m, 2H). |
| 17 | 1H NMR (300 MHz, DMSO-d6, Method M2) δ 9.27 (t, J = 5.9 Hz, 1H), 8.80 (s, 1H), 8.17-8.08 (m, 2H), 7.96-7.75 (m, 5H), |
| | 7.42-7.31 (m, 2H), 4.20-4.04 (m, 2H). |
| 18 | 1H NMR (300 MHz, DMSO-d6, Method M2) δ 9.27 (t, J = 6.0 Hz, 1H), 8.80 (d, J = 4.2 Hz, 1H), 8.15 (d, J = 9.1 Hz, 2H), |
| | 7.95-7.75 (m, 5H), 7.62-7.56 (m, 2H), 4.22-4.00 (m, 2H). |
| 19 | 1H NMR (300 MHz, DMSO-d6, Method M2) δ 9.20 (t, J = 6.1 Hz, 1H), 8.78 (d, J = 4.2 Hz, 1H), 7.87-7.64 (m, 5H), 7.59- |
| | 7.56 (m, 2H), 7.33 (m, 2H), 4.14 (td, J = 14.6, 6.0 Hz, 2H). |
| 20 | 1H NMR (300 MHz, DMSO-d6, Method M2) δ 9.20 (t, J = 6.1 Hz, 1H), 8.79 (d, J = 4.2 Hz, 1H), 7.89-7.71 (m, 5H), 7.67 (d, |
| | J = 4.2 Hz, 2H), 7.56 (d, J = 8.6 Hz, 2H), 4.14 (td, J = 14.6, 6.0 Hz, 2H). |
| 52 | 1H NMR (300 MHz, DMSO-d6, Method M2) δ 9.40 (s, 1H), 9.04 (d, J = 2.3 Hz, 1H), 8.98 (d, J = 2.3 Hz, 1H), 8.10 (d, |
| | J = 5.5 Hz, 2H), 7.91-7.79 (m, 3H), 7.36 (t, J = 8.8 Hz, 2H), 4.15 (t, 2H). |
| 53 | 1H NMR (300 MHz, DMSO-d6, Method M2) δ 9.40 (s, 1H), 9.04 (d, J = 2.3 Hz, 1H), 8.98 (d, J = 2.3 Hz, 1H), 8.13 (d, |
| | J = 5.6 Hz, 2H), 7.90-7.80 (m, 3H), 7.59 (d, J = 8.6 Hz, 2H), 4.14 (t, 2H). |
| 54 | 1H NMR (300 MHz, DMSO-d6, Method M2) δ 9.33 (s, 1H), 9.02 (d, J = 2.4 Hz, 1H), 8.96 (d, J = 2.4 Hz, 1H), 7.88-7.58 |
| | (m, 5H), 7.40-7.28 (m, 2H), 4.18 (t, J = 14.8 Hz, 2H). |
| 55 | 1H NMR (300 MHz, DMSO-d6, Method M2) δ 9.33 (s, 1H), 9.01 (d, J = 2.2 Hz, 1H), 8.96 (d, J = 2.3 Hz, 1H), 7.85-7.50 |
| | (m, 7H), 4.18 (td, J = 14.8, 5.5 Hz, 2H). |
| 98 | 1H NMR (300 MHz, DMSO-d6, Method M2) δ 9.10 (t, J = 6.1 Hz, 1H), 8.27-8.18 (m, 2H), 7.87 (d, J = 8.1 Hz, 1H), 7.82- |
| | 7.58 (m, 5H), 7.45 (d, J = 7.4 Hz, 1H), 7.36 (tt, J = 9.2, 2.3 Hz, 1H), 4.10 (td, J = 14.6, 6.3 Hz, 2H). |
| 99 | 1H NMR (300 MHz, DMSO-d6, Method M2) δ 9.11 (t, J = 6.2 Hz, 1H), 8.23 (d, J = 10.1 Hz, 2H), 8.08-7.96 (m, 4H), 7.91 (d, |
| | J = 8.2 Hz, 1H), 7.81-7.60 (m, 3H), 7.45 (d, J = 7.3 Hz, 1H), 4.10 (td, J = 14.9, 6.6 Hz, 2H). |
| 100 | 1H NMR (300 MHz, DMSO-d6, Method M2) δ 9.10 (t, J = 6.4 Hz, 1H), 8.25-8.17 (m, 2H), 7.98-7.61 (m, 6H), 7.44 (d, |
| | J = 7.2 Hz, 1H), 4.09 (td, J = 14.7, 6.3 Hz, 2H). |
| 101 | 1H NMR (300 MHz, DMSO-d6, Method M2) δ 9.04 (t, J = 6.4 Hz, 1H), 7.86 (d, J = 12.8 Hz, 1H), 7.80-7.52 (m, 7H), 7.41- |
| | 7.26 (m, 2H), 4.11 (td, J = 14.4, 6.3 Hz, 2H). |
| 102 | 1H NMR (300 MHz, DMSO-d6, Method M2) δ 9.04 (t, J = 6.3 Hz, 1H), 7.98 (s, 4H), 7.84 (d, J = 12.7 Hz, 1H), 7.80-7.58 (m, |
| | 5H), 7.38 (d, J = 7.1 Hz, 1H), 4.12 (td, J = 14.5, 6.3 Hz, 2H). |
| 103 | 1H NMR (300 MHz, DMSO-d6, Method M2) δ 9.02 (s, 1H), 7.91-7.47 (m, 8H), 7.36 (d, J = 6.6 Hz, 1H), 4.09 (t, J = 14.5 Hz, |
| | 2H). |

TABLE 2
Example VII-1 shows a compound according to formula (VII)

(VII)

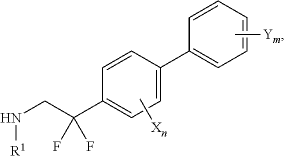

with R¹, n, X, m and Y as defined by each individual structure:

| Example | | logP | | |
|---|---|---|---|---|
| Nr. | Structure | (HCOOH) | LC-MS | NMR Data |
| VII-1 | 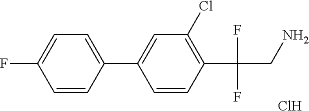 [SMILES: NCC(F)(F)c1ccc(-c2ccc(F)cc2)cc1Cl ; Cl] | 1.35 | (Method L0) Rt = 0.69 min; m/z = 286.1 (M + H)+ | ¹H NMR (400 MHz, d₆-DMSO, Method M1); δ 7.85-7.52 (m, 5H), 7.36-7.30 (t, 2H), 3.35- 3.28 (broad m, NH₂, 2H). |

NMR Peak Lists

¹H-NMR data of selected examples are written in form of ¹H-NMR-peak lists. The δ-value in ppm and the signal intensity are listed to each signal peak in round brackets. Between the δ-value—signal intensity pairs are semicolons as delimiters.

The peak list of an example has therefore the form:

δ₁ (intensity₁); δ₂ (intensity₂); . . . ; δᵢ (intensityᵢ); . . . ; δₙ (intensityₙ)

Intensity of sharp signals correlates with the height of the signals in a printed example of a NMR spectrum in cm and shows the real relations of signal intensities. From broad signals several peaks or the middle of the signal and their relative intensity in comparison to the most intensive signal in the spectrum can be shown.

Tetramethylsilane and/or the chemical shift of the used solvent, especially in the case of spectra measured in DMSO (dimethylsulfoxide), have been used for calibrating. Therefore, tetramethylsilane peak can occur but not necessarily in NMR peak lists.

The ¹H-NMR peak lists are similar to classical ¹H-NMR prints and contain therefore usually all peaks, which are listed at classical NMR-interpretation.

Additionally they can show like classical ¹H-NMR prints signals of solvents, stereoisomers of the target compounds, which are also object of the invention, and/or peaks of impurities.

The usual peaks of solvents, for example peaks of DMSO in D₆-DMSO and the peak of water, are given in the ¹H-NMR peak lists to show compound signals in the delta-range of solvents and/or water. They have usually on average a high intensity.

The peaks of stereoisomers of the target compounds and/or peaks of impurities have usually on average a lower intensity than the peaks of target compounds (for example with a purity >90%).

Such stereoisomers and/or impurities can be typical for the specific preparation process. Therefore, their peaks can help to recognize the reproduction of our preparation process via “side-products-fingerprints”.

An expert, who calculates the peaks of the target compounds with known methods (MestreC, ACD-simulation, but also with empirically evaluated expectation values) can isolate the peaks of the target compounds as needed optionally using additional intensity filters. This isolation would be similar to relevant peak picking at classical ¹H-NMR interpretation.

Further details of NMR-data description with peak lists can be found in the publication “Citation of NMR Peaklist Data within Patent Applications” (cf. Research Disclosure Database Number 564025, 2011, 16 Mar. 2011 or http://www.rdelectronic.co.uk/rd/free/rd564025.pdf).

Table with NMR Peaklists

Example 4: ¹H-NMR (400.0 MHz, d₆-DMSO):
δ = 9.202 (2.0); 9.186 (4.4); 9.171 (2.1); 8.795 (4.3); 8.785 (4.2); 8.783 (4.3); 7.935 (7.2); 7.932 (8.0); 7.870 (3.5);
7.853 (5.1); 7.850 (5.1); 7.822 (4.2); 7.815 (13.1); 7.810 (4.5); 7.798 (9.1); 7.793 (16.0); 7.786 (6.6); 7.774 (4.6);
7.767 (3.2); 7.755 (3.3); 7.744 (8.8); 7.723 (5.4); 7.583 (1.6); 7.577 (14.9); 7.572 (4.6); 7.560 (3.8); 7.555 (12.6);
7.549 (1.4); 4.284 (1.7); 4.268 (1.8); 4.247 (4.0); 4.232 (3.9); 4.211 (2.0); 4.195 (1.9); 3.331 (61.8); 2.677 (0.3);
2.672 (0.5); 2.668 (0.4); 2.543 (11.3); 2.526 (1.0); 2.512 (26.9); 2.508 (55.7); 2.503 (74.4); 2.499 (54.3); 2.494 (26.4);
2.335 (0.3); 2.330 (0.5); 2.325 (0.3); 0.000 (0.9)
Example 9: ¹H-NMR (400.0 MHz, d₆-DMSO):
δ = 9.202 (2.9); 9.187 (6.0); 9.171 (2.9); 8.794 (6.3); 8.783 (6.2); 8.317 (0.6); 7.904 (11.1); 7.868 (4.9); 7.849 (8.0);
7.839 (8.3); 7.835 (4.3); 7.826 (9.0); 7.817 (9.7); 7.809 (4.5); 7.804 (9.3); 7.796 (5.2); 7.786 (7.1); 7.774 (13.0);
7.767 (4.9); 7.755 (4.1); 7.731 (11.7); 7.710 (6.6); 7.362 (8.4); 7.340 (16.0); 7.318 (7.8); 4.281 (2.5); 4.265 (2.7);
4.245 (5.7); 4.229 (5.6); 4.208 (3.0); 4.192 (2.8); 3.330 (325.2); 2.676 (1.4); 2.671 (1.9); 2.667 (1.4); 2.507 (220.1);
2.502 (283.4); 2.498 (216.5); 2.329 (1.9); 2.325 (1.5); 2.075 (0.4); 1.299 (0.6); 0.000 (0.8)
Example 16: ¹H-NMR (400.0 MHz, d₆-DMSO):
δ = 9.158 (2.7); 9.142 (5.6); 9.127 (2.7); 8.318 (0.4); 7.894 (7.6); 7.890 (3.7); 7.881 (8.7); 7.872 (9.1); 7.864 (4.0);
7.859 (8.3); 7.778 (5.5); 7.758 (8.0); 7.733 (6.4); 7.715 (4.4); 7.669 (4.6); 7.646 (13.5); 7.631 (2.9); 7.617 (10.8);
7.416 (6.6); 7.397 (6.0); 7.364 (8.4); 7.342 (16.0); 7.320 (7.8); 4.155 (2.4); 4.139 (2.6); 4.120 (5.4); 4.104 (5.2);
4.085 (2.8); 4.069 (2.6); 3.333 (212.1); 2.676 (0.8); 2.672 (1.1); 2.668 (0.8); 2.507 (133.3); 2.503 (170.8); 2.499 (126.1);
2.334 (0.8); 2.330 (1.1); 2.325 (0.8); 1.398 (2.1); 0.008 (0.6); 0.000 (14.5)
Example 21: ¹H-NMR (400.0 MHz, d₆-DMSO):
δ = 9.049 (3.0); 9.033 (6.1); 9.018 (3.1); 8.317 (0.5); 8.169 (3.0); 8.156 (3.8); 8.148 (3.9); 8.136 (3.8); 8.127 (5.8);
8.110 (5.7); 8.075 (0.4); 8.048 (11.2); 7.890 (4.9); 7.869 (6.4); 7.770 (5.9); 7.752 (16.0); 7.731 (8.1); 7.707 (6.6);
7.686 (7.1); 7.658 (10.1); 7.637 (9.1); 7.619 (2.4); 7.394 (6.8); 7.375 (6.1); 4.263 (2.6); 4.248 (2.9); 4.227 (5.8);
4.212 (5.8); 4.191 (3.1); 4.176 (2.9); 3.330 (170.2); 2.672 (1.8); 2.503 (268.4); 2.330 (1.8); 0.000 (21.8)
Example 22: ¹H-NMR (400.0 MHz, d₆-DMSO):
δ = 9.053 (5.2); 9.038 (8.8); 9.023 (4.1); 8.319 (0.4); 8.080 (16.0); 8.026 (8.1); 7.996 (8.0); 7.941 (8.3); 7.921 (13.9);
7.901 (9.6); 7.881 (7.5); 7.850 (11.2); 7.829 (6.0); 7.785 (14.8); 7.770 (12.9); 7.764 (13.6); 7.752 (11.5); 7.729 (4.4);
7.710 (9.5); 7.692 (7.0); 7.659 (7.4); 7.640 (8.4); 7.621 (3.1); 7.391 (9.8); 7.373 (8.3); 4.271 (4.4); 4.255 (4.9);
4.235 (9.0); 4.220 (8.2); 4.199 (4.6); 4.184 (3.8); 3.340 (30.7); 3.332 (98.4); 2.677 (1.2); 2.673 (1.3); 2.508 (179.1);
2.504 (189.9); 2.499 (128.8); 2.335 (1.2); 2.330 (1.2); 2.077 (1.0); 0.007 (5.3); 0.000 (19.2)
Example 23: ¹H-NMR (400.0 MHz, d₆-DMSO):
δ = 9.051 (2.2); 9.036 (4.8); 9.020 (2.3); 7.987 (8.6); 7.941 (9.9); 7.920 (16.0); 7.876 (3.5); 7.872 (3.4); 7.849 (15.7);
7.828 (9.0); 7.792 (0.4); 7.773 (9.5); 7.752 (8.9); 7.726 (2.1); 7.708 (4.9); 7.690 (3.6); 7.656 (3.7); 7.637 (4.5);
7.618 (1.7); 7.389 (5.2); 7.371 (4.7); 4.270 (1.9); 4.255 (2.0); 4.234 (4.3); 4.219 (4.2); 4.198 (2.2); 4.183 (2.0);
3.335 (78.8); 2.677 (0.4); 2.673 (0.5); 2.669 (0.4); 2.508 (58.4); 2.504 (76.5); 2.499 (57.4); 2.331 (0.5); 2.326 (0.4);
1.357 (0.5); 1.309 (1.5); 0.008 (0.3); 0.000 (9.8)
Example 24: ¹H-NMR (400.0 MHz, d₆-DMSO):
δ = 9.053 (0.6); 9.038 (1.4); 9.023 (0.8); 8.065 (0.4); 8.042 (16.0); 8.024 (3.3); 7.900 (1.0); 7.883 (1.4); 7.879 (1.4);
7.792 (2.2); 7.771 (2.8); 7.750 (1.8); 7.728 (0.6); 7.710 (1.4); 7.691 (1.1); 7.657 (1.1); 7.638 (1.4); 7.619 (0.6);
7.394 (1.4); 7.375 (1.4); 4.276 (0.5); 4.260 (0.6); 4.239 (1.2); 4.224 (1.3); 4.204 (0.7); 4.188 (0.7); 3.333 (15.2);
3.282 (12.4); 2.507 (19.0); 2.503 (24.5); 2.499 (19.7); 0.000 (2.6)
Example 25: ¹H-NMR (400.0 MHz, d₆-DMSO):
δ = 9.050 (0.4); 9.034 (0.8); 9.019 (0.4); 8.094 (1.3); 8.091 (1.4); 8.084 (0.8); 8.063 (1.1); 8.056 (0.9); 8.046 (0.8);
8.033 (0.8); 8.029 (0.8); 7.953 (0.6); 7.949 (0.6); 7.933 (0.8); 7.928 (0.8); 7.876 (0.9); 7.872 (0.9); 7.855 (0.8);
7.851 (0.8); 7.782 (1.4); 7.766 (0.8); 7.761 (1.2); 7.747 (1.0); 7.724 (0.3); 7.706 (0.8); 7.688 (0.6); 7.655 (0.6);
7.636 (0.8); 7.383 (0.9); 7.365 (0.8); 4.250 (0.3); 4.229 (0.7); 4.214 (0.7); 4.193 (0.4); 4.177 (0.3); 4.038 (0.8);
4.020 (0.9); 3.331 (61.9); 2.671 (0.3); 2.525 (0.8); 2.511 (19.9); 2.507 (40.6); 2.503 (53.6); 2.498 (39.5); 2.494 (19.9);
2.329 (0.3); 1.989 (3.7); 1.398 (16.0); 1.193 (1.0); 1.175 (1.9); 1.157 (0.9); 0.146 (0.4); 0.008 (2.8); 0.000 (80.6);
−0.009 (3.5); −0.019 (0.3); −0.150 (0.4)
Example 26: ¹H-NMR (400.0 MHz, d₆-DMSO):
δ = 9.033 (2.0); 9.018 (4.3); 9.002 (2.0); 8.317 (0.5); 7.886 (7.1); 7.883 (7.7); 7.797 (3.0); 7.793 (2.9); 7.776 (5.1);
7.771 (6.5); 7.748 (5.6); 7.730 (11.5); 7.726 (6.1); 7.715 (11.6); 7.709 (16.0); 7.694 (5.6); 7.687 (3.6); 7.654 (3.4);
7.635 (4.1); 7.616 (1.5); 7.385 (6.4); 7.379 (13.4); 7.363 (5.8); 7.357 (11.5); 4.251 (1.7); 4.235 (1.8); 4.215 (3.9);
4.199 (3.7); 4.179 (2.0); 4.163 (1.8); 4.037 (0.8); 4.020 (0.8); 3.382 (0.4); 3.334 (435.6); 2.698 (0.3); 2.676 (1.1);
2.671 (1.5); 2.667 (1.1); 2.552 (0.7); 2.525 (60.6); 2.511 (83.9); 2.507 (168.1); 2.502 (220.1); 2.498 (161.2); 2.494 (80.1);
2.333 (1.0); 2.329 (1.4); 2.325 (1.0); 1.989 (3.3); 1.397 (1.5); 1.234 (0.6); 1.193 (0.9); 1.175 (1.7); 1.157 (0.9);
1.120 (0.4); 0.982 (0.4); 0.146 (1.7); 0.008 (14.2); 0.000 (355.5); −0.009 (15.0); −0.150 (1.7)
Example 27: ¹H-NMR (400.0 MHz, d₆-DMSO):
δ = 9.047 (0.6); 9.031 (1.3); 9.016 (0.6); 8.019 (2.3); 8.016 (2.5); 8.004 (0.5); 7.980 (16.0); 7.958 (0.4); 7.898 (1.0);
7.894 (1.0); 7.878 (1.4); 7.874 (1.4); 7.778 (2.4); 7.766 (1.4); 7.757 (1.9); 7.747 (1.7); 7.724 (0.5); 7.706 (1.4);
7.688 (1.0); 7.654 (1.0); 7.635 (1.3); 7.617 (0.4); 7.387 (1.5); 7.368 (1.3); 4.265 (0.5); 4.249 (0.6); 4.230 (1.2);
4.214 (1.2); 4.194 (0.6); 4.178 (0.6); 3.331 (60.6); 2.671 (0.4); 2.525 (1.0); 2.507 (43.0); 2.503 (56.3); 2.498 (41.8);
2.329 (0.4); 1.989 (0.3); 0.146 (0.4); 0.008 (3.3); 0.000 (84.1); −0.009 (3.8); −0.150 (0.4)
Example 28: ¹H-NMR (400.0 MHz, d₆-DMSO):
δ = 9.019 (0.4); 9.004 (0.8); 8.988 (0.4); 7.778 (1.5); 7.769 (0.8); 7.749 (1.0); 7.716 (0.6); 7.712 (0.6); 7.704 (0.9);
7.695 (1.0); 7.691 (1.0); 7.653 (0.7); 7.643 (1.6); 7.631 (2.5); 7.623 (1.2); 7.609 (2.1); 7.383 (0.9); 7.364 (0.8);
6.813 (1.9); 6.791 (1.8); 4.196 (0.7); 4.180 (0.7); 4.161 (0.4); 4.144 (0.3); 3.328 (43.3); 2.963 (16.0); 2.675 (0.4);
2.671 (0.5); 2.506 (55.8); 2.502 (72.6); 2.498 (54.5); 2.329 (0.5); 2.324 (0.4); 0.146 (0.5); 0.008 (4.4); 0.000 (100.4);
−0.008 (4.7); −0.150 (0.5)
Example 29: ¹H-NMR (400.0 MHz, d₆-DMSO):
δ = 9.015 (2.0); 9.000 (4.1); 8.984 (2.0); 8.317 (0.4); 7.768 (3.8); 7.748 (5.9); 7.738 (7.9); 7.723 (1.9); 7.704 (4.4);
7.685 (3.5); 7.654 (7.9); 7.633 (4.6); 7.625 (8.1); 7.615 (2.1); 7.604 (3.6); 7.547 (9.2); 7.525 (9.7); 7.381 (4.5);
7.362 (4.0); 6.635 (9.3); 6.613 (9.0); 6.030 (1.7); 4.226 (1.7); 4.210 (1.8); 4.190 (3.7); 4.174 (3.6); 4.153 (1.9);
4.138 (1.8); 4.038 (0.5); 4.020 (0.5); 3.858 (0.4); 3.781 (0.5); 3.413 (0.4); 3.334 (164.2); 2.719 (16.0); 2.671 (1.3);
2.502 (184.6); 2.329 (1.2); 1.989 (1.8); 1.352 (0.5); 1.314 (0.8); 1.249 (0.4); 1.232 (0.7); 1.193 (0.5); 1.175 (1.0);
1.157 (0.5); 0.146 (1.2); 0.000 (236.6); −0.150 (1.2)
Example 30: ¹H-NMR (400.0 MHz, d₆-DMSO):
δ = 10.102 (2.9); 9.030 (0.8); 9.014 (1.6); 8.999 (0.8); 7.856 (2.9); 7.770 (2.0); 7.750 (3.5); 7.724 (1.2); 7.710 (16.0);
7.683 (2.3); 7.654 (1.2); 7.634 (1.5); 7.615 (0.6); 7.382 (1.7); 7.364 (1.6); 4.246 (0.6); 4.231 (0.7); 4.211 (1.4);
4.195 (1.3); 4.174 (0.7); 4.159 (0.7); 4.055 (0.6); 4.038 (2.0); 4.020 (2.0); 4.002 (0.7); 3.330 (25.9); 2.671 (0.4);
2.507 (43.4); 2.502 (55.5); 2.498 (41.4); 2.329 (0.4); 2.071 (13.9); 1.989 (8.5); 1.972 (0.6); 1.234 (0.3); 1.193 (2.2);
1.175 (4.4); 1.157 (2.2); 0.146 (0.4); 0.008 (3.6); 0.000 (81.9); −0.008 (4.2); −0.150 (0.4)
Example 31: ¹H-NMR (400.0 MHz, d₆-DMSO):
δ = 9.059 (3.2); 9.043 (6.6); 9.028 (3.2); 8.318 (1.1); 7.785 (9.7); 7.765 (16.0); 7.746 (11.4); 7.741 (12.3); 7.714 (2.6);
7.695 (7.0); 7.677 (5.6); 7.657 (5.6); 7.638 (6.4); 7.613 (6.9); 7.590 (6.1); 7.572 (3.3); 7.568 (3.0); 7.551 (5.9);
7.535 (3.2); 7.530 (3.7); 7.514 (1.6); 7.354 (7.4); 7.335 (6.7); 7.304 (1.6); 7.294 (9.8); 7.274 (15.9); 7.253 (8.0);
7.243 (1.3); 4.290 (2.9); 4.274 (3.0); 4.254 (6.5); 4.238 (6.3); 4.218 (3.3); 4.202 (3.1); 3.330 (143.9); 2.676 (1.4);
2.672 (1.8); 2.667 (1.3); 2.507 (214.0); 2.503 (274.0); 2.498 (201.8); 2.334 (1.3); 2.329 (1.8); 2.325 (1.3); 0.146 (1.5);
0.008 (15.6); 0.000 (330.3); −0.008 (16.2); −0.150 (1.6)
Example 32: ¹H-NMR (400.0 MHz, d₆-DMSO):
δ = 9.045 (1.6); 9.029 (3.5); 9.013 (1.7); 8.317 (0.5); 8.032 (5.9); 8.028 (6.3); 7.905 (0.5); 7.887 (6.7); 7.870 (7.8);
7.865 (7.2); 7.846 (4.0); 7.829 (0.4); 7.770 (3.3); 7.750 (4.7); 7.737 (6.5); 7.726 (1.8); 7.716 (5.3); 7.709 (4.0);
7.690 (2.7); 7.657 (2.9); 7.638 (3.4); 7.619 (1.2); 7.380 (3.9); 7.362 (3.5); 4.255 (1.5); 4.240 (1.5); 4.219 (3.3);
4.203 (3.2); 4.183 (1.7); 4.167 (1.6); 3.331 (282.8); 2.676 (1.0); 2.672 (1.4); 2.667 (1.0); 2.525 (3.6); 2.511 (75.3);
2.507 (154.7); 2.503 (205.7); 2.498 (152.3); 2.494 (76.7); 2.334 (0.9); 2.329 (1.3); 2.325 (1.0); 1.989 (0.5); 1.398 (16.0);
0.008 (1.2); 0.000 (35.8); −0.008 (1.5)
Example 33: ¹H-NMR (400.0 MHz, d₆-DMSO):
δ = 9.043 (0.6); 9.027 (1.3); 9.012 (0.6); 7.968 (2.2); 7.965 (2.8); 7.945 (0.6); 7.939 (0.7); 7.934 (0.7); 7.928 (0.6);
7.914 (0.6); 7.909 (0.6); 7.844 (1.0); 7.840 (0.9); 7.824 (1.4); 7.819 (1.4); 7.769 (1.2); 7.750 (1.6); 7.732 (2.6);
7.711 (2.7); 7.690 (1.0); 7.675 (0.4); 7.667 (0.5); 7.656 (1.7); 7.638 (1.8); 7.618 (0.5); 7.600 (0.7); 7.579 (1.1);
7.574 (0.8); 7.557 (0.6); 7.553 (1.1); 7.531 (0.5); 7.385 (1.4); 7.367 (1.3); 4.256 (0.5); 4.241 (0.6); 4.220 (1.2);
4.204 (1.2); 4.184 (0.6); 4.168 (0.6); 3.331 (22.6); 2.525 (0.5); 2.512 (11.9); 2.508 (24.6); 2.503 (32.6); 2.499 (23.7);
2.494 (11.5); 1.990 (0.8); 1.397 (16.0); 1.175 (0.4); 0.000 (7.2)
Example 34: ¹H-NMR (400.0 MHz, d₆-DMSO):
δ = 9.044 (0.5); 9.028 (1.0); 9.012 (0.5); 7.956 (1.9); 7.824 (0.7); 7.820 (0.7); 7.804 (1.1); 7.800 (1.1); 7.772 (1.0);
7.752 (1.3); 7.721 (2.1); 7.709 (1.2); 7.700 (1.5); 7.691 (0.9); 7.657 (0.8); 7.638 (1.0); 7.619 (0.4); 7.506 (0.9);
7.502 (1.0); 7.483 (1.1); 7.390 (1.1); 7.371 (1.0); 7.335 (1.6); 7.328 (1.0); 7.323 (0.9); 7.311 (2.4); 4.256 (0.4);
4.240 (0.4); 4.220 (0.9); 4.205 (0.9); 4.184 (0.5); 4.169 (0.5); 4.037 (0.4); 4.020 (0.4); 3.953 (12.0); 3.330 (45.8);
2.671 (0.4); 2.667 (0.3); 2.525 (1.1); 2.511 (24.1); 2.507 (50.1); 2.502 (66.8); 2.498 (49.5); 2.494 (25.0); 2.329 (0.4);
1.989 (1.8); 1.398 (16.0); 1.193 (0.5); 1.175 (0.9); 1.157 (0.5); 0.008 (0.3); 0.000 (11.2); −0.008 (0.5)
Example 35: ¹H-NMR (400.0 MHz, d₆-DMSO):
δ = 9.047 (1.2); 9.031 (2.7); 9.015 (1.3); 8.036 (4.4); 8.032 (4.8); 7.903 (2.1); 7.899 (2.0); 7.883 (2.7); 7.879 (2.7);
7.770 (2.5); 7.751 (3.6); 7.741 (5.0); 7.727 (1.4); 7.720 (4.1); 7.710 (2.9); 7.691 (2.1); 7.657 (2.2); 7.638 (2.6);
7.620 (1.5); 7.608 (3.1); 7.603 (4.0); 7.586 (4.1); 7.580 (3.1); 7.569 (0.6); 7.385 (3.0); 7.366 (2.7); 7.356 (0.9);
7.350 (1.3); 7.344 (0.8); 7.332 (1.6); 7.327 (2.5); 7.321 (1.3); 7.309 (0.8); 7.304 (1.3); 7.298 (0.6); 4.261 (1.1);
4.245 (1.1); 4.225 (2.5); 4.209 (2.5); 4.189 (1.3); 4.173 (1.2); 3.332 (39.6); 2.672 (0.4); 2.526 (1.0); 2.512 (22.5);
2.508 (46.2); 2.503 (60.9); 2.499 (44.5); 2.494 (21.9); 2.330 (0.4); 1.990 (0.8); 1.397 (16.0); 1.176 (0.4); 0.008 (0.4);
0.000 (12.3); −0.008 (0.4)
Example 36: ¹H-NMR (400.0 MHz, d₆-DMSO):
δ = 9.050 (2.7); 9.035 (5.5); 9.019 (2.7); 8.318 (0.6); 7.769 (16.0); 7.750 (8.4); 7.742 (10.5); 7.723 (2.6); 7.704 (6.6);
7.687 (8.7); 7.671 (10.7); 7.657 (6.6); 7.649 (7.5); 7.639 (6.0); 7.620 (2.1); 7.465 (2.4); 7.459 (2.6); 7.441 (3.1);
7.436 (4.6); 7.431 (3.2); 7.414 (2.5); 7.408 (2.6); 7.381 (6.2); 7.362 (5.5); 7.269 (2.4); 7.264 (2.3); 7.248 (4.4);
7.243 (4.2); 7.227 (2.2); 7.222 (2.1); 4.269 (2.4); 4.253 (2.6); 4.232 (5.3); 4.217 (5.2); 4.196 (2.8); 4.181 (2.6);
3.331 (107.4); 2.672 (1.8); 2.507 (223.8); 2.503 (279.1); 2.329 (1.8); 1.398 (1.4); 0.000 (8.2)
Example 37: ¹H-NMR (400.0 MHz, d₆-DMSO):
δ = 8.886 (0.7); 8.871 (1.5); 8.855 (0.7); 8.317 (0.5); 7.814 (2.1); 7.809 (1.0); 7.801 (2.4); 7.792 (2.6); 7.784 (1.0);
7.779 (2.3); 7.755 (1.5); 7.736 (2.0); 7.702 (0.7); 7.684 (1.7); 7.666 (1.3); 7.637 (1.2); 7.618 (1.5); 7.599 (0.6);
7.546 (2.2); 7.525 (2.7); 7.368 (3.0); 7.353 (0.3); 7.345 (2.4); 7.340 (1.0); 7.329 (2.5); 7.323 (4.9); 7.313 (3.4);
7.301 (2.8); 4.198 (0.7); 4.183 (0.7); 4.161 (1.5); 4.146 (1.5); 4.125 (0.8); 4.109 (0.7); 3.965 (16.0); 3.891 (0.3);
3.329 (120.9); 2.675 (1.0); 2.671 (1.3); 2.667 (1.0); 2.524 (3.4); 2.506 (156.4); 2.502 (206.0); 2.498 (151.8); 2.333 (1.0);
2.329 (1.4); 2.324 (1.0); 0.000 (7.5)
Example 38: ¹H-NMR (400.0 MHz, d₆-DMSO):
δ = 9.080 (2.5); 9.064 (5.2); 9.048 (2.5); 7.785 (6.0); 7.777 (8.2); 7.763 (12.7); 7.754 (9.6); 7.746 (3.9); 7.741 (10.2);
7.720 (6.0); 7.702 (4.3); 7.667 (4.4); 7.648 (5.5); 7.620 (10.0); 7.591 (7.6); 7.573 (12.6); 7.553 (4.0); 7.390 (6.3);
7.371 (5.7); 7.346 (1.0); 7.338 (8.5); 7.333 (3.0); 7.316 (16.0); 7.298 (2.7); 7.293 (7.8); 7.286 (0.9); 4.102 (2.3);
4.087 (2.4); 4.064 (5.3); 4.049 (5.1); 4.026 (2.7); 4.010 (2.5); 3.334 (31.5); 2.676 (0.4); 2.672 (0.5); 2.668 (0.4);
2.557 (24.9); 2.525 (1.6); 2.507 (61.0); 2.503 (80.1); 2.498 (58.8); 2.334 (0.4); 2.330 (0.5); 0.000 (3.6)
Example 39: 1H-NMR (400.0 MHz, d6-DMSO):
= 9.045 (2.2); 9.029 (4.8); 9.014 (2.3); 8.318 (1.1); 7.941 (8.1); 7.937 (9.1); 7.915 (1.4); 7.907 (13.8); 7.902 (5.1);
7.891 (4.7); 7.885 (16.0); 7.878 (2.0); 7.830 (3.3); 7.826 (3.3); 7.810 (5.6); 7.805 (5.8); 7.768 (4.7); 7.751 (13.8);
7.731 (6.5); 7.707 (5.2); 7.689 (3.8); 7.655 (3.9); 7.636 (4.7); 7.617 (1.8); 7.504 (9.5); 7.484 (8.6); 7.387 (5.4);
7.368 (4.9); 4.262 (1.9); 4.247 (2.1); 4.226 (4.5); 4.211 (4.4); 4.190 (2.4); 4.175 (2.2); 3.330 (195.8); 2.676 (1.3);
2.672 (1.8); 2.667 (1.3); 2.525 (4.3); 2.520 (6.7); 2.511 (97.7); 2.507 (205.8); 2.503 (276.0); 2.498 (203.6); 2.494 (101.9);
2.334 (1.3); 2.329 (1.8); 2.325 (1.3); 1.989 (0.5); 1.398 (1.1); 0.146 (1.0); 0.008 (7.3); 0.000 (224.7); −0.009 (9.3);
−0.150 (0.9)
Example 40: 1H-NMR (400.0 MHz, d6-DMSO):
δ = 9.045 (2.4); 9.029 (5.2); 9.014 (2.4); 8.318 (1.3); 7.961 (9.5); 7.922 (9.5); 7.901 (12.0); 7.859 (3.7); 7.855 (3.5);
7.838 (5.7); 7.835 (5.6); 7.766 (13.1); 7.745 (9.5); 7.726 (2.3); 7.708 (16.0); 7.688 (12.4); 7.655 (4.2); 7.636 (5.0);
7.617 (1.8); 7.389 (5.7); 7.371 (5.1); 7.248 (3.5); 7.109 (8.5); 6.969 (4.0); 4.265 (2.1); 4.250 (2.2); 4.230 (4.8);
4.214 (4.6); 4.194 (2.4); 4.178 (2.3); 4.037 (0.4); 4.020 (0.4); 3.328 (174.7); 2.676 (2.0); 2.671 (2.8); 2.667 (2.1);
2.524 (6.7); 2.507 (327.3); 2.502 (427.4); 2.498 (311.9); 2.333 (2.0); 2.329 (2.7); 2.325 (2.0); 1.989 (1.6); 1.398 (0.8);
1.193 (0.4); 1.175 (0.8); 1.157 (0.4); 0.983 (0.3); 0.146 (1.3); 0.008 (9.8); 0.000 (286.1); −0.008 (11.5); −0.150 (1.3)
Example 41: 1H-NMR (400.0 MHz, d6-DMSO):
δ = 9.050 (2.3); 9.035 (5.0); 9.019 (2.3); 8.318 (0.7); 8.005 (16.0); 7.985 (11.7); 7.888 (3.8); 7.884 (3.8); 7.870 (14.1);
7.850 (9.1); 7.784 (9.5); 7.763 (7.6); 7.749 (6.5); 7.726 (2.0); 7.709 (5.3); 7.690 (3.9); 7.656 (4.0); 7.637 (4.8);
7.618 (1.7); 7.392 (5.5); 7.373 (4.9); 4.271 (2.0); 4.255 (2.1); 4.235 (4.6); 4.219 (4.5); 4.199 (2.3); 4.183 (2.2);
3.330 (103.9); 2.676 (1.1); 2.672 (1.6); 2.667 (1.2); 2.525 (3.8); 2.520 (5.8); 2.511 (86.0); 2.507 (178.1); 2.503 (236.3);
2.498 (172.6); 2.494 (84.9); 2.334 (1.1); 2.329 (1.5); 2.325 (1.1); 1.989 (0.4); 1.398 (1.3); 0.146 (0.8); 0.008 (5.8);
0.000 (187.2); −0.008 (7.3); −0.150 (0.8)
Example 42: 1H-NMR (400.0 MHz, d6-DMSO):
δ = 9.043 (1.2); 9.028 (2.6); 9.012 (1.3); 8.318 (0.4); 7.990 (4.7); 7.869 (2.0); 7.865 (1.9); 7.848 (2.8); 7.844 (2.9);
7.834 (2.5); 7.814 (2.9); 7.779 (4.0); 7.769 (3.1); 7.751 (7.1); 7.730 (3.6); 7.707 (2.8); 7.689 (2.0); 7.664 (2.6);
7.655 (2.4); 7.643 (4.9); 7.637 (2.9); 7.624 (2.7); 7.462 (2.1); 7.442 (1.7); 7.392 (2.9); 7.373 (2.6); 4.263 (1.1);
4.248 (1.1); 4.227 (2.4); 4.212 (2.4); 4.191 (1.3); 4.175 (1.2); 3.329 (82.6); 2.671 (1.1); 2.507 (132.2); 2.502 (172.8);
2.498 (132.2); 2.329 (1.1); 2.325 (0.9); 1.989 (0.9); 1.398 (16.0); 1.175 (0.5); 0.146 (0.5); 0.000 (100.2); −0.008 (6.4);
−0.150 (0.5)
Example 43: 1H-NMR (400.0 MHz, d6-DMSO):
δ = 9.046 (0.8); 9.030 (1.7); 9.014 (0.8); 8.317 (0.6); 7.962 (4.1); 7.952 (4.4); 7.939 (1.2); 7.858 (1.2); 7.854 (1.2);
7.838 (1.9); 7.834 (2.0); 7.766 (4.6); 7.745 (3.5); 7.725 (0.7); 7.707 (1.8); 7.688 (1.6); 7.665 (2.5); 7.655 (5.1);
7.651 (5.4); 7.636 (1.8); 7.617 (0.6); 7.391 (1.9); 7.372 (1.7); 7.242 (1.5); 7.102 (3.3); 6.963 (1.6); 4.266 (0.7);
4.250 (0.7); 4.230 (1.6); 4.215 (1.6); 4.194 (0.8); 4.178 (0.8); 4.038 (0.4); 4.020 (0.4); 3.328 (81.5); 2.676 (0.7);
2.671 (1.0); 2.667 (0.8); 2.525 (2.3); 2.520 (3.8); 2.511 (57.6); 2.507 (121.0); 2.502 (162.7); 2.498 (121.8); 2.494 (62.4);
2.333 (0.8); 2.329 (1.1); 2.325 (0.8); 1.989 (1.5); 1.398 (16.0); 1.193 (0.4); 1.175 (0.8); 1.157 (0.4); 0.146 (0.5);
0.008 (3.8); 0.000 (118.6); −0.008 (5.6); −0.150 (0.5)
Example 44: 1H-NMR (400.0 MHz, d6-DMSO):
δ = 8.882 (0.7); 8.866 (1.6); 8.851 (0.7); 7.922 (3.3); 7.901 (5.7); 7.839 (4.8); 7.818 (2.9); 7.751 (1.5); 7.732 (2.0);
7.700 (0.7); 7.682 (1.6); 7.664 (1.2); 7.635 (1.3); 7.616 (1.5); 7.597 (0.7); 7.589 (2.2); 7.569 (2.7); 7.454 (2.9);
7.410 (1.7); 7.407 (1.6); 7.390 (1.5); 7.386 (1.4); 7.312 (1.7); 7.294 (1.6); 4.210 (0.6); 4.195 (0.7); 4.174 (1.4);
4.158 (1.4); 4.137 (0.7); 4.122 (0.7); 3.980 (16.0); 3.315 (24.6); 2.675 (0.3); 2.670 (0.5); 2.666 (0.4); 2.541 (2.9);
2.524 (1.4); 2.510 (26.1); 2.506 (53.2); 2.501 (71.9); 2.497 (54.6); 2.492 (27.8); 2.333 (0.4); 2.328 (0.5); 2.323 (0.4);
0.146 (0.5); 0.021 (0.4); 0.018 (0.4); 0.008 (4.0); 0.000 (105.6); −0.008 (4.4); −0.150 (0.4)
Example 45: 1H-NMR (400.0 MHz, d6-DMSO):
δ = 9.062 (0.8); 9.046 (1.7); 9.031 (0.8); 8.257 (0.5); 8.233 (16.0); 8.217 (0.4); 8.209 (0.6); 8.103 (2.8); 8.099 (3.0);
7.964 (1.2); 7.960 (1.2); 7.944 (1.7); 7.940 (1.7); 7.826 (2.9); 7.805 (2.2); 7.768 (1.5); 7.749 (2.1); 7.727 (0.7);
7.709 (1.7); 7.690 (1.3); 7.657 (1.3); 7.638 (1.6); 7.619 (0.6); 7.392 (1.8); 7.373 (1.6); 4.280 (0.6); 4.265 (0.7);
4.245 (1.5); 4.229 (1.4); 4.209 (0.8); 4.193 (0.7); 3.333 (45.1); 2.672 (0.3); 2.543 (1.4); 2.526 (0.8); 2.508 (39.1);
2.503 (51.4); 2.499 (37.6); 2.330 (0.3); 2.076 (1.2); 0.146 (0.4); 0.014 (0.3); 0.008 (2.7); 0.000 (73.4); −0.009 (2.8);
−0.150 (0.3)
Example 46: 1H-NMR (400.0 MHz, d6-DMSO):
δ = 9.169 (1.8); 9.153 (3.7); 9.137 (1.8); 8.318 (0.6); 8.041 (5.4); 8.019 (15.8); 7.996 (16.0); 7.974 (5.8); 7.774 (10.3);
7.756 (6.1); 7.747 (8.3); 7.732 (4.8); 7.713 (3.0); 7.669 (3.1); 7.649 (3.9); 7.630 (1.4); 7.581 (0.4); 7.415 (4.5);
7.396 (4.0); 4.164 (1.5); 4.148 (1.7); 4.129 (3.5); 4.113 (3.4); 4.094 (1.8); 4.078 (1.7); 3.329 (96.6); 2.676 (1.2);
2.671 (1.7); 2.667 (1.2); 2.507 (202.9); 2.502 (265.3); 2.498 (199.7); 2.333 (1.3); 2.329 (1.8); 2.325 (1.4); 1.989 (1.0);
1.397 (2.8); 1.235 (0.4); 1.175 (0.5); 0.146 (1.7); 0.024 (0.5); 0.020 (0.4); 0.008 (12.1); 0.000 (335.8); −0.028 (0.5);
−0.040 (0.4); −0.150 (1.7)
Example 47: 1H-NMR (400.0 MHz, d6-DMSO):
= 9.171 (2.6); 9.155 (5.4); 9.139 (2.8); 9.120 (0.8); 9.105 (0.4); 8.319 (0.4); 7.979 (11.6); 7.958 (16.0); 7.850 (14.3);
7.829 (10.6); 7.777 (5.4); 7.757 (8.2); 7.735 (14.8); 7.710 (11.1); 7.669 (5.0); 7.650 (5.6); 7.632 (2.3); 7.417 (6.0);
7.398 (5.5); 7.386 (1.1); 7.368 (0.8); 4.167 (2.2); 4.151 (2.3); 4.132 (4.7); 4.116 (4.7); 4.097 (2.7); 4.081 (2.5);
4.055 (0.8); 4.036 (0.4); 4.020 (0.4); 3.568 (0.4); 3.331 (110.5); 2.672 (1.4); 2.507 (175.6); 2.503 (219.9); 2.499 (162.5);
2.329 (1.4); 1.397 (1.8); 0.146 (1.4); 0.029 (0.6); 0.024 (0.6); 0.000 (272.0); −0.150 (1.4)
Example 48: 1H-NMR (400.0 MHz, d6-DMSO):
δ = 9.164 (2.4); 9.149 (5.0); 9.133 (2.5); 8.318 (1.1); 7.963 (1.5); 7.955 (14.1); 7.950 (5.1); 7.938 (4.9); 7.933 (16.0);
7.926 (1.8); 7.778 (5.0); 7.758 (7.2); 7.733 (6.0); 7.714 (4.2); 7.686 (9.7); 7.669 (5.1); 7.658 (10.3); 7.631 (2.1);
7.509 (9.9); 7.489 (9.0); 7.416 (5.8); 7.397 (5.2); 7.366 (0.4); 4.161 (2.0); 4.146 (2.1); 4.126 (4.6); 4.110 (4.5);
4.090 (2.5); 4.074 (2.3); 4.054 (0.5); 3.568 (0.4); 3.357 (0.4); 3.329 (180.0); 2.676 (2.0); 2.671 (2.8); 2.667 (2.1);
2.569 (0.5); 2.525 (7.3); 2.520 (11.4); 2.511 (155.0); 2.507 (321.4); 2.502 (426.8); 2.498 (313.1); 2.493 (153.1);
2.459 (0.5); 2.333 (2.0); 2.329 (2.7); 2.324 (2.0); 1.398 (1.1); 0.146 (2.9); 0.034 (0.5); 0.028 (1.0); 0.008 (22.7);
0.000 (653.9); −0.009 (26.0); −0.019 (1.1); −0.022 (1.0); −0.024 (1.0); −0.048 (0.3); −0.150 (2.9)
Example 49: 1H-NMR (400.0 MHz, d6-DMSO):
δ = 9.088 (1.3); 9.073 (2.7); 9.057 (1.3); 7.972 (2.1); 7.966 (1.2); 7.950 (16.0); 7.943 (15.3); 7.927 (1.0); 7.921 (2.2);
7.784 (2.5); 7.765 (3.4); 7.736 (5.6); 7.720 (4.7); 7.701 (4.7); 7.666 (2.2); 7.647 (2.6); 7.625 (5.3); 7.605 (3.2);
7.387 (3.0); 7.368 (2.7); 5.758 (9.2); 4.113 (1.1); 4.097 (1.1); 4.075 (2.5); 4.059 (2.4); 4.038 (2.7); 4.020 (2.7);
4.003 (0.6); 3.569 (3.5); 3.332 (24.1); 2.891 (0.5); 2.732 (0.4); 2.672 (0.4); 2.576 (11.8); 2.525 (1.1); 2.512 (22.6);
2.508 (45.7); 2.503 (59.8); 2.499 (43.7); 2.494 (21.5); 2.330 (0.4); 1.990 (6.9); 1.397 (1.5); 1.193 (1.8); 1.175 (3.6);
1.158 (1.8); 0.146 (0.4); 0.008 (3.5); 0.000 (96.6); −0.008 (4.3); −0.150 (0.4)
Example 50: 1H-NMR (400.0 MHz, d6-DMSO):
δ = 9.088 (1.7); 9.073 (3.6); 9.057 (1.7); 9.032 (0.4); 7.893 (6.8); 7.888 (3.1); 7.872 (14.6); 7.833 (12.0); 7.812 (6.0);
7.784 (3.8); 7.765 (5.0); 7.738 (1.6); 7.720 (4.6); 7.706 (7.0); 7.670 (4.8); 7.647 (3.9); 7.628 (1.7); 7.618 (6.6);
7.597 (4.0); 7.573 (0.6); 7.512 (0.4); 7.428 (0.7); 7.407 (0.5); 7.387 (4.1); 7.368 (3.9); 7.348 (0.4); 5.758 (3.4);
4.115 (1.5); 4.099 (1.5); 4.077 (3.3); 4.061 (3.4); 4.038 (2.8); 4.021 (2.4); 4.008 (0.5); 4.003 (0.5); 3.569 (2.2);
3.333 (68.6); 2.732 (0.3); 2.676 (0.5); 2.672 (0.6); 2.667 (0.5); 2.628 (0.3); 2.574 (16.0); 2.525 (1.6); 2.512 (36.0);
2.507 (73.9); 2.503 (97.5); 2.498 (71.8); 2.494 (35.6); 2.480 (2.2); 2.334 (0.5); 2.330 (0.6); 2.325 (0.5); 1.989 (4.6);
1.397 (4.2); 1.193 (1.2); 1.175 (2.4); 1.157 (1.2); 0.146 (0.7); 0.008 (5.7); 0.000 (156.8); −0.008 (6.9); −0.150 (0.7)
Example 51: 1H-NMR (400.0 MHz, d6-DMSO):
δ = 8.895 (0.6); 8.879 (1.2); 8.863 (0.6); 7.970 (16.0); 7.753 (1.2); 7.733 (1.5); 7.701 (0.5); 7.682 (1.3); 7.664 (0.9);
7.636 (1.0); 7.617 (1.2); 7.597 (2.0); 7.576 (2.0); 7.471 (2.3); 7.439 (1.4); 7.419 (1.1); 7.312 (1.3); 7.294 (1.2);
4.207 (0.5); 4.191 (0.5); 4.170 (1.1); 4.155 (1.1); 4.134 (0.6); 4.118 (0.5); 3.983 (11.6); 3.331 (70.9); 3.314 (0.4);
2.671 (0.5); 2.524 (1.0); 2.506 (54.3); 2.502 (70.8); 2.498 (52.9); 2.329 (0.5); 2.325 (0.4); 0.146 (0.4); 0.007 (3.1);
0.000 (79.9); −0.008 (4.0); −0.019 (0.4); −0.150 (0.4)
Example 57: 1H-NMR (400.0 MHz, d6-DMSO):
δ = 8.907 (2.3); 8.891 (5.0); 8.876 (2.4); 7.887 (9.9); 7.858 (7.1); 7.836 (10.2); 7.830 (3.5); 7.822 (8.5); 7.813 (9.1);
7.805 (3.5); 7.800 (8.3); 7.792 (3.4); 7.788 (2.6); 7.771 (7.5); 7.767 (7.7); 7.751 (12.9); 7.730 (4.4); 7.438 (3.0);
7.436 (3.2); 7.420 (6.5); 7.417 (7.0); 7.401 (4.1); 7.398 (4.2); 7.357 (8.3); 7.352 (2.8); 7.335 (16.0); 7.318 (2.6);
7.313 (7.7); 7.185 (4.8); 7.181 (7.6); 7.172 (5.0); 7.166 (5.6); 7.162 (6.5); 7.153 (6.9); 7.149 (5.1); 7.134 (3.3);
7.130 (2.8); 4.242 (2.2); 4.227 (2.2); 4.206 (4.9); 4.191 (4.7); 4.171 (2.5); 4.155 (2.3); 3.669 (0.3); 3.659 (0.4);
3.648 (0.4); 3.640 (0.4); 3.591 (0.4); 3.570 (0.3); 3.545 (0.7); 3.508 (0.7); 3.494 (0.4); 3.483 (0.4); 3.468 (1.0);
3.459 (1.1); 3.447 (1.0); 3.439 (0.9); 3.417 (3.2); 3.401 (3.3); 3.397 (4.1); 3.390 (4.1); 3.384 (5.8); 3.379 (5.6);
3.347 (2869.6); 3.314 (7.4); 3.308 (4.4); 3.302 (3.9); 3.287 (1.9); 3.260 (1.3); 3.239 (1.0); 3.231 (0.8); 3.214 (1.2);
3.195 (0.8); 3.163 (0.7); 3.133 (0.4); 3.115 (0.4); 3.088 (0.4); 2.996 (0.7); 2.676 (4.4); 2.672 (6.3); 2.667 (4.7);
2.618 (0.4); 2.603 (0.4); 2.562 (1.2); 2.542 (32.5); 2.535 (3.3); 2.525 (15.1); 2.520 (22.9); 2.512 (358.4); 2.507 (751.2);
2.503 (1001.1); 2.498 (734.3); 2.494 (359.9); 2.468 (2.8); 2.463 (1.8); 2.452 (0.6); 2.447 (0.5); 2.442 (0.4); 2.417 (0.5);
2.405 (0.4); 2.370 (0.6); 2.334 (4.5); 2.330 (6.3); 2.325 (4.9); 2.291 (0.7); 1.434 (0.3); 1.258 (0.5); 1.234 (1.0);
0.146 (0.4); 0.008 (2.5); 0.000 (83.7); −0.009 (3.0); −0.150 (0.4)
Example 58: 1H-NMR (400.0 MHz, d6-DMSO):
δ = 9.235 (0.3); 8.955 (2.1); 8.940 (4.0); 8.924 (1.9); 7.892 (7.9); 7.889 (7.3); 7.834 (6.5); 7.828 (3.4); 7.820 (7.5);
7.816 (5.0); 7.812 (7.3); 7.803 (3.9); 7.798 (6.6); 7.789 (3.6); 7.785 (2.7); 7.769 (6.2); 7.765 (5.5); 7.735 (9.1);
7.714 (4.2); 7.685 (0.7); 7.664 (0.5); 7.625 (5.2); 7.622 (4.7); 7.605 (6.0); 7.602 (5.2); 7.501 (0.4); 7.428 (2.0);
7.425 (1.9); 7.410 (5.4); 7.407 (4.8); 7.391 (4.3); 7.388 (3.5); 7.360 (10.6); 7.355 (6.2); 7.338 (16.0); 7.321 (5.3);
7.316 (7.8); 7.308 (0.7); 7.251 (5.6); 7.246 (4.7); 7.232 (4.6); 7.228 (3.6); 7.158 (0.6); 7.139 (0.8); 7.118 (0.5);
4.254 (1.9); 4.239 (2.2); 4.219 (4.2); 4.203 (4.0); 4.183 (2.1); 4.167 (1.9); 3.382 (0.4); 3.340 (59.3); 3.335 (169.8);
3.311 (0.6); 2.676 (0.8); 2.671 (0.9); 2.667 (0.7); 2.546 (7.4); 2.542 (29.9); 2.511 (66.8); 2.507 (115.2); 2.503 (133.7);
2.498 (91.0); 2.494 (40.8); 2.334 (0.6); 2.329 (0.8); 2.325 (0.5); 0.004 (3.2); 0.000 (13.0); −0.008 (0.5)
Example 59: 1H-NMR (400.0 MHz, d6-DMSO):
δ = 8.967 (2.4); 8.952 (4.9); 8.936 (2.3); 7.893 (8.9); 7.890 (9.4); 7.843 (1.1); 7.835 (7.2); 7.830 (3.6); 7.821 (8.4);
7.813 (8.7); 7.804 (3.8); 7.799 (7.8); 7.786 (3.8); 7.783 (3.5); 7.766 (6.6); 7.762 (6.5); 7.721 (11.1); 7.700 (6.0);
7.686 (0.6); 7.665 (0.3); 7.470 (2.3); 7.466 (3.2); 7.450 (8.3); 7.446 (9.5); 7.443 (6.1); 7.439 (4.8); 7.426 (6.1);
7.421 (6.0); 7.406 (2.7); 7.401 (2.8); 7.393 (0.5); 7.385 (3.7); 7.380 (3.4); 7.366 (7.0); 7.361 (12.9); 7.348 (4.6);
7.344 (6.7); 7.339 (16.0); 7.322 (3.0); 7.317 (7.7); 7.309 (1.0); 7.287 (6.7); 7.284 (6.4); 7.269 (4.9); 7.265 (4.3);
7.159 (0.4); 7.139 (0.5); 7.119 (0.3); 4.259 (2.3); 4.243 (2.5); 4.223 (5.1); 4.208 (4.9); 4.188 (2.6); 4.172 (2.4);
3.364 (0.6); 3.338 (178.2); 3.315 (0.6); 3.309 (0.4); 3.298 (0.5); 2.998 (0.7); 2.676 (0.5); 2.672 (0.7); 2.667 (0.5);
2.542 (17.1); 2.525 (2.0); 2.512 (41.9); 2.507 (84.7); 2.503 (110.1); 2.498 (80.4); 2.494 (39.4); 2.468 (0.6); 2.463 (0.5);
2.458 (0.5); 2.454 (0.4); 2.334 (0.5); 2.330 (0.7); 2.325 (0.5); 0.008 (0.3); 0.000 (9.7); −0.009 (0.4)
Example 60: 1H-NMR (400.0 MHz, d6-DMSO):
δ = 9.250 (2.4); 9.235 (5.1); 9.219 (2.4); 7.902 (9.0); 7.899 (9.7); 7.853 (0.8); 7.845 (7.4); 7.840 (3.4); 7.831 (8.3);
7.823 (9.0); 7.814 (3.5); 7.809 (8.1); 7.801 (1.0); 7.786 (4.0); 7.783 (3.8); 7.766 (5.9); 7.762 (5.8); 7.684 (10.0);
7.664 (6.9); 7.538 (1.2); 7.521 (2.7); 7.517 (2.5); 7.500 (4.9); 7.484 (2.5); 7.479 (3.0); 7.463 (1.3); 7.369 (0.9);
7.361 (8.4); 7.356 (2.9); 7.339 (16.0); 7.322 (2.5); 7.317 (7.8); 7.165 (1.5); 7.158 (8.5); 7.139 (11.5); 7.118 (7.1);
7.111 (1.4); 4.276 (2.2); 4.261 (2.3); 4.241 (5.1); 4.225 (5.0); 4.205 (2.6); 4.189 (2.4); 3.375 (0.5); 3.358 (0.9);
3.335 (369.1); 3.301 (0.4); 2.676 (1.1); 2.671 (1.6); 2.667 (1.2); 2.542 (3.7); 2.525 (3.7); 2.520 (5.8); 2.511 (88.9);
2.507 (186.5); 2.502 (248.6); 2.498 (182.7); 2.493 (89.9); 2.334 (1.1); 2.329 (1.6); 2.325 (1.2); 1.234 (0.4); 0.008 (0.8);
0.000 (27.3); −0.009 (1.0)
Example 61: 1H-NMR (400.0 MHz, d6-DMSO):
δ = 9.345 (3.0); 9.329 (6.3); 9.314 (3.1); 9.025 (9.7); 9.019 (12.0); 8.967 (11.6); 8.961 (10.4); 7.913 (12.7); 7.840 (7.7);
7.826 (9.0); 7.819 (10.2); 7.805 (8.8); 7.785 (4.8); 7.764 (7.9); 7.710 (12.2); 7.689 (7.3); 7.365 (8.3); 7.343 (16.0);
7.322 (8.0); 4.324 (2.9); 4.308 (3.0); 4.286 (6.5); 4.271 (6.4); 4.249 (3.4); 4.234 (3.1); 3.375 (0.3); 3.340 (272.1);
3.309 (0.6); 2.673 (1.1); 2.543 (14.2); 2.504 (169.7); 2.331 (1.1); 0.000 (13.0)
Example 62: 1H-NMR (400.0 MHz, d6-DMSO):
δ = 9.165 (2.5); 9.149 (5.3); 9.133 (2.5); 8.865 (5.6); 8.855 (5.6); 8.853 (5.6); 8.302 (5.2); 8.300 (5.4); 8.282 (5.6);
8.280 (5.6); 7.902 (8.9); 7.899 (9.9); 7.841 (0.9); 7.834 (7.5); 7.828 (3.7); 7.820 (8.4); 7.811 (9.3); 7.803 (3.7);
7.798 (8.2); 7.790 (1.2); 7.771 (3.7); 7.767 (3.8); 7.756 (4.8); 7.751 (7.2); 7.746 (9.6); 7.737 (4.2); 7.724 (3.5);
7.696 (11.1); 7.675 (6.5); 7.368 (0.9); 7.361 (8.4); 7.356 (3.2); 7.339 (16.0); 7.322 (2.6); 7.316 (7.8); 4.284 (2.3);
4.268 (2.4); 4.247 (5.3); 4.231 (5.1); 4.210 (2.7); 4.195 (2.5); 3.362 (0.6); 3.358 (0.6); 3.355 (0.7); 3.336 (169.6);
3.321 (0.9); 3.316 (0.3); 2.677 (0.6); 2.672 (0.8); 2.668 (0.6); 2.543 (6.7); 2.526 (2.3); 2.521 (3.6); 2.512 (45.0);
2.508 (94.4); 2.503 (126.4); 2.499 (93.6); 2.494 (46.7); 2.467 (0.3); 2.451 (0.3); 2.447 (0.3); 2.335 (0.6); 2.330 (0.8);
2.326 (0.6); 0.008 (0.5); 0.000 (16.2); −0.008 (0.8)
Example 63: 1H-NMR (400.0 MHz, d6-DMSO):
δ = 9.143 (2.6); 9.128 (5.4); 9.112 (2.6); 8.464 (6.6); 8.459 (7.2); 8.452 (7.1); 8.447 (7.2); 7.899 (10.4); 7.846 (0.8);
7.838 (7.4); 7.833 (3.7); 7.825 (8.5); 7.816 (9.2); 7.808 (3.8); 7.803 (8.5); 7.793 (4.0); 7.789 (3.7); 7.772 (6.9);
7.768 (7.2); 7.761 (7.1); 7.756 (7.0); 7.742 (8.0); 7.737 (8.1); 7.730 (12.3); 7.709 (6.3); 7.696 (0.4); 7.491 (7.2);
7.479 (6.9); 7.472 (6.5); 7.460 (6.3); 7.372 (0.9); 7.364 (8.4); 7.342 (16.0); 7.325 (2.7); 7.320 (7.8); 4.280 (2.3);
4.265 (2.4); 4.244 (5.4); 4.229 (5.3); 4.209 (2.8); 4.193 (2.6); 3.338 (175.5); 3.316 (0.9); 3.304 (0.4); 2.998 (0.3);
2.677 (0.5); 2.673 (0.7); 2.668 (0.6); 2.543 (35.2); 2.526 (1.4); 2.512 (41.7); 2.508 (87.5); 2.503 (116.4); 2.499 (86.5);
2.495 (43.5); 2.465 (0.4); 2.335 (0.5); 2.330 (0.7); 2.326 (0.6); 0.008 (0.3); 0.000 (11.5)
Example 64: 1H-NMR (400.0 MHz, d6-DMSO):
δ = 9.093 (2.6); 9.078 (5.5); 9.062 (2.6); 8.371 (6.3); 8.366 (7.2); 8.359 (6.9); 8.354 (7.0); 7.896 (10.9); 7.845 (0.9);
7.838 (7.4); 7.832 (3.9); 7.824 (8.5); 7.815 (9.3); 7.807 (3.9); 7.802 (8.5); 7.796 (3.4); 7.775 (8.5); 7.771 (9.5);
7.761 (14.4); 7.740 (3.6); 7.511 (3.7); 7.505 (4.4); 7.492 (9.5); 7.487 (9.2); 7.468 (9.0); 7.457 (8.4); 7.449 (4.1);
7.438 (4.1); 7.367 (0.9); 7.359 (8.4); 7.355 (3.3); 7.337 (16.0); 7.315 (7.9); 4.261 (2.4); 4.245 (2.5); 4.225 (5.3);
4.210 (5.1); 4.189 (2.7); 4.174 (2.5); 3.370 (0.5); 3.336 (247.6); 3.312 (0.9); 2.997 (1.1); 2.712 (0.9); 2.676 (0.8);
2.672 (1.1); 2.667 (0.8); 2.576 (0.5); 2.562 (0.7); 2.557 (1.1); 2.542 (225.5); 2.525 (2.7); 2.511 (59.6); 2.507 (122.6);
2.503 (162.8); 2.498 (121.1); 2.494 (60.6); 2.368 (1.0); 2.334 (0.7); 2.330 (1.0); 2.325 (0.8); 1.234 (0.4); 0.008 (0.5);
0.000 (14.6)
Example 65: 1H-NMR (400.0 MHz, d6-DMSO):
δ = 9.033 (2.2); 9.017 (4.7); 9.002 (2.2); 8.314 (0.5); 7.964 (8.2); 7.961 (8.9); 7.847 (3.6); 7.843 (3.5); 7.826 (5.4);
7.822 (5.4); 7.767 (4.6); 7.748 (15.5); 7.728 (7.7); 7.706 (5.1); 7.688 (3.8); 7.654 (7.7); 7.635 (10.1); 7.617 (1.8);
7.577 (6.0); 7.570 (5.8); 7.565 (8.5); 7.558 (16.0); 7.537 (4.5); 7.389 (5.4); 7.373 (14.7); 7.261 (4.0); 7.256 (3.7);
7.241 (3.5); 7.236 (3.4); 7.188 (5.8); 4.262 (2.0); 4.247 (2.1); 4.227 (4.6); 4.211 (4.5); 4.190 (2.4); 4.175 (2.2);
4.038 (0.5); 4.021 (0.5); 3.318 (83.4); 2.680 (0.4); 2.675 (0.9); 2.671 (1.3); 2.666 (1.0); 2.662 (0.5); 2.524 (3.4);
2.519 (5.4); 2.511 (70.0); 2.506 (142.2); 2.502 (188.1); 2.497 (139.5); 2.493 (69.6); 2.337 (0.4); 2.333 (0.9); 2.328 (1.2);
2.324 (0.9); 1.988 (1.9); 1.398 (8.1); 1.193 (0.5); 1.175 (1.1); 1.158 (0.5); 0.146 (0.6); 0.008 (5.3); 0.000 (153.4);
−0.008 (6.2); −0.150 (0.7)
Example 66: 1H-NMR (400.0 MHz, d6-DMSO):
δ = 9.037 (2.8); 9.022 (6.1); 9.006 (2.9); 8.314 (1.7); 8.053 (10.2); 8.050 (10.9); 7.911 (4.8); 7.907 (4.7); 7.891 (6.2);
7.886 (6.1); 7.857 (1.6); 7.850 (3.0); 7.842 (14.8); 7.819 (14.3); 7.812 (3.3); 7.804 (1.4); 7.768 (5.7); 7.746 (16.0);
7.725 (11.1); 7.707 (6.4); 7.689 (4.7); 7.656 (4.9); 7.637 (5.8); 7.619 (2.1); 7.482 (4.5); 7.382 (6.7); 7.363 (6.0);
7.302 (9.6); 7.121 (4.9); 4.260 (2.4); 4.245 (2.5); 4.224 (5.5); 4.209 (5.4); 4.188 (2.9); 4.173 (2.7); 3.354 (0.5);
3.321 (428.2); 3.301 (1.0); 3.297 (1.0); 2.680 (0.6); 2.675 (1.4); 2.671 (1.9); 2.666 (1.4); 2.662 (0.7); 2.524 (5.1);
2.520 (7.7); 2.511 (106.7); 2.506 (220.7); 2.502 (295.1); 2.497 (218.1); 2.493 (107.7); 2.338 (0.7); 2.333 (1.4); 2.329 (2.0);
2.324 (1.5); 2.319 (0.7); 1.988 (0.5); 1.398 (5.7); 0.146 (1.0); 0.008 (7.7); 0.000 (245.5); −0.008 (9.8); −0.020 (0.3);
−0.150 (1.0)
Example 67: 1H-NMR (400.0 MHz, d6-DMSO):
δ = 9.041 (2.2); 9.026 (4.6); 9.010 (2.2); 8.091 (10.3); 8.076 (5.0); 8.045 (8.1); 8.041 (8.5); 7.907 (3.8); 7.903 (3.6);
7.887 (5.1); 7.883 (5.0); 7.819 (3.1); 7.799 (5.7); 7.766 (16.0); 7.746 (15.5); 7.725 (3.8); 7.708 (5.1); 7.690 (3.8);
7.656 (3.8); 7.637 (4.6); 7.618 (1.7); 7.398 (5.3); 7.379 (4.7); 5.755 (2.0); 4.272 (1.9); 4.256 (2.1); 4.236 (4.5);
4.220 (4.4); 4.200 (2.3); 4.184 (2.2); 3.322 (51.7); 2.677 (0.4); 2.672 (0.6); 2.668 (0.4); 2.526 (1.5); 2.512 (33.3);
2.508 (66.4); 2.503 (86.6); 2.499 (64.0); 2.494 (31.8); 2.334 (0.4); 2.330 (0.6); 2.325 (0.4); 1.397 (0.4); 1.259 (0.5);
1.250 (0.4); 1.233 (0.8); 0.146 (0.3); 0.008 (2.8); 0.000 (75.8); −0.009 (3.0); −0.150 (0.3)
Example 68: 1H-NMR (400.0 MHz, d6-DMSO):
δ = 9.027 (2.7); 9.012 (5.7); 8.996 (2.8); 8.974 (0.4); 8.313 (1.2); 7.901 (10.9); 7.844 (14.5); 7.822 (16.0); 7.804 (4.1);
7.801 (3.9); 7.784 (6.8); 7.780 (6.8); 7.766 (5.7); 7.747 (7.8); 7.731 (11.2); 7.710 (8.1); 7.687 (4.6); 7.654 (4.8);
7.635 (5.7); 7.616 (2.1); 7.586 (0.7); 7.564 (0.5); 7.513 (5.3); 7.385 (6.4); 7.366 (5.9); 7.344 (0.5); 7.329 (11.2);
7.309 (14.2); 7.287 (13.3); 7.144 (5.5); 6.548 (0.3); 4.257 (2.3); 4.241 (2.5); 4.220 (5.3); 4.205 (5.2); 4.185 (2.8);
4.169 (2.6); 3.383 (0.4); 3.319 (580.0); 3.295 (1.4); 3.276 (0.6); 3.270 (0.5); 2.675 (2.4); 2.671 (3.2); 2.666 (2.4);
2.566 (0.5); 2.506 (378.5); 2.501 (484.5); 2.497 (362.7); 2.333 (2.4); 2.328 (3.2); 2.324 (2.4); 1.988 (0.4); 1.398 (5.8);
1.236 (0.4); 0.146 (1.7); 0.000 (350.4); −0.150 (1.7)
Example 69: 1H-NMR (400.0 MHz, d6-DMSO):
δ = 9.016 (0.6); 7.970 (1.1); 7.966 (1.2); 7.850 (1.7); 7.832 (0.7); 7.828 (0.7); 7.767 (0.6); 7.747 (1.6); 7.739 (1.4);
7.735 (0.8); 7.730 (1.0); 7.725 (0.8); 7.718 (1.0); 7.707 (0.8); 7.689 (0.5); 7.654 (0.5); 7.636 (0.7); 7.553 (0.3);
7.533 (1.0); 7.516 (1.4); 7.515 (1.4); 7.513 (1.6); 7.508 (0.8); 7.387 (0.7); 7.368 (0.7); 4.224 (0.6); 4.208 (0.6);
4.187 (0.3); 4.172 (0.3); 3.316 (20.9); 2.524 (1.0); 2.510 (17.2); 2.506 (33.7); 2.501 (43.8); 2.497 (32.2); 2.492 (16.1);
1.988 (0.4); 1.398 (16.0); 0.008 (1.5); 0.000 (36.9); −0.009 (1.6)
Example 70: 1H-NMR (400.0 MHz, d6-DMSO):
δ = 9.030 (1.3); 9.014 (2.8); 8.999 (1.3); 8.313 (0.3); 8.087 (5.7); 8.082 (5.8); 8.013 (4.8); 8.009 (5.1); 7.875 (2.2);
7.872 (2.1); 7.855 (3.0); 7.851 (3.0); 7.801 (1.5); 7.796 (1.3); 7.780 (4.9); 7.775 (5.4); 7.763 (10.0); 7.741 (7.4);
7.720 (4.4); 7.706 (3.0); 7.687 (2.2); 7.655 (2.2); 7.636 (2.7); 7.617 (1.0); 7.386 (3.1); 7.367 (2.8); 4.257 (1.2);
4.241 (1.2); 4.221 (2.6); 4.205 (2.6); 4.184 (1.4); 4.169 (1.3); 3.316 (43.9); 2.675 (0.6); 2.671 (0.8); 2.666 (0.6);
2.541 (6.6); 2.524 (2.1); 2.510 (46.9); 2.506 (93.1); 2.501 (123.5); 2.497 (93.5); 2.493 (48.1); 2.333 (0.6); 2.328 (0.8);
2.324 (0.6); 2.073 (16.0); 0.008 (1.2); 0.000 (33.7); −0.008 (1.5)
Example 71: 1H-NMR (400.0 MHz, d6-DMSO):
δ = 9.032 (3.6); 9.017 (7.4); 9.001 (3.8); 8.974 (0.8); 8.960 (0.4); 8.314 (0.8); 7.933 (14.5); 7.893 (13.9); 7.890 (16.0);
7.814 (5.7); 7.794 (9.1); 7.767 (8.4); 7.747 (10.8); 7.730 (13.5); 7.708 (14.0); 7.687 (6.5); 7.655 (6.9); 7.637 (14.2);
7.620 (10.8); 7.616 (12.7); 7.585 (1.4); 7.564 (1.1); 7.543 (14.2); 7.522 (9.5); 7.508 (1.5); 7.384 (8.4); 7.366 (8.4);
7.347 (1.0); 4.255 (3.2); 4.240 (3.5); 4.220 (7.1); 4.204 (7.2); 4.183 (4.0); 4.168 (3.9); 4.138 (0.6); 4.124 (0.4);
4.039 (0.7); 4.021 (0.7); 3.378 (0.4); 3.320 (273.0); 2.671 (2.4); 2.506 (261.0); 2.502 (349.7); 2.498 (299.8); 2.329 (2.2);
1.989 (2.6); 1.398 (4.6); 1.298 (0.6); 1.259 (0.9); 1.232 (2.1); 1.193 (0.9); 1.176 (1.6); 1.158 (0.9); 0.868 (0.4);
0.852 (0.4); 0.000 (25.4)
Example 72: 1H-NMR (400.0 MHz, d6-DMSO):
δ = 9.124 (2.6); 9.108 (5.5); 9.093 (2.6); 8.410 (9.3); 8.406 (9.5); 8.314 (0.5); 8.193 (4.4); 8.189 (4.3); 8.172 (5.0);
8.168 (5.1); 7.965 (14.2); 7.943 (16.0); 7.881 (8.4); 7.860 (7.5); 7.761 (5.1); 7.741 (7.3); 7.727 (2.4); 7.709 (5.9);
7.690 (4.3); 7.656 (4.4); 7.637 (5.3); 7.618 (1.9); 7.528 (10.6); 7.507 (9.7); 7.416 (6.2); 7.398 (5.5); 4.255 (2.2);
4.239 (2.4); 4.220 (5.0); 4.205 (4.9); 4.186 (2.6); 4.170 (2.4); 3.318 (86.7); 2.675 (0.9); 2.671 (1.2); 2.667 (0.9);
2.506 (137.7); 2.502 (180.6); 2.498 (136.6); 2.333 (0.9); 2.329 (1.2); 2.324 (0.9); 1.989 (0.5); 1.398 (0.5); 0.008 (0.6);
0.000 (16.3)
Example 73: 1H-NMR (400.0 MHz, d6-DMSO):
δ = 9.128 (1.7); 9.112 (3.6); 9.096 (1.7); 8.491 (5.9); 8.487 (6.2); 8.259 (2.8); 8.255 (2.8); 8.239 (3.2); 8.234 (3.3);
8.053 (5.2); 8.048 (2.5); 8.037 (3.5); 8.031 (16.0); 8.010 (15.9); 8.005 (3.9); 7.993 (2.3); 7.988 (5.3); 7.909 (5.8);
7.888 (5.2); 7.760 (3.3); 7.740 (4.6); 7.726 (1.5); 7.709 (3.7); 7.690 (2.8); 7.656 (2.8); 7.637 (3.4); 7.618 (1.2);
7.419 (4.0); 7.400 (3.6); 4.259 (1.4); 4.243 (1.5); 4.224 (3.3); 4.209 (3.2); 4.190 (1.7); 4.174 (1.6); 4.039 (0.5);
4.021 (0.5); 3.320 (63.0); 2.676 (0.5); 2.671 (0.7); 2.667 (0.5); 2.525 (1.5); 2.520 (2.2); 2.511 (34.4); 2.507 (72.0);
2.502 (96.7); 2.498 (71.7); 2.493 (35.8); 2.334 (0.4); 2.329 (0.6); 2.324 (0.5); 1.989 (2.3); 1.398 (2.2); 1.193 (0.6);
1.176 (1.3); 1.158 (0.6); 0.000 (11.1); −0.008 (0.4)
Example 74: 1H-NMR (400.0 MHz, d6-DMSO):
δ = 9.118 (3.3); 9.103 (6.5); 9.087 (3.3); 8.367 (12.0); 8.314 (0.7); 8.159 (5.7); 8.138 (6.5); 7.898 (8.5); 7.884 (10.4);
7.876 (10.4); 7.862 (10.3); 7.855 (10.8); 7.834 (8.3); 7.761 (6.3); 7.742 (8.8); 7.728 (3.2); 7.709 (7.3); 7.691 (5.4);
7.656 (5.4); 7.637 (6.5); 7.618 (2.4); 7.416 (7.8); 7.397 (7.2); 7.382 (8.9); 7.360 (16.0); 7.338 (7.6); 4.247 (3.0);
4.231 (3.3); 4.213 (6.5); 4.197 (6.3); 4.178 (3.5); 4.163 (3.1); 3.320 (164.5); 2.671 (1.4); 2.502 (211.0); 2.329 (1.4);
1.988 (0.6); 1.398 (0.7); 1.249 (0.4); 1.235 (1.0); 1.175 (0.4); 1.133 (0.3); 0.000 (16.1)
Example 75: 1H-NMR (400.0 MHz, d6-DMSO):
δ = 9.014 (1.9); 8.999 (4.0); 8.983 (1.9); 8.313 (0.4); 7.813 (7.2); 7.766 (3.6); 7.745 (6.3); 7.722 (6.0); 7.719 (5.6);
7.703 (4.1); 7.685 (3.2); 7.667 (16.0); 7.645 (13.2); 7.634 (4.4); 7.614 (1.4); 7.383 (4.3); 7.365 (3.9); 7.053 (9.1);
7.030 (8.7); 4.239 (1.5); 4.223 (1.6); 4.203 (3.5); 4.187 (3.4); 4.167 (1.8); 4.151 (1.6); 3.924 (1.4); 3.764 (9.8);
3.752 (12.9); 3.740 (10.6); 3.320 (126.5); 3.292 (0.3); 3.202 (10.3); 3.190 (12.6); 3.178 (9.3); 2.675 (0.6); 2.670 (0.8);
2.666 (0.6); 2.510 (50.9); 2.506 (98.4); 2.501 (127.5); 2.497 (94.5); 2.493 (47.8); 2.333 (0.6); 2.328 (0.8); 2.324 (0.6);
1.988 (0.4); 1.070 (8.7); 0.146 (0.6); 0.008 (6.6); 0.000 (134.5); −0.009 (6.4); −0.150 (0.6)
Example 76: 1H-NMR (400.0 MHz, d6-DMSO):
δ = 9.070 (1.7); 9.055 (3.8); 9.039 (1.8); 7.857 (1.1); 7.849 (10.6); 7.844 (3.8); 7.833 (3.7); 7.827 (12.3); 7.820 (1.5);
7.783 (3.6); 7.763 (4.8); 7.736 (1.5); 7.718 (4.0); 7.700 (2.9); 7.664 (3.4); 7.663 (3.4); 7.654 (7.0); 7.647 (6.7);
7.625 (5.7); 7.598 (7.8); 7.577 (3.4); 7.483 (7.3); 7.463 (6.6); 7.388 (4.2); 7.370 (3.8); 4.111 (1.5); 4.095 (1.5);
4.073 (3.5); 4.057 (3.4); 4.035 (1.7); 4.019 (1.6); 3.320 (29.5); 2.672 (0.4); 2.567 (16.0); 2.542 (38.2); 2.525 (0.9);
2.520 (1.0); 2.511 (16.6); 2.507 (35.2); 2.502 (48.4); 2.498 (37.0); 2.493 (19.0); 2.329 (0.3); 0.146 (0.4); 0.008 (3.0);
0.000 (89.3); −0.009 (4.1); −0.017 (0.5); −0.150 (0.4)
Example 77: 1H-NMR (400.0 MHz, d6-DMSO):
δ = 8.879 (0.8); 8.863 (1.7); 8.847 (0.8); 7.889 (0.5); 7.882 (4.3); 7.877 (1.6); 7.865 (1.6); 7.860 (5.1); 7.853 (0.6);
7.752 (1.5); 7.733 (2.1); 7.701 (0.7); 7.683 (1.7); 7.664 (1.3); 7.636 (1.3); 7.617 (1.6); 7.598 (0.6); 7.570 (2.3);
7.550 (2.8); 7.491 (3.2); 7.471 (2.9); 7.407 (3.1); 7.363 (1.8); 7.361 (1.7); 7.343 (1.5); 7.340 (1.5); 7.314 (1.8);
7.296 (1.7); 4.207 (0.7); 4.191 (0.7); 4.170 (1.5); 4.154 (1.5); 4.133 (0.8); 4.118 (0.7); 3.971 (16.0); 3.317 (30.1);
2.541 (17.1); 2.524 (0.6); 2.506 (27.7); 2.501 (37.4); 2.497 (28.7); 0.008 (2.3); 0.000 (54.9); −0.008 (2.5); −0.016 (0.4)
Example 78: 1H-NMR (400.0 MHz, d6-DMSO):
δ = 9.003 (1.9); 8.987 (4.0); 8.972 (2.0); 8.313 (1.3); 7.764 (9.2); 7.746 (5.1); 7.720 (1.7); 7.702 (6.5); 7.682 (7.0);
7.651 (3.2); 7.631 (11.4); 7.621 (9.9); 7.611 (6.6); 7.599 (10.1); 7.573 (0.4); 7.554 (0.5); 7.509 (0.4); 7.487 (0.4);
7.470 (0.3); 7.449 (0.3); 7.382 (4.3); 7.364 (3.9); 6.636 (8.9); 6.614 (8.8); 6.479 (0.3); 4.227 (1.6); 4.212 (1.7);
4.192 (3.5); 4.176 (3.4); 4.156 (1.8); 4.140 (1.7); 3.353 (0.3); 3.314 (176.9); 3.299 (7.7); 3.283 (15.6); 3.266 (6.6);
3.237 (0.8); 3.220 (0.3); 2.674 (2.6); 2.670 (3.5); 2.666 (2.8); 2.589 (0.4); 2.523 (10.0); 2.505 (391.6); 2.501 (525.8);
2.497 (408.0); 2.426 (0.4); 2.332 (2.6); 2.328 (3.5); 2.324 (2.7); 2.073 (1.3); 1.989 (6.1); 1.973 (16.0); 1.957 (6.3);
1.932 (0.5); 1.251 (4.2); 1.236 (0.7); 0.146 (3.3); 0.039 (0.7); 0.028 (0.8); 0.008 (28.7); 0.000 (665.3); −0.008 (38.3);
−0.032 (1.4); −0.041 (0.4); −0.150 (3.4)
Example 79: 1H-NMR (400.0 MHz, d6-DMSO):
δ = 9.123 (2.9); 9.108 (6.2); 9.092 (2.9); 8.445 (10.7); 8.440 (11.0); 8.313 (2.6); 8.221 (5.1); 8.217 (5.0); 8.201 (5.8);
8.196 (5.8); 8.022 (10.9); 8.006 (5.1); 7.996 (3.5); 7.993 (3.9); 7.892 (10.4); 7.871 (9.3); 7.760 (5.9); 7.741 (8.2);
7.727 (2.7); 7.708 (7.4); 7.687 (14.4); 7.674 (16.0); 7.672 (15.9); 7.655 (5.3); 7.636 (6.1); 7.617 (2.2); 7.421 (7.1);
7.402 (6.3); 7.240 (5.5); 7.101 (12.2); 6.961 (5.9); 4.257 (2.6); 4.242 (2.7); 4.223 (5.8); 4.207 (5.7); 4.188 (3.0);
4.172 (2.8); 4.038 (0.3); 4.020 (0.3); 3.315 (112.0); 3.291 (0.9); 2.675 (1.7); 2.670 (2.3); 2.666 (1.7); 2.662 (0.9);
2.524 (5.8); 2.519 (9.0); 2.510 (118.7); 2.506 (241.3); 2.501 (323.9); 2.497 (241.0); 2.492 (119.1); 2.333 (1.6); 2.328 (2.2);
2.324 (1.6); 1.988 (1.3); 1.398 (2.2); 1.193 (0.4); 1.175 (0.7); 1.158 (0.4); 0.146 (1.1); 0.008 (7.9); 0.000 (236.0);
−0.008 (8.6); −0.150 (1.0)
Example 80: 1H-NMR (400.0 MHz, d6-DMSO):
δ = 9.070 (1.8); 9.055 (3.7); 9.039 (1.8); 7.871 (7.0); 7.851 (8.8); 7.783 (3.6); 7.764 (4.8); 7.736 (1.6); 7.718 (4.2);
7.690 (11.7); 7.671 (8.6); 7.655 (4.6); 7.650 (5.1); 7.627 (1.5); 7.610 (7.2); 7.589 (4.0); 7.389 (4.2); 7.370 (3.8);
7.236 (2.7); 7.096 (6.5); 6.956 (3.1); 5.753 (1.9); 4.113 (1.5); 4.097 (1.6); 4.075 (3.5); 4.059 (3.4); 4.036 (1.8);
4.021 (1.7); 3.317 (28.7); 2.675 (0.4); 2.671 (0.5); 2.666 (0.4); 2.574 (16.0); 2.524 (1.4); 2.511 (27.9); 2.506 (55.8);
2.502 (74.3); 2.497 (54.9); 2.493 (27.0); 2.333 (0.4); 2.328 (0.5); 2.324 (0.3); 0.008 (1.7); 0.000 (48.5); −0.009 (1.8)
Example 81: 1H-NMR (400.0 MHz, d6-DMSO):
δ = 9.152 (2.0); 9.137 (4.3); 9.121 (2.0); 8.314 (0.4); 7.963 (8.3); 7.943 (9.9); 7.776 (4.2); 7.756 (6.3); 7.733 (4.9);
7.706 (16.0); 7.686 (8.4); 7.677 (8.9); 7.649 (4.4); 7.630 (1.6); 7.419 (5.0); 7.400 (4.5); 7.243 (3.0); 7.104 (7.2);
6.964 (3.4); 4.166 (1.7); 4.150 (1.9); 4.131 (3.9); 4.115 (3.8); 4.095 (2.0); 4.080 (1.9); 3.316 (49.4); 2.675 (0.6);
2.671 (0.9); 2.666 (0.7); 2.524 (2.4); 2.510 (51.1); 2.506 (101.7); 2.501 (135.3); 2.497 (101.6); 2.493 (51.6); 2.333 (0.7);
2.328 (0.9); 2.324 (0.7); 1.398 (2.6); 0.146 (0.5); 0.008 (3.5); 0.000 (97.9); −0.008 (4.1); −0.150 (0.5)
Example 82: 1H-NMR (400.0 MHz, d6-DMSO):
δ = 9.154 (1.9); 9.138 (4.0); 9.122 (2.0); 8.003 (8.4); 7.987 (3.3); 7.984 (3.0); 7.776 (4.0); 7.756 (6.0); 7.732 (4.8);
7.706 (8.3); 7.674 (16.0); 7.659 (6.6); 7.649 (4.8); 7.639 (1.4); 7.630 (1.8); 7.420 (4.7); 7.401 (4.2); 7.220 (3.4);
7.080 (7.2); 6.941 (3.5); 4.167 (1.7); 4.151 (1.9); 4.132 (3.8); 4.116 (3.7); 4.096 (2.1); 4.081 (1.9); 3.316 (39.2);
2.675 (0.7); 2.671 (0.9); 2.666 (0.7); 2.506 (98.4); 2.502 (128.8); 2.497 (98.3); 2.333 (0.6); 2.329 (0.8); 2.324 (0.6);
1.398 (4.1); 0.146 (0.4); 0.008 (4.2); 0.000 (90.6); −0.008 (5.3); −0.149 (0.4)
Example 83: 1H-NMR (400.0 MHz, d6-DMSO):
δ = 9.126 (2.2); 9.110 (4.8); 9.095 (2.3); 8.458 (7.8); 8.454 (8.2); 8.314 (0.5); 8.237 (3.7); 8.233 (3.7); 8.216 (4.2);
8.212 (4.3); 7.994 (11.2); 7.990 (4.6); 7.973 (16.0); 7.901 (7.4); 7.880 (7.2); 7.870 (13.3); 7.849 (9.6); 7.759 (4.3);
7.740 (6.0); 7.727 (2.0); 7.708 (4.9); 7.690 (3.7); 7.655 (3.7); 7.636 (4.5); 7.617 (1.6); 7.417 (5.2); 7.398 (4.7);
4.260 (1.9); 4.245 (2.0); 4.226 (4.2); 4.210 (4.2); 4.191 (2.2); 4.175 (2.0); 3.316 (59.6); 2.675 (0.8); 2.671 (1.1);
2.666 (0.8); 2.541 (1.3); 2.524 (2.9); 2.511 (57.2); 2.506 (115.6); 2.502 (155.0); 2.497 (117.3); 2.493 (59.7); 2.333 (0.7);
2.329 (1.0); 2.324 (0.8); 0.146 (0.4); 0.008 (3.5); 0.000 (97.0); −0.008 (4.0); −0.150 (0.4)
Example 84: 1H-NMR (400.0 MHz, d6-DMSO):
δ = 9.010 (1.8); 8.994 (3.2); 8.979 (1.6); 8.313 (0.6); 8.036 (0.6); 7.910 (0.5); 7.888 (0.5); 7.802 (5.7); 7.765 (3.9);
7.746 (4.9); 7.732 (2.7); 7.708 (4.8); 7.703 (4.8); 7.683 (3.2); 7.674 (2.3); 7.660 (5.9); 7.641 (9.4); 7.620 (7.8);
7.509 (0.5); 7.489 (0.5); 7.381 (3.9); 7.363 (3.5); 7.039 (6.2); 7.017 (5.9); 6.871 (0.6); 6.850 (0.5); 6.518 (0.4);
4.236 (1.5); 4.220 (1.7); 4.200 (3.0); 4.184 (2.9); 4.163 (1.6); 4.149 (1.4); 3.316 (113.3); 3.224 (9.0); 3.212 (7.1);
3.172 (1.0); 3.127 (0.4); 3.115 (0.4); 3.103 (0.3); 2.670 (2.0); 2.599 (0.3); 2.505 (232.8); 2.501 (299.7); 2.497 (234.2);
2.465 (10.8); 2.328 (2.1); 2.233 (16.0); 2.073 (2.1); 1.988 (0.4); 1.973 (0.6); 1.356 (0.6); 1.258 (3.2); 1.251 (1.8);
1.235 (1.4); 0.000 (58.8)
Example 85: 1H-NMR (400.0 MHz, d6-DMSO):
δ = 9.128 (2.5); 9.112 (5.3); 9.097 (2.6); 8.475 (8.9); 8.472 (9.2); 8.314 (0.7); 8.249 (4.3); 8.245 (4.3); 8.228 (4.9);
8.224 (4.9); 8.059 (9.4); 8.039 (12.2); 7.913 (8.4); 7.893 (16.0); 7.874 (10.0); 7.761 (4.9); 7.741 (6.9); 7.728 (2.4);
7.710 (5.7); 7.691 (4.2); 7.656 (4.2); 7.637 (5.1); 7.619 (1.9); 7.423 (6.0); 7.404 (5.3); 4.263 (2.2); 4.247 (2.4);
4.228 (4.9); 4.212 (4.8); 4.193 (2.6); 4.178 (2.4); 3.316 (95.4); 2.675 (1.2); 2.671 (1.7); 2.666 (1.3); 2.506 (193.5);
2.502 (253.5); 2.497 (192.3); 2.333 (1.3); 2.329 (1.7); 2.324 (1.3); 2.073 (1.1); 0.146 (0.3); 0.008 (3.0); 0.000 (72.7);
−0.008 (3.4)
Example 86: 1H-NMR (400.0 MHz, d6-DMSO):
δ = 9.159 (3.1); 9.143 (6.5); 9.128 (3.1); 8.311 (0.8); 8.043 (12.0); 8.023 (15.5); 7.874 (16.0); 7.853 (12.8); 7.777 (6.5);
7.756 (11.3); 7.745 (13.6); 7.733 (8.6); 7.716 (16.0); 7.670 (5.3); 7.651 (6.7); 7.632 (2.5); 7.421 (7.6); 7.402 (6.9);
4.171 (2.7); 4.155 (3.0); 4.136 (6.2); 4.120 (6.0); 4.101 (3.3); 4.085 (3.0); 3.481 (0.4); 3.467 (0.4); 3.457 (0.4);
3.441 (0.5); 3.429 (0.6); 3.413 (0.8); 3.391 (1.8); 3.336 (1201.9); 3.267 (0.8); 3.249 (0.5); 3.238 (0.4); 3.221 (0.3);
2.677 (1.4); 2.672 (1.9); 2.668 (1.4); 2.663 (0.7); 2.559 (0.4); 2.525 (5.9); 2.512 (105.2); 2.508 (210.2); 2.503 (278.5);
2.499 (207.5); 2.494 (105.2); 2.334 (1.3); 2.330 (1.8); 2.325 (1.3); 2.073 (1.6); 0.146 (0.3); 0.008 (2.9); 0.000 (82.5);
−0.009 (3.4); −0.150 (0.3)
Example 87: 1H-NMR (400.0 MHz, d6-DMSO):
δ = 8.884 (0.8); 8.868 (1.6); 8.852 (0.7); 7.986 (2.7); 7.966 (3.8); 7.856 (3.9); 7.835 (2.9); 7.752 (1.5); 7.733 (2.0);
7.701 (0.7); 7.683 (1.7); 7.665 (1.3); 7.636 (1.3); 7.617 (1.5); 7.600 (2.7); 7.580 (2.8); 7.461 (2.9); 7.423 (1.8);
7.420 (1.7); 7.403 (1.5); 7.400 (1.4); 7.317 (1.8); 7.298 (1.6); 4.214 (0.6); 4.198 (0.7); 4.178 (1.5); 4.162 (1.4);
4.141 (0.8); 4.125 (0.7); 3.985 (16.0); 3.317 (18.1); 2.524 (0.8); 2.510 (16.1); 2.506 (32.3); 2.502 (42.9); 2.497 (32.3);
2.493 (16.4); 0.008 (0.4); 0.000 (12.0); −0.009 (0.5)
Example 88: 1H-NMR (400.0 MHz, d6-DMSO):
δ = 9.077 (1.7); 9.062 (3.6); 9.046 (1.7); 8.023 (0.3); 7.955 (6.1); 7.934 (9.0); 7.873 (0.4); 7.850 (9.4); 7.829 (6.4);
7.784 (3.5); 7.764 (4.8); 7.737 (1.8); 7.718 (10.3); 7.701 (4.7); 7.685 (4.1); 7.667 (3.1); 7.647 (3.8); 7.629 (7.8);
7.609 (4.0); 7.391 (4.1); 7.373 (3.7); 4.119 (1.6); 4.103 (1.6); 4.080 (3.5); 4.065 (3.3); 4.042 (1.8); 4.027 (1.6);
3.322 (13.1); 2.891 (0.4); 2.733 (0.4); 2.676 (0.3); 2.672 (0.4); 2.582 (16.0); 2.525 (1.3); 2.507 (43.4); 2.503 (56.3);
2.498 (42.0); 2.329 (0.3); 0.000 (5.4)
Example 89: 1H-NMR (400.0 MHz, d6-DMSO):
δ = 9.123 (2.7); 9.107 (5.7); 9.091 (2.7); 8.867 (0.5); 8.433 (9.8); 8.430 (9.8); 8.313 (0.9); 8.219 (4.7); 8.216 (4.5);
8.199 (5.3); 8.195 (5.2); 7.976 (11.1); 7.956 (13.1); 7.894 (8.8); 7.873 (7.9); 7.856 (1.4); 7.836 (1.0); 7.781 (0.4);
7.761 (5.7); 7.741 (8.3); 7.729 (14.5); 7.709 (16.0); 7.691 (4.9); 7.656 (4.7); 7.637 (6.0); 7.618 (2.6); 7.600 (0.9);
7.580 (0.9); 7.461 (1.0); 7.420 (7.0); 7.401 (6.3); 7.317 (0.6); 7.298 (0.5); 7.253 (3.8); 7.113 (8.9); 6.974 (4.2);
4.257 (2.4); 4.242 (2.6); 4.223 (5.4); 4.207 (5.3); 4.188 (2.9); 4.173 (2.9); 3.985 (4.8); 3.892 (0.6); 3.316 (94.5);
2.675 (1.7); 2.671 (2.2); 2.666 (1.7); 2.580 (0.4); 2.506 (259.2); 2.501 (332.8); 2.497 (248.3); 2.333 (1.7); 2.328 (2.2);
2.324 (1.6); 2.073 (7.4); 0.008 (1.4); 0.000 (32.1)
Example 90: 1H-NMR (400.0 MHz, d6-DMSO):
δ = 9.073 (1.7); 9.057 (3.5); 9.042 (1.7); 7.898 (6.9); 7.784 (3.5); 7.764 (4.7); 7.736 (1.7); 7.718 (4.1); 7.699 (3.3);
7.684 (6.5); 7.666 (5.6); 7.646 (10.6); 7.626 (6.5); 7.618 (6.1); 7.611 (8.3); 7.599 (2.1); 7.591 (3.8); 7.391 (4.1);
7.372 (3.7); 7.244 (2.8); 7.105 (6.0); 6.965 (2.9); 4.113 (1.5); 4.098 (1.6); 4.075 (3.4); 4.060 (3.4); 4.037 (1.8);
4.022 (1.7); 3.985 (0.4); 3.319 (31.7); 2.671 (0.6); 2.577 (16.0); 2.506 (60.6); 2.502 (78.3); 2.497 (59.2); 2.332 (0.4);
2.328 (0.5); 2.324 (0.4); 2.074 (2.0); 0.000 (5.6)
Example 91: 1H-NMR (400.0 MHz, d6-DMSO):
δ = 8.879 (0.7); 8.863 (1.6); 8.847 (0.8); 8.313 (0.4); 7.903 (3.0); 7.882 (3.7); 7.752 (1.5); 7.733 (2.1); 7.698 (3.8);
7.679 (3.7); 7.664 (1.5); 7.635 (1.4); 7.617 (1.6); 7.598 (0.6); 7.581 (2.2); 7.561 (2.7); 7.432 (3.0); 7.397 (1.8);
7.377 (1.5); 7.373 (1.5); 7.315 (1.8); 7.296 (1.6); 7.241 (1.1); 7.101 (2.6); 6.961 (1.2); 4.209 (0.7); 4.193 (0.7);
4.172 (1.5); 4.157 (1.5); 4.136 (0.8); 4.120 (0.7); 3.978 (16.0); 3.315 (39.7); 2.675 (0.7); 2.670 (0.9); 2.666 (0.7);
2.569 (0.5); 2.523 (2.9); 2.510 (52.0); 2.506 (101.9); 2.501 (134.0); 2.497 (101.4); 2.332 (0.6); 2.328 (0.9); 2.323 (0.7);
0.008 (0.5); 0.000 (12.7); −0.008 (0.6)
Example 92: 1H-NMR (400.0 MHz, d6-DMSO):
δ = 8.883 (0.8); 8.867 (1.7); 8.852 (0.8); 7.933 (1.5); 7.921 (4.3); 7.753 (1.7); 7.734 (2.4); 7.700 (0.8); 7.682 (2.0);
7.672 (0.9); 7.664 (1.6); 7.653 (2.0); 7.633 (5.0); 7.616 (2.2); 7.598 (0.7); 7.585 (2.3); 7.565 (2.8); 7.425 (3.3);
7.388 (2.0); 7.368 (1.9); 7.316 (1.9); 7.298 (1.8); 7.244 (1.4); 7.104 (2.8); 6.965 (1.4); 4.211 (0.7); 4.196 (0.8);
4.175 (1.6); 4.160 (1.6); 4.138 (0.9); 4.123 (0.8); 3.985 (16.0); 3.892 (1.3); 3.318 (15.2); 2.506 (31.3); 2.501 (40.7);
2.497 (31.3); 0.000 (3.2)
Example 93: 1H-NMR (400.0 MHz, d6-DMSO):
δ = 9.045 (2.3); 9.030 (4.8); 9.014 (2.3); 8.314 (0.4); 8.054 (5.8); 8.029 (4.9); 8.026 (4.9); 7.860 (9.6); 7.841 (16.0);
7.826 (6.4); 7.802 (5.5); 7.782 (9.9); 7.766 (4.5); 7.745 (9.0); 7.720 (4.9); 7.703 (4.9); 7.684 (3.7); 7.656 (3.8);
7.637 (4.5); 7.619 (1.6); 7.381 (5.2); 7.362 (4.6); 4.277 (1.9); 4.262 (2.0); 4.241 (4.4); 4.225 (4.2); 4.205 (2.3);
4.190 (2.1); 3.319 (98.6); 3.296 (0.4); 2.891 (0.8); 2.732 (0.6); 2.676 (0.6); 2.671 (0.9); 2.667 (0.6); 2.511 (52.0);
2.506 (101.2); 2.502 (132.5); 2.497 (98.5); 2.493 (50.2); 2.333 (0.7); 2.329 (0.9); 2.324 (0.7); 1.989 (0.8); 1.398 (0.7);
1.298 (1.7); 1.176 (0.5); 1.159 (0.8); 0.000 (4.2)
Example 94: 1H-NMR (400.0 MHz, d6-DMSO):
δ = 9.033 (0.9); 9.018 (1.9); 9.002 (0.9); 8.054 (1.8); 8.048 (1.9); 8.036 (1.9); 8.030 (1.9); 7.977 (3.4); 7.847 (1.4);
7.844 (1.4); 7.823 (2.9); 7.818 (1.4); 7.812 (1.2); 7.806 (1.2); 7.802 (1.3); 7.796 (1.2); 7.790 (1.2); 7.784 (1.1);
7.768 (1.8); 7.749 (2.5); 7.731 (3.7); 7.710 (4.0); 7.689 (1.5); 7.656 (1.6); 7.637 (1.9); 7.618 (0.7); 7.567 (2.0);
7.544 (3.2); 7.522 (1.7); 7.388 (2.1); 7.369 (1.9); 4.257 (0.8); 4.242 (0.9); 4.222 (1.8); 4.206 (1.8); 4.186 (1.0);
4.170 (0.9); 3.320 (19.4); 2.507 (25.1); 2.503 (32.5); 2.498 (24.6); 1.989 (1.1); 1.398 (16.0); 1.176 (0.6); 0.000 (1.1)
Example 95: 1H-NMR (400.0 MHz, d6-DMSO):
δ = 9.038 (1.6); 9.022 (3.1); 9.007 (1.6); 8.469 (5.7); 8.464 (6.0); 8.165 (2.9); 8.160 (2.9); 8.144 (3.5); 8.138 (3.4);
8.075 (5.7); 7.973 (0.3); 7.952 (0.5); 7.920 (2.5); 7.917 (2.5); 7.900 (3.2); 7.897 (3.3); 7.878 (5.9); 7.857 (5.2);
7.786 (0.4); 7.765 (7.0); 7.744 (5.8); 7.725 (1.5); 7.707 (3.4); 7.689 (2.5); 7.656 (2.7); 7.637 (3.2); 7.619 (1.2);
7.387 (3.5); 7.369 (3.2); 5.753 (16.0); 4.261 (1.3); 4.246 (1.5); 4.226 (3.0); 4.210 (2.9); 4.190 (1.6); 4.174 (1.4);
4.039 (0.6); 4.021 (0.6); 3.319 (30.9); 2.892 (2.0); 2.733 (1.7); 2.672 (0.4); 2.507 (51.3); 2.503 (66.0); 2.498 (51.5);
2.329 (0.4); 1.989 (2.5); 1.398 (2.5); 1.260 (0.5); 1.250 (0.5); 1.233 (2.3); 1.194 (0.7); 1.176 (1.4); 1.158 (0.7);
0.000 (2.0)
Example 96: 1H-NMR (400.0 MHz, d6-DMSO):
δ = 10.163 (0.3); 9.038 (1.6); 9.023 (3.4); 9.007 (1.7); 8.428 (3.2); 8.422 (3.5); 8.413 (3.4); 8.407 (3.4); 8.226 (1.7);
8.220 (1.7); 8.213 (2.0); 8.207 (2.3); 8.204 (2.3); 8.198 (2.1); 8.191 (2.0); 8.185 (1.8); 8.041 (6.1); 8.038 (6.2);
7.889 (2.7); 7.885 (2.6); 7.868 (3.7); 7.864 (3.6); 7.768 (3.4); 7.756 (7.1); 7.750 (5.3); 7.735 (5.0); 7.727 (1.8);
7.708 (3.8); 7.686 (5.0); 7.663 (7.0); 7.658 (3.5); 7.640 (5.9); 7.619 (1.3); 7.388 (3.9); 7.369 (3.6); 5.753 (16.0);
4.261 (1.4); 4.246 (1.5); 4.225 (3.3); 4.209 (3.2); 4.189 (1.8); 4.173 (1.6); 4.039 (0.8); 4.021 (0.8); 3.320 (55.9);
2.892 (0.4); 2.733 (0.4); 2.676 (0.3); 2.672 (0.4); 2.667 (0.3); 2.525 (1.2); 2.511 (30.3); 2.507 (58.7); 2.502 (75.9);
2.498 (56.5); 2.494 (29.0); 2.334 (0.4); 2.329 (0.5); 2.325 (0.4); 1.989 (3.6); 1.398 (9.8); 1.353 (0.7); 1.337 (0.3);
1.260 (0.4); 1.250 (0.6); 1.232 (1.1); 1.194 (1.0); 1.176 (2.0); 1.158 (1.0); 1.122 (0.3); 0.000 (2.5)
Example 97: 1H-NMR (400.0 MHz, d6-DMSO):
δ = 9.046 (1.6); 9.030 (3.2); 9.015 (1.5); 8.346 (6.6); 8.308 (16.0); 8.184 (5.7); 8.181 (5.7); 8.007 (2.6); 7.987 (3.2);
7.983 (3.1); 7.800 (5.4); 7.779 (4.5); 7.766 (3.1); 7.747 (4.1); 7.723 (1.3); 7.705 (3.4); 7.686 (2.5); 7.655 (2.6);
7.636 (3.0); 7.617 (1.1); 7.393 (3.6); 7.374 (3.2); 4.273 (1.3); 4.256 (1.4); 4.236 (3.0); 4.221 (2.9); 4.201 (1.5);
4.185 (1.4); 3.348 (0.5); 3.316 (322.1); 3.281 (0.4); 2.675 (2.6); 2.670 (3.4); 2.666 (2.5); 2.578 (0.4); 2.506 (393.2);
2.501 (508.1); 2.497 (377.1); 2.333 (2.5); 2.328 (3.3); 2.324 (2.5); 1.988 (0.5); 1.398 (1.2); 0.000 (6.7)
Example 104: 1H-NMR (400.0 MHz, d6-DMSO):
δ = 9.014 (0.9); 8.998 (1.9); 8.983 (0.9); 8.313 (0.8); 7.815 (3.2); 7.766 (1.7); 7.745 (3.1); 7.722 (2.7); 7.703 (2.0);
7.684 (1.6); 7.668 (6.3); 7.647 (6.4); 7.633 (2.2); 7.613 (0.8); 7.568 (0.3); 7.548 (0.4); 7.527 (0.3); 7.382 (1.9);
7.363 (1.7); 7.067 (3.7); 7.045 (3.6); 4.238 (0.7); 4.222 (0.9); 4.203 (1.6); 4.186 (1.6); 4.165 (0.9); 4.151 (0.8);
3.589 (5.2); 3.316 (156.9); 3.279 (2.5); 3.267 (3.0); 3.213 (2.1); 3.200 (2.7); 3.187 (1.8); 2.670 (2.8); 2.505 (354.9);
2.501 (438.9); 2.328 (3.1); 2.073 (0.7); 2.049 (16.0); 0.146 (2.3); 0.000 (453.8); −0.089 (0.3); −0.150 (2.4)
Example 105: 1H-NMR (400.0 MHz, d6-DMSO):
δ = 9.040 (0.7); 9.025 (1.6); 9.009 (0.7); 8.218 (3.3); 8.214 (3.4); 8.113 (2.7); 8.100 (3.0); 8.092 (3.7); 7.981 (2.0);
7.977 (2.1); 7.961 (1.6); 7.956 (2.1); 7.932 (1.6); 7.928 (1.6); 7.777 (2.7); 7.766 (1.6); 7.757 (2.4); 7.746 (2.1);
7.724 (0.7); 7.706 (1.6); 7.688 (1.2); 7.655 (1.2); 7.636 (1.5); 7.617 (0.5); 7.386 (1.7); 7.367 (1.6); 4.265 (0.6);
4.250 (0.7); 4.230 (1.4); 4.214 (1.4); 4.193 (0.7); 4.178 (0.7); 4.056 (0.6); 4.038 (1.8); 4.021 (1.8); 4.003 (0.6);
3.318 (34.9); 2.891 (1.4); 2.732 (1.2); 2.524 (0.9); 2.511 (19.2); 2.507 (38.2); 2.502 (50.7); 2.498 (38.3); 2.494 (19.8);
2.329 (0.3); 1.989 (7.6); 1.398 (16.0); 1.193 (2.0); 1.176 (4.0); 1.158 (2.0); 0.015 (0.3); 0.008 (2.5); 0.000 (63.5);
−0.008 (3.1)
Example 106: 1H-NMR (400.0 MHz, d6-DMSO):
δ = 9.037 (2.8); 9.022 (5.9); 9.006 (2.8); 8.315 (0.5); 8.009 (16.0); 7.985 (5.3); 7.980 (5.5); 7.880 (4.5); 7.877 (4.4);
7.860 (6.2); 7.856 (6.4); 7.767 (6.1); 7.756 (14.2); 7.749 (10.8); 7.735 (14.1); 7.728 (9.7); 7.707 (10.1); 7.688 (9.6);
7.667 (2.3); 7.655 (4.9); 7.636 (5.8); 7.617 (2.1); 7.384 (6.7); 7.366 (6.1); 4.262 (2.4); 4.247 (2.6); 4.226 (5.6);
4.211 (5.4); 4.190 (2.9); 4.175 (2.7); 3.318 (81.4); 2.675 (1.0); 2.671 (1.4); 2.666 (1.0); 2.506 (156.9); 2.502 (209.4);
2.497 (159.3); 2.333 (1.0); 2.329 (1.4); 2.324 (1.1); 1.964 (0.3); 1.398 (2.4); 0.146 (1.4); 0.023 (0.4); 0.008 (12.1);
0.000 (300.3); −0.008 (15.6); −0.150 (1.4)
Example 107: 1H-NMR (400.0 MHz, d6-DMSO):
δ = 9.122 (4.3); 9.107 (9.0); 9.091 (4.3); 8.488 (15.2); 8.484 (16.0); 8.314 (1.3); 8.254 (7.3); 8.250 (7.3); 8.233 (8.1);
8.229 (8.2); 7.974 (1.0); 7.966 (1.8); 7.956 (10.7); 7.939 (11.4); 7.933 (11.3); 7.916 (10.3); 7.897 (0.9); 7.875 (14.6);
7.854 (13.3); 7.763 (8.7); 7.744 (12.2); 7.728 (3.9); 7.710 (10.0); 7.691 (7.3); 7.658 (7.6); 7.639 (9.0); 7.620 (3.3);
7.412 (10.6); 7.393 (9.4); 4.248 (3.9); 4.233 (4.1); 4.214 (8.9); 4.198 (8.7); 4.179 (4.6); 4.163 (4.2); 3.317 (300.9);
2.675 (2.9); 2.671 (4.1); 2.666 (3.1); 2.524 (11.9); 2.506 (468.7); 2.502 (620.0); 2.497 (462.8); 2.333 (2.9); 2.328 (4.1);
2.324 (3.1); 2.288 (0.4); 1.988 (0.5); 1.398 (0.5); 1.148 (0.5); 0.146 (2.8); 0.032 (0.4); 0.024 (0.8); 0.008 (20.9);
0.000 (586.9); −0.008 (26.9); −0.038 (0.4); −0.150 (2.8)
Example 108: 1H-NMR (400.0 MHz, d6-DMSO):
δ = 9.154 (2.5); 9.138 (5.4); 9.123 (2.7); 8.315 (0.4); 7.964 (0.6); 7.956 (1.2); 7.946 (6.7); 7.929 (7.1); 7.922 (6.9);
7.906 (6.4); 7.896 (1.2); 7.888 (0.6); 7.777 (16.0); 7.758 (9.1); 7.749 (13.1); 7.732 (7.0); 7.714 (4.6); 7.671 (4.7);
7.652 (5.9); 7.633 (2.4); 7.409 (6.5); 7.390 (6.0); 4.155 (2.4); 4.139 (2.5); 4.120 (5.3); 4.104 (5.3); 4.085 (2.8);
4.069 (2.7); 3.319 (29.3); 2.676 (0.7); 2.671 (0.9); 2.667 (0.7); 2.525 (2.3); 2.511 (49.5); 2.507 (100.5); 2.502 (133.3);
2.498 (98.5); 2.494 (49.8); 2.334 (0.6); 2.329 (0.8); 2.325 (0.6); 1.398 (5.5); 0.146 (0.8); 0.008 (6.7); 0.000 (176.5);
−0.008 (7.8); −0.023 (0.5); −0.150 (0.8)
Example 109: 1H-NMR (400.0 MHz, d6-DMSO):
δ = 9.073 (0.5); 9.057 (1.1); 9.042 (0.5); 7.788 (1.7); 7.785 (1.7); 7.772 (1.7); 7.765 (3.0); 7.748 (1.4); 7.732 (2.2);
7.719 (1.5); 7.702 (1.6); 7.685 (1.2); 7.667 (1.0); 7.648 (1.2); 7.629 (0.5); 7.583 (2.0); 7.563 (1.5); 7.380 (1.4);
7.361 (1.3); 4.102 (0.5); 4.087 (0.5); 4.064 (1.2); 4.048 (1.1); 4.026 (0.6); 4.010 (0.6); 3.319 (4.8); 2.559 (5.3);
2.524 (0.5); 2.506 (19.1); 2.502 (25.2); 2.498 (19.1); 1.989 (0.5); 1.398 (16.0); 0.008 (1.1); 0.000 (30.2); −0.008 (1.6)
Example 110: 1H-NMR (400.0 MHz, d6-DMSO):
δ = 9.035 (2.2); 9.019 (4.6); 9.004 (2.2); 8.314 (0.4); 8.069 (8.2); 7.929 (3.6); 7.925 (3.5); 7.908 (4.6); 7.905 (4.5);
7.886 (1.6); 7.875 (10.8); 7.853 (10.6); 7.768 (4.5); 7.748 (13.7); 7.727 (7.8); 7.707 (5.0); 7.688 (3.6); 7.656 (3.7);
7.637 (4.5); 7.618 (1.6); 7.381 (5.2); 7.362 (4.6); 4.258 (1.9); 4.242 (2.1); 4.222 (4.4); 4.206 (4.2); 4.186 (2.3);
4.170 (2.1); 4.038 (0.5); 4.021 (0.5); 3.318 (117.8); 2.676 (0.9); 2.671 (1.2); 2.667 (0.9); 2.524 (3.7); 2.506 (142.2);
2.502 (184.7); 2.498 (136.8); 2.333 (0.9); 2.329 (1.2); 2.324 (0.9); 1.989 (2.2); 1.398 (16.0); 1.193 (0.6); 1.175 (1.2);
1.158 (0.6); 0.146 (0.8); 0.020 (0.4); 0.008 (6.5); 0.000 (167.3); −0.150 (0.8)
Example 111: 1H-NMR (400.0 MHz, d6-DMSO):
δ = 9.341 (3.5); 9.325 (7.1); 9.310 (3.4); 9.020 (10.8); 9.015 (13.2); 8.965 (12.9); 8.960 (11.4); 8.040 (13.8); 7.902 (0.9);
7.882 (12.0); 7.860 (16.0); 7.844 (8.0); 7.718 (11.9); 7.698 (9.4); 4.322 (3.3); 4.306 (3.4); 4.285 (7.3); 4.269 (7.1);
4.248 (3.8); 4.232 (3.5); 3.327 (359.7); 2.712 (1.7); 2.672 (1.4); 2.583 (0.5); 2.542 (350.2); 2.525 (4.7); 2.503 (215.4);
2.455 (0.5); 2.368 (1.8); 2.330 (1.4); 1.234 (0.3); 0.008 (1.2); 0.000 (31.8)
Example 112: 1H-NMR (400.0 MHz, d6-DMSO):
δ = 9.159 (3.8); 9.143 (8.0); 9.127 (3.8); 8.863 (8.8); 8.852 (8.9); 8.299 (8.5); 8.280 (9.0); 8.026 (15.0); 7.894 (0.9);
7.886 (1.6); 7.876 (9.4); 7.859 (16.0); 7.853 (11.3); 7.837 (14.6); 7.756 (5.9); 7.744 (5.9); 7.737 (5.8); 7.724 (5.5);
7.704 (14.1); 7.683 (11.2); 4.283 (3.6); 4.267 (3.7); 4.246 (8.2); 4.230 (8.0); 4.209 (4.2); 4.193 (3.9); 3.365 (0.7);
3.329 (438.6); 2.712 (0.6); 2.676 (0.9); 2.672 (1.3); 2.668 (1.0); 2.578 (0.3); 2.568 (0.4); 2.542 (145.0); 2.525 (3.4);
2.508 (146.4); 2.503 (193.1); 2.499 (145.4); 2.368 (0.6); 2.334 (0.9); 2.330 (1.2); 2.326 (0.9); 0.008 (1.1); 0.000 (31.3)
Example 113: 1H-NMR (400.0 MHz, d6-DMSO):
δ = 9.011 (2.3); 8.995 (5.1); 8.980 (2.4); 8.310 (1.1); 7.899 (9.2); 7.800 (3.7); 7.797 (3.6); 7.780 (5.3); 7.776 (5.4);
7.764 (4.9); 7.745 (6.5); 7.720 (2.1); 7.703 (5.5); 7.689 (10.2); 7.668 (6.8); 7.657 (5.6); 7.653 (8.6); 7.633 (5.0);
7.622 (4.4); 7.616 (6.2); 7.578 (4.2); 7.573 (3.8); 7.557 (4.6); 7.552 (4.3); 7.380 (5.6); 7.361 (5.0); 7.143 (3.8);
7.121 (6.1); 7.099 (3.5); 4.245 (2.0); 4.229 (2.1); 4.209 (4.5); 4.193 (4.4); 4.172 (2.3); 4.157 (2.2); 3.772 (12.8);
3.761 (16.0); 3.749 (13.9); 3.307 (152.4); 3.091 (13.2); 3.079 (16.0); 3.068 (12.5); 2.674 (1.4); 2.670 (2.0); 2.665 (1.5);
2.523 (4.8); 2.510 (116.8); 2.505 (240.6); 2.501 (321.7); 2.496 (237.2); 2.492 (120.3); 2.332 (1.5); 2.328 (2.1); 2.323 (1.5);
1.988 (0.5); 0.146 (0.8); 0.008 (5.7); 0.000 (173.6); −0.008 (7.4); −0.150 (0.8)
Example 114: 1H-NMR (400.0 MHz, d6-DMSO):
δ = 8.901 (2.2); 8.885 (4.7); 8.870 (2.2); 8.014 (8.6); 7.899 (0.5); 7.890 (1.1); 7.879 (7.6); 7.858 (16.0); 7.840 (11.6);
7.760 (8.4); 7.740 (6.1); 7.437 (2.7); 7.418 (6.1); 7.400 (3.5); 7.399 (3.5); 7.179 (6.9); 7.173 (4.6); 7.160 (5.9);
7.154 (6.3); 7.150 (4.5); 7.135 (2.7); 7.131 (2.3); 4.244 (2.0); 4.229 (2.1); 4.209 (4.4); 4.193 (4.3); 4.173 (2.3);
4.158 (2.1); 3.380 (0.3); 3.362 (0.8); 3.330 (313.1); 2.712 (0.9); 2.676 (0.6); 2.672 (0.8); 2.667 (0.6); 2.575 (0.4);
2.542 (197.1); 2.525 (2.8); 2.507 (90.5); 2.502 (119.8); 2.498 (90.5); 2.368 (0.9); 2.333 (0.5); 2.329 (0.8); 2.325 (0.6);
0.008 (0.5); 0.000 (14.5)
Example 115: 1H-NMR (400.0 MHz, d6-DMSO):
δ = 8.960 (3.4); 8.945 (7.1); 8.929 (3.4); 8.021 (13.4); 7.899 (0.8); 7.880 (12.7); 7.864 (10.1); 7.858 (16.0); 7.841 (8.3);
7.729 (13.0); 7.708 (10.1); 7.470 (2.9); 7.467 (4.3); 7.450 (11.4); 7.447 (13.7); 7.440 (6.8); 7.426 (7.8); 7.422 (8.2);
7.407 (3.5); 7.402 (3.6); 7.391 (0.5); 7.384 (4.7); 7.379 (4.5); 7.365 (7.7); 7.361 (7.9); 7.348 (4.1); 7.344 (4.0);
7.281 (9.1); 7.279 (9.3); 7.263 (6.6); 7.259 (6.3); 4.258 (3.2); 4.242 (3.3); 4.222 (7.2); 4.207 (7.0); 4.187 (3.7);
4.171 (3.4); 3.324 (394.7); 3.295 (0.7); 2.711 (0.4); 2.675 (1.0); 2.671 (1.5); 2.667 (1.1); 2.541 (94.2); 2.524 (4.1);
2.506 (175.3); 2.502 (231.6); 2.498 (174.1); 2.367 (0.5); 2.333 (1.1); 2.329 (1.5); 2.324 (1.1); 0.008 (0.9); 0.000 (29.1)
Example 116: 1H-NMR (400.0 MHz, d6-DMSO):
δ = 8.949 (3.3); 8.933 (6.9); 8.918 (3.3); 8.019 (13.3); 7.899 (0.8); 7.881 (13.2); 7.857 (16.0); 7.841 (8.2); 7.744 (12.5);
7.723 (9.5); 7.625 (8.3); 7.623 (8.5); 7.606 (9.7); 7.604 (9.7); 7.427 (3.2); 7.425 (3.3); 7.409 (8.6); 7.406 (8.6);
7.390 (6.7); 7.388 (6.4); 7.360 (5.1); 7.356 (6.2); 7.341 (6.6); 7.337 (7.5); 7.322 (3.0); 7.318 (3.0); 7.245 (8.2);
7.241 (8.5); 7.227 (6.9); 7.223 (6.7); 4.253 (3.1); 4.237 (3.3); 4.218 (6.9); 4.202 (6.8); 4.182 (3.6); 4.166 (3.3);
3.369 (0.5); 3.326 (495.1); 2.711 (1.3); 2.675 (1.1); 2.671 (1.6); 2.667 (1.2); 2.572 (0.3); 2.541 (292.9); 2.524 (4.5);
2.506 (192.7); 2.502 (253.5); 2.498 (193.6); 2.439 (0.4); 2.431 (0.4); 2.368 (1.4); 2.333 (1.3); 2.329 (1.7); 2.324 (1.3);
1.235 (0.3); 0.008 (0.8); 0.000 (23.6)
Example 117: 1H-NMR (400.0 MHz, d6-DMSO):
δ = 9.246 (3.6); 9.230 (7.2); 9.215 (3.7); 8.030 (14.1); 7.889 (9.6); 7.882 (9.9); 7.873 (11.3); 7.864 (13.5); 7.849 (9.1);
7.692 (10.9); 7.671 (9.1); 7.537 (1.3); 7.517 (3.4); 7.500 (5.9); 7.480 (3.9); 7.462 (1.6); 7.155 (9.8); 7.135 (16.0);
7.115 (8.6); 4.278 (3.3); 4.263 (3.6); 4.243 (7.4); 4.227 (7.2); 4.206 (3.9); 4.191 (3.5); 3.325 (410.2); 2.711 (0.8);
2.671 (1.9); 2.541 (156.1); 2.502 (287.9); 2.367 (0.8); 2.329 (1.8); 1.235 (0.5); −0.001 (17.8)
Example 118: 1H-NMR (400.0 MHz, d6-DMSO):
δ = 8.880 (0.8); 8.865 (1.6); 8.849 (0.8); 8.313 (0.8); 7.845 (1.9); 7.828 (2.1); 7.821 (2.0); 7.805 (1.8); 7.753 (1.6);
7.734 (2.1); 7.702 (0.7); 7.684 (1.7); 7.666 (1.3); 7.637 (1.3); 7.618 (1.5); 7.599 (0.6); 7.553 (2.1); 7.533 (2.8);
7.452 (3.0); 7.427 (1.9); 7.407 (1.4); 7.308 (1.8); 7.289 (1.6); 4.194 (0.7); 4.179 (0.7); 4.157 (1.6); 4.142 (1.5);
4.120 (0.8); 4.105 (0.7); 3.982 (16.0); 3.367 (0.4); 3.357 (0.5); 3.319 (369.4); 2.675 (1.7); 2.670 (2.3); 2.666 (1.8);
2.541 (1.1); 2.506 (282.0); 2.501 (363.1); 2.497 (270.2); 2.438 (0.4); 2.332 (1.7); 2.328 (2.3); 2.324 (1.7); 0.000 (37.4);
−0.008 (1.8)
Example 119: 1H-NMR (400.0 MHz, d6-DMSO):
δ = 9.072 (1.4); 9.057 (3.0); 9.041 (1.4); 8.314 (0.4); 7.784 (2.9); 7.765 (3.8); 7.731 (1.2); 7.713 (3.2); 7.695 (2.4);
7.667 (2.5); 7.648 (4.1); 7.630 (2.5); 7.626 (3.2); 7.609 (2.7); 7.600 (3.8); 7.586 (2.2); 7.578 (4.7); 7.485 (7.8);
7.468 (2.2); 7.420 (1.4); 7.414 (1.5); 7.396 (1.7); 7.391 (2.5); 7.386 (2.0); 7.375 (3.6); 7.357 (3.2); 7.244 (1.3);
7.238 (1.2); 7.223 (2.4); 7.217 (2.3); 7.202 (1.2); 7.196 (1.1); 4.114 (1.3); 4.099 (1.3); 4.076 (2.8); 4.060 (2.8);
4.038 (1.5); 4.022 (1.4); 3.318 (93.6); 2.671 (0.9); 2.548 (13.6); 2.506 (100.6); 2.502 (128.1); 2.497 (96.3); 2.328 (0.8);
2.324 (0.6); 1.398 (16.0); 0.000 (10.6)
Example 120: 1H-NMR (400.0 MHz, d6-DMSO):
δ = 9.069 (3.2); 9.054 (6.5); 9.038 (3.2); 7.865 (2.9); 7.861 (3.0); 7.846 (3.1); 7.841 (3.5); 7.835 (3.5); 7.830 (3.2);
7.816 (3.0); 7.810 (3.0); 7.784 (6.5); 7.764 (8.6); 7.737 (2.8); 7.719 (7.2); 7.701 (5.3); 7.675 (12.0); 7.658 (5.8);
7.648 (7.8); 7.637 (7.9); 7.614 (1.9); 7.609 (2.0); 7.592 (5.0); 7.586 (6.2); 7.579 (16.0); 7.558 (8.2); 7.548 (4.9);
7.527 (4.9); 7.506 (1.9); 7.386 (7.5); 7.367 (6.8); 5.754 (12.9); 4.102 (2.8); 4.087 (3.0); 4.064 (6.3); 4.049 (6.1);
4.026 (3.3); 4.011 (3.0); 3.322 (56.3); 3.178 (0.9); 3.165 (0.9); 2.891 (1.0); 2.733 (0.9); 2.671 (0.7); 2.667 (0.5);
2.559 (29.1); 2.506 (77.3); 2.502 (97.7); 2.498 (75.0); 2.329 (0.6); 1.989 (0.5); 1.398 (2.5); 0.000 (9.0)
Example 121: 1H-NMR (400.0 MHz, d6-DMSO):
δ = 9.073 (1.8); 9.057 (3.5); 9.042 (1.7); 7.783 (3.5); 7.764 (4.8); 7.742 (6.9); 7.719 (6.5); 7.699 (5.8); 7.666 (2.9);
7.647 (3.6); 7.628 (1.3); 7.589 (5.6); 7.568 (4.2); 7.536 (0.9); 7.520 (5.4); 7.502 (5.3); 7.486 (0.9); 7.384 (4.1);
7.365 (3.7); 7.298 (1.0); 7.293 (1.6); 7.275 (2.0); 7.270 (3.0); 7.252 (1.1); 7.247 (1.5); 5.754 (1.8); 4.105 (1.5);
4.090 (1.6); 4.067 (3.4); 4.052 (3.3); 4.029 (1.8); 4.013 (1.6); 3.321 (37.4); 2.891 (1.6); 2.732 (1.5); 2.671 (0.4);
2.563 (16.0); 2.502 (63.3); 2.328 (0.4); 0.000 (5.3)
Example 122: 1H-NMR (400.0 MHz, d6-DMSO):
δ = 9.069 (1.6); 9.054 (3.1); 9.038 (1.5); 8.008 (7.0); 7.955 (0.4); 7.783 (3.2); 7.764 (4.4); 7.738 (16.0); 7.718 (8.9);
7.696 (3.4); 7.668 (5.1); 7.647 (3.2); 7.628 (1.2); 7.590 (5.3); 7.570 (3.7); 7.386 (3.6); 7.368 (3.2); 5.754 (5.1);
4.103 (1.4); 4.087 (1.5); 4.065 (3.1); 4.049 (2.9); 4.027 (1.6); 4.011 (1.4); 3.319 (48.8); 3.176 (0.3); 2.891 (0.8);
2.732 (0.8); 2.671 (0.7); 2.564 (14.1); 2.506 (84.3); 2.502 (104.4); 2.498 (81.7); 2.329 (0.6); 1.398 (3.7); 0.000 (14.7)
Example 123: 1H-NMR (400.0 MHz, d6-DMSO):
δ = 9.070 (1.1); 9.054 (2.3); 9.039 (1.1); 7.781 (6.5); 7.764 (3.2); 7.736 (1.1); 7.719 (2.7); 7.701 (4.2); 7.686 (6.0);
7.666 (3.6); 7.646 (4.9); 7.628 (1.0); 7.589 (4.3); 7.568 (2.6); 7.535 (1.7); 7.515 (4.0); 7.496 (2.9); 7.473 (3.1);
7.469 (2.5); 7.456 (1.2); 7.451 (1.4); 7.390 (2.7); 7.371 (2.4); 5.753 (16.0); 4.107 (1.1); 4.094 (1.1); 4.091 (1.1);
4.082 (0.9); 4.068 (2.4); 4.053 (2.2); 4.030 (1.2); 4.015 (1.1); 3.320 (21.9); 3.178 (1.7); 3.165 (1.7); 2.891 (1.0);
2.732 (0.9); 2.566 (10.6); 2.506 (30.1); 2.502 (39.4); 2.498 (30.4); 1.989 (0.3); 1.398 (1.3); 0.000 (4.4)
Example 124: 1H-NMR (400.0 MHz, d6-DMSO):
δ = 9.069 (1.7); 9.054 (3.5); 9.038 (1.7); 7.967 (3.4); 7.962 (3.7); 7.950 (3.6); 7.944 (3.6); 7.783 (3.6); 7.765 (6.1);
7.754 (2.3); 7.748 (2.2); 7.744 (2.6); 7.738 (3.7); 7.733 (3.1); 7.727 (2.5); 7.718 (4.1); 7.700 (3.2); 7.685 (6.3);
7.664 (4.5); 7.644 (5.3); 7.579 (6.3); 7.559 (4.1); 7.549 (3.7); 7.526 (5.8); 7.504 (3.0); 7.388 (4.2); 7.369 (3.7);
5.754 (9.9); 4.102 (1.5); 4.087 (1.6); 4.064 (3.5); 4.048 (3.3); 4.039 (1.3); 4.026 (1.8); 4.010 (1.7); 3.569 (1.2);
3.320 (31.8); 3.178 (0.7); 3.164 (0.7); 2.891 (1.4); 2.732 (1.3); 2.671 (0.4); 2.560 (16.0); 2.506 (49.8); 2.502 (65.7);
2.498 (52.5); 2.334 (0.3); 2.329 (0.4); 1.989 (2.0); 1.398 (3.5); 1.193 (0.5); 1.176 (1.0); 1.158 (0.5); 0.000 (6.0)
Example 125: 1H-NMR (400.0 MHz, d6-DMSO):
δ = 9.068 (1.8); 9.053 (3.5); 9.037 (1.7); 8.314 (0.4); 7.842 (3.4); 7.837 (3.5); 7.814 (3.4); 7.809 (3.5); 7.783 (3.6);
7.763 (4.9); 7.735 (1.7); 7.714 (11.1); 7.693 (8.8); 7.673 (8.3); 7.646 (3.7); 7.632 (5.0); 7.628 (5.5); 7.611 (2.7);
7.606 (2.7); 7.589 (6.3); 7.569 (4.3); 7.383 (4.1); 7.364 (3.7); 4.102 (1.5); 4.087 (1.6); 4.064 (3.5); 4.048 (3.3);
4.026 (1.8); 4.010 (1.6); 3.568 (0.9); 3.319 (110.0); 2.675 (0.7); 2.671 (0.9); 2.666 (0.7); 2.561 (16.0); 2.506 (101.3);
2.502 (131.9); 2.497 (100.0); 2.333 (0.6); 2.328 (0.8); 2.324 (0.6); 1.988 (0.8); 1.398 (8.1); 1.175 (0.4); 0.000 (11.4);
−0.008 (0.5)
Example 126: 1H-NMR (400.0 MHz, d6-DMSO):
δ = 9.075 (1.8); 9.059 (3.6); 9.044 (1.7); 7.789 (9.9); 7.782 (8.5); 7.768 (12.1); 7.750 B (3.2); 7.721 (4.9); 7.701 (2.9);
7.667 (3.0); 7.648 (3.6); 7.629 (1.3); 7.596 (5.7); 7.575 (4.5); 7.382 (4.1); 7.363 (3.7); 4.106 (1.6); 4.090 (1.6);
4.068 (3.5); 4.052 (3.3); 4.039 (0.9); 4.029 (1.8); 4.014 (1.6); 3.569 (0.8); 3.323 (37.6); 2.672 (0.4); 2.565 (16.0);
2.507 (42.6); 2.503 (52.7); 2.499 (40.4); 2.330 (0.4); 1.989 (0.8); 1.398 (9.5); 1.176 (0.4); 0.000 (4.8)
Example 127: 1H-NMR (400.0 MHz, d6-DMSO):
δ = 9.079 (1.7); 9.064 (3.4); 9.048 (1.7); 7.785 (3.3); 7.766 (4.5); 7.735 (1.5); 7.716 (3.8); 7.698 (2.8); 7.669 (2.9);
7.650 (3.5); 7.627 (3.9); 7.605 (4.8); 7.538 (10.0); 7.519 (3.0); 7.492 (2.2); 7.471 (2.1); 7.450 (1.0); 7.400 (1.4);
7.380 (6.6); 7.361 (5.2); 7.338 (2.1); 7.333 (2.3); 7.320 (2.1); 7.301 (0.7); 5.754 (12.2); 4.124 (1.5); 4.109 (1.7);
4.086 (3.4); 4.071 (3.3); 4.048 (1.8); 4.033 (1.6); 3.569 (0.4); 3.320 (32.1); 3.177 (0.8); 3.164 (0.8); 2.892 (1.1);
2.733 (1.1); 2.671 (0.4); 2.560 (16.0); 2.505 (60.1); 2.502 (67.0); 2.329 (0.4); 1.398 (0.7); 0.000 (4.8)
Example 128: 1H-NMR (400.0 MHz, d6-DMSO):
δ = 9.074 (1.7); 9.059 (3.7); 9.043 (1.8); 7.999 (13.4); 7.983 (13.5); 7.952 (0.6); 7.785 (3.5); 7.764 (5.8); 7.755 (6.3);
7.738 (1.6); 7.720 (6.3); 7.701 (6.2); 7.667 (2.9); 7.648 (3.5); 7.630 (1.3); 7.581 (5.9); 7.560 (4.5); 7.386 (4.1);
7.367 (3.7); 5.754 (2.4); 4.101 (1.5); 4.085 (1.5); 4.063 (3.3); 4.047 (3.2); 4.025 (1.7); 4.009 (1.6); 3.319 (115.2);
2.891 (3.5); 2.731 (3.1); 2.675 (0.8); 2.671 (1.1); 2.666 (0.8); 2.560 (16.0); 2.541 (0.9); 2.506 (122.5); 2.501 (163.3);
2.497 (126.5); 2.333 (0.8); 2.328 (1.1); 2.324 (0.8); 0.000 (6.5)

Formulation Examples

An example for a formulation according to the present invention is the following:

| | |
|---|---|
| 8 mg | compound of Example 3 |
| 0.2 mL | Diethylene glycol monoethyl ether |
| 0.2 mL | Polyoxyl 35 Castor Oil |
| 1.6 mL | physiological sodium chloride solution |

An example for a preparation of such a formulation is as follows. The compound of the present invention was dissolved in 1 part diethylene glycol monoethyl ether and mixed with 1 part Polyoxyl 35 Castor Oil and 8 parts physiological sodium chloride solution.

Such a formulation is suitable for oral or parenteral application.

Formulations of other compounds of the present invention can be prepared in an analogue way and show analogue or identical compositions.

Biological Examples

A. In-Vitro Assays

Cooperia Curticei—Assay (COOPCU)

Solvent: dimethyl sulfoxide

To produce a suitable preparation, 10 mg of active compound are dissolved in 0.5 ml solvent, and the concentrate is diluted with “Ringer's solution” to the desired concentration.

Approximately 40 nematode larvae (Cooperia curticei) are transferred into a test tube containing the compound solution.

After 5 days percentage of larval mortality is recorded. 100% efficacy means all larvae are killed; 0% efficacy means no larvae are killed.

In this test, for example, the following compounds from the preparation examples showed good activity of 100% at an application rate of 20 ppm: 1, 2, 3, 5, 7, 9, 12, 13, 14, 15, 16, 17, 18, 19, 20, 21, 22, 23, 25, 26, 27, 31, 32, 33, 34, 35, 36, 37, 38, 39, 40, 41, 42, 43, 45, 46, 47, 48, 49, 50, 52, 53, 54, 55, 56, 57, 58, 59, 60, 61, 62, 63, 64, 65, 66, 67, 68, 69, 70, 71, 76, 80, 81, 82, 84, 86, 88, 89, 90, 92, 93, 94, 95, 96, 97, 98, 99, 100, 101, 102, 103, 105, 106, 107, 108, 109, 110, 111, 112, 113, 114, 115, 116, 117, 119, 120, 121, 122, 123, 124, 125, 126, 127, 128.

In this test for example, the following compounds from the preparation examples showed good activity of 90% at an application rate of 20 ppm: 51, 74, 78, 87.

In this test for example, the following compounds from the preparation examples showed good activity of 80% at an application rate of 20 ppm: 6, 10, 28, 29, 79, 91.

In this test for example, the following compounds from the preparation examples showed good activity of 100% at an application rate of 4 ppm: 4.

Haemonchus Contortus—Assay (HAEMCO)

Solvent: dimethyl sulfoxide

To produce a suitable preparation, 10 mg of active compound are dissolved in 0.5 ml solvent, and the concentrate is diluted with “Ringer's solution” to the desired concentration.

Approximately 40 larvae of the red stomach worm (Haemonchus contortus) are transferred into a test tube containing compound solution.

After 5 days percentage of larval mortality are recorded. 100% efficacy means all larvae are killed, 0% efficacy means no larvae are killed.

In this test, for example, the following compounds from the preparation examples showed good activity of 100% at an application rate of 20 ppm: 1, 2, 3, 9, 12, 16, 17, 18, 19, 23, 25, 26, 27, 32, 33, 34, 35, 36, 38, 39, 40, 42, 43, 47, 49, 50, 52, 53, 54, 55, 56, 57, 58, 59, 60, 61, 62, 63, 64, 65, 66, 67, 68, 69, 70, 71, 76, 80, 88, 90, 92, 93, 94, 95, 96, 98, 99, 100, 101, 102, 103, 105, 106, 108, 109, 110, 111, 112, 114, 115, 116, 117, 121, 122, 123, 124, 125, 126, 127, 128.

In this test, for example, the following compounds from the preparation examples showed good activity of 90% at an application rate of 20 ppm: 7, 13, 14, 15, 20, 21, 22, 37, 41, 45, 46, 48, 81, 82, 86, 107, 119, 120.

In this test, for example, the following compounds from the preparation examples showed good activity of 80% at an application rate of 20 ppm: 5, 31, 97.

In this test, for example, the following compounds from the preparation examples showed good activity of 90% at an application rate of 4 ppm: 4.

Nippostrongylus Brasiliensis—Assay (NIPOBR)

Adult Nippostrongylus brasiliensis were washed with saline buffer containing 100 U/ml penicillin, 0.1 mg/ml streptomycin and 2.5 μg/ml amphotericin B. Test compounds were dissolved in DMSO and worms were incubated in medium in a final concentration of 10 μg/ml (10 ppm) respectively 1 μg/ml (1 ppm). An aliquot of the medium was used to determine the acetylcholine esterase activity in comparison to a negative control. The principle of measuring acetylcholine esterase as readout for anthelmintic activity was described in Rapson et al (1986) and Rapson et al (1987).

For the following examples, activity (reduction of AChE compared to negative control) was 75% or higher at 10 μg/ml: 1, 2, 3, 7, 12, 13.

1, 2, 3, 5, 7, 12, 13, 17, 21, 22, 23, 25, 27, 32, 33, 35, 36, 37, 38, 39, 41, 42, 43, 47, 50, 52, 53, 55, 56, 57, 59, 61, 62, 63, 64, 65, 66, 67, 68, 69, 70, 71, 76, 80, 82, 88, 90, 93, 94, 95, 96, 98, 99, 100, 101, 103, 105, 106, 108, 109, 110, 111, 112, 114, 115, 116, 119, 120, 121, 122, 123, 124, 125, 126, 127

Dirofilaria Immitis Microfilariae—Assay (DIROM L1)

≧250 Dirofilaria immitis microfilariae, which were freshly purified from blood, were added to wells of a microtitre plate containing a nutrient medium and the test compound in DMSO. Compounds were tested in a five point concentration-response assay in duplicate. Larvae exposed to DMSO and no test compounds were used as negative controls. Larvae were evaluated after 72 h of incubation with the compound. Efficacy was determined as the reduction of motility in comparison to the negative control. Based on the evaluation of five concentrations, concentration-response curves as well as EC₅₀-values were calculated.

For the following examples, the EC50 was <10 ppm: 12, 25, 38, 43, 69, 121, 124, 125.

For the following examples, the EC50 was <1 ppm: 1, 2, 3, 33, 126.

Meloidogyne Incognita—Assay (MELGIN)

Solvent: 125.0 parts by weight of acetone

To produce a suitable preparation, 1 part by weight of active compound is mixed with the stated amount of solvent, and the concentrate is diluted with water to the desired concentration.

Vessels are filled with sand, a solution of the active ingredient, a suspension containing eggs and larvae of the southern root-knot nematode (Meloidogyne incognita) and salad seeds. The salad seeds germinate and the seedlings grow. Galls develop in the roots.

After 14 days the nematicidal activity is determined on the basis of the percentage of gall formation. 100% means no galls were found and 0% means the number of galls found on the roots of the treated plants was equal to that in untreated control plants.

In this test, for example, the following compounds from the preparation examples showed good activity of 100% at an application rate of 20 ppm: 9, 25, 27, 32, 35, 38, 43, 49, 50, 52, 53, 60, 61, 62, 63, 64, 65, 68,69,94,95,101,105,109,112,115,117,119,120,121,122,123,124,125.

In this test, for example, the following compounds from the preparation examples showed good activity of 90% at an application rate of 20 ppm: 17, 47, 70, 108, 126, 127.

B. In-Vivo Assay

Haemonchus Contortus/Trichostrongylus Colubriformis/Gerbil

Gerbils, experimentally infected with Haemonchus and/or Trichostrongylus, were treated once during late prepatency. Test compounds were formulated as solutions or suspensions and applied subcutaneously or intraperitoneally.

Efficacy was determined per group as reduction of worm count in stomach and small intestine, respectively, after necropsy compared to worm count in an infected and placebo-treated control group.

The following examples were tested and had an activity of 90% or higher at the given treatment:

| | | Trichostrongylus |
|---|---|---|
| Treatment | Haemonchus contortus | colubriformis |
| 20 mg/kg | 3, 7, 12, 32, 33, 38, 109 | 3, 7, 32, 33 |
| intraperitoneally | | |
| 10 mg/kg | 3, 32, 33, 36, 62, 70, 109 | 3, 33, 62, 109 |
| subcutaneously | | |

1. A compound of formula (I) 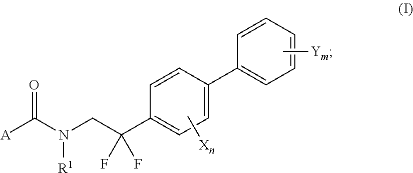
wherein
R¹ is selected from the group consisting of hydrogen, —CHO, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, C₃-C₆-halogenocycloalkyl having 1 to 5 halogen atoms, C₃-C₄-alkenyl, C₃-C₄-alkynyl, C₁-C₄-alkoxy-C₁-C₄-alkyl, C₃-C₆-cycloalkyl-C₁-C₃-alkyl, cyano-C₁-C₄-alkyl, amino-C₁-C₄-alkyl, C₁-C₄-alkylamino-C₁-C₄-alkyl, di-(C₁-C₄-alkyl)amino-C₁-C₄-alkyl, C₁-C₄-alkylcarbonyl, C₁-C₄-halogenoalkylcarbonyl having 1 to 5 halogen atoms, C₁-C₄-alkoxycarbonyl, benzyloxycarbonyl, C₁-C₄-alkoxy-C₁-C₄-alkylcarbonyl, —S (O)₂—C₁-C₄-alkyl, and —S(O)₂—C₁-C₄-halogenoalkyl having 1 to 5 halogen atoms;
n is 1, 2, or 3;
each X is independently selected from the group consisting of halogen, nitro, cyano, hydroxy, amino, —SH, —SF₅, —CHO, —OCHO, —NHCHO, —COOH, —CONH₂, —CONH(OH), —OCONH₂, (hydroxyimino)-C₁-C₆-alkyl, C₁-C₈-alkyl, C₁-C₈-halogenoalkyl having 1 to 5 halogen atoms, C₂-C₈-alkenyl, C₂-C₈-alkynyl, C₁-C₈-alkylamino, di-(C₁-C₈-alkyl)amino, C₁-C₈-alkoxy, C₁-C₈-halogenoalkoxy having 1 to 5 halogen atoms, C₂-C₈-alkenyloxy, C₂-C₈-halogenoalkenyloxy having 1 to 5 halogen atoms, C₃-C₈-alkynyloxy, C₃-C₈-halogenoalkynyloxy having 1 to 5 halogen atoms, C₃-C₈-cycloalkyl, C₃-C₈-halogenocycloalkyl having 1 to 5 halogen atoms, C₁-C₈-alkylcarbonyl, C₁-C₈-halogenoalkylcarbonyl having 1 to 5 halogen atoms, —CONH(C₁-C₈-alkyl), —CON(C₁-C₈-alkyl)₂, —CONH(OC₁-C₈-alkyl), —CON(OC₁-C₈-alkyl)(C₁-C₈-alkyl), C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, —OCONH(C₁-C₈-alkyl), —OCON(C₁-C₈-alkyl)₂, —OCONH(OC₁-C₈-alkyl), —OCO(OC₁-C₈-alkyl), —S—C₁-C₈-alkyl, —S—C₁-C₈-halogenoalkyl having 1 to 5 halogen atoms, —S(O)—C₁-C₈-alkyl, —S(O)—C₁-C₈-halogenoalkyl having 1 to 5 halogen atoms, —S(O)₂—C₁-C₈-alkyl, —S(O)₂—C₁-C₈-halogenoalkyl having 1 to 5 halogen atoms, (C₁-C₆-alkoxyimino)-C₁-C₆-alkyl, (C₂-C₆-alkenyloxyimino)-C₁-C₆-alkyl, (C₃-C₆-alkynyloxyimino)-C₁-C₆-alkyl, (benzyloxyimino)-C₁-C₆-alkyl, benzyloxy, —S-benzyl, benzylamino, phenoxy, —S-phenyl, and phenylamino;
m is 0, 1, 2, 3, or 4; and
each Y is independently selected from the group consisting of hydrogen, halogen, nitro, cyano, hydroxy, amino, —SH, —SF₅, —CHO, —OCHO, —NHCHO, —COOH, —CONH₂, —CONH(OH), —OCONH₂, (hydroxyimino)-C₁-C₆-alkyl, C₁-C₈-alkyl, C₁-C₈-halogenoalkyl having 1 to 5 halogen atoms, C₂-C₈-alkenyl, C₂-C₈-alkynyl, C₁-C₈-alkylamino, di-(C₁-C₈-alkyl)amino, C₁-C₈-alkoxy, C₁-C₈-halogenoalkoxy having 1 to 5 halogen atoms, C₂-C₈-alkenyloxy, C₂-C₈-halogenoalkenyloxy having 1 to 5 halogen atoms, C₃-C₈-alkynyloxy, C₃-C₈-halogenoalkynyloxy having 1 to 5 halogen atoms, C₃-C₈-cycloalkyl, C₃-C₈-halogenocycloalkyl having 1 to 5 halogen atoms, C₁-C₈-alkylcarbonyl, C₁-C₈-halogenoalkylcarbonyl having 1 to 5 halogen atoms, —CONH(C₁-C₈-alkyl), —CON(C₁-C₈-alkyl)₂, —CONH(OC₁-C₈-alkyl), —CON(OC₁-C₈-alkyl)(C₁-C₈-alkyl), C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, —OCONH(C₁-C₈-alkyl), —OCON(C₁-C₈-alkyl)₂, —OCONH(OC₁-C₈-alkyl), —OCO(OC₁-C₈-alkyl), —S—C₁-C₈-alkyl, —S—C₁-C₈-halogenoalkyl having 1 to 5 halogen atoms, —S(O)—C₁-C₈-alkyl, —S(O)—C₁-C₈-halogenoalkyl having 1 to 5 halogen atoms, —S(O)₂—C₁-C₈-alkyl, —S(O)₂—C₁-C₈-halogenoalkyl having 1 to 5 halogen atoms, —CH₂—S—C₁-C₈-alkyl, —CH₂—S(O)—C₁-C₈-alkyl, —CH₂—S(O)₂—C₁-C₈-alkyl, (C₁-C₆-alkoxyimino)-C₁-C₆-alkyl, (C₂-C₆-alkenyloxyimino)-C₁-C₆-alkyl, and (C₃-C₆-alkynyloxyimino)-C₁-C₆-alkyl; wherein two Y may additionally be selected from —O—C₁-C₃-alkoxy and —O—C₁-C₃-halogenalkoxy having 1 to 5 halogen atoms, with each oxy function being the connecting atom of the individual Y, thereby forming an annelated 5-, 6- or 7-membered ring with the phenyl moiety that they are connected to; or
each Y is independently selected from the group consisting of hydrogen, halogen, cyano, C₁-C₆-alkyl, C₁-C₆-halogenoalkyl having 1 to 5 halogen atoms, and a 4 to 7 membered heterocyclic ring system with 1 to 3 heteroatoms, but not O—O, O—S or 3 heteroatoms in a row, independently substituted by oxo, thiono, or 1 to 12 substituents Z independently selected from the group consisting of hydrogen, halogen, cyano, hydroxy, amino, —SH, —CHO, —COOH, —CONH₂, —CONH(OH), —OCONH₂, (hydroxyimino)-C₁-C₆-alkyl, C₁-C₆-alkyl, C₁-C₆-halogenoalkyl having 1 to 5 halogen atoms, C₂-C₆-alkenyl, C₂-C₆-alkynyl, C₁-C₆-alkylamino, di-(C₁-C₆-alkyl)amino, C₁-C₆-alkoxy, C₁-C₆-halogenoalkoxy having 1 to 5 halogen atoms, C₂-C₆-alkenyloxy, C₂-C₆-halogenoalkenyloxy having 1 to 5 halogen atoms, C₃-C₆-alkynyloxy, C₃-C₆-halogenoalkynyloxy having 1 to 5 halogen atoms, C₃-C₆-cycloalkyl, C₃-C₆-halogenocycloalkyl having 1 to 5 halogen atoms, C₁-C₆-alkylcarbonyl, C₁-C₆-halogenoalkylcarbonyl having 1 to 5 halogen atoms, —CONH(C₁-C₆-alkyl), —CON(C₁-C₆-alkyl)₂, —CONH(OC₁-C₆-alkyl), —CON(OC₁-C₆-alkyl)(C₁-C₆-alkyl), C₁-C₆-alkoxycarbonyl, C₁-C₆-halogenoalkoxycarbonyl having 1 to 5 halogen atoms, C₁-C₆-alkylcarbonyloxy, C₁-C₆-halogenoalkylcarbonyloxy having 1 to 5 halogen atoms, C₁-C₆-alkylcarbonylamino, C₁-C₆-halogenoalkylcarbonylamino having 1 to 5 halogen atoms, —OCONH(C₁-C₆-alkyl), —OCON(C₁-C₆-alkyl)₂, —OCONH(OC₁-C₆-alkyl), —OCO(OC₁-C₆-alkyl), —S—C₁-C₆-alkyl, —S—C₁-C₆-halogenoalkyl having 1 to 5 halogen atoms, —S(O)—C₁-C₆-alkyl, —S(O)—C₁-C₆-halogenoalkyl having 1 to 5 halogen atoms, —S(O)₂—C₁-C₆-alkyl, and —S(O)₂—C₁-C₆-halogenoalkyl having 1 to 5 halogen atoms; and
A represents a phenyl group of the formula (A1) 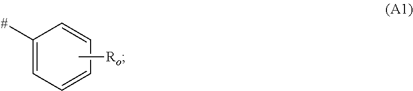
in which
# depicts the bond which connects A to the rest of the molecule;
o is 0, 1, 2, 3, 4, or 5; and
each R is independently selected from the group consisting of halogen, nitro, —OH, NH₂, SH, SF₅, CHO, OCHO, NHCHO, COOH, cyano, C₁-C₈-alkyl, C₁-C₈-halogenoalkyl having 1 to 9 halogen atoms, C₂-C₈-alkenyl, C₂-C₈-alkynyl, C₃-C₆-cycloalkyl, —S—C₁-C₈-alkyl, —S—C₁-C₈-halogenoalkyl having 1 to 5 halogen atoms, C₁-C₈-alkoxy, C₁-C₈-halogenoalkoxy having 1 to 5 halogen atoms, C₁-C₈-alkoxy-C₂-C₈-alkenyl, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, —S(O)—C₁-C₈-alkyl, —S(O)—C₁-C₈-halogenoalkyl having 1 to 5 halogen atoms, —S(O)₂—C₁-C₈-alkyl, —S(O)₂—C₁-C₈-halogenoalkyl having 1 to 5 halogen atoms, C₁-C₈-alkylsulfonamide, —NH(C₁-C₈-alkyl), N(C₁-C₈-alkyl)₂, phenyl (optionally substituted by C₁-C₆-alkoxy) and phenoxy; wherein two R bonded to adjacent carbon atoms may together represent —O(CH₂)ₚO—, wherein p represents 1 or 2;
A represents a heterocycle of the formula (Het-1) 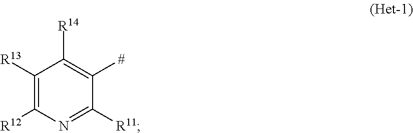
in which
# depicts the bond which connects A to the rest of the molecule;
R¹¹ is selected from the group consisting of hydrogen, halogen, hydroxy, cyano, C₁-C₄-alkyl, C₁-C₄-halogenoalkyl having 1 to 5 halogen atoms, C₁-C₄ alkoxy, —S—C₁-C₅-alkyl, S(O)—C₁-C₄-alkyl, —S(O)₂—C₁-C₄-alkyl, —S—C₂-C₅-alkenyl, —S—C₁-C₄-halogenoalkyl having 1 to 5 halogen atoms, and C₁-C₄-halogenoalkoxy having 1 to 5 halogen atoms; and
R¹², R¹³, and R¹⁴, which may be the same or be different, are selected from the group consisting of hydrogen, halogen, cyano, C₁-C₄-alkyl, C₁-C₄-halogenoalkyl having 1 to 5 halogen atoms, C₁-C₄-alkoxy, —S—C₁-C₄-alkyl, C₁-C₄-halogenoalkoxy having 1 to 5 halogen atoms, —S(O)—C₁-C₄-alkyl, and —S(O)₂—C₁-C₄-alkyl;
A represents a heterocycle of the formula (Het-2) 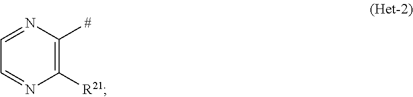
in which
# depicts the bond which connects A to the rest of the molecule; and
R²¹ is selected from the group consisting of hydrogen, halogen, C₁-C₄-alkyl and C₁-C₄-halogenoalkyl having 1 to 5 halogen atoms; or
A represents a heterocycle of the formula (Het-3) 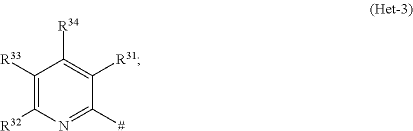
in which
# depicts the bond which connects A to the rest of the molecule;
R³¹ is selected from the group consisting of hydrogen, halogen, hydroxy, cyano, C₁-C₄-alkyl, C₁-C₄-halogenoalkyl having 1 to 5 halogen atoms, C₁-C₄ alkoxy, —S—C₁-C₅-alkyl, S(O)—C₁-C₄-alkyl, —S(O)₂—C₁-C₄-alkyl, —S—C₂—C₅-alkenyl, —S—C₁-C₄-halogenoalkyl having 1 to 5 halogen atoms, and C₁-C₄-halogenoalkoxy having 1 to 5 halogen atoms; and
R³², R³³, and R³⁴, which may be the same or be different, are selected from the group consisting of hydrogen, halogen, cyano, C₁-C₄-alkyl, C₁-C₄-halogenoalkyl having 1 to 5 halogen atoms, C₁-C₄-alkoxy, —S—C₁-C₄-alkyl, C₁-C₄-halogenoalkoxy having 1 to 5 halogen atoms, —S(O)—C₁-C₄-alkyl, and —S(O)₂—C₁-C₄-alkyl;
and salts, solvates, solvates of the salts, N-oxides, metal complexes or metalloid complexes thereof.

2. The compound according to claim 1, wherein
A represents a phenyl group of formula (A1) 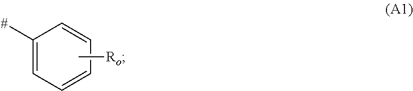
in which
# depicts the bond which connects A to the rest of the molecule;
o is 0, 1, or 2; and
each R is independently selected from the group consisting of halogen, nitro, C₁-C₄-halogenoalkyl having 1 to 5 halogen atoms, and C₁-C₄-halogenoalkoxy having 1 to 5 halogen atoms; or
A represents a heterocycle of the formula (Het-1) 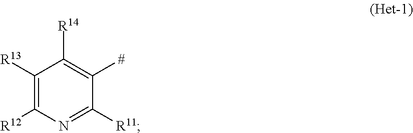
in which
# depicts the bond which connects A to the rest of the molecule;
R¹¹ is halogen or C₁-C₄-halogenoalkyl having 1 to 5 halogen atoms; and
R¹², R¹³, and R¹⁴ are hydrogen; or
A represents a heterocycle of the formula (Het-2) 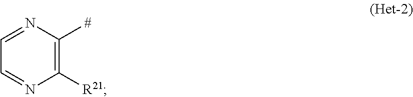
in which
# depicts the bond which connects A to the rest of the molecule; and
R²¹ is C₁-C₄-halogenoalkyl having 1 to 5 halogen atoms; or
A represents a heterocycle of the formula (Het-3) 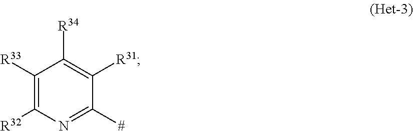
in which
# depicts the bond which connects A to the rest of the molecule;
R³¹ is C₁-C₄-halogenoalkyl having 1 to 5 halogen atoms; and
R³², R³³, and R³⁴ are hydrogen;
R¹ is selected from the group consisting of hydrogen, C₁-C₄-alkyl, C₃-C₄-alkynyl, C₃-C₆-cycloalkyl, C₁-C₄-alkylcarbonyl, C₁-C₄-alkoxy-C₁-C₄-alkyl, and C₁-C₄-alkoxycarbonyl;
n is 1 or 2;
each X is independently selected from the group consisting of halogen, nitro, cyano, C₁-C₄-alkyl, C₁-C₄-halogenoalkyl having 1 to 5 halogen atoms, C₂-C₄-alkenyl, C₂-C₄-alkynyl, C₁-C₄-alkylamino, di-(C₁-C₄-alkyl)amino, C₁-C₄-alkoxy, C₁-C₄-halogenoalkoxy having 1 to 5 halogen atoms, C₂-C₄-alkenyloxy, C₂-C₄-halogenoalkenyloxy having 1 to 5 halogen atoms, C₃-C₄-alkynyloxy, C₃-C₄-halogenoalkynyloxy having 1 to 5 halogen atoms, C₃-C₆-cycloalkyl, C₃-C₆-halogenocycloalkyl having 1 to 5 halogen atoms, C₁-C₄-alkylcarbonyl, C₁-C₄-halogenoalkylcarbonyl having 1 to 5 halogen atoms, —CONH(C₁-C₄-alkyl), —CON(C₁-C₄-alkyl)₂, —CONH(OC₁-C₄-alkyl), —CON(OC₁-C₄-alkyl)(C₁-C₄-alkyl), C₁-C₄-alkoxycarbonyl, C₁-C₄-halogenoalkoxycarbonyl having 1 to 5 halogen atoms, C₁-C₄-alkylcarbonyloxy, C₁-C₄-halogenoalkylcarbonyloxy having 1 to 5 halogen atoms, C₁-C₄-alkylcarbonylamino, C₁-C₄-halogenoalkylcarbonylamino having 1 to 5 halogen atoms, —OCONH(C₁-C₄-alkyl), —OCON(C₁-C₄-alkyl)₂, —OCONH(OC₁-C₄-alkyl), —OCO(OC₁-C₄-alkyl), —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, (C₁-C₄-alkoxyimino)-C₁-C₄-alkyl, (C₂-C₆-alkenyloxyimino)-C₁-C₄-alkyl, (C₃-C₆-alkynyloxyimino)-C₁-C₄-alkyl, (benzyloxyimino)-C₁-C₆-alkyl, benzyloxy, —S-benzyl, benzylamino, phenoxy, —S-phenyl, and phenylamino;
m is 1, 2, or 3; and
each Y is independently selected from the group consisting of hydrogen, halogen, nitro, cyano, hydroxy, amino, —SH, —SF₅, —CHO, —OCHO, —NHCHO, —COOH, —CONH₂, —CONH(OH), —OCONH₂, (hydroxyimino)-C₁-C₆-alkyl, C₁-C₆-alkyl, C₁-C₆-halogenoalkyl having 1 to 5 halogen atoms, C₂-C₆-alkenyl, C₂-C₆-alkynyl, C₁-C₆-alkylamino, di-(C₁-C₆-alkyl)amino, C₁-C₆-alkoxy, C₁-C₆-halogenoalkoxy having 1 to 5 halogen atoms, C₂-C₆-alkenyloxy, C₂-C₈-halogenoalkenyloxy having 1 to 5 halogen atoms, C₃-C₆-alkynyloxy, C₃-C₆-halogenoalkynyloxy having 1 to 5 halogen atoms, C₃-C₆-cycloalkyl, C₃-C₆-halogenocycloalkyl having 1 to 5 halogen atoms, C₁-C₆-alkylcarbonyl, C₁-C₆-halogenoalkylcarbonyl having 1 to 5 halogen atoms, —CONH(C₁-C₆-alkyl), —CON(C₁-C₆-alkyl)₂, —CONH(OC₁-C₆-alkyl), —CON(OC₁-C₆-alkyl)(C₁-C₆-alkyl), C₁-C₆-alkoxycarbonyl, C₁-C₆-halogenoalkoxycarbonyl having 1 to 5 halogen atoms, C₁-C₆-alkylcarbonyloxy, C₁-C₆-halogenoalkylcarbonyloxy having 1 to 5 halogen atoms, C₁-C₆-alkylcarbonylamino, C₁-C₆-halogenoalkylcarbonylamino having 1 to 5 halogen atoms, —OCONH(C₁-C₆-alkyl), —OCON(C₁-C₆-alkyl)₂, —OCONH(OC₁-C₆-alkyl), —OCO(OC₁-C₆-alkyl), —S—C₁-C₆-alkyl, —S—C₁-C₆-halogenoalkyl having 1 to 5 halogen atoms, —S(O)—C₁-C₆-alkyl, —S(O)—C₁-C₆-halogenoalkyl having 1 to 5 halogen atoms, —S(O)₂—C₁-C₆-alkyl, —S(O)₂—C₁-C₆-halogenoalkyl having 1 to 5 halogen atoms, —CH₂—S—C₁-C₆-alkyl, —CH₂—S(O)—C₁-C₆-alkyl, —CH₂—S(O)₂—C₁-C₆-alkyl, (C₁-C₄-alkoxyimino)-C₁-C₄-alkyl, (C₂-C₄-alkenyloxyimino)-C₁-C₄-alkyl, and (C₃-C₆-alkynyloxyimino)-C₁-C₄-alkyl; wherein two Y may additionally be selected from —O—C₁-C₃-alkoxy and —O—C₁-C₃-halogenalkoxy having 1 to 5 halogen atoms, with each oxy function being the connecting atom of the individual Y, thereby forming an annelated 5-, 6- or 7-membered ring with the phenyl moiety that they are connected to; or
each Y is independently selected from the group consisting of hydrogen, halogen, cyano, C₁-C₆-alkyl, C₁-C₆-halogenoalkyl having 1 to 5 halogen atoms, 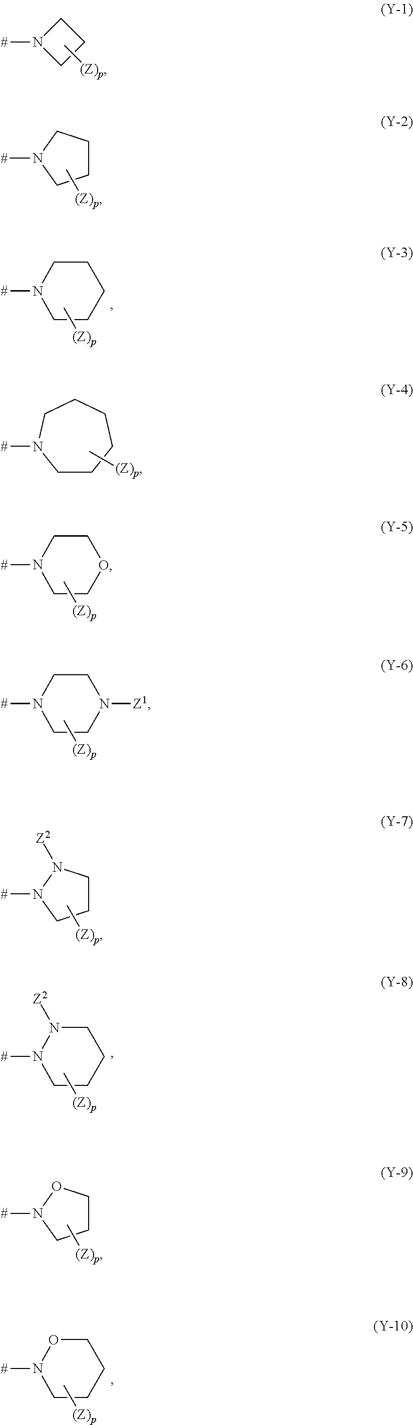 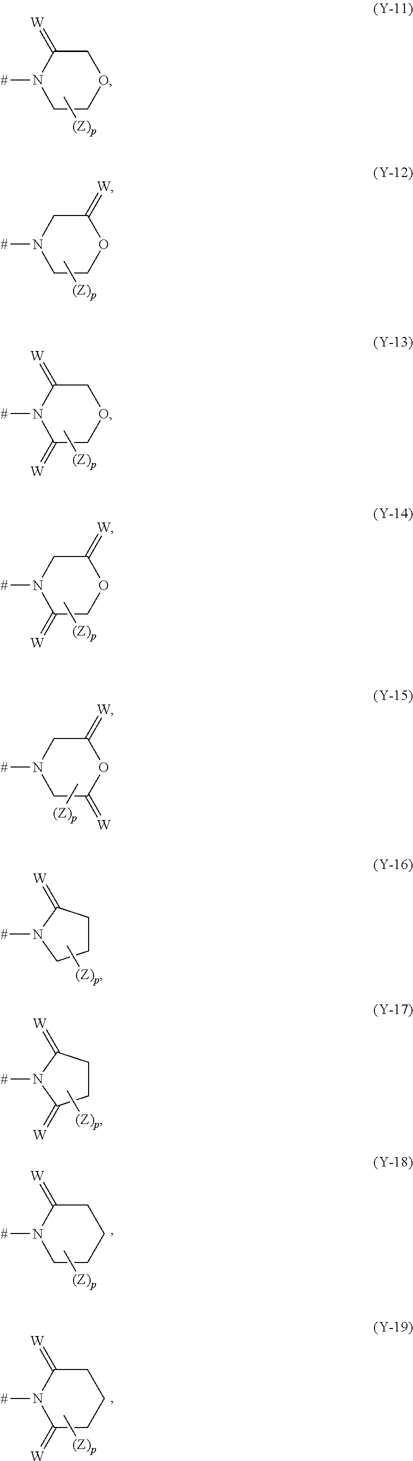 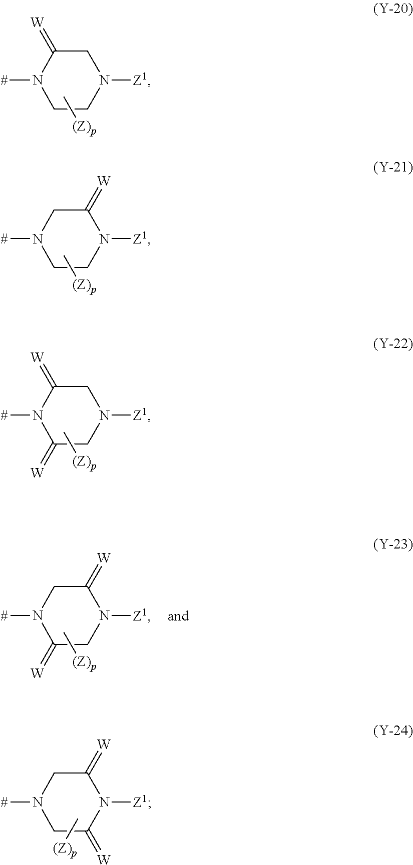
in which
# depicts the bond which connects Y to the rest of the molecule;
W is oxygen or sulfur;
p is 0, 1, 2, 3, 4, 5, or 6;
Z is independently selected from the group consisting of hydrogen, halogen, cyano, hydroxy, amino, —SH, —CHO, —COOH, —CONH₂, —CONH(OH), —OCONH₂, (hydroxyimino)-C₁-C₆-alkyl, C₁-C₆-alkyl, C₁-C₆-halogenoalkyl having 1 to 5 halogen atoms, C₁-C₆-alkylamino, di-(C₁-C₆-alkyl)amino, C₁-C₆-alkoxy, C₁-C₆-halogenoalkoxy having 1 to 5 halogen atoms, C₃-C₆-cycloalkyl, C₃-C₆-halogenocycloalkyl having 1 to 5 halogen atoms, —CONH(C₁-C₆-alkyl), —CON(C₁-C₆-alkyl)₂, —S—C₁-C₆-alkyl, —S—C₁-C₆-halogenoalkyl having 1 to 5 halogen atoms, —S(O)—C₁-C₆-alkyl, —S(O)—C₁-C₆-halogenoalkyl having 1 to 5 halogen atoms, —S(O)₂—C₁-C₆-alkyl, and —S(O)₂—C₁-C₆-halogenoalkyl having 1 to 5 halogen atoms;
Z¹ is independently selected from the group consisting of hydrogen, C₁-C₆-alkyl, C₁-C₆-halogenoalkyl having 1 to 5 halogen atoms, C₁-C₆-alkylamino, di-(C₁-C₆-alkyl)amino, C₁-C₆-alkoxy, C₁-C₆-alkylcarbonyl, C₃-C₆-cycloalkyl, C₃-C₆-halogenocycloalkyl having 1 to 5 halogen atoms, —CONH(C₁-C₆-alkyl), and —CON(C₁-C₆-alkyl)₂; and
Z² is independently selected from the group consisting of hydrogen, C₁-C₆-alkyl, C₁-C₆-halogenoalkyl having 1 to 5 halogen atoms, C₃-C₆-cycloalkyl, C₃-C₆-halogenocycloalkyl having 1 to 5 halogen atoms, —CONH(C₁-C₆-alkyl), and —CON(C₁-C₆-alkyl)₂;
and salts, solvates, solvates of the salts, N-oxides, metal complexes or metalloid complexes thereof.

3. The compound according to claim 1, wherein
A represents a phenyl group of formula (A1) 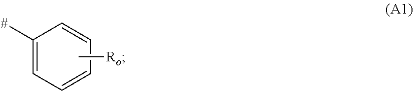
in which
# depicts the bond which connects A to the rest of the molecule;
o is 0, 1, or 2; and
each R is independently selected from the group consisting of halogen, nitro, C₁-C₄-halogenoalkyl having 1 to 5 halogen atoms, and C₁-C₄-halogenoalkoxy having 1 to 5 halogen atoms;
A represents a heterocycle of the formula (Het-1) 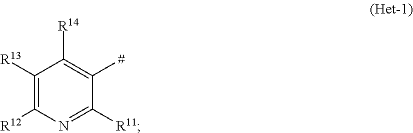
in which
# depicts the bond which connects A to the rest of the molecule;
R¹¹ is halogen or C₁-C₄-halogenoalkyl having 1 to 5 halogen atoms; and
R¹², R¹³, and R¹⁴ are hydrogen;
A represents a heterocycle of the formula (Het-2) 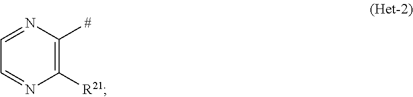
in which
# depicts the bond which connects A to the rest of the molecule; and
R²¹ is C₁-C₄-halogenoalkyl having 1 to 5 halogen atoms; or
A represents a heterocycle of the formula (Het-3) 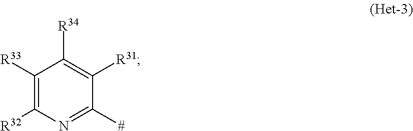
in which
# depicts the bond which connects A to the rest of the molecule;
R³¹ is C₁-C₄-halogenoalkyl having 1 to 5 halogen atoms; and
R³², R³³, and R³⁴ are hydrogen;
R¹ is selected from the group consisting of hydrogen, C₁-C₄-alkyl, C₃-C₄-alkynyl, C₃-C₆-cycloalkyl, C₁-C₄-alkylcarbonyl, C₁-C₄-alkoxy-C₁-C₄-alkyl, and C₁-C₄-alkoxycarbonyl;
n is 1 or 2;
each X is independently selected from the group consisting of halogen, cyano, C₁-C₄-alkyl, C₁-C₄-alkoxy and C₁-C₄-halogenoalkyl having 1 to 5 halogen atoms;
m is 1, 2, or 3; and
each Y is independently selected from the group consisting of hydrogen, halogen, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —CONH(C₁-C₄-alkyl), —CON(C₁-C₄-alkyl)₂, —CONH(OC₁-C₄-alkyl), —CON(OC₁-C₄-alkyl)(C₁-C₆-alkyl), —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, and C₁-C₄-alkylcarbonylamino; wherein two Y may additionally be selected from —O—C₁-C₂-alkoxy and —O—C₁-C₂-halogenalkoxy having 1 to 5 halogen atoms, with each oxy function being the connecting atom of the individual Y, thereby forming an annelated 5- or 6-membered ring with the phenyl moiety that they are connected to; or
each Y is independently selected from the group consisting of hydrogen, halogen, cyano, C₁-C₄-alkyl, C₁-C₄-halogenoalkyl having 1 to 5 halogen atoms, 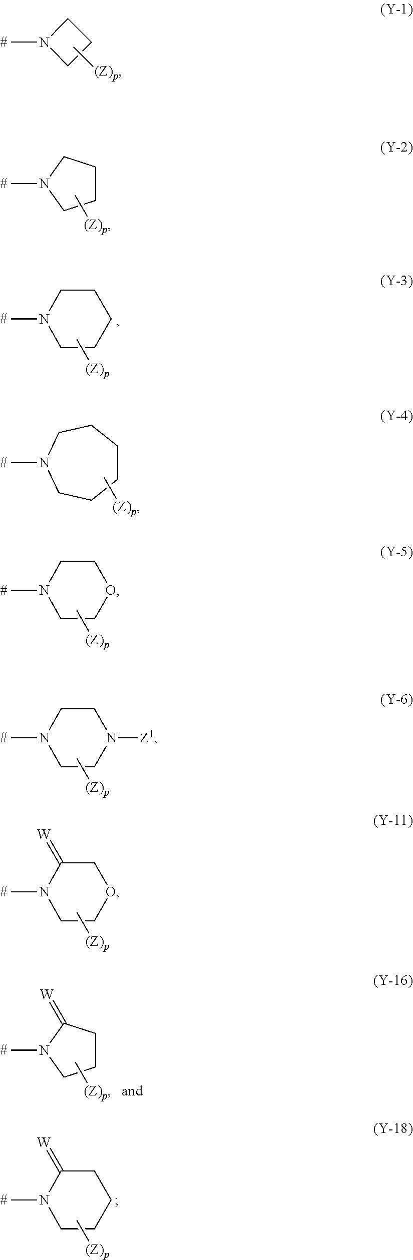
in which
# depicts the bond which connects Y to the rest of the molecule;
W is oxygen;
p is 0, 1, 2, 3, 4, 5, or 6;
Z is independently selected from the group consisting of hydrogen, halogen, cyano, hydroxy, amino, —SH, —CHO, —COOH, —CONH₂, —CONH(OH), —OCONH₂, (hydroxyimino)-C₁-C₄-alkyl, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, C₃-C₆-cycloalkyl, C₃-C₆-halogenocycloalkyl having 1 to 5 halogen atoms, —CONH(C₁-C₄-alkyl), —CON(C₁-C₄-alkyl)₂, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, and —S(O)₂—C₁-C₄-halogenoalkyl having 1 to 5 halogen atoms; and
Z¹ is independently selected from the group consisting of hydrogen, C₁-C₆-alkyl, C₁-C₆-halogenoalkyl having 1 to 5 halogen atoms, C₁-C₆-alkylamino, di-(C₁-C₆-alkyl)amino, C₁-C₆-alkoxy, C₁-C₆-alkylcarbonyl, C₃-C₆-cycloalkyl, C₃-C₆-halogenocycloalkyl having 1 to 5 halogen atoms, —CONH(C₁-C₆-alkyl), and —CON(C₁-C₆-alkyl)₂;
and salts, solvates, solvates of the salts, N-oxides, metal complexes or metalloid complexes thereof.

4. The compound according to claim 1, wherein
A represents a phenyl group of formula (A1) 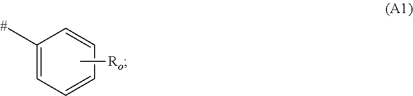
in which
# depicts the bond which connects A to the rest of the molecule;
o is 1 or 2; and
each R is independently selected from the group consisting of halogen, nitro, —CF₃, —OCF₃, and —CHF₂;
A represents a heterocycle of the formula (Het-1) 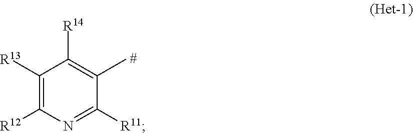
in which
# depicts the bond which connects A to the rest of the molecule;
R¹¹ is halogen or CF₃; and
R¹², R¹³, and R¹⁴ are hydrogen; or
A represents a heterocycle of the formula (Het-2) 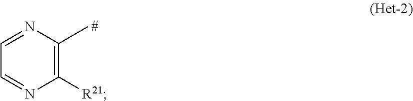
in which
# depicts the bond which connects A to the rest of the molecule; and
R²¹ is CF₃; or
A represents a heterocycle of the formula (Het-3) 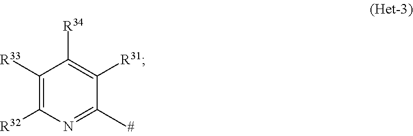
in which
# depicts the bond which connects A to the rest of the molecule;
R³¹ is CF₃; and
R³², R³³, and R³⁴ are hydrogen;
R¹ is hydrogen;
n is 1 or 2;
each X is independently selected from the group consisting of halogen, cyano, CH₃, OCH₃, and CF₃;
m is 1, 2, or 3; and
each Y is independently selected from the group consisting of hydrogen, halogen, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, and C₁-C₄-alkylcarbonylamino; wherein two Y may additionally be selected from —O—CH₂—O— and —O—CF₂—O—, with each oxy function being the connecting atom of the individual Y, thereby forming an annelated 5-membered ring with the phenyl moiety that they are connected to; or
each Y is independently selected from the group consisting of hydrogen, fluoro, chloro, cyano, CF₃, 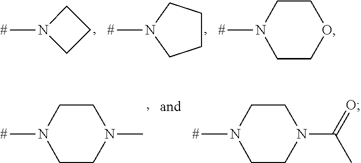
and salts, solvates, solvates of the salts, N-oxides, metal complexes or metalloid complexes thereof.

5. The compound according to claim 1, wherein
A is selected from the group consisting of 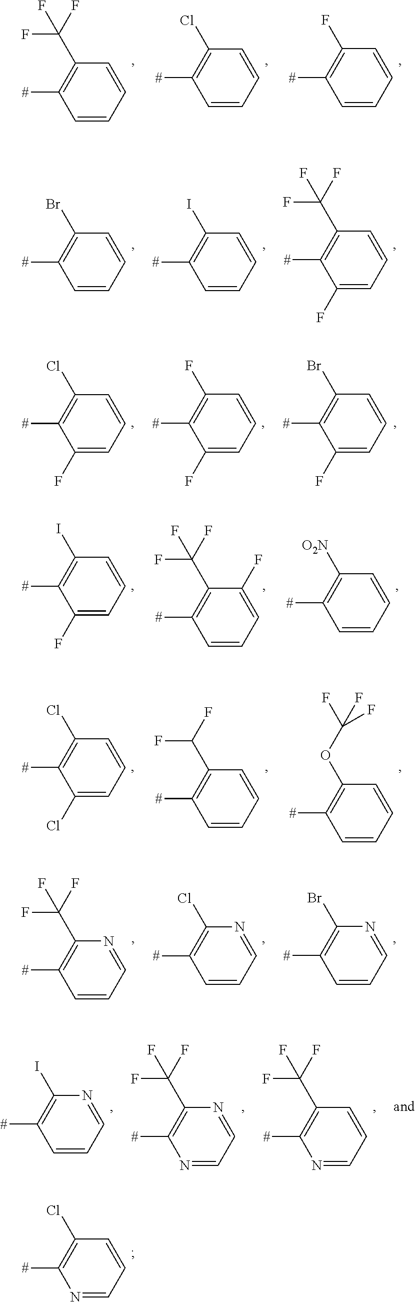
in which
# depicts the bond which connects A to the rest of the molecule;
R¹ is hydrogen;
n is 1 or 2;
each X is independently selected from the group consisting of chloro, fluoro, cyano, CH₃, OCH₃, and CF₃;
m is 1, 2, or 3; and
each Y is independently selected from the group consisting of hydrogen, halogen, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylcarbonylamino, wherein two Y may additionally be selected from —O—CH₂—O— and —O—CF₂—O—, with each oxy function being the connecting atom of the individual Y, thereby forming an annelated 5-membered ring with the phenyl moiety that they are connected to, or
each Y is independently selected from the group consisting of hydrogen, fluoro, chloro, cyano, CF₃, and 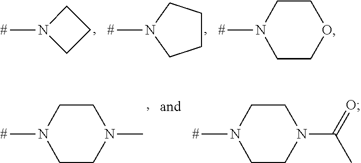
and salts, solvates, solvates of the salts, N-oxides, metal complexes or metalloid complexes thereof.

6. The compound according to claim 1, wherein
A is selected from the group consisting of 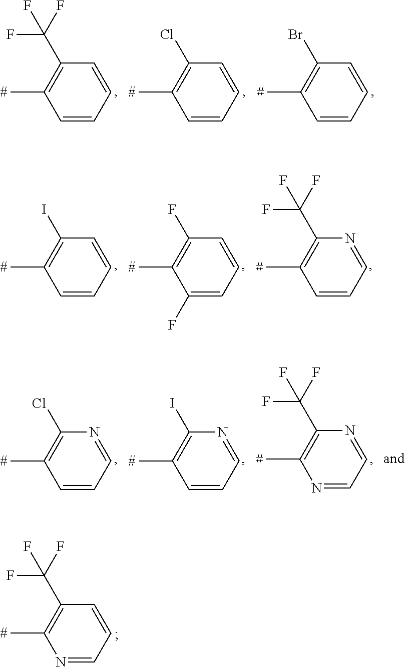
in which
# depicts the bond which connects A to the rest of the molecule;
R¹ is hydrogen;
n is 1 or 2;
each X is independently selected from the group consisting of chloro, fluoro, cyano, CH₃, OCH₃, and CF₃;
m is 1, 2, or 3; and
each Y is independently selected from the group consisting of hydrogen, fluoro, chloro, bromo, cyano, trifluoromethyl, difluoromethyl, methoxy, trifluoromethoxy, difluoromethoxy, methylamino, dimethylamino, methylsulfanyl, trifluoromethylsulfanyl, methylsulfinyl, trifluoromethylsulfinyl, methylsulfonyl, trifluoromethylsulfonyl, and acetylamino; or
each Y is independently selected from the group consisting of hydrogen, fluoro, chloro, cyano, CF₃, 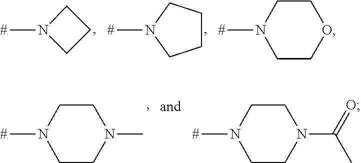
and salts, solvates, solvates of the salts, N-oxides, metal complexes or metalloid complexes thereof.

7. The compound according to claim 1, wherein
A is selected from the group consisting of 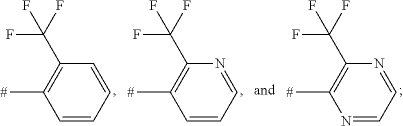
in which
# depicts the bond which connects A to the rest of the molecule;
R¹ is hydrogen;
n is 1 or 2;
each X is independently selected from the group consisting of chloro, fluoro, CH₃, and CF₃;
m is 1, 2, or 3; and
each Y is independently selected from the group consisting of hydrogen, fluoro and chloro;
and salts, solvates, solvates of the salts, N-oxides, metal complexes or metalloid complexes thereof.

8. A compound of formula (I-1) 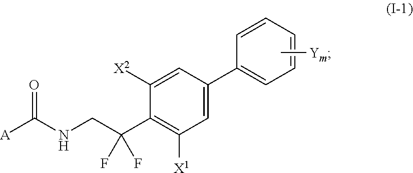
wherein
X¹ is selected from the group consisting of halogen, cyano, C₁-C₄-alkyl, C₁-C₄-alkoxy, and C₁-C₄-halogenoalkyl having 1 to 5 halogen atoms;
X² is selected from the group consisting of hydrogen, halogen, cyano, C₁-C₄-alkyl, C₁-C₄-alkoxy, and C₁-C₄-halogenoalkyl having 1 to 5 halogen atoms;
m is 1, 2, or 3;
each Y is independently selected from the group consisting of hydrogen, halogen, cyano, C₁-C₄-alkyl, C₁-C₄-halogenoalkyl having 1 to 5 halogen atoms, C₁-C₄-alkylamino, di-(C₁-C₄-alkyl)amino, C₁-C₄-alkoxy, C₁-C₄-halogenoalkoxy having 1 to 5 halogen atoms, —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, and C₁-C₄-alkylcarbonylamino; and
A is selected from the group consisting of 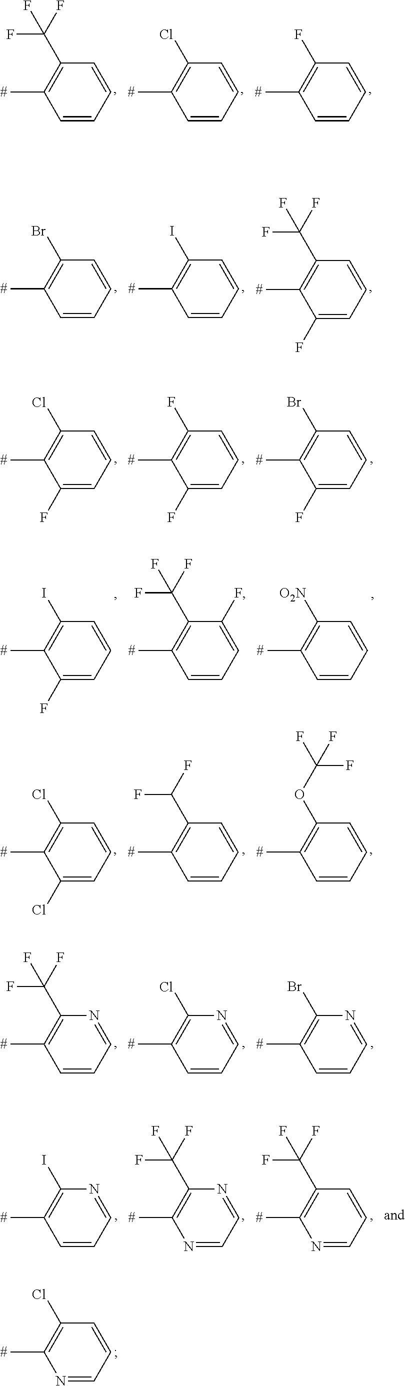
in which
# depicts the bond which connects A to the rest of the molecule;
and salts, solvates, solvates of the salts, N-oxides, metal complexes or metalloid complexes thereof.

9. A method for the control, treatment, or prevention of infections with helminths, comprising administering an effective amount of a compound of formula (I), or a salt, a solvate, a solvate of a salt, an N-oxide, a metal complex, or a metalloid complex thereof, to an animal or human in need thereof 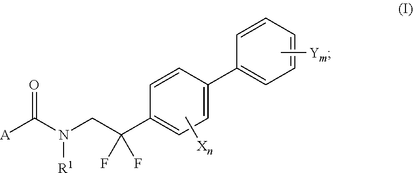
wherein
R¹ is selected from the group consisting of hydrogen, —CHO, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, C₃-C₆-halogenocycloalkyl having 1 to 5 halogen atoms, C₃-C₄-alkenyl, C₃-C₄-alkynyl, C₁-C₄-alkoxy-C₁-C₄-alkyl, C₃-C₆-cycloalkyl-C₁-C₃-alkyl, cyano-C₁-C₄-alkyl, amino-C₁-C₄-alkyl, C₁-C₄-alkylamino-C₁-C₄-alkyl, di-(C₁-C₄-alkyl)amino-C₁-C₄-alkyl, C₁-C₄-alkylcarbonyl, C₁-C₄-halogenoalkylcarbonyl having 1 to 5 halogen atoms, C₁-C₄-alkoxycarbonyl, benzyloxycarbonyl, C₁-C₄-alkoxy-C₁-C₄-alkylcarbonyl, —S(O)₂—C₁-C₄-alkyl, and —S(O)₂—C₁-C₄-halogenoalkyl having 1 to 5 halogen atoms;
n is 0, 1, 2, or 3;
each X is independently selected from the group consisting of hydrogen, halogen, nitro, cyano, hydroxy, amino, —SH, —SF₅, —CHO, —OCHO, —NHCHO, —COOH, —CONH₂, —CONH(OH), —OCONH₂, (hydroxyimino)-C₁-C₆-alkyl, C₁-C₈-alkyl, C₁-C₈-halogenoalkyl having 1 to 5 halogen atoms, C₂-C₈-alkenyl, C₂-C₈-alkynyl, C₁-C₈-alkylamino, di-(C₁-C₈-alkyl)amino, C₁-C₈-alkoxy, C₁-C₈-halogenoalkoxy having 1 to 5 halogen atoms, C₂-C₈-alkenyloxy, C₂-C₈-halogenoalkenyloxy having 1 to 5 halogen atoms, C₃-C₈-alkynyloxy, C₃-C₈-halogenoalkynyloxy having 1 to 5 halogen atoms, C₃-C₈-cycloalkyl, C₃-C₈-halogenocycloalkyl having 1 to 5 halogen atoms, C₁-C₈-alkylcarbonyl, C₁-C₈-halogenoalkylcarbonyl having 1 to 5 halogen atoms, —CONH(C₁-C₈-alkyl), —CON(C₁-C₈-alkyl)₂, —CONH(OC₁-C₈-alkyl), —CON(OC₁-C₈-alkyl)(C₁-C₈-alkyl), C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, —OCONH(C₁-C₈-alkyl), —OCON(C₁-C₈-alkyl)₂, —OCONH(OC₁-C₈-alkyl), —OCO(OC₁-C₈-alkyl), —S—C₁-C₈-alkyl, —S—C₁-C₈-halogenoalkyl having 1 to 5 halogen atoms, —S(O)—C₁-C₈-alkyl, —S(O)—C₁-C₈-halogenoalkyl having 1 to 5 halogen atoms, —S(O)₂—C₁-C₈-alkyl, —S(O)₂—C₁-C₈-halogenoalkyl having 1 to 5 halogen atoms, (C₁-C₆-alkoxyimino)-C₁-C₆-alkyl, (C₂-C₆-alkenyloxyimino)-C₁-C₆-alkyl, (C₃-C₆-alkynyloxyimino)-C₁-C₆-alkyl, (benzyloxyimino)-C₁-C₆-alkyl, benzyloxy, —S-benzyl, benzylamino, phenoxy, —S-phenyl, and phenylamino;
m is 0, 1, 2, 3, or 4; and
each Y is independently selected from the group consisting of hydrogen, halogen, nitro, cyano, hydroxy, amino, —SH, —SF₅, —CHO, —OCHO, —NHCHO, —COOH, —CONH₂, —CONH(OH), —OCONH₂, (hydroxyimino)-C₁-C₆-alkyl, C₁-C₈-alkyl, C₁-C₈-halogenoalkyl having 1 to 5 halogen atoms, C₂-C₈-alkenyl, C₂-C₈-alkynyl, C₁-C₈-alkylamino, di-(C₁-C₈-alkyl)amino, C₁-C₈-alkoxy, C₁-C₈-halogenoalkoxy having 1 to 5 halogen atoms, C₂-C₈-alkenyloxy, C₂-C₈-halogenoalkenyloxy having 1 to 5 halogen atoms, C₃-C₈-alkynyloxy, C₃-C₈-halogenoalkynyloxy having 1 to 5 halogen atoms, C₃-C₈-cycloalkyl, C₃-C₈-halogenocycloalkyl having 1 to 5 halogen atoms, C₁-C₈-alkylcarbonyl, C₁-C₈-halogenoalkylcarbonyl having 1 to 5 halogen atoms, —CONH(C₁-C₈-alkyl), —CON(C₁-C₈-alkyl)₂, —CONH(OC₁-C₈-alkyl), —CON(OC₁-C₈-alkyl)(C₁-C₈-alkyl), C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, —OCONH(C₁-C₈-alkyl), —OCON(C₁-C₈-alkyl)₂, —OCONH(OC₁-C₈-alkyl), —OCO(OC₁-C₈-alkyl), —S—C₁-C₈-alkyl, —S—C₁-C₈-halogenoalkyl having 1 to 5 halogen atoms, —S(O)—C₁-C₈-alkyl, —S(O)—C₁-C₈-halogenoalkyl having 1 to 5 halogen atoms, —S(O)₂—C₁-C₈-alkyl, —S(O)₂—C₁-C₈-halogenoalkyl having 1 to 5 halogen atoms, —CH₂—S—C₁-C₈-alkyl, —CH₂—S(O)—C₁-C₈-alkyl, —CH₂—S(O)₂—C₁-C₈-alkyl, (C₁-C₆-alkoxyimino)-C₁-C₆-alkyl, (C₂-C₆-alkenyloxyimino)-C₁-C₆-alkyl, and (C₃-C₆-alkynyloxyimino)-C₁-C₆-alkyl; wherein two Y may additionally be selected from —O—C₁-C₃-alkoxy and —O—C₁-C₃-halogenalkoxy having 1 to 5 halogen atoms, with each oxy function being the connecting atom of the individual Y, thereby forming an annelated 5-, 6- or 7-membered ring with the phenyl moiety that they are connected to; or
each Y is independently selected from the group consisting of hydrogen, halogen, cyano, C₁-C₆-alkyl, C₁-C₆-halogenoalkyl having 1 to 5 halogen atoms, and a 4 to 7 membered heterocyclic ring system with 1 to 3 heteroatoms, but not O—O, O—S or 3 heteroatoms in a row, independently substituted by oxo, thiono or 1 to 12 substituents Z independently selected from the group consisting of hydrogen, halogen, cyano, hydroxy, amino, —SH, —CHO, —COOH, —CONH₂, —CONH(OH), —OCONH₂, (hydroxyimino)-C₁-C₆-alkyl, C₁-C₆-alkyl, alkyl, C₁-C₆-halogenoalkyl having 1 to 5 halogen atoms, C₂-C₆-alkenyl, C₂-C₆-alkynyl, C₁-C₆-alkylamino, di-(C₁-C₆-alkyl)amino, C₁-C₆-alkoxy, C₁-C₆-halogenoalkoxy having 1 to 5 halogen atoms, C₂-C₆-alkenyloxy, C₂-C₆-halogenoalkenyloxy having 1 to 5 halogen atoms, C₃-C₆-alkynyloxy, C₃-C₆-halogenoalkynyloxy having 1 to 5 halogen atoms, C₃-C₆-cycloalkyl, C₃-C₆-halogenocycloalkyl having 1 to 5 halogen atoms, C₁-C₆-alkylcarbonyl, C₁-C₆-halogenoalkylcarbonyl having 1 to 5 halogen atoms, —CONH(C₁-C₆-alkyl), —CON(C₁-C₆-alkyl)₂, —CONH(OC₁-C₆-alkyl), —CON(OC₁-C₆-alkyl)(C₁-C₆-alkyl), C₁-C₆-alkoxycarbonyl, C₁-C₆-halogenoalkoxycarbonyl having 1 to 5 halogen atoms, C₁-C₆-alkylcarbonyloxy, C₁-C₆-halogenoalkylcarbonyloxy having 1 to 5 halogen atoms, C₁-C₆-alkylcarbonylamino, C₁-C₆-halogenoalkylcarbonylamino having 1 to 5 halogen atoms, —OCONH(C₁-C₆-alkyl), —OCON(C₁-C₆-alkyl)₂, —OCONH(OC₁-C₆-alkyl), —OCO(OC₁-C₆-alkyl), —S—C₁-C₆-alkyl, —S—C₁-C₆-halogenoalkyl having 1 to 5 halogen atoms, —S(O)—C₁-C₆-alkyl, —S(O)—C₁-C₆-halogenoalkyl having 1 to 5 halogen atoms, —S(O)₂—C₁-C₆-alkyl, and —S(O)₂—C₁-C₆-halogenoalkyl having 1 to 5 halogen atoms; and
A represents a phenyl group of the formula (A1) 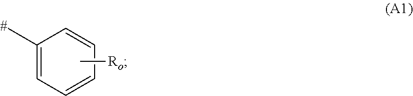
in which
# depicts the bond which connects A to the rest of the molecule;
o is 0, 1, 2, 3, 4, or 5; and
each R is independently selected from the group consisting of halogen, nitro, —OH, NH₂, SH, SF₅, CHO, OCHO, NHCHO, COOH, cyano, C₁-C₈-alkyl, C₁-C₈-halogenoalkyl having 1 to 9 halogen atoms, C₂-C₈-alkenyl, C₂-C₈-alkynyl, C₃-C₆-cycloalkyl, —S—C₁-C₈-alkyl, —S—C₁-C₈-halogenoalkyl having 1 to 5 halogen atoms, C₁-C₈-alkoxy, C₁-C₈-halogenoalkoxy having 1 to 5 halogen atoms, C₁-C₈-alkoxy-C₂-C₈-alkenyl, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, —S(O)—C₁-C₈-alkyl, —S(O)—C₁-C₈-halogenoalkyl having 1 to 5 halogen atoms, —S(O)₂—C₁-C₈-alkyl, —S(O)₂—C₁-C₈-halogenoalkyl having 1 to 5 halogen atoms, C₁-C₈-alkylsulfonamide, —NH(C₁-C₈-alkyl), N(C₁-C₈-alkyl)₂, phenyl (optionally substituted by C₁-C₆-alkoxy) and phenoxy; wherein two R bonded to adjacent carbon atoms may together represent —O(CH₂)ₚO—, wherein p represents 1 or 2;
A represents a heterocycle of the formula (Het-1) 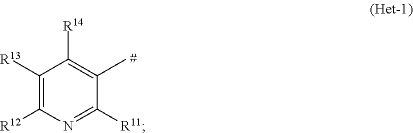
in which
# depicts the bond which connects A to the rest of the molecule;
R¹¹ is selected from the group consisting of hydrogen, halogen, hydroxy, cyano, C₁-C₄-alkyl, C₁-C₄-halogenoalkyl having 1 to 5 halogen atoms, C₁-C₄ alkoxy, —S—C₁-C₅-alkyl, S(O)—C₁-C₄-alkyl, —S(O)₂—C₁-C₄-alkyl, —S—C₂-C₅-alkenyl, —S—C₁-C₄-halogenoalkyl having 1 to 5 halogen atoms, and C₁-C₄-halogenoalkoxy having 1 to 5 halogen atoms; and
R¹², R¹³, and R¹⁴, which may be the same or be different, are selected from the group consisting of hydrogen, halogen, cyano, C₁-C₄-alkyl, C₁-C₄-halogenoalkyl having 1 to 5 halogen atoms, C₁-C₄-alkoxy, —S—C₁-C₄-alkyl, C₁-C₄-halogenoalkoxy having 1 to 5 halogen atoms, —S(O)—C₁-C₄-alkyl, and —S(O)₂—C₁-C₄-alkyl;
A represents a heterocycle of the formula (Het-2) 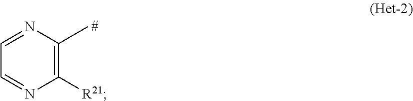
in which
# depicts the bond which connects A to the rest of the molecule; and
R²¹ is selected from the group consisting of hydrogen, halogen, C₁-C₄-alkyl, and C₁-C₄-halogenoalkyl having 1 to 5 halogen atoms; or
A represents a heterocycle of the formula (Het-3) 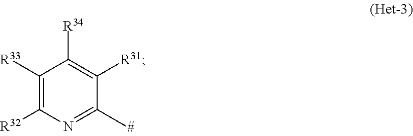
in which
# depicts the bond which connects A to the rest of the molecule;
R³¹ is selected from the group consisting of hydrogen, halogen, hydroxy, cyano, C₁-C₄-alkyl, C₁-C₄-halogenoalkyl having 1 to 5 halogen atoms, C₁-C₄ alkoxy, —S—C₁-C₅-alkyl, S(O)—C₁-C₄-alkyl, —S(O)₂—C₁-C₄-alkyl, —S—C₂-C₅-alkenyl, —S—C₁-C₄-halogenoalkyl having 1 to 5 halogen atoms, and C₁-C₄-halogenoalkoxy having 1 to 5 halogen atoms; and
R³², R³³, and R³⁴, which may be the same or be different, are selected from the group consisting of hydrogen, halogen, cyano, C₁-C₄-alkyl, C₁-C₄-halogenoalkyl having 1 to 5 halogen atoms, C₁-C₄-alkoxy, —S—C₁-C₄-alkyl, C₁-C₄-halogenoalkoxy having 1 to 5 halogen atoms, —S(O)—C₁-C₄-alkyl, and —S(O)₂—C₁-C₄-alkyl;
and salts, solvates, solvates of the salts, N-oxides, metal complexes or metalloid complexes thereof.

10. A pharmaceutical composition comprising at least one compound of formula (I) according to claim 1, or a salt, a solvate, a solvate of a salt, an N-oxide, a metal complex, or a metalloid complex thereof.

11. A method for the control, treatment, or prevention of infections with helminths, comprising administering to an animal or human in need thereof the pharmaceutical composition according to claim 10.

12-14. (canceled)

15. A method for the control, treatment or prevention of infections with helminths, comprising the step of administering an effective amount of a compound of formula (I) of anyone of claim 1, or a salt, a solvate, a solvate of a salt, an N-oxide, a metal complex, or a metalloid complex thereof, to an animal or human in need thereof.
